(19) United States
(12) Patent Application Publication
(10) Pub. No.: US 2025/0297243 A1
(43) Pub. Date: Sep. 25, 2025

(54) SINGLE CELL MULTIOMICS

(71) Applicant: BioSkryb Genomics, Inc., Durham, NC (US)

(72) Inventors: Jon Stanley ZAWISTOWSKI, Durham, NC (US); Jay A.A. WEST, Durham, NC (US); Durga ARVAPALLI, Durham, NC (US)

(21) Appl. No.: 18/860,539

(22) PCT Filed: Apr. 27, 2023
(86) PCT No.: PCT/US2023/020242

§ 371 Date: Oct. 25, 2024

Related U.S. Application Data
(60) Provisional application No. 63/335,949, filed on Apr. 28, 2022.
(60) Provisional application No. 63/403,213, filed on Sep. 1, 2022.

(51) Int. Cl. C12N 15/10

(52) U.S. Cl. CPC C12N 15/1068; C12N 15/1065

(57) ABSTRACT

Provided herein are compositions and methods for accurate and scalable single cell multiomics methods, and their applications for mutational analysis in research. diagnostics, and treatment. Further provided herein are multiomics methods for parallel analysis of DNA, RNA, and/or proteins from single cells using Primary Template-Directed Amplification (PTA) nucleic acid amplification.

### CROSS REFERENCE

This application claims the benefit of U.S. Provisional Patent Application No. 63/335,949 filed Apr. 28, 2022, and U.S. Provisional Patent Application No. 63/403,213 filed Sep. 1, 2022, both of which are incorporated herein by reference in their entirety.

### BACKGROUND

Research methods that utilize nucleic amplification, e.g., Next Generation Sequencing, provide large amounts of information on complex samples, genomes, and other nucleic acid sources. In some cases, these samples are obtained in small quantities from single cells. There is a need for highly accurate, scalable, and efficient nucleic acid amplification and sequencing methods for research, diagnostics, and treatment involving small samples, especially methods for simultaneous analysis of RNA, DNA, and proteins.

### BRIEF SUMMARY

Provided herein are methods of multiomic sample preparation comprising: isolating a single cell from a population of cells, wherein the single cell comprises RNA and genomic DNA; amplifying the RNA by RT-PCR to generate a cDNA library; contacting the genomic DNA with at least one amplification primer, at least one nucleic acid polymerase, and a mixture of nucleotides, wherein the mixture of nucleotides comprises at least one terminator nucleotide which terminates nucleic acid replication by the polymerase to generate a genomic DNA library; isolating the cDNA from the genomic DNA; and sequencing the cDNA library and the genomic DNA library. Provided herein are methods of multiomic sample preparation comprising: isolating a single cell from a population of cells, wherein the single cell comprises RNA and genomic DNA; amplifying the RNA by RT-PCR to generate a cDNA library; contacting the genomic DNA with at least one amplification primer, at least one nucleic acid polymerase, and a mixture of nucleotides, wherein the mixture of nucleotides comprises at least one terminator nucleotide which terminates nucleic acid replication by the polymerase to generate a genomic DNA library and at least one nucleotide configured for removal or digestion; isolating the cDNA from the genomic DNA; and sequencing the cDNA library and the genomic DNA library. Provided herein are methods of multiomic sample preparation comprising: isolating a single cell from a population of cells, wherein the single cell comprises RNA and genomic DNA; amplifying the RNA by RT-PCR to generate a cDNA library; contacting the genomic DNA with at least one amplification primer, at least one nucleic acid polymerase, and a mixture of nucleotides, wherein the mixture of nucleotides comprises at least one terminator nucleotide which terminates nucleic acid replication by the polymerase to generate a genomic DNA library and dUTP; isolating the cDNA from the genomic DNA; and sequencing the cDNA library and the genomic DNA library. Further provided herein are methods wherein the mixture of nucleotides comprises dUTP. Further provided herein are methods wherein the mixture of nucleotides comprises dATP, dCTP, dGTP, dTTP, and dUTP. Further provided herein are methods wherein the mixture of nucleotides comprises at least one base that is not dATP, dCTP, dGTP, dTTP. Further provided herein are methods wherein at least some of the polynucleotides of the cDNA library comprise a barcode. Further provided herein are methods wherein at least some of the polynucleotides of the cDNA library comprise a label. Further provided herein are methods wherein the cDNA is at least 90% free of the genomic DNA library after purification. Further provided herein are methods wherein the cDNA is at least 95% free of the genomic DNA library after purification. Further provided herein are methods wherein at least 90% polynucleotides of the cDNA library comprise a 5′ to 3′ bias of 0.8 to 1.2. Further provided herein are methods wherein isolating comprises capture of at least some of the cDNA library by binding to the label. Further provided herein are methods wherein isolating comprises contacting the cDNA library with an enzyme configured to digest or remove polynucleotides from the genomic DNA library. Further provided herein are methods wherein isolating comprises contacting the cDNA library with DNA glycosylase. Further provided herein are methods wherein contacting the cDNA library with the enzyme occurs on a solid support. Further provided herein are methods wherein the genomic DNA library is amplified prior to sequencing. Further provided herein are methods wherein the genomic DNA library is amplified with a uracil tolerant polymerase. Further provided herein are methods wherein the uracil tolerant polymerase comprises DNA polymerases & and 8 from S. cerevisiae, and E. coli DNA polymerase III, PolA-type polymerases, KAPA HiFi Uracil+DNA Polymerase (Q5U), KOD Multi & Epi DNA Polymerase, Taq, Taq2000, FailSafe Enzyme or PhusionU. Further provided herein are methods wherein isolating comprises nuclear lysis/denaturation. Further provided herein are methods wherein the cDNA library comprises 50-300 ng of DNA. Further provided herein are methods wherein the cDNA library comprises polynucleotides comprising a cell barcode or a sample barcode. Further provided herein are methods wherein the cDNA library comprises polynucleotides corresponding to at least 2000 genes. Further provided herein are methods wherein amplifying the cDNA library comprises contacting with labeled primers. Further provided herein are methods wherein the method further comprises addition of adapters to one or more of the cDNA library and the genomic DNA library. Further provided herein are methods wherein addition of adapters comprises contact with a ligase. Further provided herein are methods wherein addition of adapters comprises contact with a transposase or complex thereof. Further provided herein are methods wherein the transposase or complex thereof comprises Tn5. Further provided herein are methods wherein addition of adapters comprises contact with a polymerase and one or more primers. Further provided herein are methods wherein isolating comprises contacting the cDNA library with DNA glycosylase-lyase Endonuclease VIII. Further provided herein are methods wherein the genomic DNA library comprises 0.5-2.5 ng of DNA. Further provided herein are methods wherein the single cell comprises an NA12878 control. Further provided herein are methods wherein the single cell is a primary cell. Further provided herein are methods wherein the single cells originate from liver, skin, kidney, blood, or lung. Further provided herein are methods wherein the single cell is a cancer cell, neuron, glial cell, or fetal cell. Further provided herein are methods wherein the genomic DNA library is generated from 2-15 cycles of amplification. Further provided herein are methods wherein the genomic DNA library comprises polynucleotides 250-1500 bases in length. Further provided herein are methods wherein the genomic DNA library comprises an allelic balance of 70-95%. Further provided herein are methods wherein the genomic DNA library comprises an SNV sensitivity of at least 0.85%. Further provided herein are methods wherein the genomic DNA library comprises an SNV precision of at least 0.95%. Further provided herein are methods wherein the method further comprises analysis of one or more expressed proteins in the single cell. Further provided herein are methods wherein the method further comprises analysis of one or more genomic methylation patterns from the single cell. Further provided herein are methods wherein at least 98% of the polynucleotides comprise a terminator nucleotide. Further provided herein are methods wherein the terminator nucleotide is attached to the 3′ terminus of the at least some polynucleotides. Further provided herein are methods wherein the irreversible terminator is resistant to exonuclease activity. Further provided herein are methods wherein the irreversible terminator is resistant to 3′-5 exonuclease activity. Further provided herein are methods wherein the terminator nucleotide comprises adenine, guanine, cystine, or thymine. Further provided herein are methods wherein the terminator nucleotide does not comprise uridine. Further provided herein are methods wherein the terminator nucleotide is selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids. Further provided herein are methods wherein the nucleotides with modification to the alpha group are alpha-thio dideoxynucleotides. Further provided herein are methods wherein the terminator nucleotide comprises modifications of the r group of the 3′ carbon of the deoxyribose. Further provided herein are methods wherein the terminator nucleotide is selected from the group consisting of 3′ blocked reversible terminator containing nucleotides, 3′ unblocked reversible terminator containing nucleotides, terminators containing T modifications of deoxynucleotides, terminators containing modifications to the nitrogenous base of deoxynucleotides, and combinations thereof. Further provided herein are methods wherein the terminator nucleotides is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. Further provided herein are methods wherein the nucleic acid polymerase is bacteriophage phi29 (F29) polymerase, genetically modified phi29 (F29) DNA polymerase, Klenow Fragment of DNA polymerase I, phage M2 DNA polymerase, phage phiPRDI DNA polymerase, Bst DNA polymerase, Bst large fragment DNA polymerase, exo(−) Bst polymerase, exo(−)Bca DNA polymerase, Bsu DNA polymerase, VentRDNA polymerase, VentR (exo-) DNA polymerase, Deep Vent DNA polymerase, Deep Vent (exo-) DNA polymerase, IsoPol DNA polymerase, DNA polymerase I, Therminator DNA polymerase, T5 DNA polymerase, Sequenase, T7 DNA polymerase, T7-Sequenase, or T4 DNA polymerase. Further provided herein are methods wherein the nucleic acid polymerase comprises 3′->5′ exonuclease activity and the at least one terminator nucleotide inhibits the 3′->5′ exonuclease activity. Further provided herein are methods wherein the nucleic acid polymerase does not comprise 3′->5′ exonuclease activity. Further provided herein are methods wherein the polymerase is Bst DNA polymerase, exo(−) Bst polymerase, exo(−) Bca DNA polymerase, Bsu DNA polymerase, VentR (exo-) DNA polymerase, Deep Vent (exo-) DNA polymerase, Klenow Fragment (exo-) DNA polymerase, or Therminator DNA polymerase.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates a an exemplary high-level workflow of enrichment and preparation of simultaneous RNA and DNA from a single cell. RNA is reverse transcribed using oligo dT primers and a reverse transcriptase, followed by template switching and primer extension. Primary template amplification (PTA) is then used to amplify genomic DNA.

FIG. 1B illustrates graphs of nucleic acid yield for DNA (top) and RNA (bottom) from various samples (NTC=no template control). The yields of RNA and DNA isolated (in ng) for each cell used in this study. Samples where purification by streptavidin beads was omitted are highlighted in orange.

FIG. 2A illustrates graphs of allelic balance using combined RNA+DNA multiomics (left) vs. DNA only methods (right) in control (NA12878) is shown in deciles of observed allele frequency (AF) across known heterozygous positions. Each dot represents the proportion of variants that showed an AF within the bin frequency for a given cell. Barplots with error bars describe general trend for all cell-replicates for each AF bin. Allelic dropouts are called when AF is <0.1 or >0.9.

FIG. 2B illustrates a cumulative genomic coverage plot (combined RNA+DNA multiomics (left) vs. DNA only methods (right)) for each sample type performed using multiomics methods, showing the proportion of the entire genome covered (y-axis) at a given depth (x-axis). Each dot represents a cell replicate within a dataset and error plots denote the variability of coverage at a given depth.

FIG. 2C illustrates a graph of sensitivity using combined RNA+DNA multiomics (left) vs. DNA only methods (right). SNV calling sensitivity (y-axis) and precision (x-axis), with respect to GIAB NA12878 reference dataset are shown with both axes having a minimum range of 0.9 and 0.99, respectively.

FIG. 3A illustrates summarized coverage plots for all detected transcripts across the full-length chemistry (top). X axis is a normalized fraction of a transcript from 5′ to 3′, breaking regions into mean depth per percentile of transcript and y-axis are counts. Distribution of counts across coding sequence of two known housekeeping genes: GAPDH and ACTB (bottom).

FIG. 3B illustrates the proportion (averaged across all biosamples of a group) of aligned reads that matches a specific transcript feature or RNA species is reported for each dataset. Features and proportions were derived from Qualimap summarizations of our transcriptome definition file. NA12878 cells were leveraged except for the MOLM/DCIS plots. Bulk data was pulled from online repository to serve as reference from typical RNA-Seq. Conditions on the x-axis are: Bulk, IsolatedBulkRNA-StandardPrep, SingleCellRNA-StandardPrep, IsolatedBulkRNA-ResolveOME (Bioskryb Genomics, Inc.), SingleCell-ResolveOME (Bioskryb Genomics, Inc.), MOLM, and DCIS. Regions of each bar (top to bottom) are FivePrimeUTR_protein_coding, CDS_protein_coding, ThreePrimeUTR_protein_coding, intro_protein_coding, exon_lncRNA, intro_lncRNA, Other, and intergenic.

FIG. 3C illustrates graphs of various RNA quality control metrics are displayed for the UHRR and HBRR RNA controls alongside the NA12878 controls used in this study. Clockwise from the top left, the distribution of reads assigned to transcriptome, coding region features, unique genes detected, ranges of counts per million (CPM) and the median absolute deviation (MAD) of common housekeeping genes.

FIG. 3D illustrates multiomics full-transcript performance vs. an amalgam of publicly-available bulk RNA-Seq and 3′ end-counting datasets, including expressed protein-coding genes detected with multiomics chemistry compared to bulk preparation with the same workflow. Number of uniquely expressed genes across a diversity of cell line models and a primary DCIS patient sample. All sample sets were down-sampled to 75,000 reads.

FIG. 4A illustrates a copy number alterations of individual MOLM-13 cells (rows) from parental (turquoise) and resistant (salmon) cells using a bin size of 500 kb with Ginkgo. Dendrogram was generated based on distance of each bin's average fold change from 2N. b.): Representative metaphase spread of 25 total karyotypic spreads. Red circles denote abnormally amplified chromosomes.

FIG. 4B illustrates representative metaphase spread of 25 total karyotypic spreads. Red circles denote abnormally amplified chromosomes.

FIG. 5A illustrates genome views showing detection of mutual FLT3 ITD mutation in parental and quizartinib-resistant single cells.

FIG. 5B illustrates genome views of FLT3 secondary mutation N841K exclusively in quizartinib-resistant cells.
a missense mutation N841K was detected in all quizartinib resistant cells.

FIG. 5C illustrates qRT-PCR detection of mutant FLT3 K841 in treatment-naïve parental cells. qPCR cycling traces of FLT3 N841 (blue) and K841 (red) in MOLM-13 parental and quizartinib-resistant cells.

FIG. 6 illustrates a heatmap of SNVs showing statistically significant (p<0.05 by multinomial logistic regression) genotype prevalence across the MOLM-13 parental and resistant cells. Columns represent cells and rows SNV ids. Color within the tiles represent the called genotypes. Both rows and columns were subjected to unsupervised hierarchical clustering.

FIG. 7A illustrates a scatterplot showing the principal coordinate projection (PCA) of 28,134 SNVs that exhibited statistically significant (chi-square test, p<0.05) differential prevalence across the two MOLM-13 cohorts, parental (turquoise, left group) and resistant (salmon, right group).

FIG. 7B illustrates clustering of differentially-expressed genes in MOLM-13 model of drug resistance. Parental single cells (turquoise) and quizartinib-resistant (salmon) single cells comprise columns; Gene Symbol/Ensembl transcript ID comprise rows. Biotype and FDR is presented to the right of the heat map; red line indicates q<0.1.

FIG. 7C illustrates CEBPA/B transcript upregulation in single quizartinib-resistant MOLM-13 cells. Each row corresponds to a separate MOLM-13 cell. Resistant cells that also harbor 19q gains are also shown.

FIG. 7D illustrates a heatmap with transcripts in the y-axis that show a statistical (ZLM p<0.01) association with ploidy level across all cells in the MOLM-13 dataset. Color of the tiles represents the average standardized expression value at a given ploidy level. The right panel shown the output of the ZLM model testing the expression given the ploidy. Red line indicates the p<0.05 cutoff of the model. Bars are colored based on the −log 10 p-value of the ZLM model testing transcriptional differences between parental and resistant cells.

FIG. 7E illustrates an example of differential transcript utilization (DTU) between MOLM-13 parental and drug-resistant single cells.

FIG. 8A illustrates a bubble plot showing SNV-transcript expression associations (p<0.05). Top: SNVs within 5000 bases of transcriptional start site. Candidate SNVs are shown in the y-axis and genotypes in the x-axis. Size of the circle denotes the genotype prevalence of the variant in the MOLM-13 cell type set (parental or resistant). Colors of points denotes the standardized mean expression level of the transcript in the set. Lateral bars represent significance of the model testing the association between transcript expression and genotype. Red line indicates the p<0.1 cutoff of the model. Bars are colored based on the −log 10 p-value of the ZLM model testing transcriptional differences between parental and resistant cells. PABPC4 and MYC are highlighted in yellow. CEBPA SNVs were too distal (>5 kb) from transcriptional start site for significance in this plotting.

FIG. 8B illustrates parental/quizartinib-resistant SNVs proximal to CEBPA genomic locus. Stars denote mutation locations. Resistant cells show variant in 60% of cells compared to 11% in the parental line variant ‘chr19: 33,333,734—delA’ (middle star). For ‘chr19:33,361,973—insA’ we observed no mutations in the parental cells and in 50% in quizartinib-resistant cells.

FIG. 8C illustrates intronic SNV of MYC gene ‘chr8:127,739,932 G>A’ correlated with increased expression in drug-resistant MOLM-13 cells.

FIG. 8D illustrates putative promoter variants in PABPC4 ‘chr1:39,579,411 T>G’ & ‘chr1:39,579,413 T>G’ were found in half of the resistant cells only and also associated with differential expression between MOLM-13 parental and resistant cells . . .

FIG. 9 illustrates single-cell copy number alterations in primary DCIS/IDC EpCAM cohorts. Status of EpCAM presented for EpCAM High (yellow) and Low (turquoise). Two distinct classes of chromosomal loss are observed in EpCAM high (yellow) cells: 1) combined 11q, 13q, 16q/17p loss and 2) combined 13q and 16q/17p loss. Additionally, 13p gain was identified in 10/20 EpCAM high cells, while Chr. X gain encompassing the centromere and flanking P & Q segments was noted in 3 single cells.

FIG. 10A illustrates a principal component analysis of EpCAM high (circles) and EpCAM low (diamonds) primary DCIS/IDC transcriptomes where cells are colored based on the number of detected transcripts.

FIG. 10B illustrates PAM50 gene expression stratification of EpCAM high and EpCAM low DCIS/IDC transcriptomes.

FIG. 10C illustrates unsupervised clustering yields six primary blocks of differential gene expression between EpCAM high and EpCAM low clades. Average ploidy, PIK3CA genotypic status (green=N345 wildtype, pink=K345 heterozygous mutant), and cellular identity call are shown for each single cell (column). Gene biotype and FDR is presented for each transcript (row).

FIG. 10D illustrates prediction of DCIS cell identity/state using Human Cell Atlas data. Heat map showing identity score of diverse cell types (rows) for EpCAM High and EpCAM Low single cells (columns) that were used to identify cell annotations.

FIG. 10E illustrates an overlay of cellular annotation for principal component analysis of DCIS cells. EpCAM high (circles) and EpCAM low (diamonds) single cell transcriptomes, leveraging isoform counts with overlay of cell identity/state (colors).

FIG. 11 illustrates relative growth rates of parental and quizartinib-resistant MOLM-13 cells. Counts of cells over culture days after introduction of varying concentrations of quizartinib.

FIG. 12 illustrates missense variants in parental vs. resistant MOLM-13 cells. Variants (rows) identified as significantly associated through logistic regression with drug resistance are displayed, along with individual genotypes (0/0=homozygous reference, 0/1=heterozygous, 1/1=homozygous alternate, NA=not determined). Single cells (columns) are presented for parental (left) or resistant (right) cohorts. P value is shown along the right-hand side.

FIG. 13 illustrates a model of transcriptional bypass signaling through AXL upon FLT3 inhibition. Schematic illustrating that upon FLT3 inhibition by quizartinib, GAS6, the ligand for the receptor tyrosine kinase AXL, is upregulated in resistant MOLM-13 cells to drive growth and survival through PI3 kinase and AKT signaling, respectively.

FIG. 14 illustrates variants associated with DCIS expression groups. Variants (rows) identified as significantly associated through logistic regression with expression groups within EpCAM-H DCIS cells are shown, along with individual genotypes are shown (0/1=heterozygous, 1/1=homozygous alternate, NA=not determined). P value is shown along the right-hand side.

FIG. 15A illustrates an exemplary schematic of a multiomics workflow and steps of dUTP and uracil DNA glycosylase (UDG) intervention.

FIG. 15B illustrates the number of genes observed with or without UDG treatment, when dUTP was used in the PTA reaction of a multiomics workflow.

FIG. 15C illustrates intergenic background removal using the dUTP+UDG modification to the PTA workflow.

FIG. 15D illustrates allelic balance using the dUTP+UDG modification to the PTA workflow compared to a PTA workflow without dUTP+UDG.

FIG. 15E illustrates SNV calling metrics (sensitivity and precision) using the dUTP+UDG modification to the PTA workflow compared to a PTA workflow without dUTP+UDG.

### DETAILED DESCRIPTION OF THE INVENTION

There is a need to develop new scalable, accurate and efficient methods for nucleic acid amplification (including single-cell and multi-cell genome amplification) and sequencing which would overcome limitations in the current methods by increasing sequence representation, uniformity and accuracy in a reproducible manner. Provided herein are compositions and methods for providing accurate and scalable Primary Template-Directed Amplification (PTA) and sequencing in combination with additional cell analysis techniques (multiomics). Further provided herein are methods of multiomic analysis, including analysis of proteins, DNA, and RNA from single cells, and corresponding post-transcriptional or post-translational modifications in combination with PTA. Such methods and compositions facilitate highly accurate amplification of target (or “template”) nucleic acids, which increases accuracy and sensitivity of downstream applications, such as Next-Generation Sequencing.

### Definitions

Unless defined otherwise, all technical and scientific terms used herein have the same meaning as is commonly understood by one of ordinary skill in the art to which these inventions belong.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The terms “subject” or “patient” or “individual”, as used herein, refer to animals, including mammals, such as, e.g., humans, veterinary animals (e.g., cats, dogs, cows, horses, sheep, pigs, etc.) and experimental animal models of diseases (e.g., mice, rats). In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A practical Approach, Volumes I and II (D. N. Glover ed. 1985); Oligonucleotide Synthesis (MJ. Gait ed. 1984); Nucleic Acid Hybridization (B. D. Hames & S. J. Higgins eds. (1985>; Transcription and Translation (B. D. Hames & S. J. Higgins, eds. (1984»; Animal Cell Culture (R. I. Freshney, ed. (1986»; Immobilized Cells and Enzymes (IRL Press, (1986»>; B. Perbal, A practical Guide To Molecular Cloning (1984); F. M. Ausubel et al. (eds.), Current Protocols in Molecular Biology, John Wiley & Sons, Inc. (1994); among others.

The term “nucleic acid” encompasses multi-stranded, as well as single-stranded molecules. In double- or triple-stranded nucleic acids, the nucleic acid strands need not be coextensive (i.e., a double-stranded nucleic acid need not be double-stranded along the entire length of both strands). Nucleic acid templates described herein may be any size depending on the sample (from small cell-free DNA fragments to entire genomes), including but not limited to 50-300 bases, 100-2000 bases, 100-750 bases, 170-500 bases, 100-5000 bases, 50-10,000 bases, or 50-2000 bases in length. In some instances, templates are at least 50, 100, 200, 500, 1000, 2000, 5000, 10,000, 20,000 50,000, 100,000, 200,000, 500,000, 1,000,000 or more than 1,000,000 bases in length. Methods described herein provide for the amplification of nucleic acid acids, such as nucleic acid templates. Methods described herein additionally provide for the generation of isolated and at least partially purified nucleic acids and libraries of nucleic acids. In some instances, methods described herein provide for extracted nucleic acids (e.g., extracted from tissues, cells, or media). Nucleic acids include but are not limited to those comprising DNA, RNA, circular RNA, mtDNA (mitochondrial DNA), cfDNA (cell free DNA), cfRNA (cell free RNA), siRNA (small interfering RNA), cffDNA (cell free fetal DNA), mRNA, tRNA, rRNA, miRNA (microRNA), synthetic polynucleotides, polynucleotide analogues, any other nucleic acid consistent with the specification, or any combinations thereof. The length of polynucleotides, when provided, are described as the number of bases and abbreviated, such as nt (nucleotides), bp (bases), kb (kilobases), or Gb (gigabases).

The term “droplet” as used herein refers to a volume of liquid on a droplet actuator. Droplets in some instances, for example, be aqueous or non-aqueous or may be mixtures or emulsions including aqueous and non-aqueous components. For non-limiting examples of droplet fluids that may be subjected to droplet operations, see, e.g., Int. Pat. Appl. Pub. No. WO2007/120241. Any suitable system for forming and manipulating droplets can be used in the embodiments presented herein. For example, in some instances a droplet actuator is used. For non-limiting examples of droplet actuators which can be used, see, e.g., U.S. Pat. Nos. 6,911,132, 6,977,033, 6,773,566, 6,565,727, 7,163,612, 7,052,244, 7,328,979, 7,547,380, 7,641,779, U.S. Pat. Appl. Pub. Nos. US20060194331, US20030205632, US20060164490, US20070023292, US20060039823, US20080124252, US20090283407, US20090192044, US20050179746, US20090321262, US20100096266, US20110048951, Int. Pat. Appl. Pub. No. WO2007/120241. In some instances, beads are provided in a droplet, in a droplet operations gap, or on a droplet operations surface. In some instances, beads are provided in a reservoir that is external to a droplet operations gap or situated apart from a droplet operations surface, and the reservoir may be associated with a flow path that permits a droplet including the beads to be brought into a droplet operations gap or into contact with a droplet operations surface. Non-limiting examples of droplet actuator techniques for immobilizing magnetically responsive beads and/or non-magnetically responsive beads and/or conducting droplet operations protocols using beads are described in U.S. Pat. Appl. Pub. No. US20080053205, Int. Pat. Appl. Pub. No. WO2008/098236, WO2008/134153, WO2008/116221, WO2007/120241. Bead characteristics may be employed in the multiplexing embodiments of the methods described herein. Examples of beads having characteristics suitable for multiplexing, as well as methods of detecting and analyzing signals emitted from such beads, may be found in U.S. Pat. Appl. Pub. No. US20080305481, US20080151240, US20070207513, US20070064990, US20060159962, US20050277197, US20050118574. In some instances methods described herein utilize transposon-based droplet/bead processes such as those described in U.S. Pat. Nos. 11,473,138, 10,844,372, 10,590,244, 10,725,027, 9,771,575, 10,676,736, 11,479,816, 10,975,371, 11,180,752, 11,085,036, 11,111,519, 11,124,830, and 11,434,530. In some instances methods described herein utilize droplet manipulation techniques and devices such as those found in U.S. Pat. No. U.S. Pat. Nos. 10,633,701, 10,029,256, 11,517,864, 11,358,105, 11,000,849, 11,229,911, 10,569,268, 10,012,592, 9,573,099, 11,389,800, 9,475,013, 11,203,787, 10,589,274, 10,232,373, 11,312,990, 11,020,736, 11,111,519, and 11,142,791. In some instances methods described herein utilize single cell manipulation techniques such as those found in U.S. Pat. Nos. 11,124,830, and 11,365,441.

Primers and/or template switching oligonucleotides can also be affixed to solid substrate to facilitate reverse transcription and template switching of the mRNA polynucleotides. In this arrangement a portion of the RT or template switching reaction occurs in the bulk solution of the device, where the second step of the reaction occurs in proximity to the surface. In other arrangements the primer of template switch oligonucleotide is allowed to be released from the solid substrate to allow the entire reaction to occur above the surface in the solution. In a polyomic approach the primers for the multistage reaction in some instances is affixed to the solid substrate or combined with beads to accomplish combinations of multistage primers.

Certain microfluidic devices also support polyomic approaches. Devices fabricated in PDMS, as an example, often have contiguous chambers for each reaction step. Such multichambered devices are often segregated using a microvalve structure which can be controlled though the pressure with air, or a fluid such as water or inert hydrocarbon (i.e. fluorinert). In a multiomic approach each stage of the reaction can be sequestered and allowed to be conducted discretely. At the completion of a particular stage a valve between an adjacent chamber can be released on the substrates for the subsequent reaction can be added in a serial fashion. The result is the ability to emulate an sequential set of reactions, such as a multiomic (Protein/RNA/DNA/epigenomic) set of reactions using an individual cell as a input template material. Various microfluidics platforms may be used for analysis of single cells. Cells in some instances are manipulated through hydrodynamics (droplet microfluidics, inertial microfluidics, vortexing, microvalves, microstructures (e.g., microwells, microtraps)), electrical methods (dielectrophoresis (DEP), electroosmosis), optical methods (optical tweezers, optically induced dielectrophoresis (ODEP), opto-thermocapillary), acoustic methods, or magnetic methods. In some instances, the microfluidics platform comprises microwells. In some instances, the microfluidics platform comprises a PDMS (Polydimethylsiloxane)-based device. Non-limited examples of single cell analysis platforms compatible with the methods described herein are: ddSEQ Single-Cell Isolator, (Bio-Rad, Hercules, CA, USA, and Illumina, San Diego, CA, USA)); Chromium (10× Genomics, Pleasanton, CA, USA)); Rhapsody Single-Cell Analysis System (BD, Franklin Lakes, NJ, USA); Tapestri Platform (MissionBio, San Francisco, CA, USA)), Nadia Innovate (Dolomite Bio, Royston, UK); C1 and Polaris (Fluidigm, South San Francisco, CA, USA); ICELL8 Single-Cell System (Takara); MSND (Wafergen); Puncher platform (Vycap); CellRaft AIR System (CellMicrosystems); DEPArray NxT and DEP Array System (Menarini Silicon Biosystems); AVISO CellCelector (ALS); and InDrop System (1CellBio), TrapTx (Celldom), PipSeq (Fluent Bio), RNA sequencing kit (Scale Bio), and Single Cell 3.0 (Parse Bio).

As used herein, the term “unique molecular identifier (UMI)” refers to a unique nucleic acid sequence that is attached to each of a plurality of nucleic acid molecules. When incorporated into a nucleic acid molecule, an UMI in some instances is used to correct for subsequent amplification bias by directly counting UMIs that are sequenced after amplification. The design, incorporation and application of UMIs is described, for example, in Int. Pat. Appl. Pub. No. WO 2012/142213, Islam et al. Nat. Methods (2014) 11:163-166, Kivioja, T. et al. Nat. Methods (2012) 9:72-74, Brenner et al. (2000) PNAS 97 (4), 1665, and Hollas and Schuler, (2003) Conference: 3rd International Workshop on Algorithms in Bioinformatics, Volume: 2812.

As used herein, the term “barcode” refers to a nucleic acid tag that can be used to identify a sample or source of the nucleic acid material. Thus, where nucleic acid samples are derived from multiple sources, the nucleic acids in each nucleic acid sample are in some instances tagged with different nucleic acid tags such that the source of the sample can be identified. Barcodes, also commonly referred to indexes, tags, and the like, are well known to those of skill in the art. Any suitable barcode or set of barcodes can be used. See, e.g., non-limiting examples provided in U.S. Pat. No. 8,053,192 and Int. Pat. Appl. Pub. No. WO2005/068656. Barcoding of single cells can be performed as described, for example, in U.S. Pat. Appl. Pub. No. 2013/0274117.

The terms “solid surface,” “solid support” and other grammatical equivalents herein refer to any material that is appropriate for or can be modified to be appropriate for the attachment of the primers, barcodes and sequences described herein. Exemplary substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon, nitrocellulose, ceramics, resins, silica, silica-based materials (e.g., silicon or modified silicon), carbon, metals, inorganic glasses, plastics, optical fiber bundles, and a variety of other polymers. In some embodiments, the solid support comprises a patterned surface suitable for immobilization of primers, barcodes and sequences in an ordered pattern.

As used herein, the term “biological sample” includes, but is not limited to, tissues, cells, biological fluids and isolates thereof. Cells or other samples used in the methods described herein are in some instances isolated from human patients, animals, plants, soil or other samples comprising microbes such as bacteria, fungi, protozoa, etc. In some instances, the biological sample is of human origin. In some instances, the biological is of non-human origin. The cells in some instances undergo PTA methods described herein and sequencing. Variants detected throughout the genome or at specific locations can be compared with all other cells isolated from that subject to trace the history of a cell lineage for research or diagnostic purposes. In some instances, variants are confirmed through additional methods of analysis such as direct PCR sequencing.

### Single Cell Analysis

Described herein are methods and compositions for analysis of single cells. Analysis of cells in bulk provides general information about the cell population, but often is unable to detect low-frequency mutants over the background. Such mutants may comprise important properties such as drug resistance or mutations associated with cancer. In some instances, DNA, RNA, and/or proteins from the same single cell are analyzed in parallel. The analysis may include identification of epigenetic post-translational (e.g., glycosylation, phosphorylation, acetylation, ubiquination, histone modification) and/or post-transcriptional (e.g., methylation, hydroxymethylation) modifications. Such methods may comprise “Primary Template-Directed Amplification” (PTA) to obtain libraries of nucleic acids for sequencing. In some instances PTA is combined with additional steps or methods such as RT-PCR or proteome/protein quantification techniques (e.g., mass spectrometry, antibody staining, etc.). In some instances, various components of a cell are physically or spatially separated from each other during individual analysis steps. Further, in some instances multiomic methods of genomic DNA/RNA analysis require purification of genomic DNA away from RNA (or cDNA after reverse transcription). Remaining contamination of genomic DNA in a cDNA library may result in inaccurate transcriptome sequencing results.

In an exemplary workflow, proteins are first labeled with antibodies. In some instances, at least some of the antibodies comprise a tag or marker (e.g., nucleic acid/oligo tag, mass tag, or fluorescent, tag). In some instances, a portion of the antibodies comprise an oligo tag. In some instances, a portion of the antibodies comprise a fluorescent marker. In some instances antibodies are labeled by two or more tags or markers. In some instances, a portion of the antibodies are sorted based on fluorescent markers. After RT-PCR, first strand mRNA products are generated and then removed for analysis. Libraries are then generated from RT-PCR products and barcodes present on protein-specific antibodies, which are subsequently sequenced. In parallel, genomic DNA from the same cell is subjected to PTA, a library generated, and sequenced. Sequencing results from the genome, methylome, proteome, and transcriptome are in some instances pooled using bioinformatics methods. Methods described herein in some instances comprise any combination of labeling, cell sorting, affinity separation/purification, lysing of specific cell components (e.g., outer membrane, nucleus, etc.), RNA amplification, DNA amplification (e.g., PTA), or other step associated with protein, RNA, or DNA isolation or analysis. In some instances, methods described herein comprise one or more enrichment steps, such as exome enrichment.

Described herein is a first method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. Alternatively or in combination, centrifugation is used to separate RNA in the supernatant from cDNA in the cell pellet. In some instances, solid supports are used to bind to TAGs. In some instances, solid supports comprise a substantially planer surface, well, or bead. In some instances, TSOs are attached to a solid support. In some instances, use of solid supports comprising TSOs enables purification of cDNA amplicons. Purification of cDNA in some instances comprises a wash step. Remaining cDNA is in some instances fragmented and removed with UDG (uracil DNA glycosylase), and alkaline lysis is used to degrade RNA and denature the genome. After neutralization, addition of primers and PTA, amplification products are in some instances purified on SPRI (solid phase reversible immobilization) beads, and ligated to adapters to generate a gDNA library. The PTA reaction in some instances occurs in the presence of the generated cDNA library. In some instances, the PTA reaction comprises use of bases which may be cleaved or removed by an enzyme. In some instances, the enzyme comprises a glycosylase. In some instances, the PTA reaction is conducted with a plurality of dNTPs which include a nucleotide other than A, T, G, or C. In some instances, the PTA reaction is conducted with a plurality of dNTPs which include uracil. gDNA is purified on SPRI (solid phase reversible immobilization) beads, and ligated to adapters to generate a gDNA library. After PTA amplification, the cDNA in some instances is purified or isolated. RT products in some instances are isolated by pulldown, such as a pulldown with streptavidin beads. RT products in some instances are isolated by physical separation from the reaction mixture (e.g., on a bead, or a magnetic bead). In some instances, residual genomic library amplicons generated by PTA are removed (or digested) using an enzyme. In some instances, residual genomic library amplicons generated by PTA are removed using a glycosylase. In some instances, residual genomic library amplicons generated by PTA containing uracil are removed by digestion. After purification, cDNA libraries in some instances are at least 80%, 85%, 90%, 95%, 97%, 98%, 99%, 99.5%, or at least 99.9% free of genomic DNA amplicons (e.g., those generated by PTA).

Described herein is a second method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. In some instances, solid supports are used to bind to TAGs. In some instances, solid supports comprise a substantially planer surface, well, or bead. In some instances, TSOs are attached to a solid support. In some instances, use of solid supports comprising TSOs enables purification of cDNA amplicons. Purification of cDNA in some instances comprises a wash step. In some instances, solid supports are used to bind to TAGs. In some instances, solid supports comprise a substantially planer surface, well, or bead. In some instances, TSOs are attached to a solid support. In some instances, use of solid supports comprising TSOs enables purification of cDNA amplicons. Purification of cDNA in some instances comprises a wash step. In some instances, alkaline lysis is then used to degrade RNA and denature the genome. After neutralization, addition of random primers and PTA, amplification products are in some instances purified on SPRI (solid phase reversible immobilization) beads, and ligated to adapters to generate a gDNA library. The PTA reaction in some instances occurs in the presence of the generated cDNA library. In some instances, the PTA reaction comprises use of bases which may be cleaved or removed by an enzyme. In some instances, the enzyme comprises a glycosylase. In some instances, the PTA reaction is conducted with a plurality of dNTPs which include a nucleotide other than A, T, G, or C. In some instances, the PTA reaction is conducted with a plurality of dNTPs which include uracil. gDNA is purified on SPRI (solid phase reversible immobilization) beads, and ligated to adapters to generate a gDNA library. After PTA amplification, the cDNA in some instances is purified or isolated. RT products in some instances are isolated by pulldown, such as a pulldown with streptavidin beads. RT products in some instances are isolated by physical separation from the reaction mixture (e.g., on a bead, or a magnetic bead). In some instances, residual genomic library amplicons generated by PTA are removed (or digested) using an enzyme. In some instances, residual genomic library amplicons generated by PTA are removed using a glycosylase. In some instances, residual genomic library amplicons generated by PTA containing uracil are removed by digestion. After purification, cDNA libraries in some instances are at least 80%, 85%, 90%, 95%, 97%, 98%, 99%, 99.5%, or at least 99.9% free of genomic DNA amplicons (e.g., those generated by PTA).

Described herein is a third method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs) in the presence of terminator nucleotides. In some instances, TSOs comprise a molecular TAG such as biotin, which allows subsequent pull-down of cDNA RT products, and PCR amplification of RT products to generate a cDNA library. In some instances, alkaline lysis is then used to degrade RNA and denature the genome. After neutralization, addition of random primers and PTA, amplification products are in some instances purified on SPRI (solid phase reversible immobilization) beads, and ligated to adapters to generate a DNA library. The PTA reaction in some instances occurs in the presence of the generated cDNA library. In some instances, the PTA reaction comprises use of bases which may be cleaved or removed by an enzyme. In some instances, the enzyme comprises a glycosylase. In some instances, the PTA reaction is conducted with a plurality of dNTPs which include a nucleotide other than A, T, G, or C. In some instances, the PTA reaction is conducted with a plurality of dNTPs which include uracil. gDNA is purified on SPRI (solid phase reversible immobilization) beads, and ligated to adapters to generate a gDNA library. After PTA amplification, the cDNA in some instances is purified or isolated. RT products in some instances are isolated by pulldown, such as a pulldown with streptavidin beads. RT products in some instances are isolated by physical separation from the reaction mixture (e.g., on a bead, or a magnetic bead). In some instances, residual genomic library amplicons generated by PTA are removed (or digested) using an enzyme. In some instances, residual genomic library amplicons generated by PTA are removed using a glycosylase. In some instances, residual genomic library amplicons generated by PTA containing uracil are removed by digestion. After purification, cDNA libraries in some instances are at least 80%, 85%, 90%, 95%, 97%, 98%, 99%, 99.5%, or at least 99.9% free of genomic DNA amplicons (e.g., those generated by PTA).

A mixture of nucleotides may comprise at least one nucleotide configured for digestion (or removal, or reaction) by an enzyme or chemical process. In some instances, the nucleotide configured for digestion comprises dUTP. In some instances, the nucleotide configured for digestion is present in about a 1000:1, 500:1, 100:1, 50:1, 25:1, 20:1, 15:1, 10:1, 5:1, 2:1, 1:1, 1:1.5, 1:2, 1:5, 1:10, 1:20, 1:25, 1:50, 1:100, 1:500, or about a 1:1000 ratio relative to another nucleotide in the mixture. In some instances, the nucleotide configured for digestion is present in at least a 1000:1, 500:1, 100:1, 50:1, 25:1, 20:1, 15:1, 10:1, 5:1, 2:1, 1:1, 1:1.5, 1:2, 1:3, 1:5, 1:10, 1:25, 1:50, 1:100, 1:500, or at least a 1:1000 ratio relative to another nucleotide in the mixture. In some instances, the nucleotide configured for digestion is present in no more than a 1000:1, 500:1, 100:1, 50:1, 25:1, 20:1, 15:1, 10:1, 5:1, 2:1, 1:1, 1:1.5, 1:2, 1:3, 1:5, 1:10, 1:20, 1:25, 1:50, 1:100, 1:500, or no more than a 1:1000 ratio relative to another nucleotide in the mixture. In some instances, the nucleotide configured for digestion is present in about a 1000:1-1:1000 ratio, 100:1-1:100, 50:1-1:50, 50:1-1:20, 20:1-1:20, 10:1-1:10, 5:1-1:5, 3:1-1:3, 2:1-1:1, 3:1-1:1, 5:1-1:2, 5:1-1:1, 10:1-1:1, 10:1-1:2, 20:1-1:1, 20:1-1:2, 50:1-1:1, or 100:1-1:1 relative to another nucleotide in the mixture. In some instances, dUTP is present in about a 1000:1, 500:1, 100:1, 50:1, 25:1, 20:1, 15:1, 10:1, 5:1, 2:1, 1:1, 1:1.5, 1:2, 1:5, 1:10, 1:25, 1:50, 1:100, 1:500, or about a 1:1000 ratio relative to another nucleotide in the mixture. In some instances, dUTP is present in at least a 1000:1, 500:1, 100:1, 50:1, 25:1, 20:1, 15:1, 10:1, 5:1, 2:1, 1:1, 1:1.5, 1:2, 1:5, 1:10, 1:25, 1:50, 1:100, 1:500, or at least a 1:1000 ratio relative to another nucleotide in the mixture. In some instances, dUTP is present in no more than a 1000:1, 500:1, 100:1, 50:1, 25:1, 20:1, 15:1, 10:1, 5:1, 2:1, 1:1, 1:1.5, 1:2, 1:5, 1:10, 1:25, 1:50, 1:100, 1:500, or no more than a 1:1000 ratio relative to another nucleotide in the mixture. In some instances, dUTP is present in about a 1000:1-1:1000 ratio, 100:1-1:100, 50:1-1:50, 50:1-1:20, 20:1-1:20, 10:1-1:10, 5:1-1:5, 3:1-1:3, 2:1-1:1, 3:1-1:1, 5:1-1:2, 5:1-1:1, 10:1-1:1, 10:1-1:2, 20:1-1:1, 20:1-1:2, 50:1-1:1, or 100:1-1:1 relative to another nucleotide in the mixture. In some instances, the mixture comprises a dTTP to dUTP ratio of about a 1000:1, 500:1, 100:1, 50:1, 25:1, 20:1, 15:1, 10:1, 5:1, 2:1, 1:1, 1:1.5, 1:2, 1:5, 1:10, 1:25, 1:50, 1:100, 1:500, or about a 1:1000. the mixture comprises a dTTP to dUTP ratio of at least a 1000:1, 500:1, 100:1, 50:1, 25:1, 20:1, 15:1, 10:1, 5:1, 2:1, 1:1, 1:1.5, 1:2, 1:5, 1:10, 1:25, 1:50, 1:100, 1:500, or at least a 1:1000. the mixture comprises a dTTP to dUTP ratio of no more than a 1000:1, 500:1, 100:1, 50:1, 25:1, 20:1, 15:1, 10:1, 5:1, 2:1, 1:1, 1:1.5, 1:2, 1:3, 1:5, 1:10, 1:25, 1:50, 1:100, 1:500, or no more than a 1:1000. the mixture comprises a dTTP to dUTP of 1000:1-1:1000, 100:1-1:100, 50:1-1:50, 50:1-1:20, 20:1-1:20, 10:1-1:10, 5:1-1:5, 3:1-1:3, 2:1-1:1, 3:1-1:1, 5:1-1:2, 5:1-1:1, 10:1-1:1, 10:1-1:2, 20:1-1:1, 20:1-1:2, 50:1-1:1, or 100:1-1:1. In some instances, the ratio of dTTP to dUTP is selected such that the PTA reaction completes at least 5 amplification cycles in no more than 0.1, 0.5, 1, 1.5, 2, 3, 4, 5, 8, 10, or no more than 12 hours. In some instances, the ratio of dTTP to dUTP is selected such that the PTA reaction completes at least 9 amplification cycles in no more than 0.1, 0.5, 1, 1.5, 2, 3, 4, 5, 8, 10, or no more than 12 hours.

Described herein is a fourth method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. In some instances, solid supports are used to bind to TAGs. In some instances, solid supports comprise a substantially planer surface, well, or bead. In some instances, TSOs are attached to a solid support. In some instances, use of solid supports comprising TSOs enables purification of cDNA amplicons. Purification of cDNA in some instances comprises a wash step. In some instances, alkaline lysis is then used to degrade RNA and denature the genome. After neutralization, addition of random primers and PTA, amplification products are in some instances subjected to RNase and cDNA amplification using blocked and labeled primers. The PTA reaction in some instances occurs in the presence of the generated cDNA library. In some instances, the PTA reaction comprises use of bases which may be cleaved or removed by an enzyme. In some instances, the enzyme comprises a glycosylase. In some instances, the PTA reaction is conducted with a plurality of dNTPs which include a nucleotide other than A, T, G, or C. In some instances, the PTA reaction is conducted with a plurality of dNTPs which include uracil. gDNA is purified on SPRI (solid phase reversible immobilization) beads, and ligated to adapters to generate a gDNA library. After PTA amplification, the cDNA in some instances is purified or isolated. RT products in some instances are isolated by pulldown, such as a pulldown with streptavidin beads. RT products in some instances are isolated by physical separation from the reaction mixture (e.g., on a bead, or a magnetic bead). In some instances, residual genomic library amplicons generated by PTA are removed (or digested) using an enzyme. In some instances, residual genomic library amplicons generated by PTA are removed using a glycosylase. In some instances, residual genomic library amplicons generated by PTA containing uracil are removed by digestion. After purification, cDNA libraries in some instances are at least 80%, 85%, 90%, 95%, 97%, 98%, 99%, 99.5%, or at least 99.9% free of genomic DNA amplicons (e.g., those generated by PTA).

Described herein is a fifth method of single cell analysis comprising analysis of RNA and DNA from a single cell. A population of cells is contacted with an antibody library, wherein antibodies are labeled. In some instances, antibodies are labeled with either fluorescent labels, nucleic acid barcodes, or both. Labeled antibodies bind to at least one cell in the population, and such cells are sorted, placing one cell per container (e.g., a tube, vial, microwell, etc.). In some instances, the container comprises a solvent. In some instances, a region of a surface of a container is coated with a capture moiety. In some instances, the capture moiety is a small molecule, an antibody, a protein, or other agent capable of binding to one or more cells, organelles, or other cell component. In some instances, at least one cell, or a single cell, or component thereof, binds to a region of the container surface. In some instances, a nucleus binds to the region of the container. In some instances, the outer membrane of the cell is lysed, releasing mRNA into a solution in the container. In some instances, the nucleus of the cell containing genomic DNA is bound to a region of the container surface. Next, RT is often performed using the mRNA in solution as a template to generate cDNA. In some instances, template switching primers comprise from 5′ to 3′ a TSS region (transcription start site), an anchor region, a RNA BC region, and a poly dT tail. In some instances, the poly dT tail binds to poly A tail of one or more mRNAs. In some instances, template switching primers comprise from 3′ to 5′ a TSS region, an anchor region, and a poly G region. In some instances, the poly G region comprises riboG. In some instances the poly G region binds to a poly C region on an mRNA transcript. In some instances, riboG was added to the mRNA transcripts by a terminal transferase. After removal of RT PCR products for subsequent sequencing, any remaining RNA in the cell is removed by UNG. The nucleus is then lysed, and the released genomic DNA is subjected to the PTA method using random primers with an isothermal polymerase. In some instances, primers are 6-9 bases in length. In some instances, PTA generates genomic amplicons of 100-5000, 200-5000, 500-2000, 500-2500, 1000-3000, or 300-3000 bases in length. In some instances, PTA generates genomic amplicons with an average length of 100-5000, 200-5000, 500-2000, 500-2500, 1000-3000, or 300-3000 bases. In some instances, PTA generates genomic amplicons of 250-1500 bases in length. In some instances, the methods described herein generate a short fragment cDNA pool with about 500, about 750, about 1000, about 5000, or about 10,000 fold amplification. In some instances, the methods described herein generate a short fragment cDNA pool with 500-5000, 750-1500, or 250-10,000 fold amplification. PTA products are optionally subjected to additional amplification and sequenced.

### Sample Preparation and Isolation of Single Cells

Methods described herein may require isolation of single cells for analysis. Any method of single cell isolation may be used with PTA, such as mouth pipetting, micro pipetting, flow cytometry/FACS, microfluidics, methods of sorting nuclei (tetraploid or other), or manual dilution. Such methods are aided by additional reagents and steps, for example, antibody-based enrichment (e.g., circulating tumor cells), other small-molecule or protein-based enrichment methods, or fluorescent labeling. In some instances, a method of multiomic analysis described herein comprises mechanical or enzymatic dissociate of cells from larger tissues.

### Preparation and Analysis of Cell Components

Methods of multiomic analysis comprising PTA described herein may comprise one or more methods of processing cell components such as DNA, RNA, and/or proteins. In some instances, the nucleus (comprising genomic DNA) is physically separated from the cytosol (comprising mRNA), followed by a membrane-selective lysis buffer to dissolve the membrane but keep the nucleus intact. The cytosol is then separated from the nucleus using methods including micro pipetting, centrifugation, or anti-body conjugated magnetic microbeads. In another instance, an oligo-dT primer coated magnetic bead binds polyadenylated mRNA for separation from DNA. In another instance, DNA and RNA are preamplified simultaneously, and then separated for analysis. In another instance, a single cell is split into two equal pieces, with mRNA from one half processed, and genomic DNA from the other half processed.

### Multiomics

Provided herein are methods for multiomics sample preparation and/or analysis. In some instances, a method comprises one or more steps of isolating a single cell from a population of cells, wherein the single cell comprises RNA and genomic DNA; amplifying the RNA by RT-PCR to generate a cDNA library; isolating the cDNA from the genomic DNA; contacting the genomic DNA with at least one amplification primer, at least one nucleic acid polymerase, and a mixture of nucleotides; isolating the cDNA from a genomic library, and sequencing the cDNA library and the genomic DNA library. In some instances, the mixture of nucleotides comprises at least one nucleotide configured for digestion (or removal, or reaction) by an enzyme or chemical process. In some instances, the mixture of nucleotides comprises dUTP. In some instances, the mixture of nucleotides comprises at least one terminator nucleotide which terminates nucleic acid replication by the polymerase to generate a genomic DNA library. In some instances, a terminator nucleotide comprises an irreversible terminator. In some instances, an irreversible terminator inhibits or is resistant to 3′ to 5′ exonuclease activity.

Methods described herein (e.g., PTA) may be used as a replacement for any number of other known methods in the art which are used for single cell sequencing (multiomics or the like). PTA may substitute genomic DNA sequencing methods such as MDA, PicoPlex, DOP-PCR, MALBAC, or target-specific amplifications. In some instances, PTA replaces the standard genomic DNA sequencing method in a multiomics method including DR-seq (Dey et al., 2015), G&T seq (MacAulay et al., 2015), scMT-seq (Hu et al., 2016), sc-GEM (Cheow et al., 2016), scTrio-seq (Hou et al., 2016), simultaneous multiplexed measurement of RNA and proteins (Darmanis et al., 2016), scCOOL-seq (Guo et al., 2017), CITE-seq (Stoeckius et al., 2017), REAP-seq (Peterson et al., 2017), scNMT-seq (Clark et al., 2018), or SIDR-seq (Han et al., 2018). In some instances, a method described herein comprises PTA and a method of polyadenylated mRNA transcripts. In some instances, a method described herein comprises PTA and a method of non-polyadenylated mRNA transcripts. In some instances, a method described herein comprises PTA and a method of total (polyadenylated and non-polyadenylated) mRNA transcripts.

In some instances, PTA is combined with a standard RNA sequencing method to obtain genome and transcriptome data. In some instances, a multiomics method described herein comprises PTA and one of the following: Drop-seq (Macosko, et al. 2015), mRNA-seq (Tang et al., 2009), InDrop (Klein et al., 2015), MARS-seq (Jaitin et al., 2014), Smart-seq2 (Hashimshony, et al., 2012; Fish et al., 2016), CEL-seq (Jaitin et al., 2014), STRT-seq (Islam, et al., 2011), Quartz-seq (Sasagawa et al., 2013), CEL-seq2 (Hashimshony, et al. 2016), cytoSeq (Fan et al., 2015), SuPeR-seq (Fan et al., 2011), RamDA-seq (Hayashi, et al. 2018), MATQ-seq (Sheng et al., 2017), or SMARTer (Verboom et al., 2019).

Various reaction conditions and mixes may be used for generating cDNA libraries for transcriptome analysis. In some instances, an RT reaction mix is used to generate a cDNA library. In some instances, the RT reaction mixture comprises a crowding reagent, at least one primer, a template switching oligonucleotide (TSO), a reverse transcriptase, and a dNTP mix. In some instances, an RT reaction mix comprises an RNAse inhibitor. In some instances an RT reaction mix comprises one or more surfactants. In some instances an RT reaction mix comprises Tween-20 and/or Triton-X. In some instances an RT reaction mix comprises Betaine. In some instances an RT reaction mix comprises one or more salts. In some instances an RT reaction mix comprises a magnesium salt (e.g., magnesium chloride) and/or tetramethylammonium chloride. In some instances an RT reaction mix comprises gelatin. In some instances an RT reaction mix comprises PEG (PEG1000, PEG2000, PEG4000, PEG6000, PEG8000, or PEG of other length).

Multiomic methods described herein may provide both genomic and RNA transcript information from a single cell (e.g., a combined or dual protocol). In some instances, genomic information from the single cell is obtained from the PTA method, and RNA transcript information is obtained from reverse transcription to generate a cDNA library. In some instances, a whole transcript method is used to obtain the cDNA library. In some instances, 3′ or 5′ end counting is used to obtain the cDNA library. In some instances, cDNA libraries are not obtained using UMIs. In some instances, a multiomic method provides RNA transcript information from the single cell for at least 500, 1000, 2000, 5000, 8000, 10,000, 12,000, or at least 15,000 genes. In some instances, a multiomic method provides RNA transcript information from the single cell for about 500, 1000, 2000, 5000, 8000, 10,000, 12,000, or about 15,000 genes. In some instances, a multiomic method provides RNA transcript information from the single cell for 100-12,000 1000-10,000, 2000-15,000, 5000-15,000, 10,000-20,000, 8000-15,000, or 10,000-15,000 genes. In some instances, a multiomic method provides genomic sequence information for at least 80%, 90%, 92%, 95%, 97%, 98%, or at least 99% of the genome of the single cell. In some instances, a multiomic method provides genomic sequence information for about 80%, 90%, 92%, 95%, 97%, 98%, or about 99% of the genome of the single cell. RNA may be amplified in the multiomics methods described herein. In some instances, RNA is amplified to isolate mRNA transcripts. In some instances, template-switching polynucleotides are used. In some instances, amplification of RNA uses labeled primers. In some instances, a label comprises biotin. In some instances, at least some of the cDNA polynucleotides are isolated with affinity binding to the label. In some instances, multiomics methods comprise amplification of RNA to generate a cDNA library. In some instances, a cDNA library is generated having at least 10, 20, 30, 50, 75, 100, 125, 150, 175, 200, 225, 250, 300, 350, 400, or at least 500 ng of DNA. In some instances, a cDNA library is generated having 10-500, 20-500, 30-500, 50-500, 50-400, 50-300, 100-500, 100-400, 100-300, 100-200, 200-500, 300-500, or 400-750 ng of DNA. In some instances, at least some polynucleotides in the cDNA library comprise a barcode. In some instances, the cDNA comprises polynucleotides corresponding to at least 100, 500, 1000, 1500, 2000, 2500, 3000, 3500, or at least 4000 genes. In some instances, the cDNA comprises a 5′ to 3′ transcript bias of 0.5-1.5, 0.6-1.5, 0.7-1.5, 0.8-1.5, 0.9-1.5, 0.8-1.5, 1-1.5, 1-2.0, 1.2-2.0, 0.5-2.0.

Multiomic methods may comprise analysis of single cells from a population of cells. In some instances, at least 5, 10, 20, 50, 100, 200, 500, 1000, 2000, 5000, or at least 8000 cells are analyzed. In some instances, about 5, 10, 20, 50, 100, 200, 500, 1000, 2000, 5000, or about 8000 cells are analyzed. In some instances, 5-100, 10-100, 50-500, 100-500, 100-1000, 50-5000, 100-5000, 500-1000, 500-10000, 1000-10000, or 5000-20,000 cells are analyzed.

Multiomic methods may generate yields of genomic DNA from the PTA reaction based on the type of single cell. In some instances, the amount of DNA generated from a single cell is about 0.1, 1, 1.5, 2, 3, 5, or about 10 micrograms. In some instances, the amount of DNA generated from a single cell is about 0.1, 1, 1.5, 2, 3, 5, or about 10 femtograms. In some instances, the amount of DNA generated from a single cell is at least 0.1, 1, 1.5, 2, 3, 5, or at least 10 micrograms. In some instances, the amount of DNA generated from a single cell is at least 0.1, 1, 1.5, 2, 3, 5, or at least 10 femtograms. In some instances, the amount of DNA generated from a single cell is about 0.1-10, 1-10, 1.5-10, 2-20, 2-50, 1-3, or 0.5-3.5 micrograms. In some instances, the amount of DNA generated from a single cell is about 0.1-10, 1-10, 1.5-10, 2-20, 2-4, 1-3, or 0.5-4 femtograms. In some instances, the amount of DNA generated from a single cell is about 0.5-2.5, 0.5-3, 0.5-5, 0.2-5, 1-2.5, or 1-5 ng of DNA. In some instances, the amount of DNA generated from a single cell is at least 0.25, 0.5, 0.75, 1, 1.25, 1.5, 1.75, 2, 2.25, 2.5, 2.75, 3, 3.25, 3.5, 4, or at least 5 ng of DNA.

DNA libraries may comprise an allelic balance. In some instances, the allelic balance is 50-100, 60-100, 70-100, 80-100, 60-95, 70-95, 80-95, 85-95, 90-95, 90-98, 90-99, 85-99, or 95-99 percent. In some instances, the allelic balance is at least 50, 60, 70, 80, 83, 85, 87, 90, 92, 95, 98, or at least 99 percent.

DNA libraries may comprise a sensitivity for one or more SNVs. In some instances, the sensitivity is 0.50-1, 0.60-1, 0.70-1, 0.80-1, 0.60-0.95, 0.70-0.95, 0.80-0.95, 0.85-0.95, 0.90-0.95, 0.90-0.98, 0.90-0.99, 0.85-0.99, or 0.95-0.99. In some instances, the sensitivity is at least 0.50, 0.60, 0.70, 0.80, 0.83, 0.85, 0.87, 0.90, 0.92, 0.95, 0.98, or at least 0.99.

DNA libraries may comprise a precision for one or more SNVs. In some instances, the precision is 0.50-1, 0.60-1, 0.70-1, 0.80-1, 0.60-0.95, 0.70-0.95, 0.80-0.95, 0.85-0.95, 0.90-0.95, 0.90-0.98, 0.90-0.99, 0.85-0.99, or 0.95-0.99. In some instances, the precision is at least 0.50, 0.60, 0.70, 0.80, 0.83, 0.85, 0.87, 0.90, 0.92, 0.95, 0.98, or at least 0.99.

### Methylome Analysis

Described herein are methods comprising PTA, wherein sites of methylated DNA in single cells are determined using the PTA method. In some instances, methylome analysis comprises identifying the location of methylated bases (e.g, methylC, hydroxymethylC). In some instances, these methods further comprise parallel analysis of the transcriptome, methylome, and/or proteome of the same cell. Methods of detecting methylated genomic bases include selective restriction with methylation-sensitive endonucleases, followed by processing with the PTA method. Sites cut by such enzymes are determined from sequencing, and methylated bases are identified. In another instance, bisulfite treatment of genomic DNA libraries converts unmethylated cytosines to uracil. Libraries are then in some instances amplified with methylation-specific primers which selectively anneal to methylated sequences. Alternatively, non-methylation-specific PCR is conducted, followed by one or more methods to discriminate between bisulfite-reacted bases, including direct pyrosequencing, MS-SnuPE, HRM, COBRA, MS-SSCA, or base-specific cleavage/MALDI-TOF. In some instances, genomic DNA samples are split for parallel analysis of the genome (or an enriched portion thereof) and methylome analysis. In some instances, analysis of the genome and methylome comprises enrichment of genomic fragments (e.g., exome, or other targets) or whole genome sequencing. In some instances, methylated bases in a genomic sample are identified by (a) conversion of a methylated base to a different base, or (b) conversion of a non-methylated base to a different base. Such conversions in some instances are performed on whole genomes or genomic fragments. The resulting sequences are then compared to a reference sequence (obtained without conversion/treatment) to identify which bases are methylated. In some instances, a conversion method (or process) comprises treatment with a deamination reagent. In some instances, a conversion method comprises treatment with bisulfate. In some instances, one or more enzymes are used to selectively discriminate between methylated and unmethylated bases. In some instances, enzymes comprises TET (ten eleven translocation) family enzymes. In some instances, a TET family enzyme comprises TET2. In some instances, enzymes comprise T4-BGT. In some instances, a conversion method comprises treatment with a reagent to protect methylcytosines (e.g., TET2 for oxidation), followed by treatment with an enzyme to deaminate unprotected cytosines (e.g., APOBEC). Additional reagents which differentiate methylated and non-methylated bases are also consistent with the methods disclosed herein. In some instances, unmethylated cytosines are converted to uracil. In some instances, amplification of these uracil-containing modified genomes results in conversion of uracil to thymine. In some instances, amplification comprises use of uracil tolerant polymerases described herein. In some instances, adapters described herein are modified to replace cytosines with methylcytosines or other base which resists conversion.

### Bioinformatics

The data obtained from single-cell analysis methods utilizing PTA described herein may be compiled into a database. Described herein are methods and systems of bioinformatic data integration. Data from the proteome, genome, transcriptome, methylome or other data is in some instances combined/integrated into a database and analyzed. Bioinformatic data integration methods and systems in some instances comprise one or more of protein detection (FACS and/or NGS), mRNA detection, and/or genome variance detection. In some instances, this data is correlated with a disease state or condition. In some instances, data from a plurality of single cells is compiled to describe properties of a larger cell population, such as cells from a specific sample, region, organism, or tissue. In some instances, protein data is acquired from fluorescently labeled antibodies which selectively bind to proteins on a cell. In some instances, a method of protein detection comprises grouping cells based on fluorescent markers and reporting sample location post-sorting. In some instances, a method of protein detection comprises detecting sample barcodes, detecting protein barcodes, comparing to designed sequences, and grouping cells based on barcode and copy number. In some instances, protein data is acquired from barcoded antibodies which selectively bind to proteins on a cell. In some instances, transcriptome data is acquired from sample and RNA specific barcodes. In some instances, a method of mRNA detection comprises detecting sample and RNA specific barcodes, aligning to genome, aligning to RefSeq/Encode, reporting Exon/Intro/Intergenic sequences, analyzing exon-exon junctions, grouping cells based on barcode and expression variance and clustering analysis of variance and top variable genes. In some instances, genomic data is acquired from sample and DNA specific barcodes. In some instances, a method of genome variance detection comprises detecting sample and DNA specific barcodes, aligning to the genome, determine genome recovery and SNV mapping rate, filtering reads on exon-exon junctions, generating variant call file (VCF), and clustering analysis of variance and top variable mutations.

### Mutations

In some instances, the methods (e.g., multiomic PTA) described herein result in higher detection sensitivity and/or lower rates of false positives for the detection of mutations. In some instances a mutation is a difference between an analyzed sequence (e.g., using the methods described herein) and a reference sequence. Reference sequences are in some instances obtained from other organisms, other individuals of the same or similar species, populations of organisms, or other areas of the same genome. In some instances, mutations are identified on a plasmid or chromosome. In some instances, a mutation is an SNV (single nucleotide variation), SNP (single nucleotide polymorphism), or CNV (copy number variation, or CNA/copy number aberration). In some instances, a mutation is base substitution, insertion, or deletion. In some instances, a mutation is a transition, transversion, nonsense mutation, silent mutation, synonymous or non-synonymous mutation, non-pathogenic mutation, missense mutation, or frameshift mutation (deletion or insertion). In some instances, PTA results in higher detection sensitivity and/or lower rates of false positives for the detection of mutations when compared to methods such as in-silico prediction, ChIP-seq, GUIDE-seq, circle-seq, HTGTS (High-Throughput Genome-Wide Translocation Sequencing), IDLV (integration-deficient lentivirus), Digenome-seq, FISH (fluorescence in situ hybridization), or DISCOVER-seq.

### Primary Template-Directed Amplification

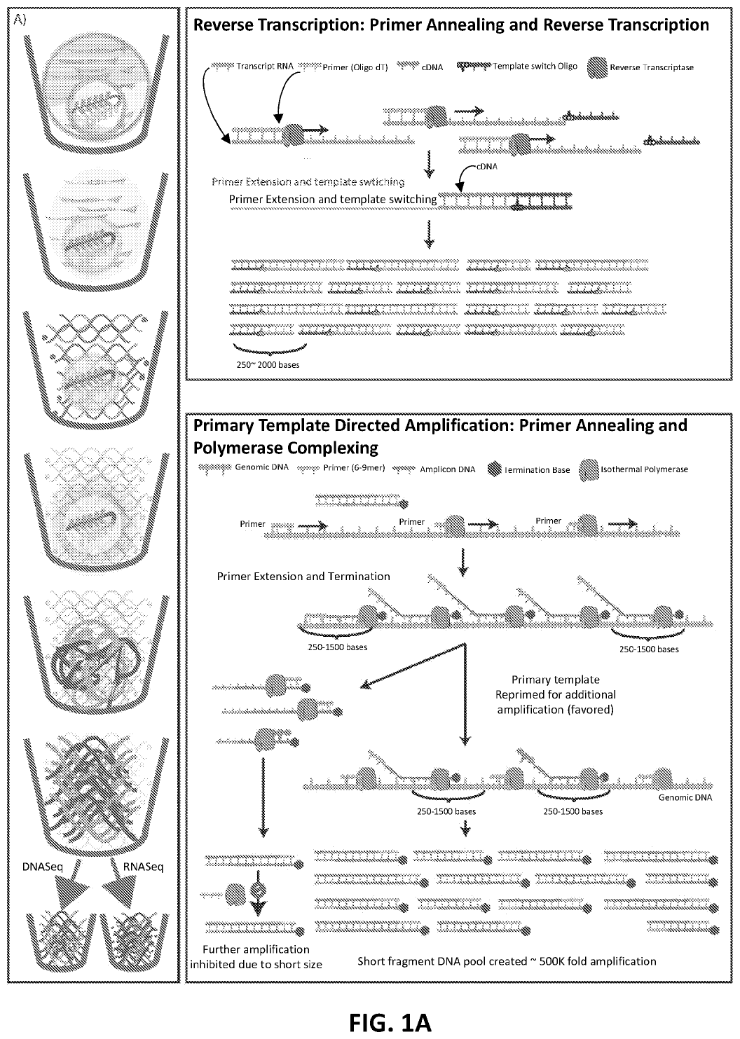

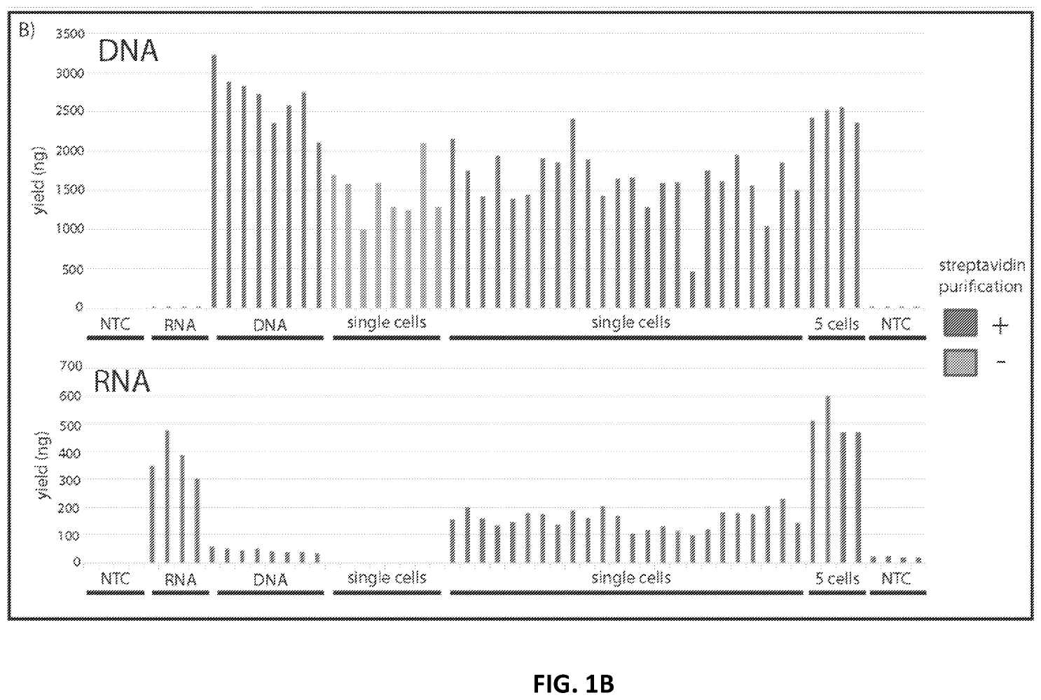

Described herein are nucleic acid amplification methods, such as “Primary Template-Directed Amplification (PTA).” In some instances, PTA is combined with other analysis workflows for multiomic analysis. For example, one embodiment of the PTA method described herein are schematically represented in FIG. 1A. With the PTA method, amplicons are preferentially generated from the primary template (“direct copies”) using a polymerase (e.g., a strand displacing polymerase). Consequently, errors are propagated at a lower rate from daughter amplicons during subsequent amplifications compared to MDA. The result is an easily executed method that, unlike existing WGA protocols, can amplify low DNA input including the genomes of single cells with high coverage breadth and uniformity in an accurate and reproducible manner. Moreover, the terminated amplification products can undergo direction ligation after removal of the terminators, allowing for the attachment of a cell barcode to the amplification primers so that products from all cells can be pooled after undergoing parallel amplification reactions. In some instances, template nucleic acids are not bound to a solid support. In some instances, direct copies of template nucleic acids are not bound to a solid support. In some instances, one or more primers are not bound to a solid support. In some instances, no primers are not bound to a solid support. In some instances, a primer is attached to a first solid support, and a template nucleic acid is attached to a second solid support, wherein the first and the second solid supports are not the same. In some instances, PTA is used to analyze single cells from a larger population of cells. In some instances, PTA is used to analyze more than one cell from a larger population of cells, or an entire population of cells.

Described herein are methods employing nucleic acid polymerases with strand displacement activity for amplification. In some instances, such polymerases comprise strand displacement activity and low error rate. In some instances, such polymerases comprise strand displacement activity and proofreading exonuclease activity, such as 3′->5′ proofreading activity. In some instances, nucleic acid polymerases are used in conjunction with other components such as reversible or irreversible terminators, or additional strand displacement factors. In some instances, the polymerase has strand displacement activity, but does not have exonuclease proofreading activity. For example, in some instances such polymerases include bacteriophage phi29 (@29) polymerase, which also has very low error rate that is the result of the 3′->5′ proofreading exonuclease activity (see, e.g., U.S. Pat. Nos. 5,198,543 and 5,001,050). In some instances, non-limiting examples of strand displacing nucleic acid polymerases include, e.g., genetically modified phi29 (29) DNA polymerase, Klenow Fragment of DNA polymerase I (Jacobsen et al., Eur. J. Biochem. 45:623-627 (1974)), phage M2 DNA polymerase (Matsumoto et al., Gene 84:247 (1989)), phage phiPRD1 DNA polymerase (Jung et al., Proc. Natl. Acad. Sci. USA 84:8287 (1987); Zhu and Ito, Biochim. Biophys. Acta. 1219:267-276 (1994)), Bst DNA polymerase (e.g., Bst large fragment DNA polymerase (Exo (−) Bst; Aliotta et al., Genet. Anal. (Netherlands) 12:185-195 (1996)), exo(−)Bca DNA polymerase (Walker and Linn, Clinical Chemistry 42:1604-1608 (1996)), Bsu DNA polymerase, Vent_{R} DNA polymerase including Vent_{R} (exo-) DNA polymerase (Kong et al., J. Biol. Chem. 268:1965-1975 (1993)), Deep Vent DNA polymerase including Deep Vent (exo-) DNA polymerase, IsoPol DNA polymerase, DNA polymerase I, Therminator DNA polymerase, T5 DNA polymerase (Chatterjee et al., Gene 97:13-19 (1991)), Sequenase (U.S. Biochemicals), T7 DNA polymerase, T7-Sequenase, T7 gp5 DNA polymerase, PRDI DNA polymerase, T4 DNA polymerase (Kaboord and Benkovic, Curr. Biol. 5:149-157 (1995)). Additional strand displacing nucleic acid polymerases are also compatible with the methods described herein. The ability of a given polymerase to carry out strand displacement replication can be determined, for example, by using the polymerase in a strand displacement replication assay (e.g., as disclosed in U.S. Pat. No. 6,977,148). Such assays in some instances are performed at a temperature suitable for optimal activity for the enzyme being used, for example, 32° C. for phi29 DNA polymerase, from 46° C. to 64° C. for exo(−) Bst DNA polymerase, or from about 60° C. to 70° C. for an enzyme from a hyperthermophylic organism. Another useful assay for selecting a polymerase is the primer-block assay described in Kong et al., J. Biol. Chem. 268:1965-1975 (1993). The assay consists of a primer extension assay using an M13 ssDNA template in the presence or absence of an oligonucleotide that is hybridized upstream of the extending primer to block its progress. Other enzymes capable of displacement the blocking primer in this assay are in some instances useful for the disclosed method. In some instances, polymerases incorporate dNTPs and terminators at approximately equal rates. In some instances, the ratio of rates of incorporation for dNTPs and terminators for a polymerase described herein are about 1:1, about 1.5:1, about 2:1, about 3:1 about 4:1 about 5:1, about 10:1, about 20:1 about 50:1, about 100:1, about 200:1, about 500:1, or about 1000:1. In some instances, the ratio of rates of incorporation for dNTPs and terminators for a polymerase described herein are 1:1 to 1000:1, 2:1 to 500:1, 5:1 to 100:1, 10:1 to 1000:1, 100:1 to 1000:1, 500:1 to 2000:1, 50:1 to 1500:1, or 25:1 to 1000:1. In some instances, nucleobases or nucleobase analogs are added which can be selective removed. In some instances, nucleobases are removed using an enzyme. In some instances, the enzyme comprises UDG. In some instances, the nucleobase comprises dU. In some instances, the nucleobase is present a ratio relative to another nucleotide in the mixture. In some instances, the nucleobase is present a ratio of no more than 0.2:1, 0.5:1, 0.7:1, 0.8:1, 1:1, 1:1.5, 1:2, 1:2.5, 1:3, or no more than 1:5 in the mixture. In some instances, the nucleobase is present a ratio of at least 0.2:1, 0.5:1, 0.7:1, 0.8:1, 1:1, 1:1.5, 1:2, 1:2.5, 1:3, or at least 1:5 in the mixture. In some instances, dU is present a ratio of no more than 0.2:1, 0.5:1, 0.7:1, 0.8:1, 1:1, 1:1.5, 1:2, 1:2.5, 1:3, or no more than 1:5 to dT in the mixture. In some instances, dU is present a ratio of at least 0.2:1, 0.5:1, 0.7:1, 0.8:1, 1:1, 1:1.5, 1:2, 1:2.5, 1:3, or at least 1:5 to dT in the mixture.

Described herein are methods of amplification wherein strand displacement can be facilitated through the use of a strand displacement factor, such as, e.g., helicase. Such factors are in some instances used in conjunction with additional amplification components, such as polymerases, terminators, or other component. In some instances, a strand displacement factor is used with a polymerase that does not have strand displacement activity. In some instances, a strand displacement factor is used with a polymerase having strand displacement activity. Without being bound by theory, strand displacement factors may increase the rate that smaller, double stranded amplicons are reprimed. In some instances, any DNA polymerase that can perform strand displacement replication in the presence of a strand displacement factor is suitable for use in the PTA method, even if the DNA polymerase does not perform strand displacement replication in the absence of such a factor. Strand displacement factors useful in strand displacement replication in some instances include (but are not limited to) BMRF1 polymerase accessory subunit (Tsurumi et al., J. Virology 67 (12): 7648-7653 (1993)), adenovirus DNA-binding protein (Zijderveld and van der Vliet, J. Virology 68 (2): 1158-1164 (1994)), herpes simplex viral protein ICP8 (Boehmer and Lehman, J. Virology 67 (2): 711-715 (1993); Skaliter and Lehman, Proc. Natl. Acad. Sci. USA 91 (22): 10665-10669 (1994)); single-stranded DNA binding proteins (SSB; Rigler and Romano, J. Biol. Chem. 270:8910-8919 (1995)); phage T4 gene 32 protein (Villemain and Giedroc, Biochemistry 35:14395-14404 (1996); T7 helicase-primase; T7 gp2.5 SSB protein; Tte-UvrD (from Thermoanaerobacter tengcongensis), calf thymus helicase (Siegel et al., J. Biol. Chem. 267:13629-13635 (1992)); bacterial SSB (e.g., E. coli SSB), Replication Protein A (RPA) in eukaryotes, human mitochondrial SSB (mtSSB), and recombinases, (e.g., Recombinase A (RecA) family proteins, T4 UvsX, T4 UvsY, Sak4 of Phage HK620, Rad51, Dmc1, or Radb). Combinations of factors that facilitate strand displacement and priming are also consistent with the methods described herein. For example, a helicase is used in conjunction with a polymerase. In some instances, the PTA method comprises use of a single-strand DNA binding protein (SSB, T4 gp32, or other single stranded DNA binding protein), a helicase, and a polymerase (e.g., SauDNA polymerase, Bsu polymerase, Bst2.0, GspM, GspM2.0, GspSSD, or other suitable polymerase). In some instances, reverse transcriptases are used in conjunction with the strand displacement factors described herein. In some instances, reverse transcriptases are used in conjunction with the strand displacement factors described herein. In some instances, amplification is conducted using a polymerase and a nicking enzyme (e.g., “NEAR”), such as those described in U.S. Pat. No. 9,617,586. In some instances, the nicking enzyme is Nt.BspQI, Nb.BbvCi, Nb.BsmI, Nb.BsrDI, Nb.BtsI, Nt. AlwI, Nt.BbvCI, Nt.BstNBI, Nt. CviPII, Nb.Bpu10I, or Nt. Bpu 10I.

Described herein are amplification methods comprising use of terminator nucleotides, polymerases, and additional factors or conditions. For example, such factors are used in some instances to fragment the nucleic acid template(s) or amplicons during amplification. In some instances, such factors comprise endonucleases. In some instances, factors comprise transposases. In some instances, mechanical shearing is used to fragment nucleic acids during amplification. In some instances, nucleotides are added during amplification that may be fragmented through the addition of additional proteins or conditions. For example, uracil is incorporated into amplicons; treatment with uracil D-glycosylase fragments nucleic acids at uracil-containing positions. Additional systems for selective nucleic acid fragmentation are also in some instances employed, for example an engineered DNA glycosylase that cleaves modified cytosine-pyrene base pairs. (Kwon, et al. Chem Biol. 2003, 10 (4), 351) Uracil tolerant polymerases are also in some instances used. In some instances, use of uracil tolerant polymerases results in improved results for multiomics methods, such as those described herein.

Transposase-based library preparation (i.e., “tagmentation”) may be used with the methods and compositions described herein. In some instances, after PTA the library is exposed to one or more transposomes. In some instances, transposomes comprise a transposase (e.g., Tn5, MuA, or other enzyme). In some instances, transposes simultaneously cleave and tag polynucleotides in the library. In some instances, tags comprise polynucleotides. In some instances, tags comprise one or more of barcodes, adapters, primer sites, or other region. In some instances, transposomes are linked to a solid support. In some instances, the solid support comprises a bead, planar surface, or other structure.

Nanoball sequencing may be used in combination with the multiomics methods described herein (e.g., PTA). Rolling circle amplification (RCA) in some instances is used to amplify fragments of genomic DNA into DNA nanoballs. In some instances, amplification uses a uracil tolerant polymerase. The DNA nanoballs are adsorbed onto a flow cell and the fluorescence at each position is determined and used to identify the base. Libraries in some instances prepared with a desired insert sizes and sequenced using nanoball sequencing. Circularized adaptors were compatible for nanoball sequencing. In some instances a library preparation method described herein employs a transposition complex formed by a hyperactive Tn5 transposase and a Tn5-type transposon end. In some instances a library preparation method described herein employs a transposition complex formed by a MuA transposase and a Mu transposon end comprising R1 and R2 end sequences. In some instances, a transposition system is used which inserts a transposon end in a random or in a pseudorandom manner to 5′-tag and fragment a target DNA. In some instances, transposition systems comprise Staphylococcus aureus Tn552, Ty1, Transposon Tn7, Tn10 and IS10, Mariner transposase, Tcl, Tn3, bacterial insertion sequences, retroviruses, or retrotransposon of yeast. In some instances, a transposase described herein comprises a wild-type or mutant transposase, wild-type or mutant Tn5 transposase, (e.g., EZ-Tn5™ transposase, HYPERMU™ MuA transposase). In some instances, a transposase or complex there comprises Nextera™ tagment DNA enzyme 1 (TDE1, Illumina). In some instances, a transposase comprises a mutant or variant of a wild type transposase. In some instances, a variant comprises a sequence having at least 50%, 60%, 70%, 75%, 80%, 85%, 90%, 95%, 97%, 98%, or least 99% identity with the wild type sequence. In some instances a transposase comprises a Tn5 variant having at least 50%, 60%, 70%, 75%, 80%, 85%, 90%, 95%, 97%, 98%, or least 99% identity with the wild type sequence. In some instances, a Tn5 variant comprises one or more mutations at positions 42, 54, 56, 372, 450, 451, or 454. In some instances, a Tn5 variant comprises two or more mutations at positions 42, 54, 56, 372, 450, 451, or 454. In some instances, a Tn5 variant comprises three or more mutations at positions 42, 54, 56, 372, 450, 451, or 454.

Ligation-based library preparation may be used with the methods and compositions described herein (e.g., Sequencing by synthesis). Adapters (e.g., Y-adapters) in some instances are ligated to the ends of amplicons obtained herein to generate a library for sequencing. In some instances, the library is amplified prior to sequencing by use of a uracil tolerant polymerase. In some instances, an adapter comprises one or more of a yoke region, a first non-complementary region, an index region, a unique molecular identifier region, a second non-complementary region, a primer region, and a graft region. In some instances, a graft region is configured to bind to a sequencing instrument flowcell. In some instances, an adapter comprises a truncated (or “stubby”/universal) adapter. In some instances, a truncated adapter comprises one or more of a yoke region, a first non-complementary region, a unique molecular identifier region, a second non-complementary region, and a primer region. In some instances, one or more of an index region and a graft region are added to a truncated adapter by amplification after the adapter is ligated to amplicons. In some instances truncated adapters are used such as those described in Glenn et al. PeerJ. 2019; 7: e7786.

Described herein are amplification methods comprising use of terminator nucleotides, which terminate nucleic acid replication thus decreasing the size of the amplification products. Such terminators are in some instances used in conjunction with polymerases, strand displacement factors, or other amplification components described herein. In some instances, terminator nucleotides reduce or lower the efficiency of nucleic acid replication. Such terminators in some instances reduce extension rates by at least 99.9%, 99%, 98%, 95%, 90%, 85%, 80%, 75%, 70%, or at least 65%. Such terminators in some instances reduce extension rates by 50%-90%, 60%-80%, 65%-90%, 70%-85%, 60%-90%, 70%-99%, 80%-99%, or 50%-80%. In some instances terminators reduce the average amplicon product length by at least 99.9%, 99%, 98%, 95%, 90%, 85%, 80%, 75%, 70%, or at least 65%. Terminators in some instances reduce the average amplicon length by 50%-90%, 60%-80%, 65%-90%, 70%-85%, 60%-90%, 70%-99%, 80%-99%, or 50%-80%. In some instances, amplicons comprising terminator nucleotides form loops or hairpins which reduce a polymerase's ability to use such amplicons as templates. Use of terminators in some instances slows the rate of amplification at initial amplification sites through the incorporation of terminator nucleotides (e.g., dideoxynucleotides that have been modified to make them exonuclease-resistant to terminate DNA extension), resulting in smaller amplification products. By producing smaller amplification products than the currently used methods (e.g., average length of 50-2000 nucleotides in length for PTA methods as compared to an average product length of >10,000 nucleotides for MDA methods) PTA amplification products in some instances undergo direct ligation of adapters without the need for fragmentation, allowing for efficient incorporation of cell barcodes and unique molecular identifiers (UMI).

Terminator nucleotides are present at various concentrations depending on factors such as polymerase, template, or other factors. For example, the amount of terminator nucleotides in some instances is expressed as a ratio of non-terminator nucleotides to terminator nucleotides in a method described herein. Such concentrations in some instances allow control of amplicon lengths. In some instances, the ratio of terminator to non-terminator nucleotides is modified for the amount of template present or the size of the template. In some instances, the ratio of ratio of terminator to non-terminator nucleotides is reduced for smaller samples sizes (e.g., femtogram to picogram range). In some instances, the ratio of non-terminator to terminator nucleotides is about 2:1, 5:1, 7:1, 10:1, 20:1, 50:1, 100:1, 200:1, 500:1, 1000:1, 2000:1, or 5000:1. In some instances the ratio of non-terminator to terminator nucleotides is 2:1-10:1, 5:1-20:1, 10:1-100:1, 20:1-200:1, 50:1-1000:1, 50:1-500:1, 75:1-150:1, or 100: 1-500:1. In some instances, at least one of the nucleotides present during amplification using a method described herein is a terminator nucleotide. Each terminator need not be present at approximately the same concentration; in some instances, ratios of each terminator present in a method described herein are optimized for a particular set of reaction conditions, sample type, or polymerase. Without being bound by theory, each terminator may possess a different efficiency for incorporation into the growing polynucleotide chain of an amplicon, in response to pairing with the corresponding nucleotide on the template strand. For example, in some instances a terminator pairing with cytosine is present at about 3%, 5%, 10%, 15%, 20%, 25%, or 50% higher concentration than the average terminator concentration. In some instances a terminator pairing with thymine is present at about 3%, 5%, 10%, 15%, 20%, 25%, or 50% higher concentration than the average terminator concentration. In some instances a terminator pairing with guanine is present at about 3%, 5%, 10%, 15%, 20%, 25%, or 50% higher concentration than the average terminator concentration. In some instances a terminator pairing with adenine is present at about 3%, 5%, 10%, 15%, 20%, 25%, or 50% higher concentration than the average terminator concentration. In some instances a terminator pairing with uracil is present at about 3%, 5%, 10%, 15%, 20%, 25%, or 50% higher concentration than the average terminator concentration. Any nucleotide capable of terminating nucleic acid extension by a nucleic acid polymerase in some instances is used as a terminator nucleotide in the methods described herein. In some instances, a reversible terminator is used to terminate nucleic acid replication. In some instances, a non-reversible terminator is used to terminate nucleic acid replication. In some instances, non-limited examples of terminators include reversible and non-reversible nucleic acids and nucleic acid analogs, such as, e.g., 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, or any combination thereof. In one embodiment, terminator nucleotides are dideoxynucleotides. Other nucleotide modifications that terminate nucleic acid replication and may be suitable for practicing the invention include, without limitation, any modifications of the r group of the 3′ carbon of the deoxyribose such as inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. In some instances, terminators are polynucleotides comprising 1, 2, 3, 4, or more bases in length. In some instances, terminators do not comprise a detectable moiety or tag (e.g., mass tag, fluorescent tag, dye, radioactive atom, or other detectable moiety). In some instances, terminators do not comprise a chemical moiety allowing for attachment of a detectable moiety or tag (e.g., “click” azide/alkyne, conjugate addition partner, or other chemical handle for attachment of a tag). In some instances, all terminator nucleotides comprise the same modification that reduces amplification to at region (e.g., the sugar moiety, base moiety, or phosphate moiety) of the nucleotide. In some instances, at least one terminator has a different modification that reduces amplification. In some instances, all terminators have a substantially similar fluorescent excitation or emission wavelengths. In some instances, terminators without modification to the phosphate group are used with polymerases that do not have exonuclease proofreading activity. Terminators, when used with polymerases which have 3′->5′ proofreading exonuclease activity (such as, e.g., phi29) that can remove the terminator nucleotide, are in some instances further modified to make them exonuclease-resistant. For example, dideoxynucleotides are modified with an alpha-thio group that creates a phosphorothioate linkage which makes these nucleotides resistant to the 3′->5′ proofreading exonuclease activity of nucleic acid polymerases. Such modifications in some instances reduce the exonuclease proofreading activity of polymerases by at least 99.5%, 99%, 98%, 95%, 90%, or at least 85%. Non-limiting examples of other terminator nucleotide modifications providing resistance to the 3′->5′ exonuclease activity include in some instances: nucleotides with modification to the alpha group, such as alpha-thio dideoxynucleotides creating a phosphorothioate bond, C3 spacer nucleotides, locked nucleic acids (LNA), inverted nucleic acids, 2′ Fluoro bases, 3′ phosphorylation, 2′-O-Methyl modifications (or other 2′-O-alkyl modification), propyne-modified bases (e.g., deoxycytosine, deoxyuridine), L-DNA nucleotides, L-RNA nucleotides, nucleotides with inverted linkages (e.g., 5′-5′ or 3′-3′), 5′ inverted bases (e.g., 5′ inverted 2′,3′-dideoxy dT), methylphosphonate backbones, and trans nucleic acids. In some instances, nucleotides with modification include base-modified nucleic acids comprising free 3′ OH groups (e.g., 2-nitrobenzyl alkylated HOMedU triphosphates, bases comprising modification with large chemical groups, such as solid supports or other large moiety). In some instances, a polymerase with strand displacement activity but without 3′->5′exonuclease proofreading activity is used with terminator nucleotides with or without modifications to make them exonuclease resistant. Such nucleic acid polymerases include, without limitation, Bst DNA polymerase, Bsu DNA polymerase, Deep Vent (exo-) DNA polymerase, Klenow Fragment (exo-) DNA polymerase, Therminator DNA polymerase, and Vent_{R} (exo-).

### Primers and Amplicon Libraries

Described herein are amplicon libraries resulting from amplification of at least one target nucleic acid molecule. Such libraries are in some instances generated using the methods described herein, such as those using terminators. In some instances, terminators are used in combination with A, C, T, G, and U nucleotides. In some instances, amplicons generated by methods described herein comprise uracil. Such methods comprise use of strand displacement polymerases or factors, terminator nucleotides (reversible or irreversible), or other features and embodiments described herein. In some instances, amplicon libraries generated by use of terminators described herein are further amplified in a subsequent amplification reaction (e.g., PCR). In some instances, subsequent amplification reactions do not comprise terminators. In some instances, amplicon libraries comprise polynucleotides, wherein at least 50%, 60%, 70%, 80%, 90%, 95%, or at least 98% of the polynucleotides comprise at least one terminator nucleotide. In some instances, the amplicon library comprises the target nucleic acid molecule from which the amplicon library was derived. The amplicon library comprises a plurality of polynucleotides, wherein at least some of the polynucleotides are direct copies (e.g., replicated directly from a target nucleic acid molecule, such as genomic DNA, RNA, or other target nucleic acid). For example, at least 5%, 10%, 20%, 30%, 40%, 50%, 60%, 70%, 80%, 90%, 95% or more than 95% of the amplicon polynucleotides are direct copies of the at least one target nucleic acid molecule. In some instances, at least 5% of the amplicon polynucleotides are direct copies of the at least one target nucleic acid molecule. In some instances, at least 10% of the amplicon polynucleotides are direct copies of the at least one target nucleic acid molecule. In some instances, at least 15% of the amplicon polynucleotides are direct copies of the at least one target nucleic acid molecule. In some instances, at least 20% of the amplicon polynucleotides are direct copies of the at least one target nucleic acid molecule. In some instances, at least 50% of the amplicon polynucleotides are direct copies of the at least one target nucleic acid molecule. In some instances, 3%-5%, 3-10%, 5%-10%, 10%-20%, 20%-30%, 30%-40%, 5%-30%, 10%-50%, or 15%-75% of the amplicon polynucleotides are direct copies of the at least one target nucleic acid molecule. In some instances, at least some of the polynucleotides are direct copies of the target nucleic acid molecule, or daughter (a first copy of the target nucleic acid) progeny. For example, at least 5%, 10%, 20%, 30%, 40%, 50%, 60%, 70%, 80%, 90%, 95% or more than 95% of the amplicon polynucleotides are direct copies of the at least one target nucleic acid molecule or daughter progeny. In some instances, at least 5% of the amplicon polynucleotides are direct copies of the at least one target nucleic acid molecule or daughter progeny. In some instances, at least 10% of the amplicon polynucleotides are direct copies of the at least one target nucleic acid molecule or daughter progeny. In some instances, at least 20% of the amplicon polynucleotides are direct copies of the at least one target nucleic acid molecule or daughter progeny. In some instances, at least 30% of the amplicon polynucleotides are direct copies of the at least one target nucleic acid molecule or daughter progeny. In some instances, 3%-5%, 3%-10%, 5%-10%, 10%-20%, 20%-30%, 30%-40%, 5%-30%, 10%-50%, or 15%-75% of the amplicon polynucleotides are direct copies of the at least one target nucleic acid molecule or daughter progeny. In some instances, direct copies of the target nucleic acid are 50-2500, 75-2000, 50-2000, 25-1000, 50-1000, 500-2000, or 50-2000 bases in length. In some instances, daughter progeny are 1000-5000, 2000-5000, 1000-10,000, 2000-5000, 1500-5000, 3000-7000, or 2000-7000 bases in length. In some instances, the average length of PTA amplification products is 25-3000 nucleotides in length, 50-2500, 75-2000, 50-2000, 25-1000, 50-1000, 500-2000, or 50-2000 bases in length. In some instance, amplicons generated from PTA are no more than 5000, 4000, 3000, 2000, 1700, 1500, 1200, 1000, 700, 500, or no more than 300 bases in length. In some instance, amplicons generated from PTA are 1000-5000, 1000-3000, 200-2000, 200-4000, 500-2000, 750-2500, or 1000-2000 bases in length. Amplicon libraries generated using the methods described herein in some instances comprise at least 1000, 2000, 5000, 10,000, 100,000, 200,000, 500,000 or more than 500,000 amplicons comprising unique sequences. In some instances, the library comprises at least 100, 200, 300, 400, 500, 600, 700, 800, 900, 1000, 1100, 1200, 1300, 1400, 1500, 2000, 2500, 3000, or at least 3500 amplicons. In some instances, at least 5%, 10%, 15%, 20%, 25%, 30% or more than 30% of amplicon polynucleotides having a length of less than 1000 bases are direct copies of the at least one target nucleic acid molecule. In some instances, at least 5%, 10%, 15%, 20%, 25%, 30% or more than 30% of amplicon polynucleotides having a length of no more than 2000 bases are direct copies of the at least one target nucleic acid molecule. In some instances, at least 5%, 10%, 15%, 20%, 25%, 30% or more than 30% of amplicon polynucleotides having a length of 3000-5000 bases are direct copies of the at least one target nucleic acid molecule. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are no more than 700-1200 bases in length. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are 700-1200 bases in length, and the daughter amplicons are 2500-6000 bases in length. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule or daughter amplicons. The number of direct copies may be controlled in some instances by the number of PCR amplification cycles. In some instances, no more than 30, 25, 20, 15, 13, 11, 10, 9, 8, 7, 6, 5, 4, or 3 PCR cycles are used to generate copies of the target nucleic acid molecule. In some instances, about 30, 25, 20, 15, 13, 11, 10, 9, 8, 7, 6, 5, 4, or about 3 PCR cycles are used to generate copies of the target nucleic acid molecule. In some instances, 3, 4, 5, 6, 7, or 8 PCR cycles are used to generate copies of the target nucleic acid molecule. In some instances, 2-4, 2-5, 2-7, 2-8, 2-10, 2-15, 3-5, 3-10, 3-15, 4-10, 4-15, 5-10 or 5-15 PCR cycles are used to generate copies of the target nucleic acid molecule. Amplicon libraries generated using the methods described herein are in some instances subjected to additional steps, such as adapter ligation and further PCR amplification. In some instances, such additional steps precede a sequencing step.

Methods described herein may additionally comprise one or more enrichment or purification steps. In some instances, one or more polynucleotides (such as cDNA, PTA amplicons, or other polynucleotide) are enriched during a method described herein. In some instances, polynucleotide probes are used to capture one or more polynucleotides. In some instances, probes are configured to capture one or more genomic exons. In some instances, a library of probes comprises at least 1000, 2000, 5000, 10,000, 50,000, 100,000, 200,000, 500,000, or more than 1 million different sequences. In some instances, a library of probes comprises sequences capable of binding to at least 10, 20, 50, 100, 200, 500, 1000, 2000, 5000, 10,000 or more than 10,000 genes. In some instances, probes comprise a moiety for capture by a solid support, such as biotin. In some instances, an enrichment step occurs after a PTA step. In some instances, an enrichment step occurs before a PTA step. In some instances, probes are configured to bind genomic DNA libraries. In some instances, probes are configured to bind cDNA libraries.

Amplicon libraries of polynucleotides generated from the PTA methods and compositions (terminators, polymerases, etc.) described herein in some instances have increased uniformity. Uniformity, in some instances, is described using a Lorenz curve, or other such method. Such increases in some instances lead to lower sequencing reads needed for the desired coverage of a target nucleic acid molecule (e.g., genomic DNA, RNA, or other target nucleic acid molecule). For example, no more than 50% of a cumulative fraction of polynucleotides comprises sequences of at least 80% of a cumulative fraction of sequences of the target nucleic acid molecule. In some instances, no more than 50% of a cumulative fraction of polynucleotides comprises sequences of at least 60% of a cumulative fraction of sequences of the target nucleic acid molecule. In some instances, no more than 50% of a cumulative fraction of polynucleotides comprises sequences of at least 70% of a cumulative fraction of sequences of the target nucleic acid molecule. In some instances, no more than 50% of a cumulative fraction of polynucleotides comprises sequences of at least 90% of a cumulative fraction of sequences of the target nucleic acid molecule. In some instances, uniformity is described using a Gini index (wherein an index of 0 represents perfect equality of the library and an index of 1 represents perfect inequality). In some instances, amplicon libraries described herein have a Gini index of no more than 0.55, 0.50, 0.45, 0.40, or 0.30. In some instances, amplicon libraries described herein have a Gini index of no more than 0.50. In some instances, amplicon libraries described herein have a Gini index of no more than 0.40. Such uniformity metrics in some instances are dependent on the number of reads obtained. For example, no more than 100 million, 200 million, 300 million, 400 million, or no more than 500 million reads are obtained. In some instances, the read length is about 50,75, 100, 125, 150, 175, 200, 225, or about 250 bases in length. In some instances, uniformity metrics are dependent on the depth of coverage of a target nucleic acid. For example, the average depth of coverage is about 10×, 15×, 20×, 25×, or about 30×. In some instances, the average depth of coverage is 10-30×, 20-50×, 5-40×, 20-60×, 5-20×, or 10-20×. In some instances, amplicon libraries described herein have a Gini index of no more than 0.55, wherein about 300 million reads was obtained. In some instances, amplicon libraries described herein have a Gini index of no more than 0.50, wherein about 300 million reads was obtained. In some instances, amplicon libraries described herein have a Gini index of no more than 0.45, wherein about 300 million reads was obtained. In some instances, amplicon libraries described herein have a Gini index of no more than 0.55, wherein no more than 300 million reads was obtained. In some instances, amplicon libraries described herein have a Gini index of no more than 0.50, wherein no more than 300 million reads was obtained. In some instances, amplicon libraries described herein have a Gini index of no more than 0.45, wherein no more than 300 million reads was obtained. In some instances, amplicon libraries described herein have a Gini index of no more than 0.55, wherein the average depth of sequencing coverage is about 15×. In some instances, amplicon libraries described herein have a Gini index of no more than 0.50, wherein the average depth of sequencing coverage is about 15×. In some instances, amplicon libraries described herein have a Gini index of no more than 0.45, wherein the average depth of sequencing coverage is about 15×. In some instances, amplicon libraries described herein have a Gini index of no more than 0.55, wherein the average depth of sequencing coverage is at least 15×. In some instances, amplicon libraries described herein have a Gini index of no more than 0.50, wherein the average depth of sequencing coverage is at least 15×. In some instances, amplicon libraries described herein have a Gini index of no more than 0.45, wherein the average depth of sequencing coverage is at least 15×. In some instances, amplicon libraries described herein have a Gini index of no more than 0.55, wherein the average depth of sequencing coverage is no more than 15×. In some instances, amplicon libraries described herein have a Gini index of no more than 0.50, wherein the average depth of sequencing coverage is no more than 15×. In some instances, amplicon libraries described herein have a Gini index of no more than 0.45, wherein the average depth of sequencing coverage is no more than 15×. Uniform amplicon libraries generated using the methods described herein are in some instances subjected to additional steps, such as adapter ligation and further PCR amplification. In some instances, such additional steps precede a sequencing step.

Primers comprise nucleic acids used for priming the amplification reactions described herein. Such primers in some instances include, without limitation, random deoxynucleotides of any length with or without modifications to make them exonuclease resistant, random ribonucleotides of any length with or without modifications to make them exonuclease resistant, modified nucleic acids such as locked nucleic acids, DNA or RNA primers that are targeted to a specific genomic region, and reactions that are primed with enzymes such as primase. In the case of whole genome PTA, it is preferred that a set of primers having random or partially random nucleotide sequences be used. In a nucleic acid sample of significant complexity, specific nucleic acid sequences present in the sample need not be known and the primers need not be designed to be complementary to any particular sequence. Rather, the complexity of the nucleic acid sample results in a large number of different hybridization target sequences in the sample, which will be complementary to various primers of random or partially random sequence. The complementary portion of primers for use in PTA are in some instances fully randomized, comprise only a portion that is randomized, or be otherwise selectively randomized. The number of random base positions in the complementary portion of primers in some instances, for example, is from 20% to 100% of the total number of nucleotides in the complementary portion of the primers. In some instances, the number of random base positions in the complementary portion of primers is 10% to 90%, 15-95%, 20%-100%, 30%-100%, 50%-100%, 75-100% or 90-95% of the total number of nucleotides in the complementary portion of the primers. In some instances, the number of random base positions in the complementary portion of primers is at least 10%, 20%, 30%, 40%, 50%, 60%, 70%, 80%, or at least 90% of the total number of nucleotides in the complementary portion of the primers. Sets of primers having random or partially random sequences are in some instances synthesized using standard techniques by allowing the addition of any nucleotide at each position to be randomized. In some instances, sets of primers are composed of primers of similar length and/or hybridization characteristics. In some instances, the term “random primer” refers to a primer which can exhibit four-fold degeneracy at each position. In some instances, the term “random primer” refers to a primer which can exhibit three-fold degeneracy at each position. Random primers used in the methods described herein in some instances comprise a random sequence that is 3, 4, 5, 6, 7, 8, 10, 11, 12, 13, 14, 15, 16, 17, 18, 19, 20, or more bases in length. In some instances, primers comprise random sequences that are 3-20, 5-15, 5-20, 6-12, or 4-10 bases in length. Primers may also comprise non-extendable elements that limit subsequent amplification of amplicons generated thereof. For example, primers with non-extendable elements in some instances comprise terminators. In some instances, primers comprise terminator nucleotides, such as 1, 2, 3, 4, 5, 10, or more than 10 terminator nucleotides. Primers need not be limited to components which are added externally to an amplification reaction. In some instances, primers are generated in-situ through the addition of nucleotides and proteins which promote priming. For example, primase-like enzymes in combination with nucleotides is in some instances used to generate random primers for the methods described herein. Primase-like enzymes in some instances are members of the DnaG or AEP enzyme superfamily. In some instances, a primase-like enzyme is TthPrimPol. In some instances, a primase-like enzyme is T7 gp4 helicase-primase. Such primases are in some instances used with the polymerases or strand displacement factors described herein. In some instances, primases initiate priming with deoxyribonucleotides. In some instances, primases initiate priming with ribonucleotides.

The PTA amplification can be followed by selection for a specific subset of amplicons. Such selections are in some instances dependent on size, affinity, activity, hybridization to probes, or other known selection factor in the art. In some instances, selections precede or follow additional steps described herein, such as adapter ligation and/or library amplification. In some instances, selections are based on size (length) of the amplicons. In some instances, smaller amplicons are selected that are less likely to have undergone exponential amplification, which enriches for products that were derived from the primary template while further converting the amplification from an exponential into a quasi-linear amplification process (FIG. 1A). In some instances, amplicons comprising 50-2000, 25-5000, 40-3000, 50-1000, 200-1000, 300-1000, 400-1000, 400-600, 600-2000, or 800-1000 bases in length are selected. Size selection in some instances occurs with the use of protocols, e.g., utilizing solid-phase reversible immobilization (SPRI) on carboxylated paramagnetic beads to enrich for nucleic acid fragments of specific sizes, or other protocol known by those skilled in the art. Optionally or in combination, selection occurs through preferential ligation and amplification of smaller fragments during PCR while preparing sequencing libraries, as well as a result of the preferential formation of clusters from smaller sequencing library fragments during sequencing (e.g., sequencing by synthesis, nanopore sequencing, or other sequencing method). Other strategies to select for smaller fragments are also consistent with the methods described herein and include, without limitation, isolating nucleic acid fragments of specific sizes after gel electrophoresis, the use of silica columns that bind nucleic acid fragments of specific sizes, and the use of other PCR strategies that more strongly enrich for smaller fragments. Any number of library preparation protocols may be used with the PTA methods described herein. In some instances, library preparation comprises amplification with a uracil tolerant polymerase. Amplicons generated by PTA are in some instances ligated to adapters (optionally with removal of terminator nucleotides). In some instances, amplicons generated by PTA comprise regions of homology generated from transposase-based fragmentation which are used as priming sites. In some instances, libraries are prepared by fragmenting nucleic acids mechanically or enzymatically. In some instances, libraries are prepared using tagmentation via transposomes. In some instances, libraries are prepared via ligation of adapters, such as Y-adapters, universal adapters, or circular adapters. The non-complementary portion of a primer used in PTA can include sequences which can be used to further manipulate and/or analyze amplified sequences. An example of such a sequence is a “detection tag”. Detection tags have sequences complementary to detection probes and are detected using their cognate detection probes. There may be one, two, three, four, or more than four detection tags on a primer. There is no fundamental limit to the number of detection tags that can be present on a primer except the size of the primer. In some instances, there is a single detection tag on a primer. In some instances, there are two detection tags on a primer. When there are multiple detection tags, they may have the same sequence or they may have different sequences, with each different sequence complementary to a different detection probe. In some instances, multiple detection tags have the same sequence. In some instances, multiple detection tags have a different sequence.

Another example of a sequence that can be included in the non-complementary portion of a primer is an “address tag” that can encode other details of the amplicons, such as the location in a tissue section. In some instances, a cell barcode comprises an address tag. An address tag has a sequence complementary to an address probe. Address tags become incorporated at the ends of amplified strands. If present, there may be one, or more than one, address tag on a primer. There is no fundamental limit to the number of address tags that can be present on a primer except the size of the primer. When there are multiple address tags, they may have the same sequence or they may have different sequences, with each different sequence complementary to a different address probe. The address tag portion can be any length that supports specific and stable hybridization between the address tag and the address probe. In some instances, nucleic acids from more than one source can incorporate a variable tag sequence. This tag sequence can be up to 100 nucleotides in length, preferably 1 to 10 nucleotides in length, most preferably 4, 5 or 6 nucleotides in length and comprises combinations of nucleotides. In some instances, a tag sequence is 1-20, 2-15, 3-13, 4-12, 5-12, or 1-10 nucleotides in length For example, if six base-pairs are chosen to form the tag and a permutation of four different nucleotides is used, then a total of 4096 nucleic acid anchors (e.g. hairpins), each with a unique 6 base tag can be made.

Primers described herein may be present in solution or immobilized on a solid support. In some instances, primers bearing sample barcodes and/or UMI sequences can be immobilized on a solid support. The solid support can be, for example, one or more beads. In some instances, individual cells are contacted with one or more beads having a unique set of sample barcodes and/or UMI sequences in order to identify the individual cell. In some instances, lysates from individual cells are contacted with one or more beads having a unique set of sample barcodes and/or UMI sequences in order to identify the individual cell lysates. In some instances, extracted nucleic acid from individual cells are contacted with one or more beads having a unique set of sample barcodes and/or UMI sequences in order to identify the extracted nucleic acid from the individual cell. The beads can be manipulated in any suitable manner as is known in the art, for example, using droplet actuators as described herein. The beads may be any suitable size, including for example, microbeads, microparticles, nanobeads and nanoparticles. In some embodiments, beads are magnetically responsive; in other embodiments beads are not significantly magnetically responsive. Non-limiting examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® available from Invitrogen Group, Carlsbad, CA), fluorescent microparticles and nanoparticles, coated magnetic microparticles and nanoparticles, ferromagnetic microparticles and nanoparticles, coated ferromagnetic microparticles and nanoparticles, and those described in U.S. Pat. Appl. Pub. No. US20050260686, US20030132538, US20050118574, 20050277197, 20060159962. Beads may be pre-coupled with an antibody, protein or antigen, DNA/RNA probe or any other molecule with an affinity for a desired target. In some embodiments, primers bearing sample barcodes and/or UMI sequences can be in solution. In certain embodiments, a plurality of droplets can be presented, wherein each droplet in the plurality bears a sample barcode which is unique to a droplet and the UMI which is unique to a molecule such that the UMI are repeated many times within a collection of droplets. In some embodiments, individual cells are contacted with a droplet having a unique set of sample barcodes and/or UMI sequences in order to identify the individual cell. In some embodiments, lysates from individual cells are contacted with a droplet having a unique set of sample barcodes and/or UMI sequences in order to identify the individual cell lysates. In some embodiments, extracted nucleic acid from individual cells are contacted with a droplet having a unique set of sample barcodes and/or UMI sequences in order to identify the extracted nucleic acid from the individual cell.

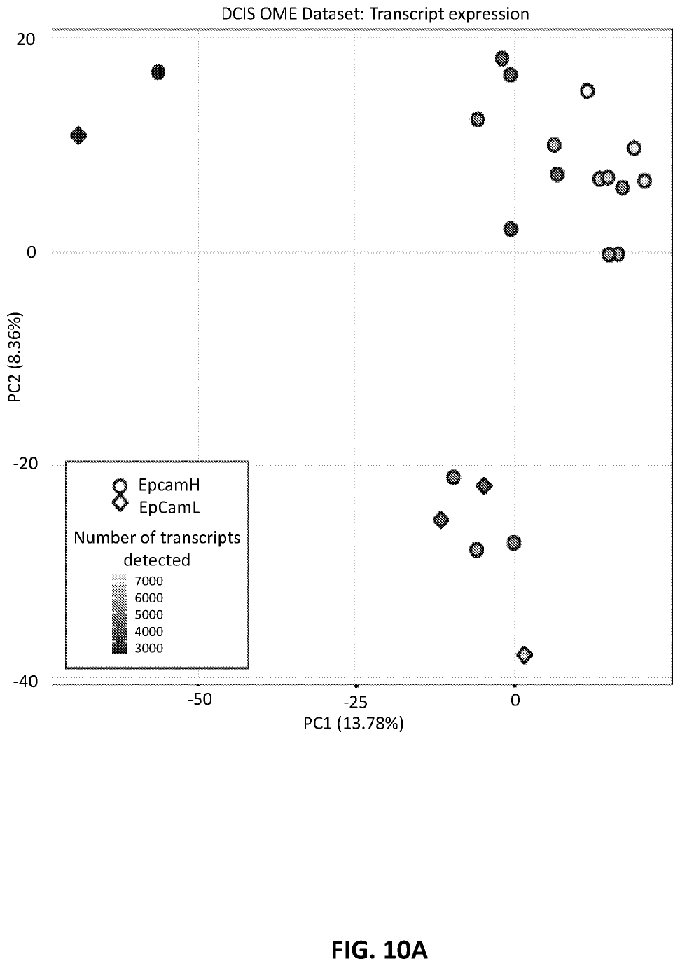

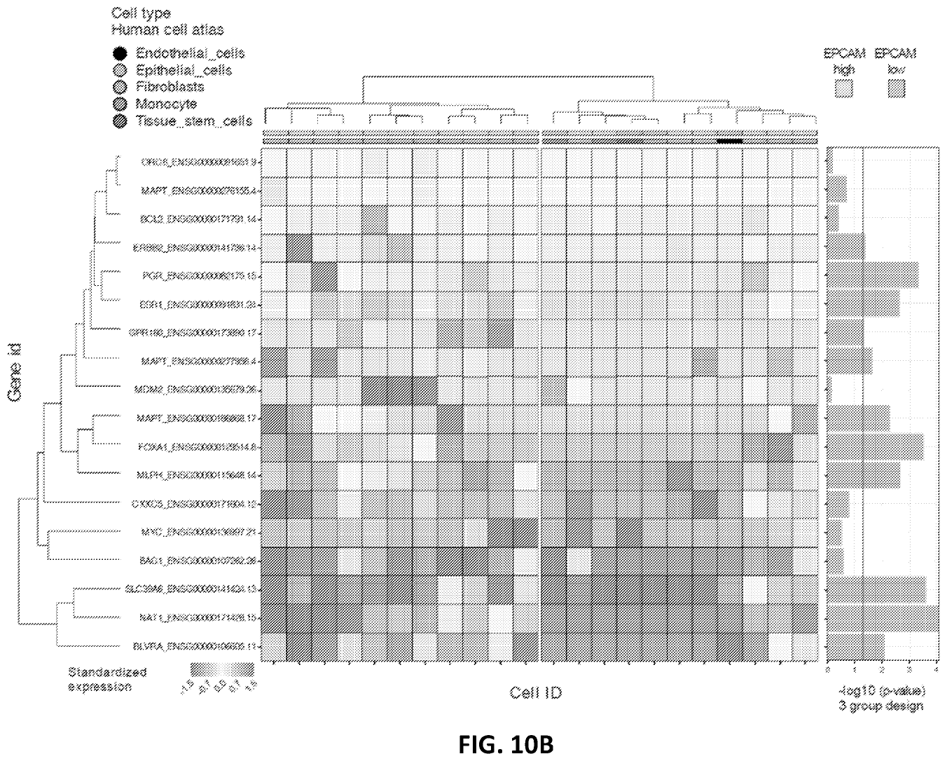

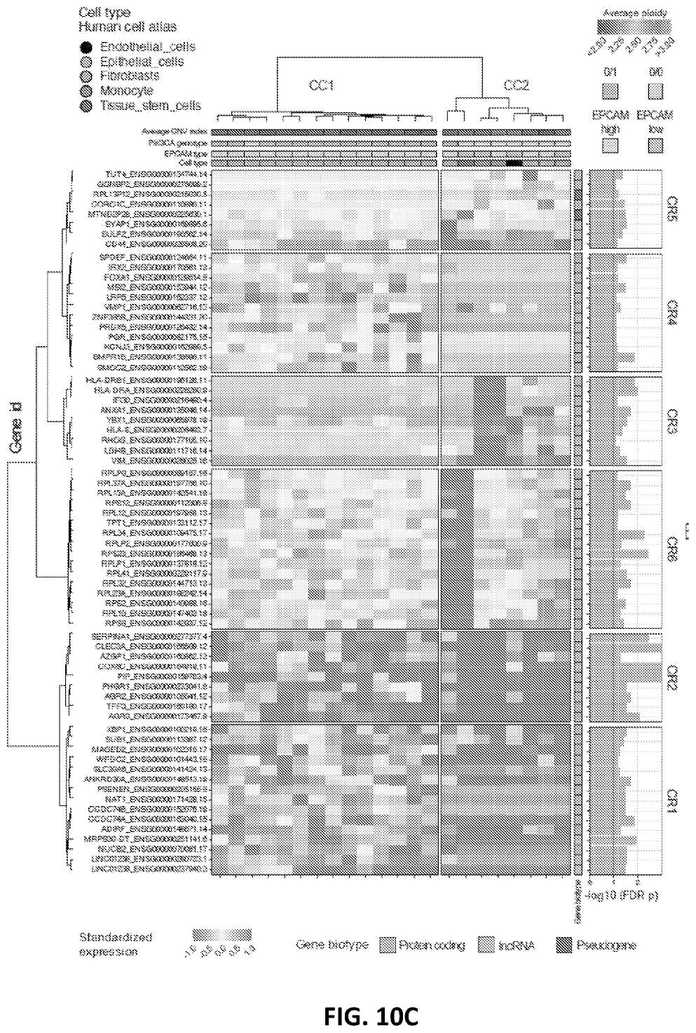

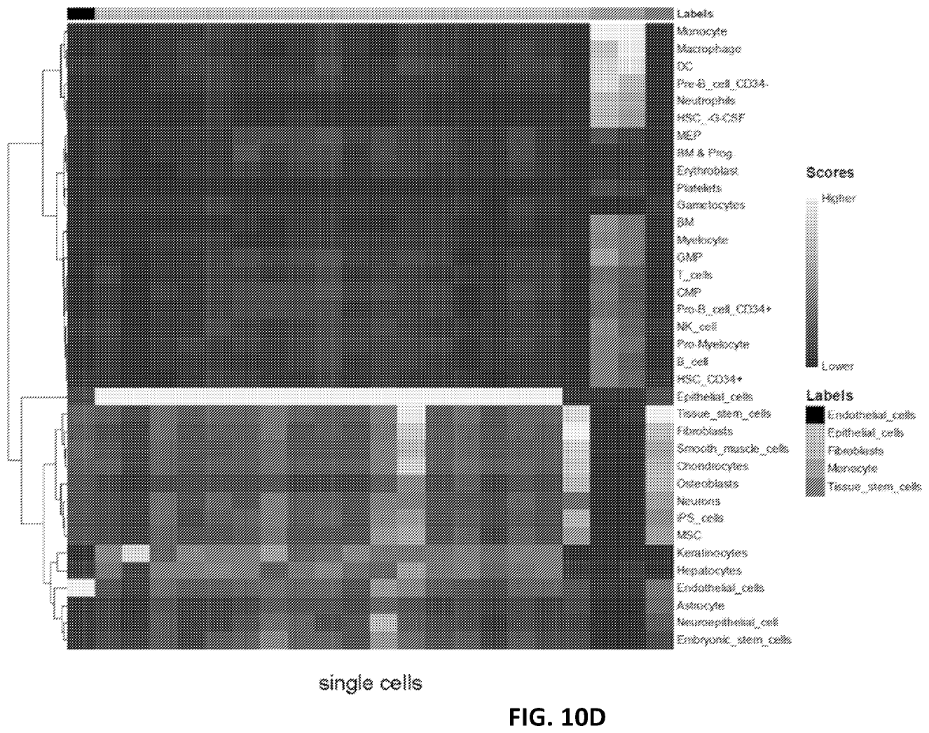

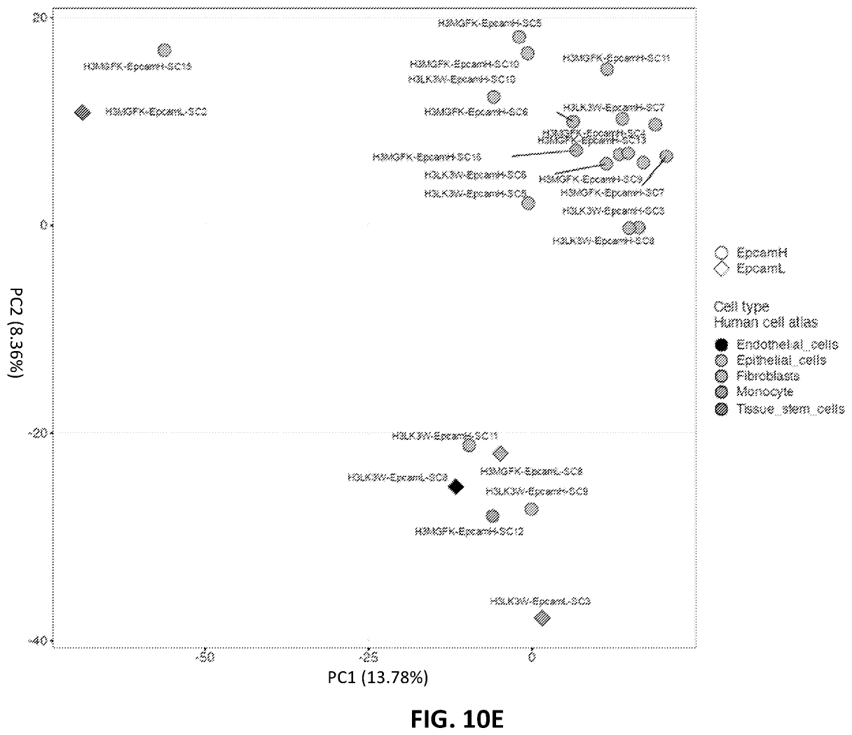

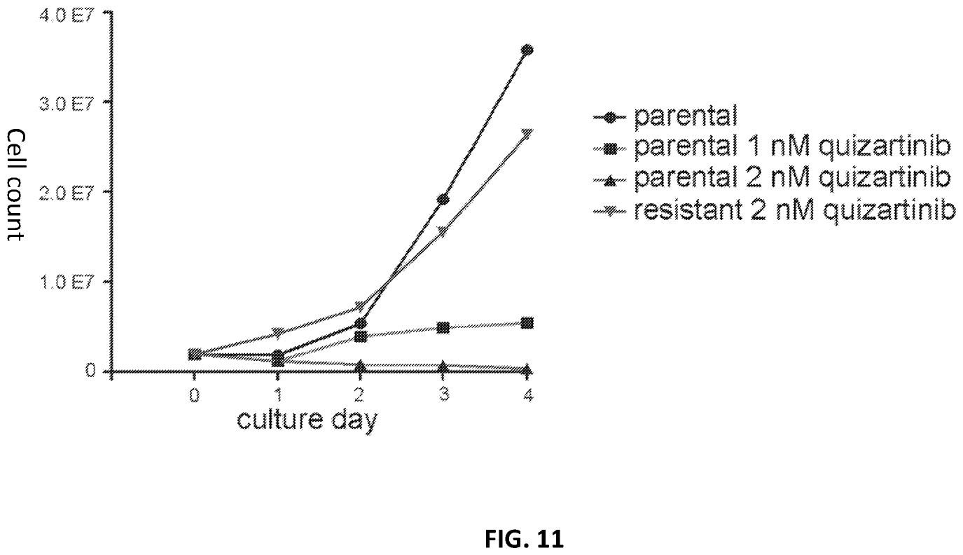

PTA primers may comprise a sequence-specific or random primer, a cell barcode and/or a unique molecular identifier (UMI) (see, e.g., FIGS. 10A (linear primer) and 10B (hairpin primer)). In some instances, a primer comprises a sequence-specific primer. In some instances, a primer comprises a random primer. In some instances, a primer comprises a cell barcode. In some instances, a primer comprises a sample barcode. In some instances, a primer comprises a unique molecular identifier. In some instances, primers comprise two or more cell barcodes. Such barcodes in some instances identify a unique sample source, or unique workflow. Such barcodes or UMIs are in some instances 5, 6, 7, 8, 9, 10, 11, 12, 15, 20, 25, 30, or more than 30 bases in length. Primers in some instances comprise at least 1000, 10,000, 50,000, 100,000, 250,000, 500,000, 106, 107, 108, 109, or at least 1010 unique barcodes or UMIs. In some instances primers comprise at least 8, 16, 96, or 384 unique barcodes or UMIs. In some instances a standard adapter is then ligated onto the amplification products prior to sequencing; after sequencing, reads are first assigned to a specific cell based on the cell barcode. Suitable adapters that may be utilized with the PTA method include, e.g., xGen® Dual Index UMI adapters available from Integrated DNA Technologies (IDT). Reads from each cell is then grouped using the UMI, and reads with the same UMI may be collapsed into a consensus read. The use of a cell barcode allows all cells to be pooled prior to library preparation, as they can later be identified by the cell barcode. The use of the UMI to form a consensus read in some instances corrects for PCR bias, improving the copy number variation (CNV) detection (FIGS. 11A and 11B). In addition, sequencing errors may be corrected by requiring that a fixed percentage of reads from the same molecule have the same base change detected at each position. This approach has been utilized to improve CNV detection and correct sequencing errors in bulk samples. In some instances, UMIs are used with the methods described herein, for example, U.S. Pat. No. 8,835,358 discloses the principle of digital counting after attaching a random amplifiable barcode. Schmitt. et al and Fan et al. disclose similar methods of correcting sequencing errors. In some instances, a library is generated for sequencing using primers. In some instances, the library comprises fragments of 200-700 bases, 100-1000, 300-800, 300-550, 300-700, or 200-800 bases in length. In some instances, the library comprises fragments of at least 50, 100, 150, 200, 300, 500, 600, 700, 800, or at least 1000 bases in length. In some instances, the library comprises fragments of about 50, 100, 150, 200, 300, 500, 600, 700, 800, or about 1000 bases in length.

The methods described herein may further comprise additional steps, including steps performed on the sample or template. Such samples or templates in some instance are subjected to one or more steps prior to PTA. In some instances, samples comprising cells are subjected to a pre-treatment step. For example, cells undergo lysis and proteolysis to increase chromatin accessibility using a combination of freeze-thawing, Triton X-100, Tween 20, and Proteinase K. Other lysis strategies are also be suitable for practicing the methods described herein. Such strategies include, without limitation, lysis using other combinations of detergent and/or lysozyme and/or protease treatment and/or physical disruption of cells such as sonication and/or alkaline lysis and/or hypotonic lysis. In some instances, the primary template or target molecule(s) is subjected to a pre-treatment step. In some instances, the primary template (or target) is denatured using sodium hydroxide, followed by neutralization of the solution. Other denaturing strategies may also be suitable for practicing the methods described herein. Such strategies may include, without limitation, combinations of alkaline lysis with other basic solutions, increasing the temperature of the sample and/or altering the salt concentration in the sample, addition of additives such as solvents or oils, other modification, or any combination thereof. In some instances, additional steps include sorting, filtering, or isolating samples, templates, or amplicons by size. In some instances, cells are lysed with mechanical (e.g., high pressure homogenizer, bead milling) or non-mechanical (physical, chemical, or biological). In some instances, physical lysis methods comprise heating, osmotic shock, and/or cavitation. In some instances, chemical lysis comprises alkali and/or detergents. In some instances, biological lysis comprises use of enzymes. Combinations of lysis methods are also compatible with the methods described herein. Non-limited examples of lysis enzymes include recombinant lysozyme, serine proteases, and bacterial lysins. In some instances, lysis with enzymes comprises use of lysozyme, lysostaphin, zymolase, cellulose, protease or glycanase. For example, after amplification with the methods described herein, amplicon libraries are enriched for amplicons having a desired length. In some instances, amplicon libraries are enriched for amplicons having a length of 50-2000, 25-1000, 50-1000, 75-2000, 100-3000, 150-500, 75-250, 170-500, 100-500, or 75-2000 bases. In some instances, amplicon libraries are enriched for amplicons having a length no more than 75, 100, 150, 200, 500, 750, 1000, 2000, 5000, or no more than 10,000 bases. In some instances, amplicon libraries are enriched for amplicons having a length of at least 25, 50, 75, 100, 150, 200, 500, 750, 1000, or at least 2000 bases.

Methods and compositions described herein may comprise buffers or other formulations. Such buffers are in some instances used for PTA, RT, or other method described herein. Such buffers in some instances comprise surfactants/detergent or denaturing agents (Tween-20, DMSO, DMF, pegylated polymers comprising a hydrophobic group, or other surfactant), salts (potassium or sodium phosphate (monobasic or dibasic), sodium chloride, potassium chloride, TrisHCI, magnesium chloride or sulfate, Ammonium salts such as phosphate, nitrate, or sulfate, EDTA), reducing agents (DTT, THP, DTE, beta-mercaptoethanol, TCEP, or other reducing agent) or other components (glycerol, hydrophilic polymers such as PEG). In some instances, buffers are used in conjunction with components such as polymerases, strand displacement factors, terminators, or other reaction component described herein. In some instances, buffers are used in conjunction with components such as polymerases, strand displacement factors, terminators, or other reaction component described herein. Buffers may comprise one or more crowding agents. In some instances, crowding reagents include polymers. In some instances, crowding reagents comprise polymers such as polyols. In some instances, crowding reagents comprise polyethylene glycol polymers (PEG). In some instances, crowding reagents comprise polysaccharides. Without limitation, examples of crowding reagents include ficoll (e.g., ficoll PM 400, ficoll PM 70, or other molecular weight ficoll), PEG (e.g., PEG1000, PEG 2000, PEG4000, PEG6000, PEG8000, or other molecular weight PEG), dextran (dextran 6, dextran 10, dextran 40, dextran 70, dextran 6000, dextran 138k, or other molecular weight dextran).

The nucleic acid molecules amplified (e.g., by uracil tolerant polymerases) according to the methods described herein may be sequenced and analyzed using methods known to those of skill in the art. Non-limiting examples of the sequencing methods which in some instances are used include, e.g., sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (Int. Pat. Appl. Pub. No. WO2006/073504), multiplex sequencing (U.S. Pat. Appl. Pub. No. US2008/0269068; Porreca et al., 2007, Nat. Methods 4:931), polymerized colony (POLONY) sequencing (U.S. Pat. Nos. 6,432,360, 6,485,944 and 6,511,803, and Int. Pat. Appl. Pub. No. WO2005/082098), nanogrid rolling circle sequencing (ROLONY) (U.S. Pat. No. 9,624,538), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout), high-throughput sequencing methods such as, e.g., methods using Roche 454, Illumina Solexa, AB-SOLID, Helicos, Polonator platforms and the like, and light-based sequencing technologies (Landegren et al. (1998) Genome Res. 8:769-76; Kwok (2000) Pharmacogenomics 1:95-100; and Shi (2001) Clin. Chem. 47:164-172). In some instances, the amplified nucleic acid molecules are shotgun sequenced. Sequencing of the sequencing library is in some instances performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis (array/colony-based or nanoball based).

Sequencing libraries generated using the methods described herein (e.g., PTA or RNAseq) may be sequenced to obtain a desired number of sequencing reads. In some instances, libraries are generated from a single cell or sample comprising a single cell (alone or part of a multiomics workflow). In some instances, libraries are sequenced to obtain at least 0.1, 0.2, 0.4, 0.5, 0.7, 0.8, 0.9, 1, 1.1, 1.2, 1.5, 2, 5, or at least 10 million reads. In some instances, libraries are sequenced to obtain no more than 0.1, 0.2, 0.4, 0.5, 0.7, 0.8, 0.9, 1, 1.1, 1.2, 1.5, 2, 5, or no more than 10 million reads. In some instances, libraries are sequenced to obtain about 0.1, 0.2, 0.4, 0.5, 0.7, 0.8, 0.9, 1, 1.1, 1.2, 1.5, 2, 5, or about 10 million reads. In some instances, libraries are sequenced to obtain 0.1-10, 0.1-5, 0.1-1, 0.2-1, 0.3-1.5, 0.5-1, 1-5, or 0.5-5 million reads per sample. In some instances, the number of reads is dependent on the size of the genome. In some in instances samples comprising bacterial genomes are sequenced to obtain 0.5-1 million reads. In some instances, libraries are sequenced to obtain at least 2, 4, 10, 20, 50, 100, 200, 300, 500, 700, or at least 900 million reads. In some instances, libraries are sequenced to obtain no more than 2, 4, 10, 20, 50, 100, 200, 300, 500, 700, or no more than 900 million reads. In some instances, libraries are sequenced to obtain about 2, 4, 10, 20, 50, 100, 200, 300, 500, 700, or about 900 million reads. In some in instances samples comprising mammalian genomes are sequenced to obtain 500-600 million reads. In some instances, the type of sequencing library (cDNA libraries or genomic libraries) are identified during sequencing. In some instances, cDNA libraries and genomic libraries are identified during sequencing with unique barcodes.

The term “cycle” when used in reference to a polymerase-mediated amplification reaction is used herein to describe steps of dissociation of at least a portion of a double stranded nucleic acid (e.g., a template from an amplicon, or a double stranded template, denaturation). hybridization of at least a portion of a primer to a template (annealing), and extension of the primer to generate an amplicon. In some instances, the temperature remains constant during a cycle of amplification (e.g., an isothermal reaction). In some instances, the number of cycles is directly correlated with the number of amplicons produced. In some instances, the number of cycles for an isothermal reaction is controlled by the amount of time the reaction is allowed to proceed.

### Methods and Applications

Described herein are methods of identifying mutations in cells with the methods of multiomic analysis PTA, such as single cells. Use of the PTA method in some instances results in improvements over known methods, for example, MDA. PTA in some instances has lower false positive and false negative variant calling rates than the MDA method. Genomes, such as NA12878 platinum genomes, are in some instances used to determine if the greater genome coverage and uniformity of PTA would result in lower false negative variant calling rate. Without being bound by theory, it may be determined that the lack of error propagation in PTA decreases the false positive variant call rate. The amplification balance between alleles with the two methods is in some cases estimated by comparing the allele frequencies of the heterozygous mutation calls at known positive loci. In some instances, amplicon libraries generated using PTA are further amplified by PCR. In some instances, PTA is used in a workflow with additional analysis methods, such as RNAseq, methylome analysis or other method described herein.

Cells analyzed using the methods described herein in some instances comprise tumor cells. For example, circulating tumor cells can be isolated from a fluid taken from patients, such as but not limited to, blood, bone marrow, urine, saliva, cerebrospinal fluid, pleural fluid, pericardial fluid, ascites, or aqueous humor. The cells are then subjected to the methods described herein (e.g. PTA) and sequencing to determine mutation burden and mutation combination in each cell. These data are in some instances used for the diagnosis of a specific disease or as tools to predict treatment response. Similarly, in some instances cells of unknown malignant potential in some instances are isolated from fluid taken from patients, such as but not limited to, blood, bone marrow, urine, saliva, cerebrospinal fluid, pleural fluid, pericardial fluid, ascites, aqueous humor, blastocoel fluid, or collection media surrounding cells in culture. In some instances, a sample is obtained from collection media surrounding embryonic cells. After utilizing the methods described herein and sequencing, such methods are further used to determine mutation burden and mutation combination in each cell. These data are in some instances used for the diagnosis of a specific disease or as tools to predict progression of a premalignant state to overt malignancy. In some instances, cells can be isolated from primary tumor samples. The cells can then undergo PTA and sequencing to determine mutation burden and mutation combination in each cell. These data can be used for the diagnosis of a specific disease or are as tools to predict the probability that a patient's malignancy is resistant to available anti-cancer drugs. By exposing samples to different chemotherapy agents, it has been found that the major and minor clones have differential sensitivity to specific drugs that does not necessarily correlate with the presence of a known “driver mutation,” suggesting that combinations of mutations within a clonal population determine its sensitivities to specific chemotherapy drugs. Without being bound by theory, these findings suggest that a malignancy may be easier to eradicate if premalignant lesions that have not yet expanded are and evolved into clones are detected whose increased number of genome modification may make them more likely to be resistant to treatment. See, Ma et al., 2018, “Pan-cancer genome and transcriptome analyses of 1,699 pediatric leukemias and solid tumors.” A single-cell genomics protocol is in some instances used to detect the combinations of somatic genetic variants in a single cancer cell, or clonotype, within a mixture of normal and malignant cells that are isolated from patient samples. This technology is in some instances further utilized to identify clonotypes that undergo positive selection after exposure to drugs, both in vitro and/or in patients. By comparing the surviving clones exposed to chemotherapy compared to the clones identified at diagnosis, a catalog of cancer clonotypes can be created that documents their resistance to specific drugs. PTA methods in some instances detect the sensitivity of specific clones in a sample composed of multiple clonotypes to existing or novel drugs, as well as combinations thereof, where the method can detect the sensitivity of specific clones to the drug. This approach in some instances shows efficacy of a drug for a specific clone that may not be detected with current drug sensitivity measurements that consider the sensitivity of all cancer clones together in one measurement. When the PTA described herein are applied to patient samples collected at the time of diagnosis in order to detect the cancer clonotypes in a given patient's cancer, a catalog of drug sensitivities may then be used to look up those clones and thereby inform oncologists as to which drug or combination of drugs will not work and which drug or combination of drugs is most likely to be efficacious against that patient's cancer. The PTA may be used for analysis of samples comprising groups of cells. In some instances, a sample comprises neurons or glial cells. In some instances, the sample comprises nuclei.

Described herein are methods of measuring the gene expression alteration in combination with the mutagenicity of an environmental factor. For example, cells (single or a population) are exposed to a potential environmental condition. For example, cells such originating from organs (liver, pancreas, lung, colon, thyroid, or other organ), tissues (skin, or other tissue), blood, or other biological source are in some instances used with the method. In some instances, an environmental condition comprises heat, light (e.g. ultraviolet), radiation, a chemical substance, or any combination thereof. After an amount of exposure to the environmental condition, in some instances minutes, hours, days, or longer, single cells are isolated and subjected to the PTA method. In some instances, molecular barcodes and unique molecular identifiers are used to tag the sample. The sample is sequenced and then analyzed to identify gene expression alterations and or resulting from mutations resulting from exposure to the environmental condition. In some instances, such mutations are compared with a control environmental condition, such as a known non-mutagenic substance, vehicle/solvent, or lack of an environmental condition. Such analysis in some instances not only provides the total number of mutations caused by the environmental condition, but also the locations and nature of such mutations. Patterns are in some instances identified from the data, and may be used for diagnosis of diseases or conditions. In some instances, patterns are used to predict future disease states or conditions. In some instances, the methods described herein measure the mutation burden, locations, and patterns in a cell after exposure to an environmental agent, such as, e.g., a potential mutagen or teratogen. This approach in some instances is used to evaluate the safety of a given agent, including its potential to induce mutations that can contribute to the development of a disease. For example, the method could be used to predict the carcinogenicity or teratogenicity of an agent to specific cell types after exposure to a specific concentration of the specific agent.

Described herein are methods of identifying gene expression alteration in combination with the mutations in animal, plant or microbial cells that have undergone genome editing (e.g., using CRISPR technologies). Such cells in some instances can be isolated and subjected to PTA and sequencing to determine mutation burden and mutation combination in each cell. The per-cell mutation rate and locations of mutations that result from a genome editing protocol are in some instances used to assess the safety of a given genome editing method.

Described herein are methods of determining gene expression alteration in combination with the mutations in cells that are used for cellular therapy, such as but not limited to the transplantation of induced pluripotent stem cells, transplantation of hematopoietic or other cells that have not be manipulated, or transplantation of hematopoietic or other cells that have undergone genome edits. The cells can then undergo PTA and sequencing to determine mutation burden and mutation combination in each cell. The per-cell mutation rate and locations of mutations in the cellular therapy product can be used to assess the safety and potential efficacy of the product.

Cells for use with the PTA method may be fetal cells, such as embryonic cells. In some embodiments, PTA is used in conjunction with non-invasive preimplantation genetic testing (NIPGT). In a further embodiment, cells can be isolated from blastomeres that are created by in vitro fertilization. The cells can then undergo PTA and sequencing to determine the burden and combination of potentially disease predisposing genetic variants in each cell. The gene expression alteration in combination with the mutation profile of the cell can then be used to extrapolate the genetic predisposition of the blastomere to specific diseases prior to implantation. In some instances embryos in culture shed nucleic acids that are used to assess the health of the embryo using low pass genome sequencing. In some instances, embryos are frozen-thawed. In some instances, nucleic acids obtained from blastocyte culture conditioned medium (BCCM), blastocoel fluid (BF), or a combination thereof. In some instances, PTA analysis of fetal cells is used to detect chromosomal abnormalities, such as fetal aneploidy. In some instances, PTA is used to detect diseases such as Down's or Patau syndromes. In some instances, frozen blastocytes are thawed and cultured for a period of time before obtaining nucleic acids for analysis (e.g., culture media, BF, or a cell biopsy). In some instances, blastocytes are cultured for no more than 4, 6, 8, 12, 16, 24, 36, 48, or no more than 64 hours prior to obtaining nucleic acids for analysis.

In another embodiment, microbial cells (e.g., bacteria, fungi, protozoa) can be isolated from plants or animals (e.g., from microbiota samples [e.g., GI microbiota, skin microbiota, etc.] or from bodily fluids such as, e.g., blood, bone marrow, urine, saliva, cerebrospinal fluid, pleural fluid, pericardial fluid, ascites, or aqueous humor). In addition, microbial cells may be isolated from indwelling medical devices, such as but not limited to, intravenous catheters, urethral catheters, cerebrospinal shunts, prosthetic valves, artificial joints, or endotracheal tubes. The cells can then undergo PTA and sequencing to determine the identity of a specific microbe, as well as to detect the presence of microbial genetic variants that predict response (or resistance) to specific antimicrobial agents. These data can be used for the diagnosis of a specific infectious disease and/or as tools to predict treatment response.

Described herein are methods generating amplicon libraries from samples comprising short nucleic acid using the PTA methods described herein. In some instances, PTA leads to improved fidelity and uniformity of amplification of shorter nucleic acids. In some instances, nucleic acids are no more than 2000 bases in length. In some instances, nucleic acids are no more than 1000 bases in length. In some instances, nucleic acids are no more than 500 bases in length. In some instances, nucleic acids are no more than 200, 400, 750, 1000, 2000 or 5000 bases in length. In some instances, samples comprising short nucleic acid fragments include but at not limited to ancient DNA (hundreds, thousands, millions, or even billions of years old), FFPE (Formalin-Fixed Paraffin-Embedded) samples, cell-free DNA, or other sample comprising short nucleic acids.

Described herein are methods of amplifying a target nucleic acid molecule, the method comprising: a) bringing into contact a sample comprising the target nucleic acid molecule, one or more amplification primers, a nucleic acid polymerase, and a mixture of nucleotides which comprises one or more terminator nucleotides which terminate nucleic acid replication by the polymerase, and b) incubating the sample under conditions that promote replication of the target nucleic acid molecule to obtain a plurality of terminated amplification products, wherein the replication proceeds by strand displacement replication. In one embodiment of any of the above methods, the method further comprises isolating from the plurality of terminated amplification products the products which are between about 50 and about 2000 nucleotides in length. In one embodiment of any of the above methods, the method further comprises isolating from the plurality of terminated amplification products the products which are between about 400 and about 600 nucleotides in length. In one embodiment of any of the above methods, the method further comprises: c) repairing ends and A-tailing, and d) ligating the molecules obtained in step (c) to adaptors, and thereby generating a library of amplification products. In some embodiments, the method further comprises removal of the terminator nucleotides from the terminated amplification products. In one embodiment of any of the above methods, the method further comprises sequencing the amplification products. In one embodiment of any of the above methods, the amplification is performed under substantially isothermic conditions. In one embodiment of any of the above methods, the nucleic acid polymerase is a DNA polymerase.

In one embodiment of any of the above methods, the DNA polymerase is a strand displacing DNA polymerase. In one embodiment of any of the above methods, the nucleic acid polymerase is selected from bacteriophage phi29 (29) polymerase, genetically modified phi29 (@29) DNA polymerase, Klenow Fragment of DNA polymerase I, phage M2 DNA polymerase, phage phiPRD1 DNA polymerase, Bst DNA polymerase, Bst large fragment DNA polymerase, exo(−) Bst polymerase, exo(−)Bca DNA polymerase, Bsu DNA polymerase, Vent_{R} DNA polymerase, Vent_{R} (exo-) DNA polymerase, Deep Vent DNA polymerase, Deep Vent (exo-) DNA polymerase, IsoPol DNA polymerase, DNA polymerase I, Therminator DNA polymerase, T5 DNA polymerase, Sequenase, T7 DNA polymerase, T7-Sequenase, and T4 DNA polymerase. In one embodiment of any of the above methods, the nucleic acid polymerase has 3′->5′ exonuclease activity and the terminator nucleotides inhibit such 3′->5′ exonuclease activity. In one specific embodiment, the terminator nucleotides are selected from nucleotides with modification to the alpha group (e.g., alpha-thio dideoxynucleotides creating a phosphorothioate bond), C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids. In one embodiment of any of the above methods, the nucleic acid polymerase does not have 3′->5′ exonuclease activity. In one specific embodiment, the polymerase is selected from Bst DNA polymerase, exo(−) Bst polymerase, exo(−) Bca DNA polymerase, Bsu DNA polymerase, Vent_{R} (exo-) DNA polymerase, Deep Vent (exo-) DNA polymerase, Klenow Fragment (exo-) DNA polymerase, and Therminator DNA polymerase. In one specific embodiment, the terminator nucleotides comprise modifications of the r group of the 3′ carbon of the deoxyribose. In one specific embodiment, the terminator nucleotides are selected from 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, and combinations thereof. In one specific embodiment, the terminator nucleotides are selected from dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. In one embodiment of any of the above methods, the amplification primers are between 4 and 70 nucleotides long. In one embodiment of any of the above methods, the amplification products are between about 50 and about 2000 nucleotides in length. In one embodiment of any of the above methods, the target nucleic acid is DNA (e.g., a cDNA or a genomic DNA). In one embodiment of any of the above methods, the amplification primers are random primers. In one embodiment of any of the above methods, the amplification primers comprise a barcode. In one specific embodiment, the barcode comprises a cell barcode. In one specific embodiment, the barcode comprises a sample barcode. In one embodiment of any of the above methods, the amplification primers comprise a unique molecular identifier (UMI). In one embodiment of any of the above methods, the method comprises denaturing the target nucleic acid or genomic DNA before the initial primer annealing. In one specific embodiment, denaturation is conducted under alkaline conditions followed by neutralization. In one embodiment of any of the above methods, the sample, the amplification primers, the nucleic acid polymerase, and the mixture of nucleotides are contained in a microfluidic device. In one embodiment of any of the above methods, the sample, the amplification primers, the nucleic acid polymerase, and the mixture of nucleotides are contained in a droplet. In one embodiment of any of the above methods, the sample is selected from tissue(s) samples, cells, biological fluid samples (e.g., blood, urine, saliva, lymphatic fluid, cerebrospinal fluid (CSF), amniotic fluid, pleural fluid, pericardial fluid, ascites, aqueous humor), bone marrow samples, semen samples, biopsy samples, cancer samples, tumor samples, cell lysate samples, forensic samples, archaeological samples, paleontological samples, infection samples, production samples, whole plants, plant parts, microbiota samples, viral preparations, soil samples, marine samples, freshwater samples, household or industrial samples, and combinations and isolates thereof. In one embodiment of any of the above methods, the sample is a cell (e.g., an animal cell [e.g., a human cell], a plant cell, a fungal cell, a bacterial cell, and a protozoal cell). In one specific embodiment, the cell is lysed prior to the replication. In one specific embodiment, cell lysis is accompanied by proteolysis. In one specific embodiment, the cell is selected from a cell from a preimplantation embryo, a stem cell, a fetal cell, a tumor cell, a suspected cancer cell, a cancer cell, a cell subjected to a gene editing procedure, a cell from a pathogenic organism, a cell obtained from a forensic sample, a cell obtained from an archeological sample, and a cell obtained from a paleontological sample. In one embodiment of any of the above methods, the sample is a cell from a preimplantation embryo (e.g., a blastomere [e.g., a blastomere obtained from an eight-cell stage embryo produced by in vitro fertilization]). In one specific embodiment, the method further comprises determining the presence of disease predisposing germline or somatic variants in the embryo cell. In one embodiment of any of the above methods, the sample is a cell from a pathogenic organism (e.g., a bacterium, a fungus, a protozoan). In one specific embodiment, the pathogenic organism cell is obtained from fluid taken from a patient, microbiota sample (e.g., GI microbiota sample, vaginal microbiota sample, skin microbiota sample, etc.) or an indwelling medical device (e.g., an intravenous catheter, a urethral catheter, a cerebrospinal shunt, a prosthetic valve, an artificial joint, an endotracheal tube, etc.). In one specific embodiment, the method further comprises the step of determining the identity of the pathogenic organism. In one specific embodiment, the method further comprises determining the presence of genetic variants responsible for resistance of the pathogenic organism to a treatment. In one embodiment of any of the above methods, the sample is a tumor cell, a suspected cancer cell, or a cancer cell. In one specific embodiment, the method further comprises determining the presence of one or more diagnostic or prognostic mutations. In one specific embodiment, the method further comprises determining the presence of germline or somatic variants responsible for resistance to a treatment. In one embodiment of any of the above methods, the sample is a cell subjected to a gene editing procedure. In one specific embodiment, the method further comprises determining the presence of unplanned mutations caused by the gene editing process. In one embodiment of any of the above methods, the method further comprises determining the history of a cell lineage. In a related aspect, the invention provides a use of any of the above methods for identifying low frequency sequence variants (e.g., variants which constitute ≥0.01% of the total sequences).

In a related aspect, the invention provides a kit comprising a nucleic acid polymerase, one or more amplification primers, a mixture of nucleotides comprising one or more terminator nucleotides, and optionally instructions for use. In one embodiment of the kits of the invention, the nucleic acid polymerase is a strand displacing DNA polymerase. In one embodiment of the kits of the invention, the nucleic acid polymerase is selected from bacteriophage phi29 (29) polymerase, genetically modified phi29 (@29) DNA polymerase, Klenow Fragment of DNA polymerase I, phage M2 DNA polymerase, phage phiPRD1 DNA polymerase, Bst DNA polymerase, Bst large fragment DNA polymerase, exo(−) Bst polymerase, exo(−)Bca DNA polymerase, Bsu DNA polymerase, Vent_{R} DNA polymerase, Vent_{R} (exo-) DNA polymerase, Deep Vent DNA polymerase, Deep Vent (exo-) DNA polymerase, IsoPol DNA polymerase, DNA polymerase I, Therminator DNA polymerase, T5 DNA polymerase, Sequenase, T7 DNA polymerase, T7-Sequenase, and T4 DNA polymerase. In one embodiment of the kits of the invention, the nucleic acid polymerase has 3′->5′ exonuclease activity and the terminator nucleotides inhibit such 3′->5′ exonuclease activity (e.g., nucleotides with modification to the alpha group [e.g., alpha-thio dideoxynucleotides], C3 spacer nucleotides, locked nucleic acids (LNA), inverted nucleic acids, 2′ fluoro nucleotides, 3′ phosphorylated nucleotides, 2′-O-Methyl modified nucleotides, trans nucleic acids). In one embodiment of the kits of the invention, the nucleic acid polymerase does not have 3′->5′ exonuclease activity (e.g., Bst DNA polymerase, exo(−) Bst polymerase, exo(−) Bca DNA polymerase, Bsu DNA polymerase, Vent_{R} (exo-) DNA polymerase, Deep Vent (exo-) DNA polymerase, Klenow Fragment (exo-) DNA polymerase, Therminator DNA polymerase). In one specific embodiment, the terminator nucleotides comprise modifications of the r group of the 3′ carbon of the deoxyribose. In one specific embodiment, the terminator nucleotides are selected from 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, and combinations thereof. In one specific embodiment, the terminator nucleotides are selected from dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof.

Described herein are methods of amplifying a genome, the method comprising: a) bringing into contact a sample comprising the genome, a plurality of amplification primers (e.g., two or more primers), a nucleic acid polymerase, and a mixture of nucleotides which comprises one or more terminator nucleotides which terminate nucleic acid replication by the polymerase, and b) incubating the sample under conditions that promote replication of the genome to obtain a plurality of terminated amplification products, wherein the replication proceeds by strand displacement replication. In one embodiment of any of the above methods, the method further comprises isolating from the plurality of terminated amplification products the products which are between about 50 and about 2000 nucleotides in length. In one embodiment of any of the above methods, the method further comprises isolating from the plurality of terminated amplification products the products which are between about 400 and about 600 nucleotides in length. In one embodiment of any of the above methods, the method further comprises: c) repairing ends and A-tailing, and d) ligating the molecules obtained in step (c) to adaptors, and thereby generating a library of amplification products. In one embodiment of any of the above methods, the method further comprises sequencing the amplification products. In one embodiment of any of the above methods, the amplification is performed under substantially isothermic conditions. In one embodiment of any of the above methods, the nucleic acid polymerase is a DNA polymerase.

In one embodiment of any of the above methods, the DNA polymerase is a strand displacing DNA polymerase. In one embodiment of any of the above methods, the nucleic acid polymerase is selected from bacteriophage phi29 (Φ29) polymerase, genetically modified phi29 (Φ29) DNA polymerase, Klenow Fragment of DNA polymerase I, phage M2 DNA polymerase, phage phiPRD1 DNA polymerase, Bst DNA polymerase, Bst large fragment DNA polymerase, exo(−) Bst polymerase, exo(−)Bca DNA polymerase, Bsu DNA polymerase, Vent_{R} DNA polymerase, Vent_{R} (exo-) DNA polymerase, Deep Vent DNA polymerase, Deep Vent (exo-) DNA polymerase, IsoPol DNA polymerase, DNA polymerase I, Therminator DNA polymerase, T5 DNA polymerase, Sequenase, T7 DNA polymerase, T7-Sequenase, and T4 DNA polymerase. In one embodiment of any of the above methods, the nucleic acid polymerase has 3′->5′ exonuclease activity and the terminator nucleotides inhibit such 3′->5′ exonuclease activity. In one specific embodiment, the terminator nucleotides are selected from nucleotides with modification to the alpha group (e.g., alpha-thio dideoxynucleotides creating a phosphorothioate bond), C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids. In one embodiment of any of the above methods, the nucleic acid polymerase does not have 3′->5′ exonuclease activity. In one specific embodiment, the polymerase is selected from Bst DNA polymerase, exo(−) Bst polymerase, exo(−) Bca DNA polymerase, Bsu DNA polymerase, Vent_{R} (exo-) DNA polymerase, Deep Vent (exo-) DNA polymerase, Klenow Fragment (exo-) DNA polymerase, and Therminator DNA polymerase. In one specific embodiment, the terminator nucleotides comprise modifications of the r group of the 3′ carbon of the deoxyribose. In one specific embodiment, the terminator nucleotides are selected from 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, and combinations thereof. In one specific embodiment, the terminator nucleotides are selected from dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. In one embodiment of any of the above methods, the amplification primers are between 4 and 70 nucleotides long. In one embodiment of any of the above methods, the amplification products are between about 50 and about 2000 nucleotides in length. In one embodiment of any of the above methods, the target nucleic acid is DNA (e.g., a cDNA or a genomic DNA). In one embodiment of any of the above methods, the amplification primers are random primers. In one embodiment of any of the above methods, the amplification primers comprise a barcode. In one specific embodiment, the barcode comprises a cell barcode. In one specific embodiment, the barcode comprises a sample barcode. In one embodiment of any of the above methods, the amplification primers comprise a unique molecular identifier (UMI). In one embodiment of any of the above methods, the method comprises denaturing the target nucleic acid or genomic DNA before the initial primer annealing. In one specific embodiment, denaturation is conducted under alkaline conditions followed by neutralization. In one embodiment of any of the above methods, the sample, the amplification primers, the nucleic acid polymerase, and the mixture of nucleotides are contained in a microfluidic device. In one embodiment of any of the above methods, the sample, the amplification primers, the nucleic acid polymerase, and the mixture of nucleotides are contained in a droplet. In one embodiment of any of the above methods, the sample is selected from tissue(s) samples, cells, biological fluid samples (e.g., blood, urine, saliva, lymphatic fluid, cerebrospinal fluid (CSF), amniotic fluid, pleural fluid, pericardial fluid, ascites, aqueous humor), bone marrow samples, semen samples, biopsy samples, cancer samples, tumor samples, cell lysate samples, forensic samples, archaeological samples, paleontological samples, infection samples, production samples, whole plants, plant parts, microbiota samples, viral preparations, soil samples, marine samples, freshwater samples, household or industrial samples, and combinations and isolates thereof. In one embodiment of any of the above methods, the sample is a cell (e.g., an animal cell [e.g., a human cell], a plant cell, a fungal cell, a bacterial cell, and a protozoal cell). In one specific embodiment, the cell is lysed prior to the replication. In one specific embodiment, cell lysis is accompanied by proteolysis. In one specific embodiment, the cell is selected from a cell from a preimplantation embryo, a stem cell, a fetal cell, a tumor cell, a suspected cancer cell, a cancer cell, a cell subjected to a gene editing procedure, a cell from a pathogenic organism, a cell obtained from a forensic sample, a cell obtained from an archeological sample, and a cell obtained from a paleontological sample. In one embodiment of any of the above methods, the sample is a cell from a preimplantation embryo (e.g., a blastomere [e.g., a blastomere obtained from an eight-cell stage embryo produced by in vitro fertilization]). In one specific embodiment, the method further comprises determining the presence of disease predisposing germline or somatic variants in the embryo cell. In one embodiment of any of the above methods, the sample is a cell from a pathogenic organism (e.g., a bacterium, a fungus, a protozoan). In one specific embodiment, the pathogenic organism cell is obtained from fluid taken from a patient, microbiota sample (e.g., GI microbiota sample, vaginal microbiota sample, skin microbiota sample, etc.) or an indwelling medical device (e.g., an intravenous catheter, a urethral catheter, a cerebrospinal shunt, a prosthetic valve, an artificial joint, an endotracheal tube, etc.). In one specific embodiment, the method further comprises the step of determining the identity of the pathogenic organism. In one specific embodiment, the method further comprises determining the presence of genetic variants responsible for resistance of the pathogenic organism to a treatment. In one embodiment of any of the above methods, the sample is a tumor cell, a suspected cancer cell, or a cancer cell. In one specific embodiment, the method further comprises determining the presence of one or more diagnostic or prognostic mutations. In one specific embodiment, the method further comprises determining the presence of germline or somatic variants responsible for resistance to a treatment. In one embodiment of any of the above methods, the sample is a cell subjected to a gene editing procedure. In one specific embodiment, the method further comprises determining the presence of unplanned mutations caused by the gene editing process. In one embodiment of any of the above methods, the method further comprises determining the history of a cell lineage. In a related aspect, the invention provides a use of any of the above methods for identifying low frequency sequence variants (e.g., variants which constitute ≥0.01% of the total sequences).

In a related aspect, the invention provides a kit comprising a reverse transcriptase, a nucleic acid polymerase, one or more amplification primers, a mixture of nucleotides comprising one or more terminator nucleotides, and optionally instructions for use. In one embodiment of the kits of the invention, the nucleic acid polymerase is a strand displacing DNA polymerase. In some instances, the reverse transcriptase perform template switching. In some instances, the reverse transcriptase is a variant of MMLV (Moloney Murine Leukemia Virus), HIV-1, AMV (avian myeloblastosis virus), telomerase RT, FIV (feline immunodeficiency virus), or XMRV (Xenotropic murine leukemia virus-related virus. Non-limiting examples of reverse transcriptases include SuperScript I (Thermo), SuperScript II (Thermo), SuperScript III (Thermo), SuperScript IV (Thermo), OmniScript (Qiagen), SensiScript (Qiagen), PrimeScript (Takara), Maxima H-(Thermo), AcuuScript Hi-Fi (Agilent), iScript (Bio-Rad), eAMV (Merck KGaA), qScript (Quanta Biosciences), SmartScribe (Clontech), or GoScript (Promega). In some embodiments, a kit comprises dNTPs and uracil. In one embodiment of the kits of the invention, the nucleic acid polymerase is selected from bacteriophage phi29 (29) polymerase, genetically modified phi29 (@29) DNA polymerase, Klenow Fragment of DNA polymerase I, phage M2 DNA polymerase, phage phiPRD1 DNA polymerase, Bst DNA polymerase, Bst large fragment DNA polymerase, exo(−) Bst polymerase, exo(−)Bca DNA polymerase, Bsu DNA polymerase, Vent_{R} DNA polymerase, Vent_{R} (exo-) DNA polymerase, Deep Vent DNA polymerase, Deep Vent (exo-) DNA polymerase, IsoPol DNA polymerase, DNA polymerase I, Therminator DNA polymerase, T5 DNA polymerase, Sequenase, T7 DNA polymerase, T7-Sequenase, and T4 DNA polymerase. In one embodiment of the kits of the invention, the nucleic acid polymerase has 3′->5′ exonuclease activity and the terminator nucleotides inhibit such 3′->5′ exonuclease activity (e.g., nucleotides with modification to the alpha group [e.g., alpha-thio dideoxynucleotides], C3 spacer nucleotides, locked nucleic acids (LNA), inverted nucleic acids, 2′ fluoro nucleotides, 3′ phosphorylated nucleotides, 2′-O-Methyl modified nucleotides, trans nucleic acids). In one embodiment of the kits of the invention, the nucleic acid polymerase does not have 3′->5′ exonuclease activity (e.g., Bst DNA polymerase, exo(−) Bst polymerase, exo(−) Bca DNA polymerase, Bsu DNA polymerase, Vent_{R} (exo-) DNA polymerase, Deep Vent (exo-) DNA polymerase, Klenow Fragment (exo-) DNA polymerase, Therminator DNA polymerase). In one specific embodiment, the terminator nucleotides comprise modifications of the r group of the 3′ carbon of the deoxyribose. In one specific embodiment, the terminator nucleotides are selected from 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, and combinations thereof. In one specific embodiment, the terminator nucleotides are selected from dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. In some instances, a kit comprises at least one enzyme stabilizer, neutralization buffer, denaturing buffer, or combination thereof. In some instances, a kit comprises one or more modules. In some instances, a kit comprises a genome module and a transcriptome module.

Methods described herein (e.g., PTA multiomics) may comprise chromatin analysis. In some instances, chromatin analysis comprises analysis of chromatin accessibility (mapping). In some instances, chromatin analysis comprises ATAC, mChIP, ChiP-MS, ChroP, HiC, or other chromatin analysis method. In some instances, methods of measuring chromatin accessibility comprise use of transposes such as Tn5 (See, Buenrostro et al., Curr Protoc Mol Biol. 2015; 109:21.29.1-21.29-9. In some instances, chromatin-bound genomic DNA is treated with a transposase to generate fragments. In some instances, PTA amplification is conducted on transposase fragmented genomic DNA. Such methods in some instances combined with other multiomic analysis such as transcriptome, methylome, proteome, or other technique described herein. In some instances, chromatin analysis comprises crosslinking (e.g., formaldehyde) of chromatin-bound genomic DNA prior to fragmentation with transposes or other fragmentation method (e.g., sonication, digestion).

### EXAMPLES

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### Example 1: Design and Execution of a Multiomics Workflow

### Overview

Discovering genomic variation in the absence of information about transcriptional consequence of that variation or, conversely, a transcriptional signature without understanding underlying genomic contributions, hinders understanding of molecular mechanisms of disease. To assess this genomic and transcriptomic coordination, a multiomics method was developed to extract this information out of the individual cell. The workflow unifies template-switching full-transcript RNA-Seq chemistry and whole genome amplification (WGA), followed by affinity purification of first-strand cDNA and subsequent separation of the RNA/DNA fractions for sequencing library preparation. In the multiomics methodology the attributes of primary template-directed amplification (PTA) are leveraged to enable accurate assessment of single-nucleotide variation as a DNA feature—which is not achieved with other workflows to assess DNA+RNA information in the same cell.

A single-well integration of single-cell transcriptome and genome amplification where a standard PTA reaction was modified to include a reverse transcription (RT) step prior to single-cell genome amplification was designed and executed, and designated as multiomic enrichment (ResolveOME, Bioskryb Genomics, Inc.). In this workflow, PTA amplifies the genomes of single cells immediately after the RT reaction is concluded in a single-well reaction. Using template switch-based reverse transcription, barcoded first-strand cDNA molecules were created that were affinity purified and pre-amplified prior to RNA-Seq sequencing library creation. The net result from the combined amplification reaction was a biotin labeled cDNA pool derived primarily from the cytosolic transcripts, available for streptavidin purification, and a pool of amplified genomic material from the single cell. In alternative embodiments, magnetic beads with attached RT primers can be used for direct removal of the cDNA amplicon library. At the conclusion of the genome amplification reaction the cDNA fraction is separated from the amplified genome material whereby libraries from each pool were created. The resulting sequencing data offered the ability to define both genomic and transcriptomic plasticity at single-cell resolution. Specifically, the delineation of isoform expression, combined with ability to annotate the underlying structural variation and single nucleotide changes from the genome of the same cell (FIG. 1A), allowed the assessment of genomic “penetrance”, and the definition of mechanisms that drive single-cell fate.

Prior multiomic efforts pioneered the pairing of genomic and transcriptomic information from the same single cell but have the primary shortcoming of incomplete genome coverage and associated non-uniformity of coverage-leaving uncovered genomic valleys that may harbor deleterious single nucleotide variants that would remain undetected. Indeed, multiple displacement amplification (MDA) drives the genomic amplification of G&T-seq and DR-Seq has genomic amplification uniformity comparable to that of MALBAC, both of which are outperformed by PTA in terms of genomic coverage, allelic balance and SNV calling metrics. In one example, definition of clonal evolution at the SNV/CNV level in a primary patient sample was accomplished utilizing G&T-seq, yet was limited to a candidate gene survey of exome-level data whereby clusters where defined by 59 oncogenes and another studying employing G&T-seq limited their analysis to the RNA workflow of the method to take advantage of the low input requirement, without assessment of genomic level data. Thus, addressed herein is an unmet need to add genome-wide, high sensitivity and high precision SNV calling capability to a joint DNA/RNA single-cell methodology. Further, the importance of these measurements is demonstrated, whereby single nucleotide variation fundamentally affects cell state and tumor progression.

Provided herein are the utility of these unified “-omic” layers, highlighting heterogenous genomic variation and consequential phenotypic alterations in single cells that both are correlated with the development of resistance to a targeted therapeutic in a cell line model of acute myeloid leukemia, and in oncogenic mechanisms in primary breast cancer cells whereby the insights gained could not be inferred by a single dataset (genome or transcriptome) alone.

### Amplification Product Yield of RNA+DNA Multiomics Workflow

Prior to demonstrating biological utility of the multiomics method described herein, in a cell line drug resistance model and in a primary patient sample, the technical performance of the methodology using a benchmark cell line 1000 Genomes cell line, NA12878 was examined. The RNA and DNA arms of the protocol were first assessed using metrics from the template-switching RNA-Seq chemistry or PTA chemistry in isolation to compare to the metrics when the chemistries were unified in the combined multiomics protocol.

Multiomics data with FACS-sorted NA12878 single cells was generated with purified total NA12878 RNA or genomic DNA as amplification controls using the workflow shown in FIG. 1A. Efficiency of the yield of the PTA product and cDNA products from the unified protocol are shown in FIG. 1B. Approximately 1-1.5 μg of DNA amplification product from single cell genomes and approximately 100-200 ng of cDNA product representing the single cell transcriptome was obtained. Importantly, no-template control (NTC) reactions showed lack of detectable product and additionally there was negligible (<50 ng) yield in the DNA fraction from control RNA input using Qubit fluorometer (ThermoFisher). Low-level background amplification of the genomic DNA control input in the cDNA fraction was observed, due to known promiscuity of reverse transcriptase in the absence of mRNA template. By contrast, this background amplification does not occur in reactions with single cells as the genome material is sequestered in the non-lysed nucleus during the reverse transcription workflow of multiomics.

### PTA Modifications

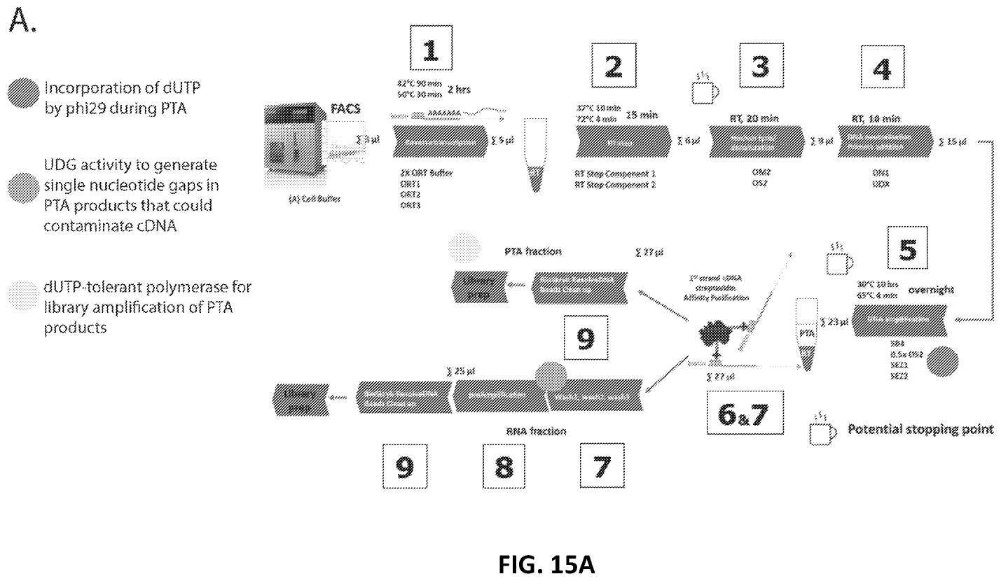

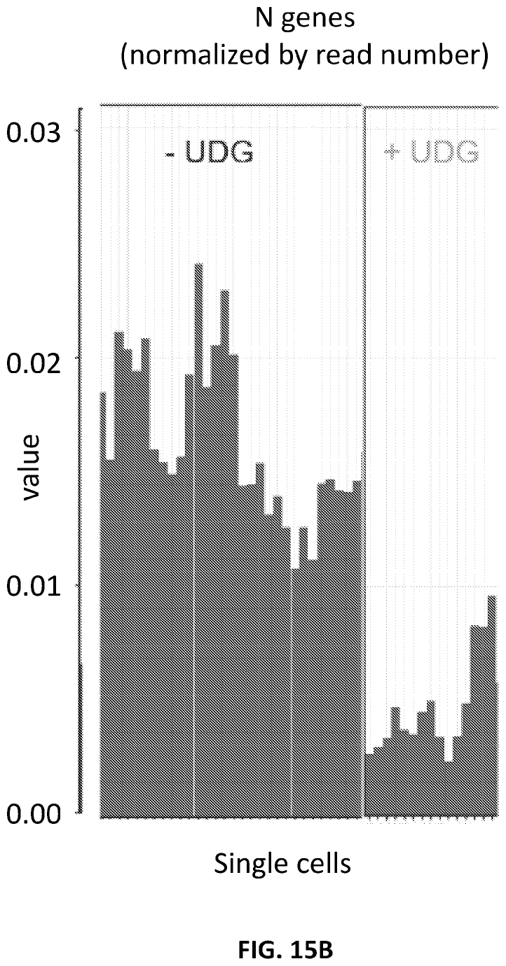

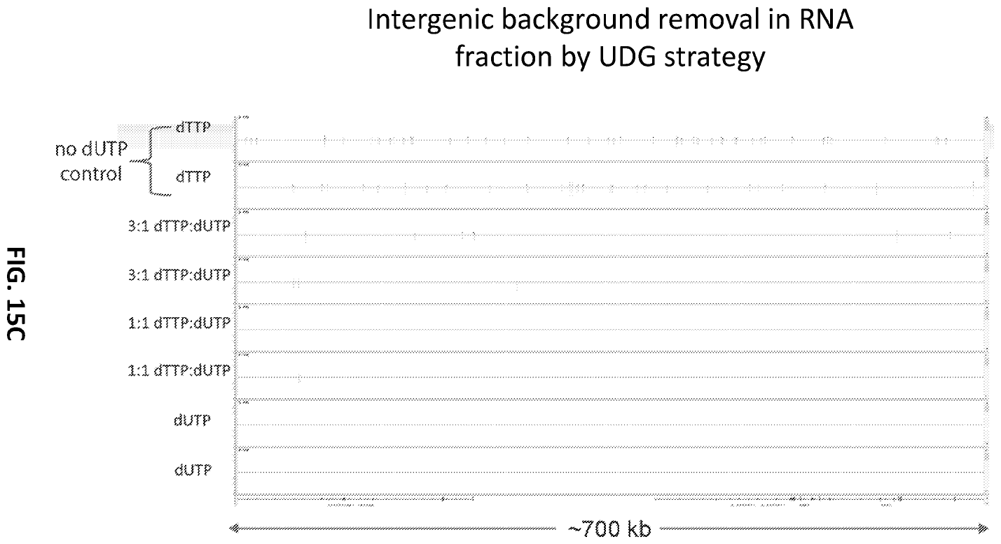

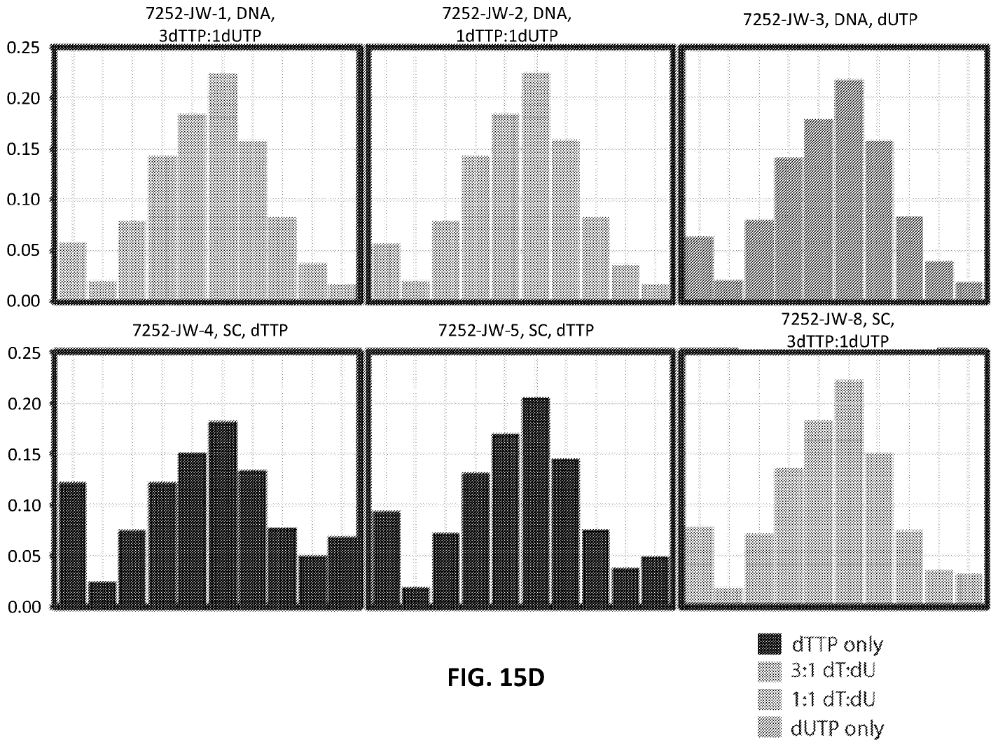

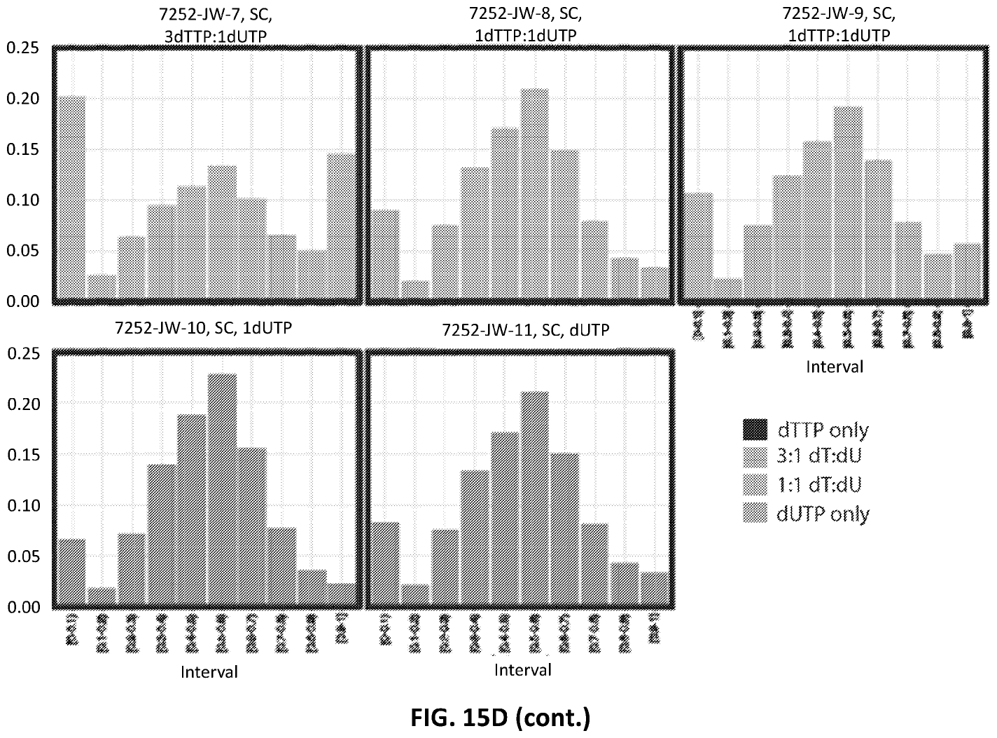

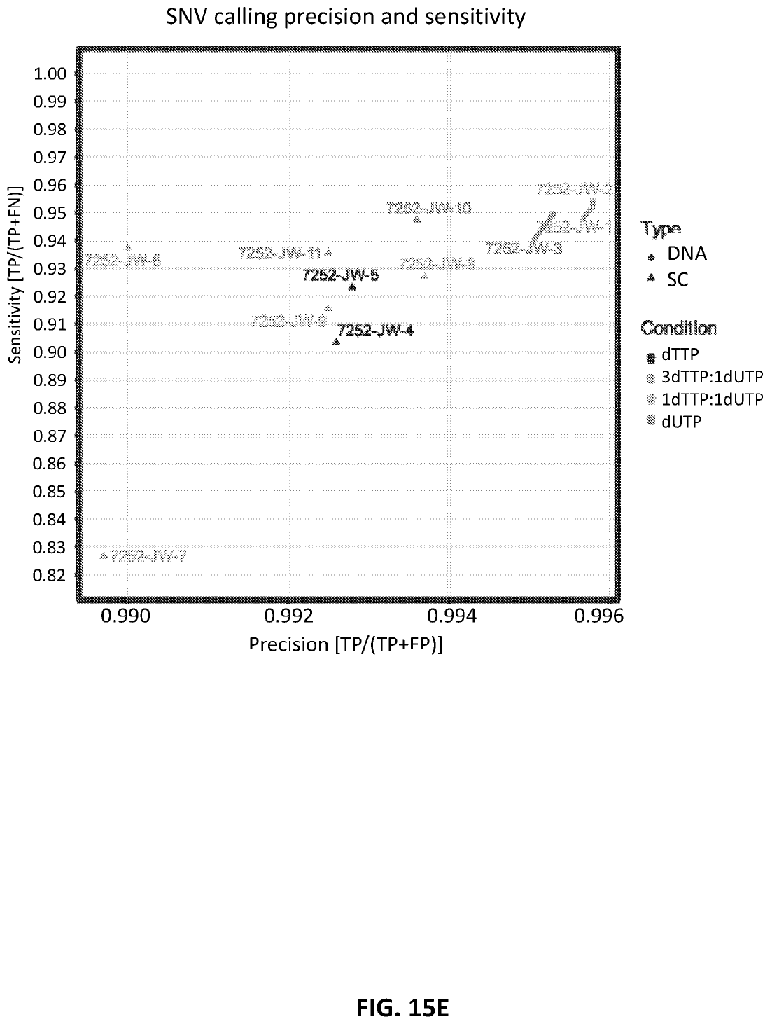

The PTA method was modified for use in a multiomics workflow (FIGS. 15A-15D). After reverse transcription has completed, dUTP was added to the normal nucleotide mix (dATP, dCTP, dGTP, dTTP) during phi29 amplification (red dot), resulting in PTA amplification products derived from the original single-cell or low-input template DNA being “marked” with dUTP (FIG. 15A). A UDG incubation step occurred on beads after affinity purification and washes of the cDNA, to digest the background dUTP-marked PTA product prior to preamplification of the cDNA (green dot). For library preparation, the cDNA libraries utilized a normal high-fidelity polymerase, however, the PTA-derived libraries representing the DNA arm of the multiomics workflow used a uracil tolerant polymerase in order to amplify the library ligation products of uracil-containing PTA product (yellow dot). The number of expressed genes detected was reduced following UDG treatment; indicating that transcript counts in the absence of UDG treatment were likely compounded by DNA (PTA) background. IGV visualization (700 kb region, harboring 3 genes) of intergenic read background removal upon UDG scheme (FIG. 15C). Each row was a single-cell (NA12878) Multiomic RNA fraction library. DNA background reads was seen in the top two control RNA libraries when PTA was performed lacking dUTP, and these background reads progressively diminished as more dUTP is included during PTA. The ratio of nucleotides was 1:1 dUTP:dTTP; PTA reactions containing dUTP exclusively with no dTTP were slower kinetically. The DNA background removal benefits of increased dUTP in the PTA reaction (C) did not adversely affect allelic balance (FIG. 15D) and SNV calling precision and sensitivity metrics (FIG. 15E). Reagents may be used with the methods and compositions described herein to identify

Some polymerases stall or have reduced efficiency when amplifying templates comprising uracil. Uracil tolerant polymerases may be used with the methods described herein to amplify uracil-containing templates (e.g., with PTA). In some instances, a uracil tolerant polymerase maintains at least 50, 60, 70, 80, 85, 90, 95, 97, or 99% polymerase activity when amplifying a template comprising uracil as compared to a template without uracil. In some instances a uracil tolerant polymerase is derived from archaea, yeast, or bacterial species. In some instances a uracil tolerant polymerase comprises DNA polymerases ¿ and 8 from S. cerevisiae, and E. coli DNA polymerase III, PolA-type polymerases such as Taq, KAPA HiFi Uracil+DNA Polymerase (Kapa biosystems, Q5U), KOD Multi & Epi DNA Polymerase, FastStart Taq (Roche), Taq2000 (Agilent Technologies), FailSafe Enzyme (Epicentre) or Thermo PhusionU. In some instances, a uracil tolerant polymerase comprises a sequence having at least 70%, 75%, 80%, 85%, 90%, 95%, 97%, or 99% identity with DNA polymerases & and 8 from S. cerevisiae, and E. coli DNA polymerase III, PolA-type polymerases such as Taq, KAPA HiFi Uracil+DNA Polymerase (Kapa biosystems, Q5U), KOD Multi & Epi DNA Polymerase, FastStart Taq (Roche), Taq2000 (Agilent Technologies), FailSafe Enzyme (Epicentre) or Thermo PhusionU. In some instance a uracil tolerant polymerase comprises a modification to one or more amino acid residues in the dUTP binding pocket.

### Comparative Genomic Performance of Multiomics Workflow

As default practice prior to passing single cell samples to deep sequencing for SNV analysis low-pass QC sequencing was performed, and as part of the analysis pipeline, an estimation of library complexity with the PreSeq count algorithm determined. QC standards set for genomic DNA only (product solution for PTA) are >3.0E9 PreSeq count value upon low-pass sequencing, an empirically-defined proxy for genomic coverage and uniformity that predicts high-depth sequencing will yield strong allelic balance and high sensitivity and precision of single nucleotide variant calling. The average PreSeq count of single cells from Table 1A was 3.76E9 with a standard deviation of +/−2.27E8. The overall robust performance of single cells and genomic DNA controls warranted subsequent deep sequencing for metric comparison of classical PTA to PTA from the multiomic workflow.

TABLE 1A

| | | % | % | % | % | Insert | |
|---|---|---|---|---|---|---|---|
| Sample Name | PreSeq Count | chrM | Chimeras | Aligned | Error | Size | Total Reads |
| MOLM13-DNA- | 3,378,959,196 | 1.41 | 10.55 | 99.89 | 0.81 | 209 | 533,283,408 |
| P_SC10 | | | | | | | |
| MOLM13-DNA- | 3,047,245,546 | 1.22 | 13.93 | 99.82 | 0.91 | 271 | 547,092,936 |
| P_SC11 | | | | | | | |
| MOLM13-DNA- | 3,478,446,000 | 0.86 | 12.35 | 99.85 | 0.84 | 244 | 480,104,446 |
| R_SC12 | | | | | | | |
| MOLM13-DNA- | 3,654,038,904 | 0.62 | 13.6 | 99.83 | 0.88 | 271 | 385,229,788 |
| R_SC13 | | | | | | | |
| MOLM13-DNA- | 3,254,232,372 | 1.32 | 13.77 | 99.84 | 0.88 | 271 | 573,459,994 |
| R_SC14 | | | | | | | |
| MOLM13-DNA- | 3,542,833,429 | 1.27 | 13.51 | 99.84 | 0.88 | 270 | 507,961,822 |
| R_SC15 | | | | | | | |
| MOLM13-DNA- | 3,632,455,433 | 1.31 | 13.53 | 99.84 | 0.87 | 268 | 447,380,990 |
| R_SC16 | | | | | | | |
| MOLM13-DNA- | 3,654,615,714 | 0.74 | 12.93 | 99.84 | 0.87 | 268 | 377,431,838 |
| R_SC17 | | | | | | | |
| MOLM13-DNA- | 3,367,781,398 | 1.33 | 14 | 99.84 | 0.86 | 271 | 487,173,864 |
| R_SC18 | | | | | | | |
| MOLM13-DNA- | 3,727,151,763 | 1.24 | 14.07 | 99.82 | 0.91 | 271 | 362,465,154 |
| R_SC19 | | | | | | | |
| MOLM13-DNA- | 3,679,483,372 | 0.84 | 12.59 | 99.81 | 0.88 | 270 | 509,963,664 |
| P_SC20 | | | | | | | |
| MOLM13-DNA- | 3,756,601,103 | 1.12 | 13.31 | 99.79 | 0.94 | 271 | 426,871,548 |
| R_SC21 | | | | | | | |
| MOLM13-DNA- | 3,643,238,259 | 0.95 | 12.69 | 99.82 | 0.87 | 266 | 510,327,482 |
| R_SC22 | | | | | | | |
| MOLM13-DNA- | 3,654,228,480 | 0.98 | 12.39 | 99.76 | 0.92 | 271 | 472,760,368 |
| P_SC23 | | | | | | | |
| MOLM13-DNA- | 3,716,521,996 | 0.74 | 12.16 | 99.81 | 0.87 | 271 | 448,199,656 |
| P_SC24 | | | | | | | |
| MOLM13-DNA- | 3,721,188,640 | 0.81 | 12.25 | 99.8 | 0.88 | 271 | 475,216,948 |
| P_SC25 | | | | | | | |
| MOLM13-DNA- | 3,561,816,152 | 0.91 | 12.36 | 99.8 | 0.87 | 260 | 373,985,162 |
| P_SC26 | | | | | | | |
| MOLM13-DNA- | 3,612,063,548 | 1.21 | 12.62 | 99.8 | 0.91 | 270 | 481,944,292 |
| P_SC27 | | | | | | | |
| MOLM13-DNA- | 3,663,289,670 | 0.73 | 11.79 | 99.8 | 0.87 | 237 | 439,568,964 |
| P_SC28 | | | | | | | |
| MOLM13-DNA- | 3,689,642,274 | 1.83 | 12.84 | 99.85 | 0.87 | 271 | 444,353,302 |
| P_SC29 | | | | | | | |
| DCIS1a-DNA- | 3,798,856,998 | 1.27 | 11.65 | 99.84 | 0.85 | 234 | 396,346,406 |
| EpCAM-H_SC10 | | | | | | | |
| DCIS1a-DNA- | 3,903,824,812 | 1.27 | 11.96 | 99.85 | 0.84 | 271 | 361,861,448 |
| EpCAM-H_SC11 | | | | | | | |
| DCIS1a-DNA- | 3,853,848,682 | 0.93 | 11.98 | 99.83 | 0.87 | 268 | 344,868,038 |
| EpCAM-H_SC16 | | | | | | | |
| DCIS1a-DNA- | 3,826,217,668 | 0.76 | 11.9 | 99.84 | 0.84 | 271 | 587,467,028 |
| EpCAM-H_SC3 | | | | | | | |
| DCIS1a-DNA- | 3,860,673,146 | 0.6 | 10.86 | 99.84 | 0.86 | 267 | 491,703,926 |
| EpCAM-H_SC4 | | | | | | | |
| DCIS1a-DNA- | 3,823,230,915 | 0.65 | 11.37 | 99.85 | 0.85 | 271 | 517,419,546 |
| EpCAM-H_SC5 | | | | | | | |
| DCIS1a-DNA- | 4,041,611,753 | 0.77 | 11.04 | 99.84 | 0.87 | 271 | 457,263,284 |
| EpCAM-H_SC6 | | | | | | | |
| DCIS1a-DNA- | 3,872,528,492 | 0.38 | 11.02 | 99.83 | 0.87 | 266 | 425,639,760 |
| EpCAM-H_SC7 | | | | | | | |
| DCIS1a-DNA- | 3,742,075,389 | 0.46 | 10.45 | 99.87 | 0.83 | 242 | 497,436,780 |
| EpCAM-H_SC8 | | | | | | | |
| DCIS1a-DNA- | 2,669,576,486 | 1.41 | 11.64 | 99.84 | 0.86 | 248 | 472,307,222 |
| EpCAM-H_SC9 | | | | | | | |
| DCIS1a-DNA- | 3,883,582,122 | 0.26 | 11.14 | 99.84 | 0.85 | 237 | 526,130,674 |
| EpCAM-L_SC3 | | | | | | | |
| DCIS1a-DNA- | 3,903,158,379 | 0.18 | 11.71 | 99.87 | 0.82 | 231 | 405,982,650 |
| EpCAM-L_SC8 | | | | | | | |
| NA12878a-DNA_SC10 | 3,804,223,358 | 0.5 | 11.17 | 99.83 | 0.86 | 270 | 341,176,288 |
| NA12878a-DNA_SC11 | 3,879,433,072 | 0.54 | 11.18 | 99.85 | 0.84 | 271 | 391,778,760 |
| NA12878a-DNA_SC12 | 3,851,844,096 | 0.42 | 11.54 | 99.84 | 0.85 | 271 | 381,523,468 |
| NA12878a-DNA_SC13 | 3,896,350,430 | 0.53 | 10.55 | 99.83 | 0.86 | 270 | 488,312,070 |
| NA12878a-DNA_SC16 | 3,754,881,507 | 0.63 | 11.12 | 99.83 | 0.87 | 270 | 476,517,794 |
| NA12878a-DNA_SC2 | 3,696,174,079 | 0.6 | 11.5 | 99.8 | 0.88 | 270 | 404,730,258 |
| NA12878a-DNA_SC3 | 3,600,119,598 | 0.4 | 11.24 | 99.81 | 0.89 | 271 | 384,398,982 |
| NA12878a-DNA_SC4 | 3,838,000,444 | 0.86 | 11.52 | 99.84 | 0.84 | 271 | 459,405,210 |
| NA12878a-DNA_SC5 | 3,855,303,158 | 0.51 | 11.46 | 99.85 | 0.83 | 271 | 464,258,980 |
| NA12878a-DNA_SC9 | 3,653,170,378 | 0.54 | 11.02 | 99.86 | 0.83 | 265 | 454,950,012 |
| DCIS1b-EpCAM- | 3,858,356,596 | 0.43 | 12.6 | 99.86 | 0.82 | 270 | 1,002,712,220 |
| H_SC1 | | | | | | | |
| DCIS1b-EpCAM- | 3,821,814,480 | 0.77 | 11.54 | 99.85 | 0.81 | 233 | 795,376,626 |
| H_SC10 | | | | | | | |
| DCIS1b-EpCAM- | 3,793,373,465 | 0.86 | 11.93 | 99.86 | 0.8 | 265 | 919,124,034 |
| H_SC11 | | | | | | | |
| DCIS1b-EpCAM- | 3,907,483,851 | 0.32 | 11.51 | 99.85 | 0.81 | 240 | 878,459,470 |
| H_SC12 | | | | | | | |
| DCIS1b-EpCAM- | 3,822,942,159 | 0.62 | 11.63 | 99.84 | 0.82 | 237 | 1,034,254,730 |
| H_SC13 | | | | | | | |
| DCIS1b-EpCAM- | 3,825,779,695 | 0.57 | 11.6 | 99.82 | 0.9 | 271 | 204,956,332 |
| H_SC15 | | | | | | | |
| DCIS1b-EpCAM- | 3,781,245,576 | 0.95 | 10.43 | 99.86 | 0.81 | 216 | 1,125,854,978 |
| H_SC16 | | | | | | | |
| DCIS1b-EpCAM- | 3,834,539,384 | 1.01 | 12.68 | 99.84 | 0.82 | 271 | 865,697,460 |
| H_SC4 | | | | | | | |
| DCIS1b-EpCAM- | 3,916,117,911 | 1.07 | 12.47 | 99.86 | 0.83 | 270 | 1,008,802,612 |
| H_SC5 | | | | | | | |
| DCIS1b-EpCAM- | 3,815,192,273 | 1.21 | 11.6 | 99.79 | 0.91 | 239 | 263,486,188 |
| H_SC6 | | | | | | | |
| DCIS1b-EpCAM- | 3,894,130,144 | 1.13 | 11.67 | 99.81 | 0.93 | 271 | 237,920,488 |
| H_SC7 | | | | | | | |
| DCIS1b-EpCAM- | 3,906,070,366 | 0.84 | 12.86 | 99.88 | 0.82 | 270 | 803,195,790 |
| H_SC9 | | | | | | | |
| NA12878b-DNA_SC1 | 3,957,877,510 | 0.34 | 11.55 | 99.8 | 0.86 | 270 | 476,648,274 |
| NA12878b-DNA_SC10 | 3,930,104,125 | 0.34 | 11.58 | 99.85 | 0.83 | 238 | 416,467,862 |
| NA12878b-DNA_SC11 | 3,979,659,349 | 0.44 | 11.68 | 99.82 | 0.85 | 267 | 443,612,012 |
| NA12878b-DNA_SC12 | 3,970,854,286 | 0.65 | 11.69 | 99.84 | 0.85 | 238 | 473,077,904 |
| NA12878b-DNA_SC15 | 3,957,961,760 | 0.69 | 11.53 | 99.85 | 0.82 | 239 | 607,659,734 |
| NA12878b-DNA_SC2 | 3,965,896,761 | 0.53 | 11.55 | 99.82 | 0.84 | 243 | 544,683,908 |
| NA12878b-DNA_SC3 | 3,910,878,320 | 0.2 | 11.35 | 99.82 | 0.85 | 271 | 435,263,252 |
| NA12878b-DNA_SC4 | 3,927,002,572 | 0.68 | 11.78 | 99.83 | 0.83 | 268 | 428,068,212 |
| NA12878b-DNA_SC7 | 3,944,483,107 | 0.49 | 11.32 | 99.83 | 0.83 | 270 | 325,712,176 |
| NA12878b-DNA_SC9 | 4,027,651,804 | 0.4 | 11.92 | 99.83 | 0.85 | 270 | 250,082,914 |
| DCIS1c-EpCAM- | 3,818,963,492 | 0.23 | 11.68 | 99.85 | 0.81 | 271 | 1,082,729,250 |
| L_SC2 | | | | | | | |
| DCIS1c-EpCAM- | 3,909,620,601 | 0.21 | 11.87 | 99.83 | 0.84 | 266 | 1,319,911,956 |
| L_SC8 | | | | | | | |

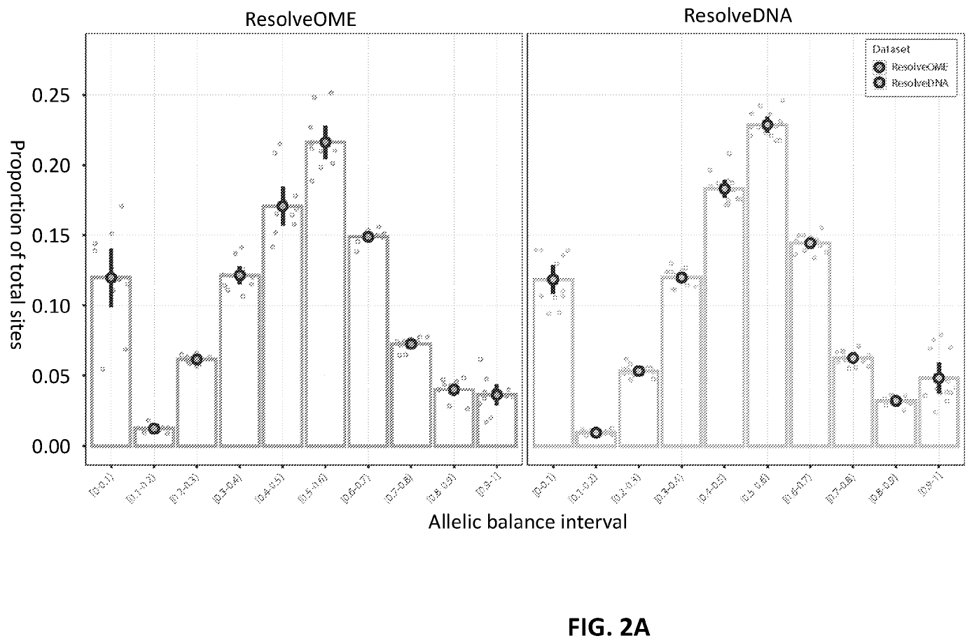

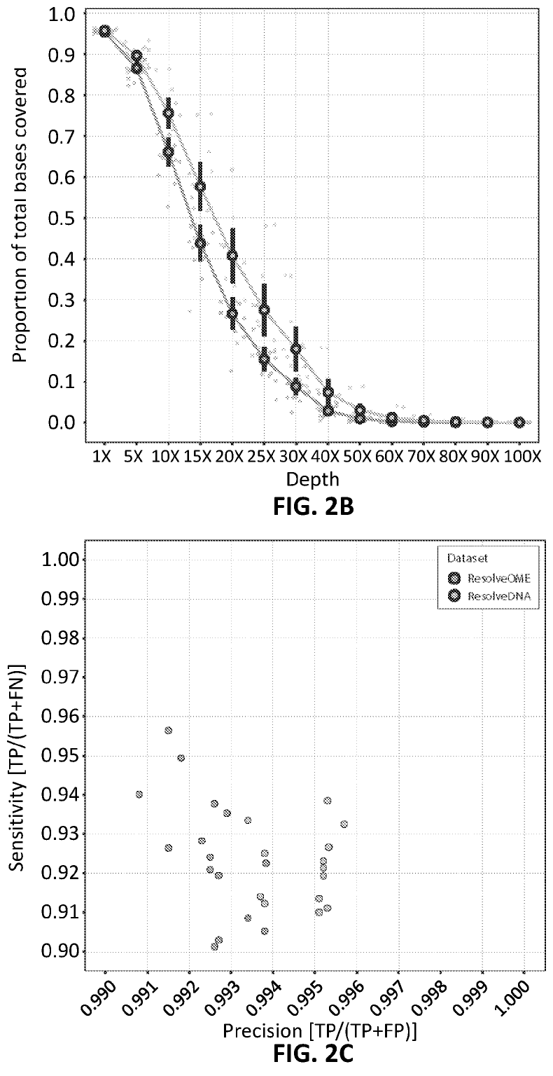

Upon high-depth sequencing (2×150 bp, down-sampling to 4.5E8 total reads, ˜20× genome depth) and processing through our pipeline, allelic balance was reviewed, (ability to represent both alleles through enrichment and a strength of genomic PTA methodology). The inverse of allelic drop out (ADO) is allelic balance, which is the proportion of known heterozygous loci that are called heterozygous following sequencing. Variants within these loci have allele frequencies between 10% and 90% at each locus. A review of allelic balance of the multiomics workflow showed 85.5% (+/−3.4%), which is closely comparable to the 88.2% (+/−4%) for genomic DNA only workflow, across 10 replicates each (FIG. 2A). Genomic coverage at a range of depths did not significantly differ (FIG. 2B) between the workflows. Lastly, it was critical to demonstrate that the allelic balance and coverage obtained from the multiomics workflow culminated in the ability to call SNVs with confidence. FIG. 2C highlights individual multiomics NA12878 cells with a SNV calling sensitivity range of 0.90-0.95 and with precision >0.99, akin to genomic DNA-only data. Collectively, these data suggest that, despite the upstream reverse transcription chemistry modifications to generate transcriptome data, amplification performance of single-cell genomes by PTA persists in performance.

### Comparative Transcriptomic Performance of Multiomies Workflow

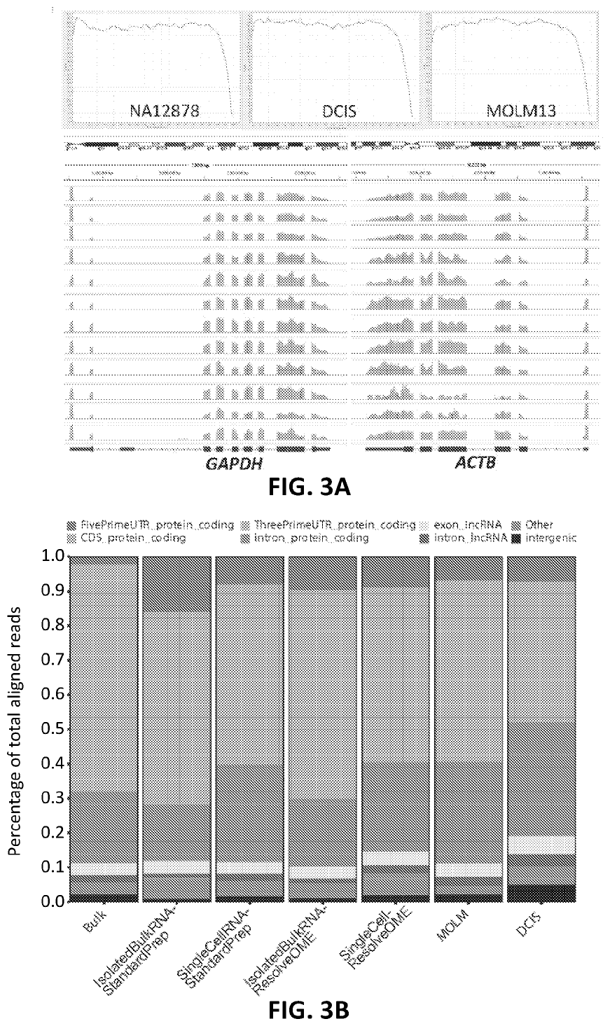

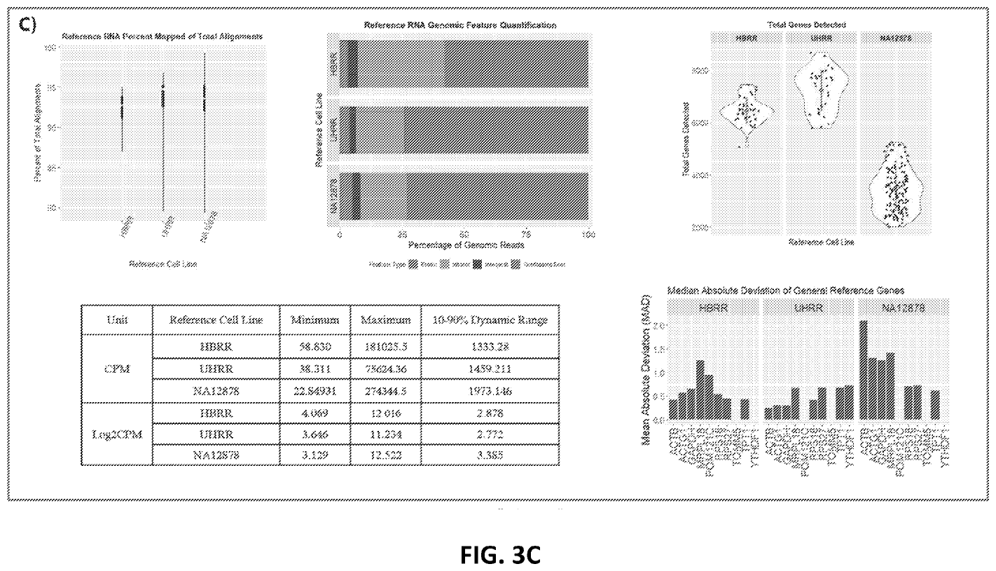

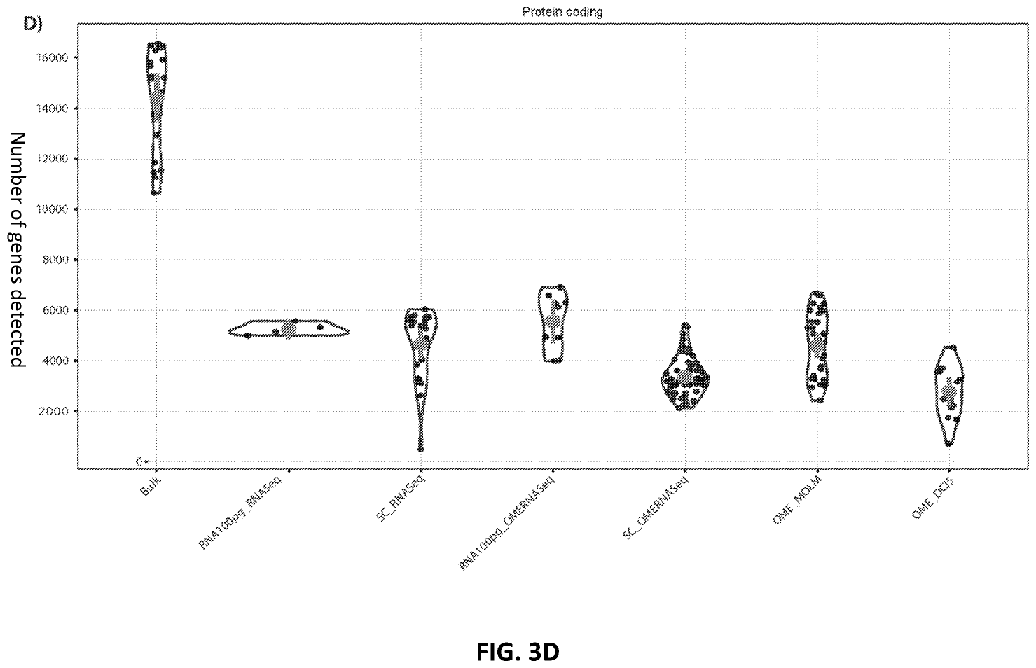

In choosing a transcriptomic scheme to unite with PTA one goal was to be as comprehensive as possible in capturing the diversity of RNA-based modes of oncogenic and drug resistance mechanisms, and, equally as importantly, to enable the ascertainment of genomic lesions manifesting at the RNA level. A template-switching reverse transcription scheme was designed for the multiomics workflow that captured full-transcript information as opposed to either 5′ or 3′ end counting to enhance ability to detect isoforms and identify fusions. This chemistry enables even coverage across transcripts and as shown in FIG. 3A, where increased coverage of the 5′ region (top) which typically is affected by degradation (or reverse transcriptase performance) proportional to the distance from 3′-polyA, is shown. This confirms behavior of the template-switching chemistry in the RNA arm workflow. The distribution of read depth across gene bodies of a set of housekeeping genes is presented in FIG. 3A (bottom), with all exons equally represented. Feature quantification in the across our defined transcriptome is shown in FIG. 3B, highlighting the ability to identify a variety of transcript bodies. Progression of the performance is shown in this figure from what is observed in a bulk dataset (bar 1, aggregated datasets) vs. features such as bulk isolation (bars 2 and 4) against library prep methods: standalone mRNA-stranded (bars 2 and 3) and multiomics combined library prep (bars 4 and 5). Most notably, increased 5′ coding and intronic regions in the multiomics chemistry was observed overall, with intergenic background routinely below 5% of aligned reads, providing a broader space for isoform detection.

As further performance benchmarking of cell quality post mapping to reference transcriptome, performance patterns were established of common metrics with well characterized Human Brain Reference RNA (HBRR) and Universal Human Reference RNA (UHRR) as additions to the NA12878 cell line and displayed composite features in FIG. 3C. Read and genomic feature mapping percentages were identified, as well as total genes discovered as criterion for evaluating sequencing quality. The dynamic range of expression and expression patterns in well-known housekeeping genes was also examined, and various markers of DNA contamination, sample degradation, and/or bias as a percentage of exonic (more than 55%), and intergenic mapping (less than 5%) as characteristics of the multiomics RNA fraction were computed. Another important metric for measuring the quality of single cell experiments was the number of genes found (>0 counts) per cell. For NA12878 cells there was an average of approximately 2500, whereas the average number of HBRR and UHRR genes discovered was around 6 and 7 thousand, respectively. Lastly, median absolute deviation (MAD) and percent coefficient of variation (CV) scores were calculated on normalized CPM values for general use housekeeping genes for cross-tissue studies. These metrics measure reproducibility and are robust approaches to measuring sample variability. Overall, comparable monotonous expression metrics across housekeeping genes of examined, as well as MAD values ranging from 0.25 to 1 for our HBRR and UHRR benchmarks were observed, suggesting these genes exhibit little variability in expression across cells. NA12878, demonstrated slightly more irregularity, which without being bound by theory may imply higher variability or unsuitable housekeeping genes. Correspondingly, CV rates varied from 14 to 30 percent, despite NA12878 exhibiting more variation. For each cell, the dynamic range of expressed genes was around 1300 (HBRR), 1400 (UHRR), and 1900 (NA12878) CPM.

FIG. 3D shows multiomics full-transcript performance vs. an amalgam of publicly-available bulk RNA-Seq and 3′ end-counting datasets (See Methods), highlighting the increased 5′ UTR and gene body coverage that occurs by definition relative to 3′ end-counting. The relative types of other RNA species detected with the multiomics chemistry, including lncRNAs, snRNAs, and pseudogenes are shown. Relative proportions of features were concordant between the template-switching RT chemistry in isolation vs. in the combined RNA/DNA workflow in multiomics, and overall concordance was observed between purified RNA input template vs. single cells, with the exception that single cells revealed more intronic reads of protein coding genes than did the purified RNA input. In all single cells analyzed in Tables 1B-1 and 1B-2, mitochondrial read percentage was <10%, with most cells averaging less than 5%, indicating that single-cell lysis was optimal for capturing mRNA and other polyadenylated transcripts and that the amplified cells were healthy.

TABLE 1B-1

| bulk_{—} | | | total. | reads. | total. | | | | overlapping. |
|---|---|---|---|---|---|---|---|---|---|
| sc | molecule | cell_line | input | aligned | alignments | exonic | intronic | intergenic | exon |
| bulk | RNA | NA12878 | 111982 | 106846 | 141318 | 67871 | 22726 | 2728 | 3643 |
| bulk | RNA | NA12878 | 160782 | 142094 | 154588 | 18529 | 73521 | 43815 | 3024 |
| bulk | RNA | NA12878 | 140426 | 133910 | 182310 | 10526 | 7405 | 988 | 3999 |
| bulk | RNA | NA12878 | 129376 | 124326 | 160948 | 98690 | 6704 | 831 | 3884 |
| bulk | RNA | NA12878 | 136722 | 131808 | 169050 | 105443 | 6197 | 961 | 4125 |
| bulk | RNA | NA12878 | 135112 | 130128 | 166204 | 103493 | 6913 | 913 | 4057 |
| sc | RNA | NA12878 | 122118 | 114636 | 149060 | 78748 | 18269 | 3057 | 3871 |
| sc | RNA | NA12878 | 121338 | 117168 | 151682 | 83646 | 16463 | 1883 | 3983 |
| sc | RNA | NA12878 | 118964 | 112087 | 152697 | 76246 | 16658 | 2685 | 3604 |
| sc | RNA | NA12878 | 110644 | 106639 | 136866 | 77158 | 14263 | 1407 | 3578 |
| sc | RNA | NA12878 | 143912 | 136541 | 169835 | 99176 | 17745 | 2444 | 4568 |
| sc | RNA | NA12878 | 121504 | 115879 | 150802 | 74453 | 24615 | 3135 | 3931 |
| sc | RNA | NA12878 | 144626 | 136420 | 165574 | 81838 | 31608 | 8792 | 4293 |
| sc | RNA | NA12878 | 130496 | 123579 | 151006 | 85721 | 19493 | 3093 | 4249 |
| sc | RNA | NA12878 | 118214 | 113371 | 144518 | 81924 | 14857 | 2147 | 3597 |
| sc | RNA | NA12878 | 129322 | 124358 | 163340 | 88167 | 18044 | 2007 | 4039 |
| sc | RNA | NA12878 | 132124 | 125034 | 149716 | 78679 | 24883 | 8253 | 4004 |
| sc | RNA | NA12878 | 128216 | 122825 | 156627 | 83410 | 21313 | 2815 | 4071 |
| sc | RNA | NA12878 | 124238 | 115933 | 146241 | 56731 | 35040 | 11854 | 3145 |
| sc | RNA | NA12878 | 112982 | 108011 | 151101 | 70197 | 19180 | 3443 | 3637 |
| sc | RNA | NA12878 | 128944 | 120676 | 161147 | 71217 | 25221 | 8907 | 3526 |
| sc | RNA | NA12878 | 148522 | 140398 | 188764 | 96096 | 22268 | 2912 | 4718 |
| sc | RNA | HBRR | 142396 | 133160 | 189868 | 72659 | 40530 | 4206 | 3976 |
| sc | RNA | HBRR | 140702 | 133160 | 165570 | 78957 | 37160 | 4146 | 4286 |
| sc | RNA | HBRR | 142816 | 134463 | 192603 | 72971 | 41155 | 4073 | 3988 |
| sc | RNA | HBRR | 142896 | 134357 | 183723 | 73477 | 41903 | 4159 | 4032 |
| sc | RNA | HBRR | 148176 | 138925 | 192628 | 77596 | 41405 | 4078 | 4230 |
| sc | RNA | HBRR | 148120 | 139804 | 192584 | 74651 | 45370 | 4269 | 3990 |
| sc | RNA | UHRR | 153988 | 146990 | 179179 | 101870 | 25508 | 2753 | 4746 |
| sc | RNA | UHRR | 158422 | 151785 | 178137 | 100862 | 32054 | 3232 | 4797 |
| sc | RNA | UHRR | 153772 | 146590 | 173628 | 103020 | 24343 | 2590 | 4822 |
| sc | RNA | UHRR | 158164 | 148954 | 179554 | 102533 | 26983 | 2898 | 4729 |
| sc | RNA | HBRR | 137574 | 128871 | 192359 | 65776 | 42080 | 4455 | 3444 |
| sc | RNA | HBRR | 140364 | 132257 | 158163 | 71291 | 45036 | 4346 | 3587 |
| sc | RNA | HBRR | 139028 | 131062 | 180890 | 68430 | 43716 | 4363 | 3491 |
| bulk | RNA | NA12878 | 143978 | 136784 | 216400 | 103723 | 6164 | 991 | 4093 |
| bulk | RNA | NA12878 | 143722 | 136331 | 224271 | 102199 | 6450 | 1001 | 3977 |
| sc | RNA | NA12878 | 144980 | 135564 | 228417 | 83320 | 24918 | 2786 | 4005 |
| sc | RNA | NA12878 | 140544 | 137523 | 178383 | 84321 | 33982 | 2346 | 4209 |
| sc | RNA | NA12878 | 145180 | 132673 | 168014 | 80819 | 32462 | 4271 | 3987 |
| sc | RNA | NA12878 | 143670 | 137319 | 156855 | 88397 | 31651 | 3398 | 4290 |
| sc | RNA | NA12878 | 134768 | 133779 | 157409 | 79499 | 36392 | 4688 | 3989 |
| sc | RNA | NA12878 | 137936 | 128515 | 189755 | 76312 | 30484 | 3555 | 3730 |
| sc | RNA | NA12878 | 144234 | 131374 | 216266 | 83585 | 21947 | 2279 | 4209 |
| sc | RNA | NA12878 | 132162 | 124697 | 226673 | 72121 | 25851 | 2027 | 3676 |
| sc | RNA | NA12878 | 133524 | 126383 | 185224 | 69435 | 35414 | 4116 | 3683 |
| sc | RNA | NA12878 | 143500 | 135461 | 182395 | 83406 | 31021 | 3717 | 4465 |
| sc | RNA | NA12878 | 151438 | 140328 | 165606 | 91145 | 30500 | 2840 | 4505 |
| sc | RNA | NA12878 | 130816 | 123078 | 147245 | 74934 | 32092 | 4608 | 3835 |
| sc | RNA | NA12878 | 127082 | 120176 | 240436 | 67613 | 22962 | 2354 | 3415 |
| sc | RNA | UHRR | 138566 | 131354 | 222876 | 83525 | 20513 | 2425 | 4140 |
| sc | RNA | UHRR | 148376 | 141908 | 210176 | 93082 | 24140 | 2672 | 4415 |
| sc | RNA | UHRR | 149260 | 142977 | 197463 | 94311 | 25247 | 2927 | 4306 |
| sc | RNA | HBRR | 149692 | 141335 | 205105 | 77382 | 42238 | 4414 | 4244 |
| sc | RNA | HBRR | 148820 | 140776 | 193856 | 84758 | 35262 | 3041 | 4357 |
| sc | RNA | HBRR | 145386 | 138027 | 192103 | 77421 | 40470 | 4013 | 4309 |
| sc | RNA | UHRR | 163098 | 157847 | 181373 | 106155 | 33267 | 3030 | 5141 |
| sc | RNA | UHRR | 157622 | 151952 | 181530 | 107047 | 25235 | 2539 | 4958 |
| sc | RNA | UHRR | 160140 | 151850 | 179606 | 108116 | 24306 | 2431 | 5080 |
| sc | RNA | NA12878 | 126142 | 117944 | 156564 | 78164 | 20970 | 2337 | 3777 |
| sc | RNA | NA12878 | 141286 | 131697 | 209189 | 89423 | 16523 | 2415 | 4261 |
| sc | RNA | NA12878 | 146528 | 137691 | 186618 | 93643 | 21118 | 2372 | 4708 |
| sc | RNA | NA12878 | 138288 | 129261 | 174250 | 81689 | 27719 | 3284 | 4171 |
| sc | RNA | NA12878 | 144914 | 136295 | 188013 | 88923 | 24216 | 2963 | 4756 |
| sc | RNA | NA12878 | 133040 | 125035 | 172667 | 78929 | 26431 | 1292 | 4339 |
| sc | RNA | NA12878 | 146740 | 135709 | 176506 | 91173 | 25333 | 3479 | 4071 |
| sc | RNA | NA12878 | 138130 | 129626 | 172454 | 92768 | 16749 | 1580 | 4091 |
| bulk | RNA | NA12878 | 100760 | 92681 | 184311 | 62633 | 6971 | 868 | 2744 |
| bulk | RNA | NA12878 | 107446 | 99225 | 202576 | 66537 | 7103 | 801 | 3034 |
| sc | RNA | NA12878 | 132740 | 125738 | 167834 | 90236 | 16007 | 2010 | 4337 |
| sc | RNA | NA12878 | 131602 | 124040 | 158900 | 83280 | 21667 | 2546 | 4102 |
| sc | RNA | NA12878 | 128620 | 121367 | 169355 | 82838 | 18594 | 1854 | 4107 |
| sc | RNA | NA12878 | 126352 | 119347 | 162791 | 86849 | 13847 | 1381 | 3357 |
| sc | RNA | NA12878 | 134156 | 126688 | 176754 | 92174 | 13631 | 1532 | 3967 |
| sc | RNA | NA12878 | 125816 | 118443 | 159207 | 78562 | 20826 | 2490 | 4458 |
| sc | RNA | NA12878 | 132586 | 124957 | 152707 | 91420 | 15639 | 1502 | 4124 |
| sc | RNA | NA12878 | 118670 | 111002 | 156832 | 74414 | 17292 | 2715 | 3701 |
| bulk | RNA | NA12878 | 138622 | 133430 | 165617 | 103108 | 12545 | 1525 | 4215 |
| bulk | RNA | NA12878 | 96724 | 92938 | 114088 | 72165 | 8489 | 1061 | 3024 |
| sc | RNA | NA12878 | 113328 | 108475 | 151609 | 72767 | 17461 | 3083 | 3460 |
| sc | RNA | NA12878 | 102354 | 97609 | 122702 | 64882 | 19071 | 2260 | 3330 |
| sc | RNA | NA12878 | 110128 | 105336 | 122053 | 75753 | 17225 | 1789 | 3311 |
| sc | RNA | NA12878 | 110028 | 105739 | 130703 | 75741 | 16065 | 1885 | 3288 |
| sc | RNA | NA12878 | 90036 | 86032 | 104630 | 60934 | 13870 | 1487 | 2601 |
| sc | RNA | NA12878 | 104292 | 99673 | 126801 | 67364 | 18530 | 1640 | 3208 |
| sc | RNA | NA12878 | 77024 | 73031 | 89320 | 50079 | 12920 | 1350 | 2370 |
| sc | RNA | NA12878 | 80158 | 75682 | 88106 | 50423 | 15589 | 1626 | 2524 |
| sc | RNA | NA12878 | 79398 | 75557 | 101959 | 50129 | 13513 | 2067 | 2440 |
| sc | RNA | NA12878 | 95670 | 91545 | 116468 | 58654 | 20393 | 2076 | 2811 |
| sc | RNA | NA12878 | 116776 | 112440 | 138904 | 76132 | 21116 | 2262 | 3653 |
| sc | RNA | NA12878 | 107766 | 103440 | 133453 | 69993 | 18893 | 1928 | 3096 |
| sc | RNA | NA12878 | 102684 | 98505 | 126691 | 59018 | 24165 | 4366 | 3778 |
| sc | RNA | NA12878 | 101714 | 97289 | 120383 | 59214 | 25036 | 2506 | 3118 |
| sc | RNA | NA12878 | 126382 | 121125 | 147308 | 82514 | 23359 | 2213 | 3569 |
| sc | RNA | NA12878 | 126314 | 121195 | 156459 | 87591 | 16410 | 1632 | 3517 |
| sc | RNA | NA12878 | 116218 | 108082 | 183166 | 72804 | 13054 | 1517 | 3132 |
| sc | RNA | NA12878 | 118604 | 110813 | 160513 | 68867 | 20379 | 5017 | 4363 |
| sc | RNA | NA12878 | 120336 | 112402 | 161540 | 76312 | 16318 | 1762 | 3795 |
| sc | RNA | NA12878 | 110522 | 102852 | 172510 | 67106 | 13709 | 2278 | 3416 |
| sc | RNA | NA12878 | 114190 | 107902 | 152723 | 74024 | 14981 | 2584 | 3488 |
| sc | RNA | NA12878 | 116670 | 107937 | 136282 | 69313 | 23128 | 2899 | 3485 |
| sc | RNA | NA12878 | 110966 | 103027 | 135605 | 73093 | 13293 | 2035 | 3510 |
| sc | RNA | NA12878 | 107620 | 99223 | 131167 | 72053 | 9809 | 1831 | 3270 |
| sc | RNA | NA12878 | 114226 | 105769 | 170667 | 67855 | 16492 | 1819 | 3199 |
| sc | RNA | NA12878 | 116384 | 106922 | 164565 | 71262 | 14228 | 2758 | 3424 |
| sc | RNA | NA12878 | 107744 | 99961 | 158155 | 69215 | 10695 | 1864 | 3407 |
| sc | RNA | NA12878 | 116598 | 108628 | 148294 | 73968 | 16704 | 3017 | 3237 |
| sc | RNA | NA12878 | 110282 | 103075 | 137635 | 69704 | 15205 | 3778 | 3568 |
| sc | RNA | NA12878 | 118408 | 109895 | 138791 | 72362 | 20209 | 2768 | 3659 |
| sc | RNA | NA12878 | 117148 | 107397 | 143382 | 66036 | 24216 | 3056 | 3757 |
| sc | RNA | NA12878 | 118866 | 108362 | 155308 | 66890 | 21653 | 4367 | 3548 |
| sc | RNA | NA12878 | 116610 | 105645 | 157171 | 68813 | 16598 | 3146 | 3144 |
| sc | RNA | NA12878 | 125668 | 113642 | 156008 | 73868 | 19276 | 4188 | 3532 |
| sc | RNA | NA12878 | 120784 | 110723 | 163032 | 73717 | 17637 | 2464 | 3544 |
| sc | RNA | NA12878 | 114684 | 103595 | 137658 | 64496 | 22441 | 3273 | 3251 |
| sc | RNA | NA12878 | 110700 | 100357 | 145931 | 71217 | 11485 | 1519 | 3008 |
| sc | RNA | NA12878 | 111690 | 103308 | 156098 | 63508 | 20221 | 3325 | 3057 |
| sc | RNA | NA12878 | 122000 | 112154 | 141235 | 76596 | 18234 | 2675 | 3902 |
| sc | RNA | NA12878 | 119194 | 109972 | 149290 | 73169 | 17588 | 3478 | 3458 |
| sc | RNA | NA12878 | 119014 | 110267 | 142615 | 75842 | 16573 | 2929 | 3661 |
| sc | RNA | NA12878 | 114046 | 104513 | 145301 | 66024 | 19688 | 3668 | 3518 |
| sc | RNA | NA12878 | 127530 | 120122 | 157982 | 86848 | 14502 | 2090 | 4098 |
| sc | RNA | NA12878 | 104572 | 94766 | 175564 | 50996 | 22803 | 2312 | 2903 |
| sc | RNA | NA12878 | 119448 | 108317 | 139138 | 74790 | 15640 | 2840 | 3772 |
| sc | RNA | NA12878 | 118904 | 103913 | 145759 | 35008 | 51416 | 6701 | 2536 |
| sc | RNA | NA12878 | 119136 | 111326 | 153893 | 76442 | 17317 | 2126 | 3408 |
| sc | RNA | NA12878 | 109002 | 99049 | 152519 | 55166 | 25830 | 2828 | 2769 |
| sc | RNA | NA12878 | 106770 | 97327 | 122323 | 62465 | 20407 | 2740 | 2978 |
| sc | RNA | NA12878 | 110346 | 101914 | 121316 | 70419 | 17942 | 1943 | 2990 |
| sc | RNA | NA12878 | 135554 | 124403 | 157873 | 79492 | 25168 | 4540 | 3854 |
| sc | RNA | NA12878 | 120270 | 112391 | 153542 | 75968 | 18376 | 2244 | 3177 |
| sc | RNA | NA12878 | 118782 | 107713 | 138224 | 65138 | 26804 | 3877 | 3175 |
| sc | RNA | HBRR | 137840 | 122472 | 224322 | 59916 | 37062 | 4033 | 3184 |
| sc | RNA | HBRR | 141724 | 128960 | 226946 | 63924 | 39646 | 4203 | 3487 |
| sc | RNA | HBRR | 134784 | 119371 | 235263 | 59597 | 32964 | 3512 | 3288 |
| sc | RNA | UHRR | 140734 | 128770 | 191224 | 81969 | 23691 | 2630 | 3896 |
| sc | RNA | UHRR | 140658 | 130183 | 189551 | 84169 | 23234 | 2397 | 3898 |
| sc | RNA | UHRR | 146628 | 133046 | 221912 | 81913 | 24338 | 2819 | 3912 |
| sc | RNA | HBRR | 136628 | 125022 | 205062 | 62321 | 40136 | 4255 | 3395 |
| sc | RNA | HBRR | 135382 | 123994 | 207964 | 60650 | 40388 | 4204 | 3291 |
| sc | RNA | HBRR | 90 | 80 | 122 | 52 | 20 | 0 | 3 |
| sc | RNA | UHRR | 137672 | 126694 | 188278 | 81587 | 22468 | 2679 | 3702 |
| sc | RNA | UHRR | 141402 | 131800 | 196341 | 84061 | 23823 | 2776 | 4032 |
| sc | RNA | UHRR | 144448 | 132149 | 200465 | 82133 | 26780 | 2942 | 3795 |
| sc | RNA | HBRR | 113834 | 104116 | 156574 | 54317 | 32530 | 3552 | 2805 |
| sc | RNA | HBRR | 131850 | 117908 | 171888 | 61530 | 37539 | 4065 | 3046 |
| sc | RNA | HBRR | 129554 | 116980 | 174938 | 59221 | 38515 | 4034 | 2915 |
| sc | RNA | UHRR | 147708 | 136874 | 199646 | 92942 | 19407 | 2394 | 4077 |
| sc | RNA | UHRR | 144330 | 134765 | 188675 | 93265 | 17594 | 2363 | 4218 |
| sc | RNA | UHRR | 146428 | 131089 | 202553 | 88106 | 17343 | 2233 | 3978 |
| sc | RNA | HBRR | 130786 | 121305 | 178129 | 64920 | 37511 | 3789 | 3650 |
| sc | RNA | HBRR | 155292 | 142202 | 199334 | 70564 | 50917 | 4997 | 3991 |
| sc | RNA | HBRR | 136016 | 126734 | 186320 | 67722 | 39148 | 4025 | 3710 |
| sc | RNA | UHRR | 160566 | 154019 | 183197 | 99487 | 36235 | 3240 | 4615 |
| sc | RNA | UHRR | 152090 | 144216 | 171512 | 98217 | 27532 | 2906 | 4415 |
| sc | RNA | UHRR | 152704 | 144822 | 188846 | 97957 | 25617 | 2930 | 4445 |
| sc | RNA | HBRR | 109206 | 100261 | 150237 | 54303 | 29651 | 2865 | 3026 |
| sc | RNA | HBRR | 108644 | 98954 | 155633 | 50367 | 31288 | 3265 | 3009 |
| sc | RNA | HBRR | 109950 | 100546 | 159716 | 50590 | 32561 | 3195 | 2861 |
| sc | RNA | UHRR | 126138 | 116638 | 153232 | 80558 | 17749 | 1966 | 3833 |
| sc | RNA | UHRR | 128772 | 120228 | 156904 | 82815 | 19354 | 2014 | 4104 |
| sc | RNA | UHRR | 136726 | 126628 | 159514 | 85577 | 22942 | 2476 | 4080 |
| sc | RNA | HBRR | 138318 | 129024 | 167190 | 69010 | 42614 | 4572 | 3792 |
| sc | RNA | HBRR | 139380 | 129432 | 178906 | 66904 | 43664 | 4392 | 3645 |
| sc | RNA | HBRR | 145512 | 135581 | 184305 | 71099 | 45442 | 4564 | 3793 |
| sc | RNA | UHRR | 159122 | 151246 | 183888 | 105483 | 25080 | 2875 | 4919 |
| sc | RNA | UHRR | 152006 | 143872 | 173380 | 99883 | 24394 | 2787 | 4705 |
| sc | RNA | UHRR | 153664 | 146284 | 175794 | 101498 | 25274 | 2530 | 4845 |
| sc | RNA | HBRR | 134420 | 125450 | 173204 | 65346 | 42022 | 4383 | 3477 |
| sc | RNA | HBRR | 147664 | 134924 | 204874 | 66295 | 46658 | 4611 | 3761 |
| sc | RNA | HBRR | 140818 | 130446 | 180226 | 66436 | 45287 | 4519 | 3557 |
| sc | RNA | UHRR | 155146 | 146944 | 171188 | 100702 | 27965 | 2913 | 4599 |
| sc | RNA | UHRR | 148482 | 140838 | 167348 | 98067 | 24498 | 2610 | 4512 |
| sc | RNA | UHRR | 144666 | 137465 | 164347 | 94877 | 24126 | 2554 | 4621 |
| sc | RNA | HBRR | 128916 | 112136 | 174807 | 57495 | 35065 | 3853 | 3134 |
| sc | RNA | HBRR | 130106 | 113057 | 179909 | 56796 | 36521 | 3722 | 3159 |
| sc | RNA | HBRR | 143404 | 130388 | 215337 | 64686 | 41370 | 4554 | 3657 |
| bulk | RNA | NA12878 | 143350 | 126900 | 179425 | 99062 | 6312 | 942 | 3979 |
| bulk | RNA | NA12878 | 141664 | 117129 | 167179 | 90528 | 5900 | 932 | 3699 |
| sc | RNA | NA12878 | 132590 | 127675 | 162095 | 93353 | 14085 | 1590 | 4554 |
| sc | RNA | NA12878 | 142098 | 122816 | 181668 | 85002 | 11528 | 2564 | 4235 |
| sc | RNA | NA12878 | 139124 | 129376 | 174528 | 91028 | 13165 | 2659 | 4751 |
| sc | RNA | NA12878 | 131144 | 125538 | 165028 | 94165 | 11649 | 1492 | 4125 |
| sc | RNA | NA12878 | 148550 | 117786 | 169358 | 79033 | 18028 | 2431 | 3947 |
| sc | RNA | NA12878 | 144128 | 129895 | 179340 | 93403 | 13926 | 1862 | 4367 |
| sc | RNA | NA12878 | 138168 | 131439 | 157113 | 93749 | 18724 | 1939 | 4471 |
| sc | RNA | NA12878 | 141168 | 129360 | 198168 | 88944 | 14198 | 1810 | 4569 |
| sc | RNA | NA12878 | 126778 | 113809 | 229231 | 70930 | 14810 | 1684 | 3450 |
| sc | RNA | NA12878 | 148272 | 127713 | 164545 | 93937 | 13748 | 1764 | 4125 |
| sc | RNA | NA12878 | 123396 | 111943 | 157058 | 73739 | 18668 | 2914 | 3776 |
| sc | RNA | NA12878 | 131706 | 119684 | 201722 | 82145 | 11446 | 1559 | 3971 |
| sc | RNA | NA12878 | 129742 | 118824 | 187066 | 85268 | 8156 | 1574 | 4137 |
| sc | RNA | NA12878 | 127436 | 112675 | 222674 | 67891 | 15663 | 1801 | 3398 |
| sc | RNA | NA12878 | 149770 | 138698 | 253538 | 83851 | 16896 | 3672 | 4995 |
| sc | RNA | NA12878 | 142150 | 128962 | 183466 | 91323 | 12276 | 2046 | 4652 |
| sc | RNA | UHRR | 159332 | 126409 | 158970 | 85888 | 22087 | 2471 | 3991 |
| sc | RNA | UHRR | 148282 | 127745 | 157771 | 88996 | 21063 | 2312 | 4099 |
| sc | RNA | UHRR | 141896 | 122444 | 153106 | 83927 | 20795 | 2153 | 4014 |

TABLE 1B-2

| | | exonic_{—} | intronic_{—} | intergenic_{—} | overlapping. | | | |
|---|---|---|---|---|---|---|---|---|
| | reads. | Percent of | Percent of | Percent of | exon_Percent | | | |
| | aligned_Percent | Genomic | Genomic | Genomic | of Genomic | | | |
| sample_name | Input Total | Alignments | Alignments | Alignments | Alignments | X5..bias | X3..bias | X5..3..bias |
| H3L73C- | 95.41354861 | 69.99319363 | 23.43659764 | 2.813299233 | 3.756909496 | 0.67 | 100 | 100 |
| NA12878- | | | | | | | | |
| RNA- | | | | | | | | |
| 100pg | | | | | | | | |
| H3L73C- | 88.37680835 | 13.34086933 | 52.93507765 | 31.54677476 | 2.177278258 | 0.74 | 100 | 100 |
| NA12878- | | | | | | | | |
| RNA-NTC | | | | | | | | |
| H3L73C- | 95.35983365 | 89.46795852 | 6.293557709 | 0.839707632 | 3.398776135 | 0.74 | 100 | 100 |
| NA12878- | | | | | | | | |
| RNA- | | | | | | | | |
| 100pg | | | | | | | | |
| H3L73C- | 96.09664853 | 89.62936726 | 6.088512292 | 0.7547067 | 3.527413745 | 0.75 | 100 | 100 |
| NA12878- | | | | | | | | |
| RNA- | | | | | | | | |
| 100pg | | | | | | | | |
| H3L73C- | 96.40584544 | 90.33377311 | 5.309014273 | 0.823295581 | 3.533917036 | 0.72 | 100 | 100 |
| NA12878- | | | | | | | | |
| RNA- | | | | | | | | |
| 100pg | | | | | | | | |
| H3L73C- | 96.31120848 | 89.70063098 | 5.991714048 | 0.791325752 | 3.516329219 | 0.74 | 100 | 100 |
| NA12878- | | | | | | | | |
| RNA- | | | | | | | | |
| 100pg | | | | | | | | |
| H3L73C- | 93.87313909 | 75.75929578 | 17.57564096 | 2.940978402 | 3.724084853 | 0.79 | 100 | 100 |
| NA12878- | | | | | | | | |
| RNA-SC2 | | | | | | | | |
| H3L73C- | 96.56331899 | 78.92993631 | 15.53479594 | 1.776834159 | 3.758433593 | 0.74 | 100 | 100 |
| NA12878- | | | | | | | | |
| RNA-SC2 | | | | | | | | |
| H3L73C- | 94.21925961 | 76.86631113 | 16.79352374 | 2.706844233 | 3.6333209 | 0.74 | 100 | 100 |
| NA12878- | | | | | | | | |
| RNA-SC3 | | | | | | | | |
| H3L73C- | 96.38028271 | 80.03443769 | 14.79472232 | 1.459452731 | 3.711387258 | 0.76 | 100 | 100 |
| NA12878- | | | | | | | | |
| RNA-SC3 | | | | | | | | |
| H3L73C- | 94.87811996 | 80.02388387 | 14.31822033 | 1.972033276 | 3.685862522 | 0.76 | 100 | 100 |
| NA12878- | | | | | | | | |
| RNA-SC4 | | | | | | | | |
| H3L73C- | 95.37052278 | 70.14999906 | 23.19237944 | 2.953813104 | 3.703808393 | 0.74 | 100 | 100 |
| NA12878- | | | | | | | | |
| RNA-SC4 | | | | | | | | |
| H3L73C- | 94.32605479 | 64.67822115 | 24.98043958 | 6.948494835 | 3.392844441 | 0.74 | 100 | 100 |
| NA12878- | | | | | | | | |
| RNA-SC5 | | | | | | | | |
| H3L73C- | 94.69945439 | 76.15853442 | 17.31849035 | 2.747965457 | 3.775009773 | 0.71 | 100 | 100 |
| NA12878- | | | | | | | | |
| RNA-SC5 | | | | | | | | |
| H3L73C- | 95.90319252 | 79.9063643 | 14.49109973 | 2.094123385 | 3.508412582 | 0.72 | 100 | 100 |
| NA12878- | | | | | | | | |
| RNA-SC5 | | | | | | | | |
| H3L73C- | 96.16151931 | 78.54031374 | 16.07383059 | 1.787861781 | 3.597993889 | 0.74 | 100 | 100 |
| NA12878- | | | | | | | | |
| RNA-SC5 | | | | | | | | |
| H3L73C- | 94.63382883 | 67.93272261 | 21.48438512 | 7.125773837 | 3.457118435 | 0.71 | 100 | 100 |
| NA12878- | | | | | | | | |
| RNA-SC6 | | | | | | | | |
| H3L73C- | 95.79537655 | 74.73411642 | 19.09613024 | 2.522198031 | 3.647555305 | 0.76 | 100 | 100 |
| NA12878- | | | | | | | | |
| RNA-SC6 | | | | | | | | |
| H3L73C- | 93.31524976 | 53.13383909 | 32.81820736 | 11.10236958 | 2.945583966 | 0.78 | 100 | 100 |
| NA12878- | | | | | | | | |
| RNA-SC7 | | | | | | | | |
| H3L73C- | 95.6001841 | 72.77543361 | 19.88450812 | 3.569466187 | 3.770592077 | 0.7 | 100 | 100 |
| NA12878- | | | | | | | | |
| RNA-SC7 | | | | | | | | |
| H3L73C- | 93.58791413 | 65.41411395 | 23.16594869 | 8.18124202 | 3.238695337 | 0.7 | 100 | 100 |
| NA12878- | | | | | | | | |
| RNA-SC8 | | | | | | | | |
| H3L73C- | 94.53010328 | 76.27029859 | 17.67385749 | 2.311221169 | 3.74462276 | 0.73 | 100 | 100 |
| NA12878- | | | | | | | | |
| RNA-SC8 | | | | | | | | |
| H3LK3W- | 93.51386275 | 59.86520668 | 33.3934795 | 3.465407717 | 3.275906106 | 0.68 | 100 | 100 |
| HBRR | | | | | | | | |
| H3LK3W- | 94.63973504 | 63.39432673 | 29.83564701 | 3.328810348 | 3.441215907 | 0.71 | 100 | 100 |
| HBRR | | | | | | | | |
| H3LK3W- | 94.15121555 | 59.72075589 | 33.68197926 | 3.333415175 | 3.263849673 | 0.61 | 100 | 100 |
| HBRR | | | | | | | | |
| H3LK3W- | 94.02432538 | 59.46136229 | 33.9100598 | 3.365676413 | 3.26290149 | 0.71 | 100 | 100 |
| HBRR | | | | | | | | |
| H3LK3W- | 93.75674873 | 60.9509147 | 32.52323088 | 3.203229937 | 3.322624481 | 0.67 | 100 | 100 |
| HBRR | | | | | | | | |
| H3LK3W- | 94.38563327 | 58.19379482 | 35.36794512 | 3.32787652 | 3.110383536 | 0.65 | 100 | 100 |
| HBRR | | | | | | | | |
| H3LK3W- | 95.45549004 | 75.52807373 | 18.91204579 | 2.041118945 | 3.518761538 | 0.69 | 100 | 100 |
| HBRR | | | | | | | | |
| H3LK3W- | 95.81055661 | 71.5612473 | 22.74220441 | 2.29309305 | 3.403455249 | 0.76 | 100 | 100 |
| HBRR | | | | | | | | |
| H3LK3W- | 95.32944879 | 76.43850863 | 18.06195511 | 1.921721387 | 3.577814877 | 0.73 | 100 | 100 |
| HBRR | | | | | | | | |
| H3LK3W- | 94.17693027 | 74.76356795 | 19.67508367 | 2.113122799 | 3.448225575 | 0.76 | 100 | 100 |
| HBRR | | | | | | | | |
| H3LK3W- | 93.67395002 | 56.82346335 | 36.35264135 | 3.848645847 | 2.975249449 | 0.66 | 100 | 100 |
| HBRR | | | | | | | | |
| H3LK3W- | 94.22430253 | 57.37244487 | 36.2433607 | 3.497505231 | 2.8866892 | 0.71 | 100 | 100 |
| HBRR | | | | | | | | |
| H3LK3W- | 94.27021895 | 57.025 | 36.43 | 3.635833333 | 2.909166667 | 0.66 | 100 | 100 |
| HBRR | | | | | | | | |
| H3LK3W- | 95.0034033 | 90.21666333 | 5.361351993 | 0.861956493 | 3.560028181 | 0.71 | 100 | 100 |
| NA12878- | | | | | | | | |
| 100pg | | | | | | | | |
| H3LK3W- | 94.8574331 | 89.94253126 | 5.676467741 | 0.88095259 | 3.500048404 | 0.73 | 100 | 100 |
| NA12878- | | | | | | | | |
| 100pg | | | | | | | | |
| H3LK3W- | 93.50531108 | 72.43390797 | 21.6623634 | 2.421997931 | 3.481730694 | 0.75 | 100 | 100 |
| NA12878- | | | | | | | | |
| SC1 | | | | | | | | |
| H3LK3W- | 97.85049522 | 67.53351808 | 27.21651796 | 1.87893447 | 3.37102949 | 0.72 | 100 | 100 |
| NA12878- | | | | | | | | |
| SC11 | | | | | | | | |
| H3LK3W- | 91.38517702 | 66.49635097 | 26.70912217 | 3.514098355 | 3.280428504 | 0.74 | 100 | 100 |
| NA12878- | | | | | | | | |
| SC12 | | | | | | | | |
| H3LK3W- | 95.57945291 | 69.2028872 | 24.7784493 | 2.660174109 | 3.358489384 | 0.76 | 100 | 100 |
| NA12878- | | | | | | | | |
| SC13 | | | | | | | | |
| H3LK3W- | 99.26614627 | 63.81976109 | 29.21456554 | 3.763406332 | 3.202267035 | 0.71 | 100 | 100 |
| NA12878- | | | | | | | | |
| SC14 | | | | | | | | |
| H3LK3W- | 93.17002088 | 66.89282177 | 26.72136464 | 3.116206906 | 3.269606683 | 0.66 | 100 | 100 |
| NA12878- | | | | | | | | |
| SC15 | | | | | | | | |
| H3LK3W- | 91.08393305 | 74.61613998 | 19.59203714 | 2.034458132 | 3.757364756 | 0.73 | 100 | 100 |
| NA12878- | | | | | | | | |
| SC16 | | | | | | | | |
| H3LK3W- | 94.35162906 | 69.56450446 | 24.93465156 | 1.9551483 | 3.545695684 | 0.75 | 100 | 100 |
| NA12878- | | | | | | | | |
| SC2 | | | | | | | | |
| H3LK3W- | 94.65189779 | 61.63891059 | 31.437753 | 3.653859811 | 3.2694766 | 0.72 | 100 | 100 |
| NA12878- | | | | | | | | |
| SC3 | | | | | | | | |
| H3LK3W- | 94.39790941 | 68.02600135 | 25.3007528 | 3.031588219 | 3.641657627 | 0.72 | 100 | 100 |
| NA12878- | | | | | | | | |
| SC4 | | | | | | | | |
| H3LK3W- | 92.66366434 | 70.66051632 | 23.64524382 | 2.201721064 | 3.4925188 | 0.7 | 100 | 100 |
| NA12878- | | | | | | | | |
| SC5 | | | | | | | | |
| H3LK3W- | 94.08482143 | 64.89533987 | 27.79274091 | 3.990681482 | 3.321237735 | 0.74 | 100 | 100 |
| NA12878- | | | | | | | | |
| SC6 | | | | | | | | |
| H3LK3W- | 94.56571348 | 70.17873453 | 23.83334717 | 2.443328074 | 3.544590218 | 0.76 | 100 | 100 |
| NA12878- | | | | | | | | |
| SC8 | | | | | | | | |
| H3LK3W- | 94.79526002 | 75.5178431 | 18.5465132 | 2.192526423 | 3.743117275 | 0.74 | 100 | 100 |
| UHRR | | | | | | | | |
| H3LK3W- | 95.64080444 | 74.87953406 | 19.41935017 | 2.149482338 | 3.55163343 | 0.74 | 100 | 100 |
| UHRR | | | | | | | | |
| H3LK3W- | 95.7905668 | 74.38303981 | 19.91229661 | 2.308523476 | 3.396140105 | 0.71 | 100 | 100 |
| UHRR | | | | | | | | |
| H3LK3W- | 94.41720332 | 60.32367202 | 32.92692434 | 3.440964156 | 3.308439483 | 0.74 | 100 | 100 |
| HBRR | | | | | | | | |
| H3LK3W- | 94.59481253 | 66.51964401 | 27.67426894 | 2.386632972 | 3.41945408 | 0.72 | 100 | 100 |
| HBRR | | | | | | | | |
| H3LK3W- | 94.93830217 | 61.34154168 | 32.06484277 | 3.179545689 | 3.414069866 | 0.63 | 100 | 100 |
| HBRR | | | | | | | | |
| H3LK3W- | 96.78046328 | 71.92414274 | 22.53968684 | 2.05294289 | 3.483227524 | 0.75 | 100 | 100 |
| UHRR | | | | | | | | |
| H3LK3W- | 96.40278641 | 76.58303465 | 18.05349874 | 1.816438807 | 3.547027808 | 0.74 | 100 | 100 |
| UHRR | | | | | | | | |
| H3LK3W- | 94.82327963 | 77.26269 | 17.36974123 | 1.737259974 | 3.630308791 | 0.74 | 100 | 100 |
| UHRR | | | | | | | | |
| H3LK7N- | 93.50097509 | 74.26649438 | 19.92436911 | 2.220469748 | 3.588666768 | 0.83 | 100 | 100 |
| NA12878- | | | | | | | | |
| SC17 | | | | | | | | |
| H3LK7N- | 93.2130572 | 79.40100513 | 14.67120101 | 2.144341248 | 3.783452611 | 0.76 | 100 | 100 |
| NA12878- | | | | | | | | |
| SC18 | | | | | | | | |
| H3LK7N- | 93.96907076 | 76.85672311 | 17.33242505 | 1.946799517 | 3.86405233 | 0.7 | 100 | 100 |
| NA12878- | | | | | | | | |
| SC19 | | | | | | | | |
| H3LK7N- | 93.47231864 | 69.9015086 | 23.71922679 | 2.810128099 | 3.56913651 | 0.74 | 100 | 100 |
| NA12878- | | | | | | | | |
| SC20 | | | | | | | | |
| H3LK7N- | 94.05233449 | 73.57642854 | 20.03673733 | 2.451637459 | 3.935196677 | 0.76 | 100 | 100 |
| NA12878- | | | | | | | | |
| SC21 | | | | | | | | |
| H3LK7N- | 93.98301263 | 71.11297312 | 23.81364255 | 1.164058347 | 3.909325981 | 0.74 | 100 | 100 |
| NA12878- | | | | | | | | |
| SC22 | | | | | | | | |
| H3LK7N- | 92.48262233 | 73.49342233 | 20.4206165 | 2.804378668 | 3.281582511 | 0.72 | 100 | 100 |
| NA12878- | | | | | | | | |
| SC23 | | | | | | | | |
| H3LK7N- | 93.84348078 | 80.53616696 | 14.54057714 | 1.37167066 | 3.551585235 | 0.78 | 100 | 100 |
| NA12878- | | | | | | | | |
| SC24 | | | | | | | | |
| H3LK7N- | 91.98193728 | 85.54550918 | 9.52114292 | 1.185533217 | 3.747814685 | 0.76 | 100 | 100 |
| NA12878- | | | | | | | | |
| 100pg | | | | | | | | |
| H3LK7N- | 92.3487147 | 85.88189739 | 9.168118748 | 1.033881897 | 3.916101968 | 0.68 | 100 | 100 |
| NA12878- | | | | | | | | |
| 100pg | | | | | | | | |
| H3LK7N- | 94.72502637 | 80.14566125 | 14.21707079 | 1.785238476 | 3.852029488 | 0.79 | 100 | 100 |
| NA12878- | | | | | | | | |
| SC1 | | | | | | | | |
| H3LK7N- | 94.25388672 | 74.62699942 | 19.41574443 | 2.281464223 | 3.675791926 | 0.75 | 100 | 100 |
| NA12878- | | | | | | | | |
| SC2 | | | | | | | | |
| H3LK7N- | 94.3609081 | 77.13538126 | 17.31397763 | 1.726369503 | 3.824271601 | 0.79 | 100 | 100 |
| NA12878- | | | | | | | | |
| SC3 | | | | | | | | |
| H3LK7N- | 94.45596429 | 82.37285885 | 13.1333346 | 1.309824155 | 3.183982397 | 0.69 | 100 | 100 |
| NA12878- | | | | | | | | |
| SC4 | | | | | | | | |
| H3LK7N- | 94.43334625 | 82.81283692 | 12.24663983 | 1.376410551 | 3.5641127 | 0.74 | 100 | 100 |
| NA12878- | | | | | | | | |
| SC5 | | | | | | | | |
| H3LK7N- | 94.13985503 | 73.88090581 | 19.58508878 | 2.341634066 | 4.192371351 | 0.79 | 100 | 100 |
| NA12878- | | | | | | | | |
| SC6 | | | | | | | | |
| H3LK7N- | 94.24599882 | 81.1288104 | 13.87851089 | 1.3329192 | 3.659759507 | 0.79 | 100 | 100 |
| NA12878- | | | | | | | | |
| SC7 | | | | | | | | |
| H3LK7N- | 93.53838375 | 75.83824219 | 17.62295917 | 2.766963576 | 3.771835062 | 0.79 | 100 | 100 |
| NA12878- | | | | | | | | |
| SC8 | | | | | | | | |
| H3LVCY- | 96.25456277 | 84.93735224 | 10.33420378 | 1.25625036 | 3.472193619 | 0.72 | 100 | 100 |
| NA12878- | | | | | | | | |
| 100pg | | | | | | | | |
| H3LVCY- | 96.08576982 | 85.16149589 | 10.01781942 | 1.252079916 | 3.568604775 | 0.69 | 100 | 100 |
| NA12878- | | | | | | | | |
| 100pg | | | | | | | | |
| H3LVCY- | 95.71773966 | 75.1950481 | 18.04362877 | 3.1858718 | 3.575451323 | 0.73 | 100 | 100 |
| NA12878- | | | | | | | | |
| SC1 | | | | | | | | |
| H3LVCY- | 95.36412842 | 72.45904202 | 21.29814726 | 2.523927052 | 3.718883665 | 0.8 | 100 | 100 |
| NA12878- | | | | | | | | |
| SC10 | | | | | | | | |
| H3LVCY- | 95.64869969 | 77.23750484 | 17.56255225 | 1.824058402 | 3.3758845 | 0.75 | 100 | 100 |
| NA12878- | | | | | | | | |
| SC11 | | | | | | | | |
| H3LVCY- | 96.10190133 | 78.10041349 | 16.565442 | 1.943719774 | 3.390424731 | 0.72 | 100 | 100 |
| NA12878- | | | | | | | | |
| SC12 | | | | | | | | |
| H3LVCY- | 95.55288996 | 77.23723571 | 17.58099681 | 1.884855245 | 3.296912234 | 0.72 | 100 | 100 |
| NA12878- | | | | | | | | |
| SC13 | | | | | | | | |
| H3LVCY- | 95.57108887 | 74.23684733 | 20.42053294 | 1.807321858 | 3.535297877 | 0.77 | 100 | 100 |
| NA12878- | | | | | | | | |
| SC14 | | | | | | | | |
| H3LVCY- | 94.81590154 | 75.05957823 | 19.36479863 | 2.023411622 | 3.552211514 | 0.83 | 100 | 100 |
| NA12878- | | | | | | | | |
| SC15 | | | | | | | | |
| H3LVCY- | 94.41602834 | 71.86653744 | 22.21857986 | 2.3174938 | 3.5973889 | 0.77 | 100 | 100 |
| NA12878- | | | | | | | | |
| SC16 | | | | | | | | |
| H3LVCY- | 95.16234666 | 73.55793922 | 19.82861084 | 3.033059913 | 3.580390028 | 0.75 | 100 | 100 |
| NA12878- | | | | | | | | |
| SC2 | | | | | | | | |
| H3LVCY- | 95.68830354 | 69.88109705 | 24.29647104 | 2.473371935 | 3.349059976 | 0.76 | 100 | 100 |
| NA12878- | | | | | | | | |
| SC3 | | | | | | | | |
| H3LVCY- | 96.28690827 | 73.79777633 | 20.46857885 | 2.192646588 | 3.540998226 | 0.72 | 100 | 100 |
| NA12878- | | | | | | | | |
| SC4 | | | | | | | | |
| H3LVCY- | 95.9857469 | 74.53199872 | 20.11819827 | 2.053029496 | 3.296773507 | 0.72 | 100 | 100 |
| NA12878- | | | | | | | | |
| SC5 | | | | | | | | |
| H3LVCY- | 95.93023256 | 64.62272931 | 26.45986401 | 4.780623474 | 4.136783208 | 0.7 | 100 | 100 |
| NA12878- | | | | | | | | |
| SC6 | | | | | | | | |
| H3LVCY- | 95.64956643 | 65.88557314 | 27.85677727 | 2.788348132 | 3.469301466 | 0.75 | 100 | 100 |
| NA12878- | | | | | | | | |
| SC7 | | | | | | | | |
| H3LVCY- | 95.84038866 | 73.90085531 | 20.92069321 | 1.981998119 | 3.196453361 | 0.77 | 100 | 100 |
| NA12878- | | | | | | | | |
| SC8 | | | | | | | | |
| H3LVCY- | 95.94740092 | 80.24828218 | 15.03435639 | 1.495190105 | 3.222171324 | 0.75 | 100 | 100 |
| NA12878- | | | | | | | | |
| SC9 | | | | | | | | |
| H3LVVH- | 92.99936327 | 80.44018695 | 14.42319379 | 1.67611345 | 3.460505817 | 0.73 | 100 | 100 |
| NA12878- | | | | | | | | |
| SC1 | | | | | | | | |
| H3LVVH- | 93.43108158 | 69.82641494 | 20.66290836 | 5.086893922 | 4.423782775 | 0.73 | 100 | 100 |
| NA12878- | | | | | | | | |
| SC2 | | | | | | | | |
| H3LVVH- | 93.40679431 | 77.72108324 | 16.61930806 | 1.794534918 | 3.865073788 | 0.76 | 100 | 100 |
| NA12878- | | | | | | | | |
| SC3 | | | | | | | | |
| H3LVVH- | 93.06020521 | 77.57111977 | 15.8469061 | 2.633252032 | 3.948722098 | 0.7 | 100 | 100 |
| NA12878- | | | | | | | | |
| SC4 | | | | | | | | |
| H3LVVH- | 94.49338821 | 77.85689494 | 15.75670246 | 2.717797154 | 3.668605446 | 0.75 | 100 | 100 |
| NA12878- | | | | | | | | |
| SC5 | | | | | | | | |
| H3LVVH- | 92.51478529 | 70.13711105 | 23.40298507 | 2.933468252 | 3.526435619 | 0.75 | 100 | 100 |
| NA12878- | | | | | | | | |
| SC6 | | | | | | | | |
| H3LVVH- | 92.8455563 | 79.50854445 | 14.45975786 | 2.213616734 | 3.818080952 | 0.75 | 100 | 100 |
| NA12878- | | | | | | | | |
| SC7 | | | | | | | | |
| H3LVVH- | 92.19754692 | 82.85477732 | 11.27950968 | 2.105493141 | 3.760219864 | 0.77 | 100 | 100 |
| NA12878- | | | | | | | | |
| SC8 | | | | | | | | |
| H3LVVH- | 92.59625654 | 75.93017401 | 18.45465227 | 2.0354725 | 3.579701225 | 0.83 | 100 | 100 |
| NA12878- | | | | | | | | |
| SC9 | | | | | | | | |
| H3LVVH- | 91.8700165 | 77.73584082 | 15.52055153 | 3.00855223 | 3.735055415 | 0.79 | 100 | 100 |
| NA12878- | | | | | | | | |
| SC10 | | | | | | | | |
| H3LVVH- | 92.7763959 | 81.25638347 | 12.55561686 | 2.188281424 | 3.999718247 | 0.74 | 100 | 100 |
| NA12878- | | | | | | | | |
| SC11 | | | | | | | | |
| H3LVVH- | 93.16454828 | 76.31388895 | 17.23376597 | 3.112683903 | 3.339661185 | 0.69 | 100 | 100 |
| NA12878- | | | | | | | | |
| SC12 | | | | | | | | |
| H3LVVH- | 93.46493535 | 75.55579643 | 16.48149152 | 4.095170993 | 3.867541055 | 0.76 | 100 | 100 |
| NA12878- | | | | | | | | |
| SC13 | | | | | | | | |
| H3LVVH- | 92.810452 | 73.09440595 | 20.41354371 | 2.796016081 | 3.696034263 | 0.77 | 100 | 100 |
| NA12878- | | | | | | | | |
| SC14 | | | | | | | | |
| H3LVVH- | 91.67634104 | 68.03276155 | 24.94823057 | 3.148405708 | 3.870602174 | 0.74 | 100 | 100 |
| NA12878- | | | | | | | | |
| SC17 | | | | | | | | |
| H3LVVH- | 91.16315851 | 69.34624396 | 22.44811213 | 4.527359058 | 3.678284849 | 0.74 | 100 | 100 |
| NA12878- | | | | | | | | |
| SC18 | | | | | | | | |
| H3LVVH- | 90.59686133 | 75.04062115 | 18.10012977 | 3.430715041 | 3.42853404 | 0.72 | 100 | 100 |
| NA12878- | | | | | | | | |
| SC19 | | | | | | | | |
| H3LVVH- | 90.43034026 | 73.23524746 | 19.11088198 | 4.152125635 | 3.501744924 | 0.77 | 100 | 100 |
| NA12878- | | | | | | | | |
| SC20 | | | | | | | | |
| H3LVVH- | 91.67025434 | 75.71434441 | 18.11487028 | 2.530761488 | 3.640023829 | 0.74 | 100 | 100 |
| NA12878- | | | | | | | | |
| SC21 | | | | | | | | |
| H3LVVH- | 90.33082209 | 69.00846347 | 24.01108484 | 3.501995485 | 3.478456254 | 0.71 | 100 | 100 |
| NA12878- | | | | | | | | |
| SC22 | | | | | | | | |
| H3LVVH- | 90.6567299 | 81.64371941 | 13.16649279 | 1.741393344 | 3.448394456 | 0.79 | 100 | 100 |
| NA12878- | | | | | | | | |
| SC24 | | | | | | | | |
| H3LVVH- | 92.49529949 | 70.47752217 | 22.44010165 | 3.689893576 | 3.392482605 | 0.8 | 100 | 100 |
| NA12878- | | | | | | | | |
| SC25 | | | | | | | | |
| H3LVVH- | 91.9295082 | 75.53324721 | 17.98100723 | 2.637884959 | 3.847860601 | 0.75 | 100 | 100 |
| NA12878- | | | | | | | | |
| SC26 | | | | | | | | |
| H3LVVH- | 92.26303337 | 74.89687081 | 18.00333698 | 3.560132251 | 3.539659955 | 0.78 | 100 | 100 |
| NA12878- | | | | | | | | |
| SC27 | | | | | | | | |
| H3LVVH- | 92.65044449 | 76.60421191 | 16.73955861 | 2.958436443 | 3.697793041 | 0.79 | 100 | 100 |
| NA12878- | | | | | | | | |
| SC28 | | | | | | | | |
| H3LVVH- | 91.64109219 | 71.07149777 | 21.19313656 | 3.948416543 | 3.786949127 | 0.77 | 100 | 100 |
| NA12878- | | | | | | | | |
| SC29 | | | | | | | | |
| H3LVVH- | 94.1911707 | 80.76028939 | 13.4854656 | 1.943499042 | 3.810745969 | 0.77 | 100 | 100 |
| NA12878- | | | | | | | | |
| SC30 | | | | | | | | |
| H3LVVH- | 90.62272884 | 64.54046118 | 28.85944263 | 2.926063736 | 3.67403245 | 0.77 | 100 | 100 |
| NA12878- | | | | | | | | |
| SC31 | | | | | | | | |
| H3LVVH- | 90.68130065 | 77.06972239 | 16.11673296 | 2.926567878 | 3.886976773 | 0.77 | 100 | 100 |
| NA12878- | | | | | | | | |
| SC32 | | | | | | | | |
| H3LVVH- | 87.39235013 | 36.59589592 | 53.74813142 | 7.004944544 | 2.65102811 | 0.62 | 100 | 100 |
| NA12878- | | | | | | | | |
| SC33 | | | | | | | | |
| H3LVVH- | 93.44446683 | 76.98629309 | 17.44030294 | 2.141137845 | 3.432266121 | 0.73 | 100 | 100 |
| NA12878- | | | | | | | | |
| SC34 | | | | | | | | |
| H3LVVH- | 90.86897488 | 63.70722807 | 29.82920097 | 3.265852898 | 3.19771806 | 0.72 | 100 | 100 |
| NA12878- | | | | | | | | |
| SC35 | | | | | | | | |
| H3LVVH- | 91.15575536 | 70.5102156 | 23.0353313 | 3.092899876 | 3.361553223 | 0.76 | 100 | 100 |
| NA12878- | | | | | | | | |
| SC36 | | | | | | | | |
| H3LVVH- | 92.35858119 | 75.48073831 | 19.23167621 | 2.082663408 | 3.204922074 | 0.73 | 100 | 100 |
| NA12878- | | | | | | | | |
| SC37 | | | | | | | | |
| H3LVVH- | 91.77375806 | 70.31330161 | 22.2619279 | 4.01578007 | 3.408990394 | 0.76 | 100 | 100 |
| NA12878- | | | 3 | | | | | |
| SC38 | | | | | | | | |
| H3LVVH- | 93.44890663 | 76.14694532 | 18.41928532 | 2.249285822 | 3.184483536 | 0.78 | 100 | 100 |
| NA12878- | | | | | | | | |
| SC39 | | | | | | | | |
| H3LVVH- | 90.681248 | 65.79994747 | 27.07638847 | 3.916398974 | 3.207265087 | 0.74 | 100 | 100 |
| NA12878- | | | | | | | | |
| SC40 | | | | | | | | |
| H3MG2V- | 88.85084156 | 57.50371899 | 35.569845 | 3.870627189 | 3.05580882 | 0.62 | 100 | 100 |
| HBRR | | | | | | | | |
| H3MG2V- | 90.99376252 | 57.45461082 | 35.63365091 | 3.777637965 | 3.134100306 | 0.66 | 100 | 100 |
| HBRR | | | | | | | | |
| H3MG2V- | 88.56466643 | 59.98027395 | 33.17599461 | 3.534586005 | 3.309145439 | 0.63 | 100 | 100 |
| HBRR | | | | | | | | |
| H3MG2V- | 91.498856 | 73.06526661 | 21.11760826 | 2.344321038 | 3.472804093 | 0.76 | 100 | 100 |
| UHRR | | | | | | | | |
| H3MG2V- | 92.55285871 | 74.02856691 | 20.43483614 | 2.108216503 | 3.428380446 | 0.71 | 100 | 100 |
| UHRR | | | | | | | | |
| H3MG2V- | 90.73710342 | 72.50092935 | 21.54148448 | 2.495087713 | 3.462498451 | 0.73 | 100 | 100 |
| UHRR | | | | | | | | |
| H3MG2V- | 91.50540153 | 56.60039779 | 36.45181505 | 3.864422789 | 3.083364364 | 0.69 | 100 | 100 |
| HBRR | | | | | | | | |
| H3MG2V- | 91.58824659 | 55.88162126 | 37.212645 | 3.87347627 | 3.03225747 | 0.61 | 100 | 100 |
| HBRR | | | | | | | | |
| H3MG2V- | 88.8888888 | 69.33333333 | 26.66666667 | 0 | 4 | NA | 100 | 100 |
| HBRR | | | | | | | | |
| H3MG2V- | 92.02597478 | 73.87717773 | 20.3448151 | 2.4258394 | 3.352167771 | 0.73 | 100 | 100 |
| UHRR | | | | | | | | |
| H3MG2V- | 93.20943127 | 73.29281903 | 20.77128309 | 2.420395494 | 3.515502389 | 0.74 | 100 | 100 |
| UHRR | | | | | | | | |
| H3MG2V- | 91.48551728 | 71.01859058 | 23.15607436 | 2.543882404 | 3.281452659 | 0.73 | 100 | 100 |
| UHRR | | | | | | | | |
| H3MG2V- | 91.46300754 | 58.27754174 | 34.90193554 | 3.810995236 | 3.009527488 | 0.69 | 100 | 100 |
| HBRR | | | | | | | | |
| H3MG2V- | 89.42586272 | 57.94876625 | 35.35411565 | 3.828404596 | 2.868713505 | 0.65 | 100 | 100 |
| HBRR | | | | | | | | |
| H3MG2V- | 90.29439462 | 56.57066437 | 36.79132636 | 3.853465157 | 2.784544109 | 0.65 | 100 | 100 |
| HBRR | | | | | | | | |
| H3MG2V- | 92.66525848 | 78.22083824 | 16.3331089 | 2.014812321 | 3.431240532 | 0.74 | 100 | 100 |
| UHRR | | | | | | | | |
| H3MG2V- | 93.37282616 | 79.41502044 | 14.98126703 | 2.012091281 | 3.591621253 | 0.79 | 100 | 100 |
| UHRR | | | | | | | | |
| H3MG2V- | 89.52454449 | 78.9056063 | 15.53197206 | 1.999820885 | 3.562600752 | 0.75 | 100 | 100 |
| UHRR | | | | | | | | |
| H3MG2V- | 92.75075314 | 59.08801311 | 34.14125785 | 3.448621098 | 3.322107946 | 0.65 | 100 | 100 |
| HBRR | | | | | | | | |
| H3MG2V- | 91.57071839 | 54.0848784 | 39.02612881 | 3.830028589 | 3.058964198 | 0.68 | 100 | 100 |
| HBRR | | | | | | | | |
| H3MG2V- | 93.17580285 | 59.09166267 | 34.1590681 | 3.512063174 | 3.237206056 | 0.61 | 100 | 100 |
| HBRR | | | | | | | | |
| H3MG2V- | 95.92254898 | 69.29173893 | 25.2373291 | 2.256628847 | 3.214303127 | 0.77 | 100 | 100 |
| UHRR | | | | | | | | |
| H3MG2V- | 94.82280229 | 73.80852183 | 20.68986248 | 2.183813031 | 3.31780266 | 0.7 | 100 | 100 |
| UHRR | | | | | | | | |
| H3MG2V- | 94.83838013 | 74.80545861 | 19.5625778 | 2.237512314 | 3.394451275 | 0.75 | 100 | 100 |
| UHRR | | | | | | | | |
| H3MGFK- | 91.80905811 | 60.44075909 | 33.00239301 | 3.188825199 | 3.368022706 | 0.76 | 100 | 100 |
| HBRR | | | | | | | | |
| H3MGFK- | 91.08096167 | 57.28144298 | 35.58325467 | 3.713223169 | 3.422079178 | 0.74 | 100 | 100 |
| HBRR | | | | | | | | |
| H3MGFK- | 91.44702137 | 56.71079624 | 36.50049884 | 3.581557501 | 3.207147421 | 0.63 | 100 | 100 |
| HBRR | | | | | | | | |
| H3MGFK- | 92.46856617 | 77.38074655 | 17.04896932 | 1.888459839 | 3.681824294 | 0.81 | 100 | 100 |
| UHRR | | | | | | | | |
| H3MGFK- | 93.36501724 | 76.47732415 | 17.87287486 | 1.859872376 | 3.789928616 | 0.77 | 100 | 100 |
| UHRR | | | | | | | | |
| H3MGFK- | 92.61442593 | 74.36628286 | 19.93656311 | 2.151640235 | 3.545513795 | 0.79 | 100 | 100 |
| UHRR | | | | | | | | |
| H3MGFK- | 93.28070099 | 57.51408474 | 35.51521819 | 3.810381038 | 3.160316032 | 0.71 | 100 | 100 |
| HBRR | | | | | | | | |
| H3MGFK- | 92.86267757 | 56.40908899 | 36.81463682 | 3.703047932 | 3.073226255 | 0.75 | 100 | 100 |
| HBRR | | | | | | | | |
| H3MGFK- | 93.17513332 | 56.92565133 | 36.38328876 | 3.654181812 | 3.036878093 | 0.69 | 100 | 100 |
| HBRR | | | | | | | | |
| H3MGFK- | 95.05033873 | 76.23972766 | 18.12701923 | 2.077957747 | 3.555295359 | 0.79 | 100 | 100 |
| UHRR | | | | | | | | |
| H3MGFK- | 94.64889544 | 75.80159218 | 18.51270026 | 2.115065 | 3.570642564 | 0.81 | 100 | 100 |
| UHRR | | | | | | | | |
| H3MGFK- | 95.19731362 | 75.66177402 | 18.84052569 | 1.885990742 | 3.611709543 | 0.77 | 100 | 100 |
| UHRR | | | | | | | | |
| H3MGFK- | 93.32688588 | 56.71017461 | 36.46856667 | 3.803762974 | 3.017495748 | 0.64 | 100 | 100 |
| HBRR | | | | | | | | |
| H3MGFK- | 91.37230469 | 54.64248918 | 38.45703688 | 3.800535751 | 3.099938183 | 0.72 | 100 | 100 |
| HBRR | | | | | | | | |
| H3MGFK- | 92.63446434 | 55.45622251 | 37.80248583 | 3.772151687 | 2.969139976 | 0.68 | 100 | 100 |
| HBRR | | | | | | | | |
| H3MGFK- | 94.71336676 | 73.94825928 | 20.5354717 | 2.139096336 | 3.377172692 | 0.77 | 100 | 100 |
| UHRR | | | | | | | | |
| H3MGFK- | 94.85190124 | 75.61821925 | 18.89009693 | 2.01253788 | 3.479145944 | 0.77 | 100 | 100 |
| UHRR | | | | | | | | |
| H3MGFK- | 95.02232729 | 75.19298134 | 19.1206074 | 2.024124649 | 3.662286611 | 0.73 | 100 | 100 |
| UHRR | | | | | | | | |
| H3MGFK- | 86.98377238 | 57.75663757 | 35.22456729 | 3.870533517 | 3.148261625 | 0.66 | 100 | 100 |
| HBRR | | | | | | | | |
| H3MGFK- | 86.89606936 | 56.68376614 | 36.44883131 | 3.714645003 | 3.15275754 | 0.65 | 100 | 100 |
| HBRR | | | | | | | | |
| H3MGFK- | 90.92354467 | 56.60951981 | 36.20467852 | 3.98540261 | 3.200399065 | 0.68 | 100 | 100 |
| HBRR | | | | | | | | |
| H3MGFK- | 88.52459016 | 89.81549481 | 5.722834217 | 0.854073167 | 3.607597806 | 0.72 | 100 | 100 |
| NA12878- | | | | | | | | |
| 100pg | | | | | | | | |
| H3MGFK- | 82.68085046 | 89.57935463 | 5.83817374 | 0.922233547 | 3.660238079 | 0.72 | 100 | 100 |
| NA12878- | | | | | | | | |
| 100pg | | | | | | | | |
| H3MGFK- | 96.29308394 | 82.18995968 | 12.40073251 | 1.399869698 | 4.009438115 | 0.79 | 100 | 100 |
| NA12878- | | | | | | | | |
| SC1 | | | | | | | | |
| H3MGFK- | 86.43049163 | 82.26344976 | 11.15659689 | 2.481394381 | 4.098558972 | 0.78 | 100 | 100 |
| NA12878- | | | | | | | | |
| SC10 | | | | | | | | |
| H3MGFK- | 92.99330094 | 81.56411566 | 11.79627788 | 2.38255244 | 4.257054022 | 0.73 | 100 | 100 |
| NA12878- | | | | | | | | |
| SC11 | | | | | | | | |
| H3MGFK- | 95.72530958 | 84.5052095 | 10.45400293 | 1.338945177 | 3.701842396 | 0.77 | 100 | 100 |
| NA12878- | | | | | | | | |
| SC12 | | | | | | | | |
| H3MGFK- | 79.29047459 | 76.40541769 | 17.42862943 | 2.350177399 | 3.815775481 | 0.78 | 100 | 100 |
| NA12878- | | | | | | | | |
| SC13 | | | | | | | | |
| H3MGFK- | 90.12475022 | 82.25136054 | 12.2633368 | 1.639690731 | 3.845611934 | 0.71 | 100 | 100 |
| NA12878- | | | | | | | | |
| SC14 | | | | | | | | |
| H3MGFK- | 95.12984193 | 78.85820513 | 15.74993902 | 1.631015368 | 3.76084049 | 0.78 | 100 | 100 |
| NA12878- | | | | | | | | |
| SC15 | | | | | | | | |
| H3MGFK- | 91.63549813 | 81.21182239 | 12.96372385 | 1.65265109 | 4.171802668 | 0.73 | 100 | 100 |
| NA12878- | | | | | | | | |
| SC16 | | | | | | | | |
| H3MGFK- | 89.77030715 | 78.05312851 | 16.29729075 | 1.853115303 | 3.796465436 | 0.75 | 100 | 100 |
| NA12878- | | | | | | | | |
| SC2 | | | | | | | | |
| H3MGFK- | 86.13426675 | 82.70995122 | 12.10488316 | 1.553172381 | 3.631993238 | 0.78 | 100 | 100 |
| NA12878- | | | | | | | | |
| SC3 | | | | | | | | |
| H3MGFK- | 90.71849979 | 74.4109307 | 18.83810812 | 2.940553195 | 3.810407984 | 0.77 | 100 | 100 |
| NA12878- | | | | | | | | |
| SC4 | | | | | | | | |
| H3MGFK- | 90.87209391 | 82.87345769 | 11.54750255 | 1.572825133 | 4.006214627 | 0.79 | 100 | 100 |
| NA12878- | | | | | | | | |
| SC5 | | | | | | | | |
| H3MGFK- | 91.5848376 | 86.01200383 | 8.227164977 | 1.587733898 | 4.173097292 | 0.76 | 100 | 100 |
| NA12878- | | | | | | | | |
| SC6 | | | | | | | | |
| H3MGFK- | 88.41693085 | 76.49431569 | 17.64785416 | 2.029227181 | 3.828602977 | 0.76 | 100 | 100 |
| NA12878- | | | | | | | | |
| SC7 | | | | | | | | |
| H3MGFK- | 92.60733124 | 76.63644506 | 15.44226516 | 3.356060468 | 4.565229313 | 0.73 | 100 | 100 |
| NA12878- | | | | | | | | |
| SC8 | | | | | | | | |
| H3MGFK- | 90.72247626 | 82.79735623 | 11.12994914 | 1.854991523 | 4.217703111 | 0.75 | 100 | 100 |
| NA12878- | | | | | | | | |
| SC9 | | | | | | | | |
| H3MGFK- | 79.33685638 | 75.05264906 | 19.30057586 | 2.159266671 | 3.487508411 | 0.77 | 100 | 100 |
| UHRR | | | | | | | | |
| H3MGFK- | 86.15003844 | 76.41109299 | 18.08448528 | 1.985060531 | 3.519361209 | 0.75 | 100 | 100 |
| UHRR | | | | | | | | |
| H3MGFK- | 86.29136833 | 75.68559551 | 18.75298722 | 1.941581221 | 3.619836052 | 0.71 | 100 | 100 |
| UHRR | | | | | | | | |

### Example 2: Multiomics Approach to Analysis of Oncogenic and Drug Resistance Mechanisms

### Overview

Cancer is a disease of remarkable variation and heterogeneity between the individual cells comprising the bulk tumor tissue. While a multitude of studies have described these changes across the evolution of cancer, etiology is still driven by speculation in most cancers. This is borne out in the molecular complexity underlying the resiliency of cancer cells in drug resistance, whereby single nucleotide variation (SNV) and copy number variation (CNV) at the genomic level contributes to resistance in concert with transcriptional adaptation. While one of these modes can be a dominant driver, there is increasing evidence that the modes are not mutually exclusive and instead can synergize to change cell state leading to resistance. It will therefore become important to assay these multiple “-omic” tiers (genomic and transcriptomic) in single cells, as bulk sequencing provides an incomplete view of the inherent heterogeneity in each of these tiers. Cancer's evolution is driven through a complex molecular orchestration, where the interdependence of genomic and transcriptomic changes occurring in each cell convey some of the major fitness advantages that drive expansion and drug resistance. The nature of current genomic and transcriptomic assays muddle the underlying clonal structure by reducing genomic data to tissue-based averages. Recent methods aimed at simultaneously monitoring both RNA and DNA in single cells have made this linking possible, but contain uneven genome coverage and low allelic balance, limiting the ability to assess single nucleotide variation genome-wide with accuracy.

To overcome this challenge, the PTA workflow was enhanced and extended a second modality of transcriptome enrichment. The method is differentiated through enhanced genome coverage and uniformity, along with allelic balance, wherein both copies of the genome are equivalently and uniformly amplified. This is an underlying attribute that allows both CNV and SNV detection from an amplified genome of a sample as finite as a single cell with high accuracy. The ability of PTA to provide this degree of uniformity and accuracy stems from the unfavored recopying of synthesized strands, driven by nucleotide terminators that limit the size of the amplicons, and coincidentally this amplicon-size distribution (500-1500 bp) is suitable for the natural distribution of transcript lengths.

NA12878 cells are relatively transcriptionally quiescent. Following the general multiomic procedure of Example 1, uniquely expressed genes in single cells from our DCIS and MOLM-13 material were also assessed (FIG. 3D). First rarefaction analysis was performed by down-sampling the RNA libraries to 75k reads, finding only a nominal benefit of doubling the read number regarding genes detected. Isoform detection and coverage still increased proportional to reads. At 75K reads per cell the benchmark cell line NA12878 averaged ˜4500 expressed genes detected while MOLM-13 AML cells averaged ˜5000-5500. FACS-enriched single cells from a primary DCIS/IDC tumor specimen yielded less expressed genes than the cell line models, averaging ˜3500, without being bound by theory, potentially owing to sample integrity of the primary singulated cells and the increased number of workflow steps from surgical resection to FACS.

### Generation of a Drug Resistance Model in MOLM-13 Acute Myeloid Leukemia Cells

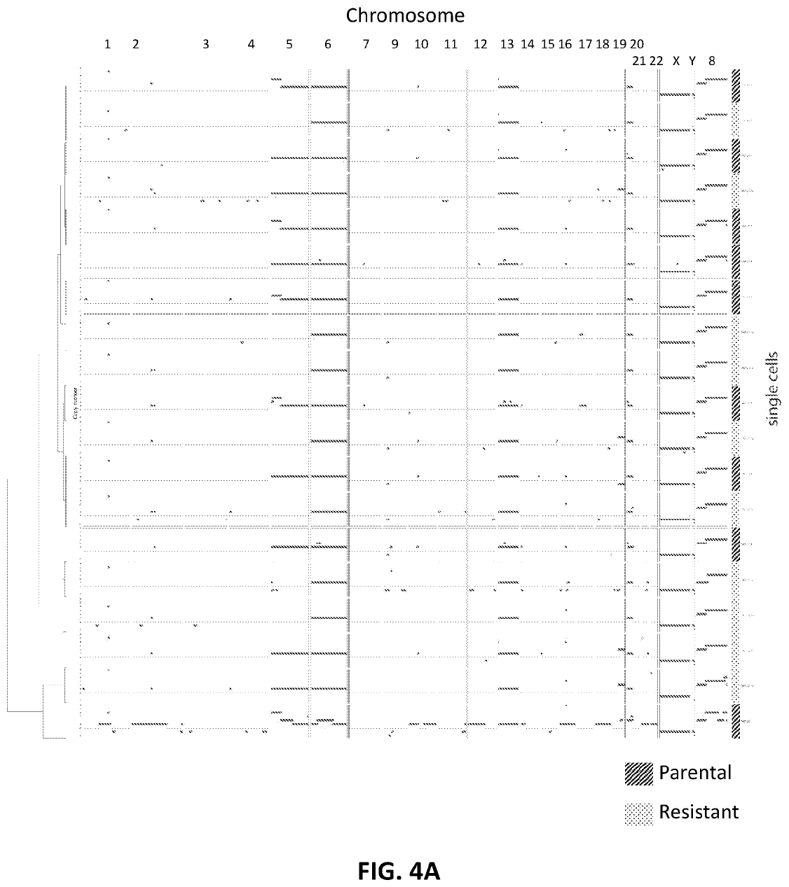

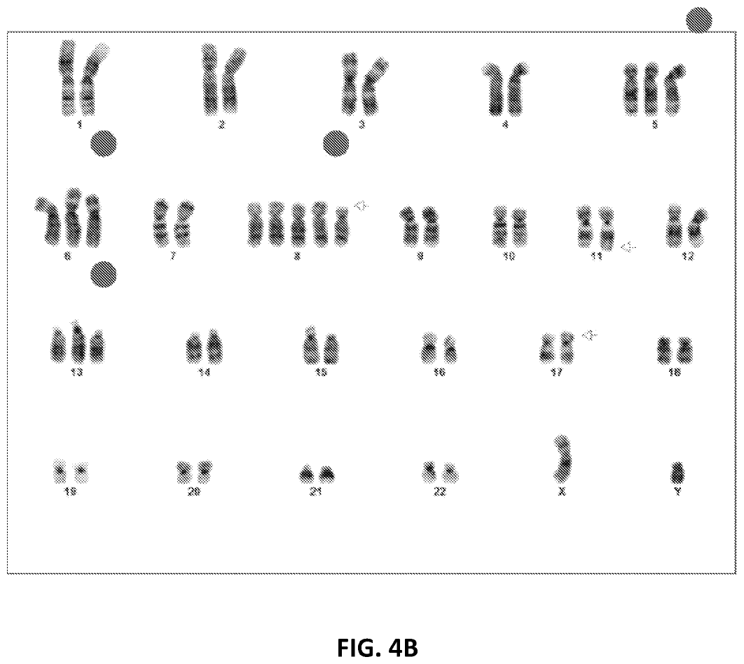

DNA and RNA performance metrics of multiomics on control cells was expanded to generate unified genomic and transcriptomic information from a model of drug resistance. Prior to looking at heterogenous effects of drug resistance, the chemistry was evaluated to confirm it regenerated MOLM-13's known genomic features. Cells were first karyotypically assessed to match published reports and provide context for interpreting CNV analysis. The combined copy number analysis of all MOLM-13 cells used in this study are found in FIG. 4A. Prior to drug resistance modeling, MOLM-13 line exhibited hallmarks of the initial cell line establishment including trisomies of Chr.6 and Chr. 13 (49,2n., XY,+6,+8, +13, 49,2n., XY, +6,+8, ins (11;9) (q23;p22p23), ins (11;9) (q23;p22p23), del (14) (q23.3;q31.3). The MOLM-13 line exhibited (FIG. 4B) additional gains including the presentation of trisomy 5 and pentasomy 8 concomitant with other translocations (52, XY, +5, +6, +8, +8, +del (8p), add (11q), +13, add (17p)).

To demonstrate the utility of concurrent genomic and transcriptomic information in single cells in the context of drug resistance, a model was created by exploiting the presence of an internal tandem duplication (ITD) mutation in MOLM-13 cells. Since the ITD mutation, found in ˜20% of AML patients, hyperactivates FLT3 signaling and results in poor prognosis and relapse, non-resistant, drug-sensitive cells were treated with a continual dose of 2 nM quizartinib. This drug is a selective type II kinase inhibitor targeting FLT3. Resistance emerged following initial marked growth inhibition/apoptosis (See Methods, FIG. 11).

### Distinction in Single-Cell CNV Profiles Among Parental and Quizartinib-Resistant MOLM-13 Cells

As an initial assessment of single-cell genomic variation in the MOLM-13 quizartinib resistance model CNV analysis was performed following the multiomics workflow on 9 parental “P” and 10 quizartinib-resistant “R” cells. Utilizing sequencing data to yield ˜25× coverage and a 500 kb window size, copy number gain was evident for chromosomes 5, 6, 8, and 13 (FIG. 4A) and concordant with our karyotypic data for the parental cells (FIG. 4B).

Single-cell CNV heterogeneity immediately emerged from the data. Within the “P” cohort, gain to 3N was observed for 9/9 cells for Chr. 5, yet 5/9 cells showed additional 5p gains. Most relevant, heterogenous copy number variation between “P” and “R” single cells was observed. No resistant cells exhibited the additional 5p gain found in the parental cohort, and furthermore, 7/10 resistant cells did not have any amplification of Chr. 5 as a diploid 2n state, suggesting that this was selected for to mediate drug resistance in part by expression consequences on multiple Chr.5-resident genes. In addition to this general implication of Chr. 5 as a candidate contributor to quizartinib resistance, 19q gain uniquely in 4/10 resistance cells was observed. Taken together, a CNV paradigm for the MOLM-13 resistance model was defined that was used as context for the SNV and transcriptional layers to be subsequently defined by multiomics methods described herein.

### Acquisition of a Secondary FLT3 Mutation as a Key Driver of Drug Resistance

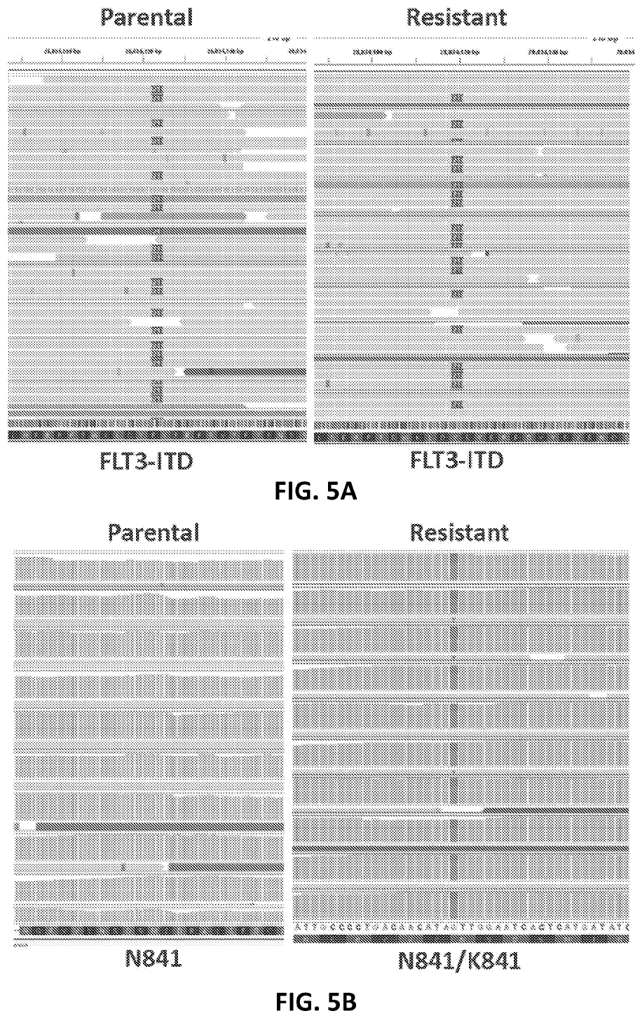

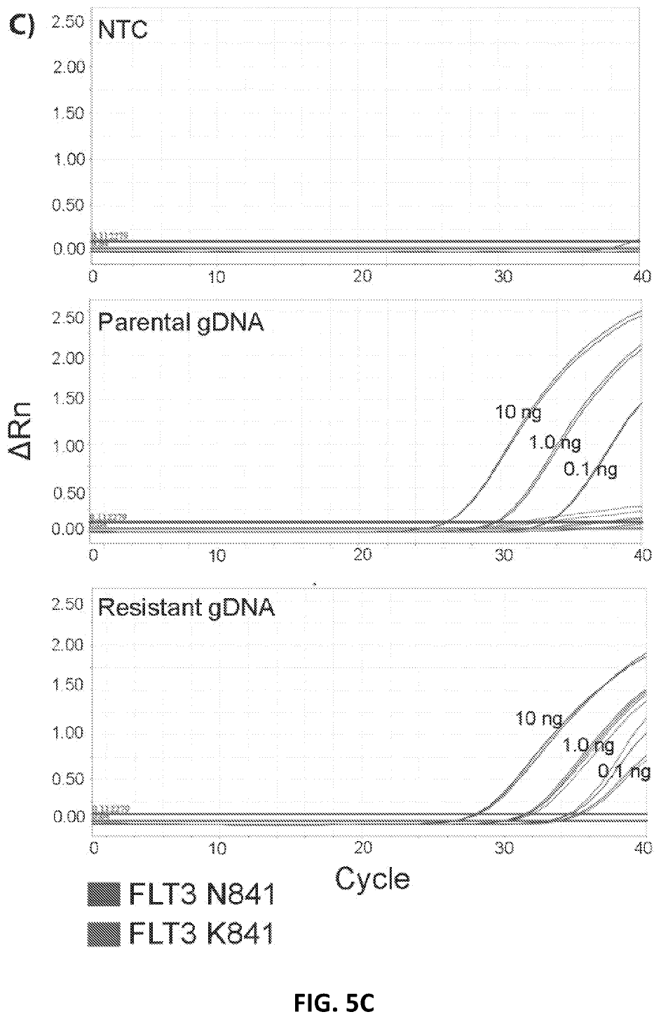

Candidate key drivers of quizartinib resistance were determined beyond gross CNV at the increased level of genomic resolution of the SNV. All parental and resistant single cells harbored FLT3 ITD (FIG. 5A). In contrast, a missense mutation N841K was detected in all quizartinib resistant cells (FIG. 5B). FLT3 N841K has previously been detected in AML patients, resides in the activation loop of FLT3, and furthermore, mutation of the residue corresponding to N841 in the closely-related receptor tyrosine kinase KIT is activating. Without being bound by theory, this suggests that N841K is a chief secondary mutation to ITD and is plausibly contributing to quizartinib resistance in this model by preventing efficiency of drug binding.

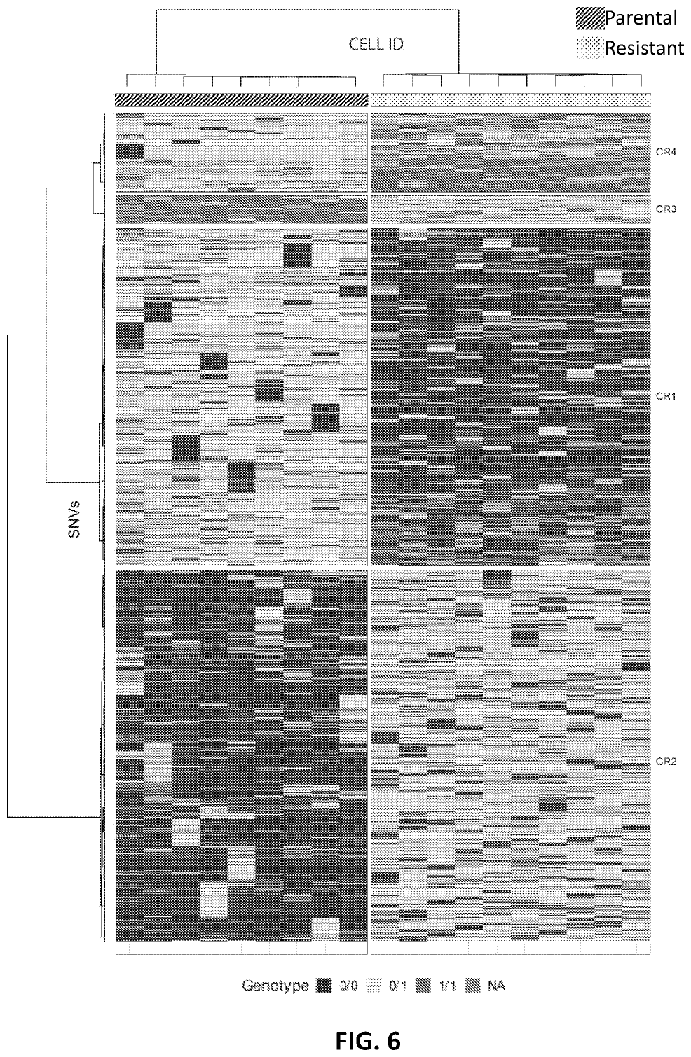

To assess whether the N841K FLT3 secondary mutation may have arisen de novo or was an existing genetic variant clone in the parental population a custom quantitative PCR-based genotyping assay was employed to distinguish between the two scenarios. This probe set, emitting fluorescence of differing wavelengths for allelic discrimination between N841 and K841 upon probe binding and dequenching, was employed in qPCR assays of genomic DNA isolated from either parental or quizartinib-resistant MOLM-13 cells. In parental cells, while amplification of N841 dominated, a low but detectable level of K841 presented (FIG. 5C). Resistant cells displayed a contrasting scenario, whereby there was equal signal from N841 and K841. These data suggest that FL.73 K841 existed as an extremely rare clone in the original MOLM-13 cell line which upon the selective pressure of quizartinib was enriched to domination of the resistant cell line likely due to its ability to affect drug binding-thus highlighting our cell line model's emulation of clonal selection in patient tumors. While this variation independently makes a compelling case, with the increased biomarker resolution, well-defined groups were identified by the heatmap in FIG. 6 that showcases differential genotypes across the two groups.

### Heterogenous SNV in MOLM-13 Quizartinib Resistance

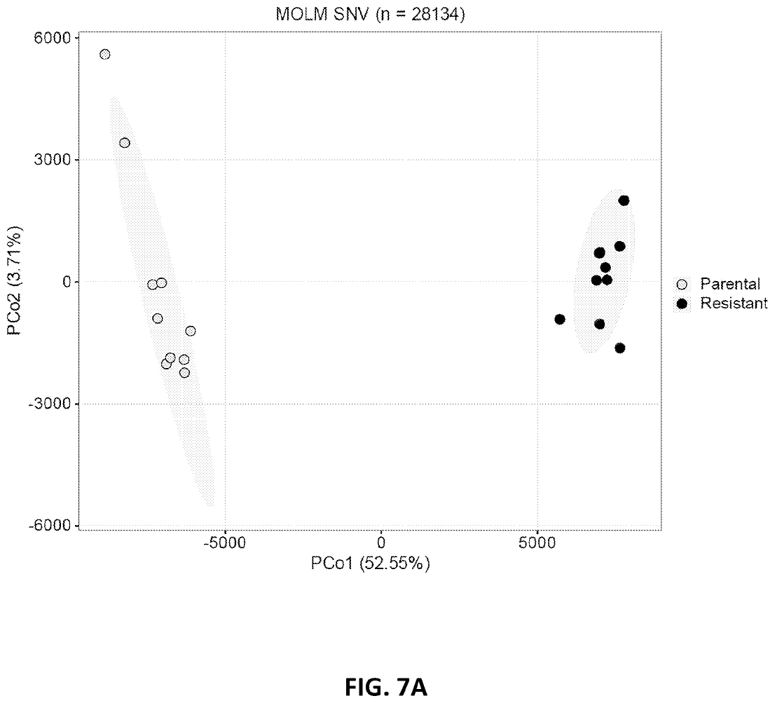

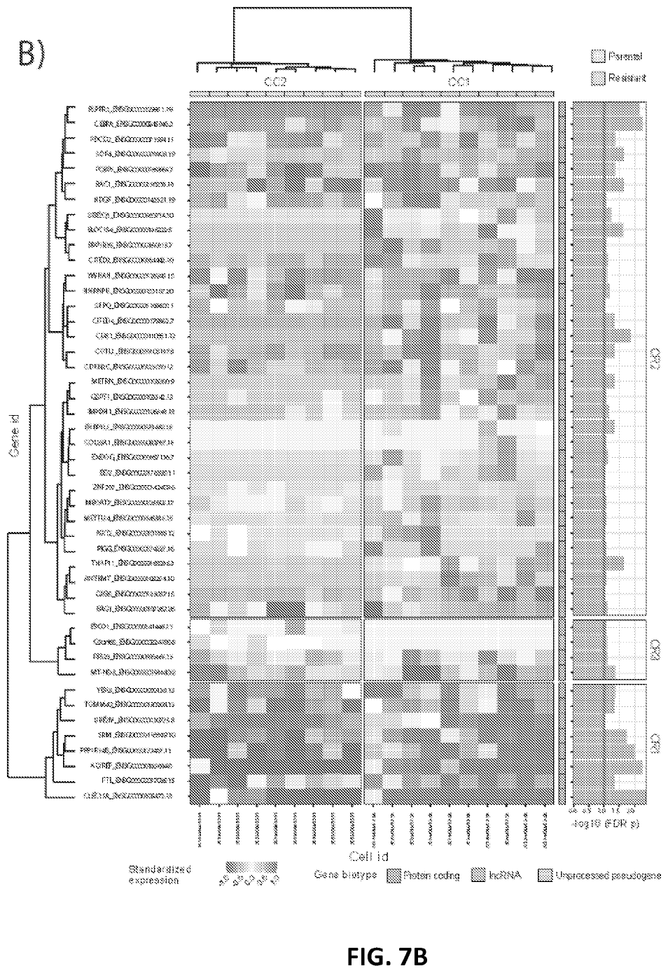

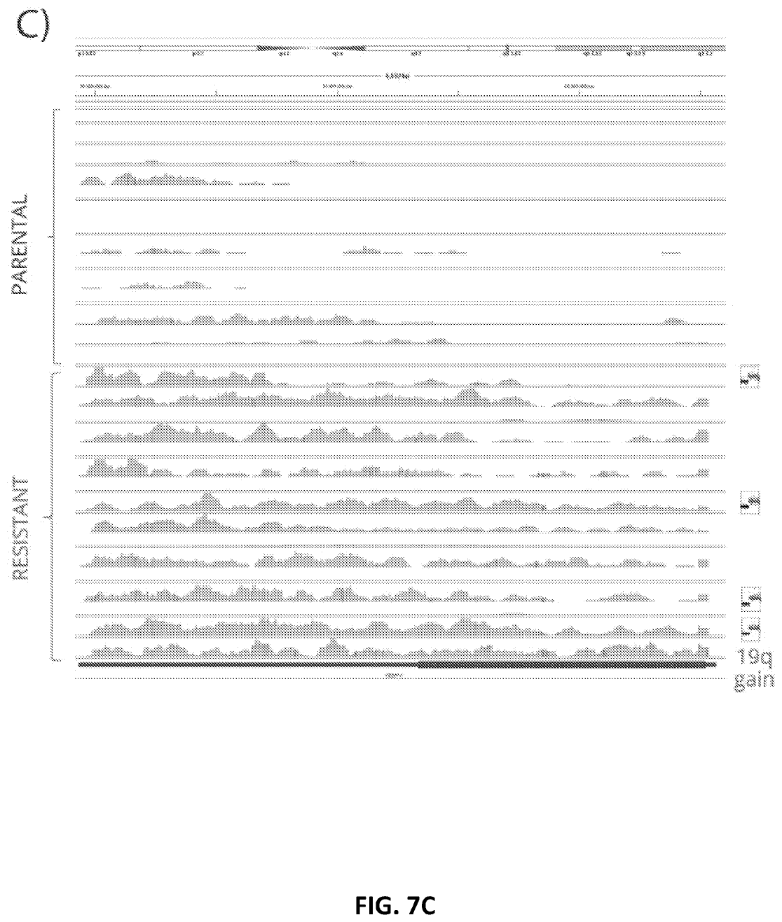

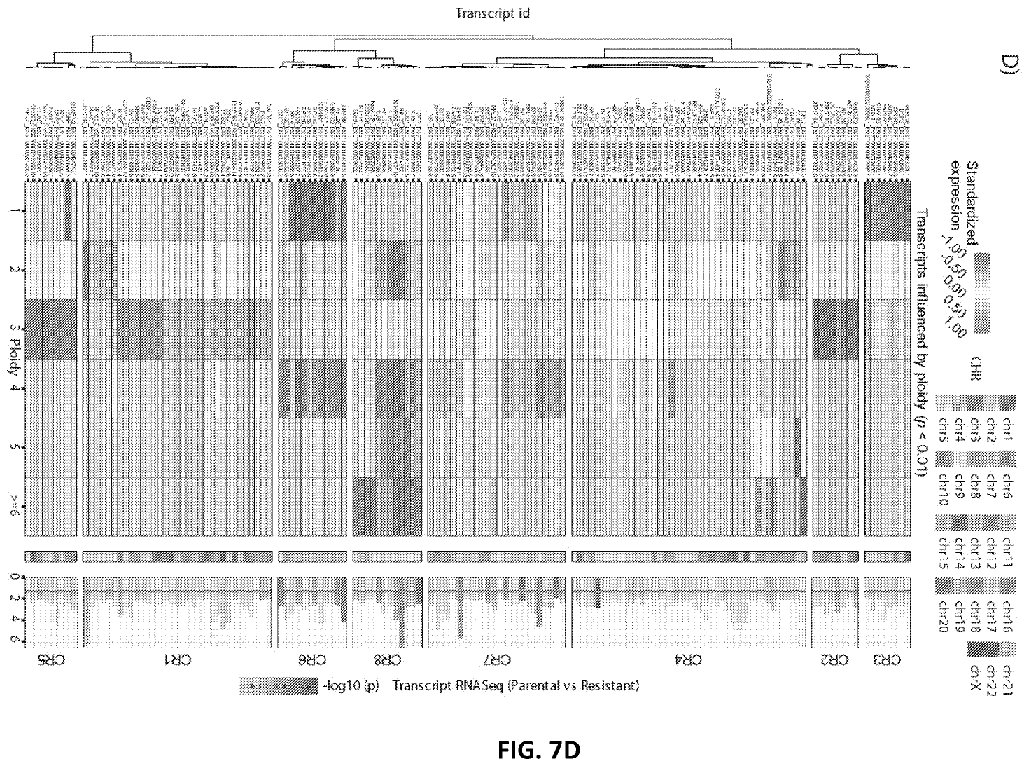

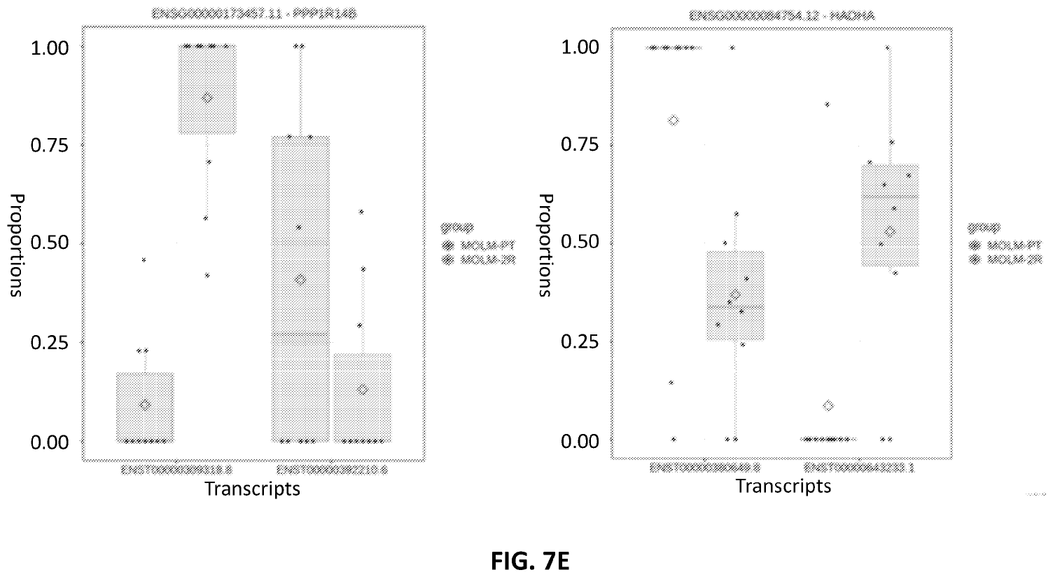

A candidate list of genes representing multiple functional classes—signaling, epigenetic, tumor suppressor, spliceosome, cohesion complex genes—previously implicated in AML pathogenesis for SNV was interrogated. With no resistant-specific coding sequence changes in single cells identified with this candidate approach other than the FLT3 secondary mutation, an unbiased search was conducted for mutations that may be contributing to quizartinib resistance and for those mutations representing subclones and not found in all resistant cells. The variant call file was first stratified by rarer functional class of mutation, stop codon gain and frameshift mutation, due to the increased likelihood of deleterious functional consequences. A heterozygous nonsense mutation in the splicing and mRNA stability factor CELF4 in 7/10 quizartinib-resistant cells was identified where the change was not identified in any single cells of the parental cohort. Frameshift mutations were identified in the metabolic enzyme ADSSI at K291 (c.870dupC) in 8/10 quizartinib resistant and 0/9 parental cells and in the GTP-binding protein RRAGC at A57 (c. 167dupG) in 5/10 resistant cells and in 0/9 parental cells. Although initially prioritizing these variants, no expression of their cognate transcripts was detected (FIG. 7B). This suggested that either these genes were lowly expressed in MOLM-13 cells, unexpressed at the time of cell capture and extraction, and/or beyond our limit of detection with multiomics. These findings motivated us to more comprehensively quantify the single nucleotide variation in our model, as well as to prioritize genomic variants associated with gene expression, which multiomics uniquely enables for single cells.

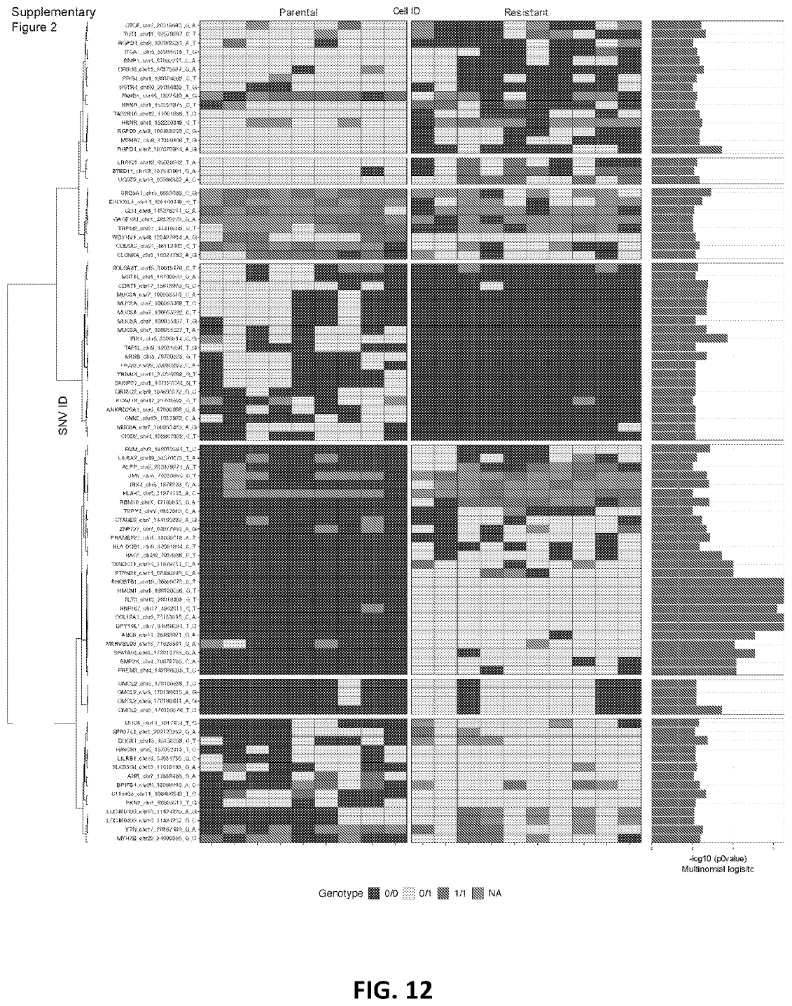

A variant filtering/prioritization strategy was then employed to identify single nucleotide variation present in quizartinib-resistant single cells but not in parental single cells. From this analysis (see Methods), multinomial logistic regression analysis and a Wald test was used to yield 6444 SNVs that were differentially prevalent between parental and resistant single cells (p<0.05). FIG. 6 presents this statistically significant genotypic variation in a heat map and allows visualization of conversion of homozygous reference (0/0) to heterozygous (1/0, 0/1) or homozygous alternate (1/1) alleles in the resistant cells, and, conversely, loss of heterozygous genotypes in the resistant cells to homozygous reference. Additional filtration by allowed us to focus on missense variations differing in parental vs. resistant line in FIG. 12. As a prioritized missense mutation of biological interest with validated mRNA expression, A109V was found in the E3 ubiquitin ligase gene RNF167, and found in all 10 quizartinib-resistant cells but not present in cells of the parental cohort.

In addition to prioritizing coding sequence variation above, variant filtration (See details in Methods) allowed us to discern a remarkable degree of single nucleotide variation in intergenic space occurring in our quizartinib resistance model. 8601 intergenic SNVs were cataloged in parental cells vs. 2167 in our quizartinib resistant cell cohort present in at least 25% of all cells within the group. This group-specific variation shows context of both selection of existing genomic variation in response to drug treatment and in de novo mutation and an exemplification of the high degree of plasticity in the genome (FIG. 6).

### Molm-13 Quizartinib-Resistant Cells Exhibit a Distinct Transcriptional Signature Including Adaptive Bypass

At the SNV level, there was distinction between parental and resistant MOLM-13 single cells in principal coordinate analysis (p<0.05, FIG. 7A). The same trend was seen in the multiomics transcriptomes of the two MOLM-13 single cell cohorts (data not shown). FIG. 7B illustrates a dendrogram highlighting differentially expressed transcripts between the P and R single cells and labeled by biotype indicating the categorical nature of the upregulated or downregulated transcript. Two specific examples are highlighted where both DNA and RNA-level contributions to drug resistance in this model.

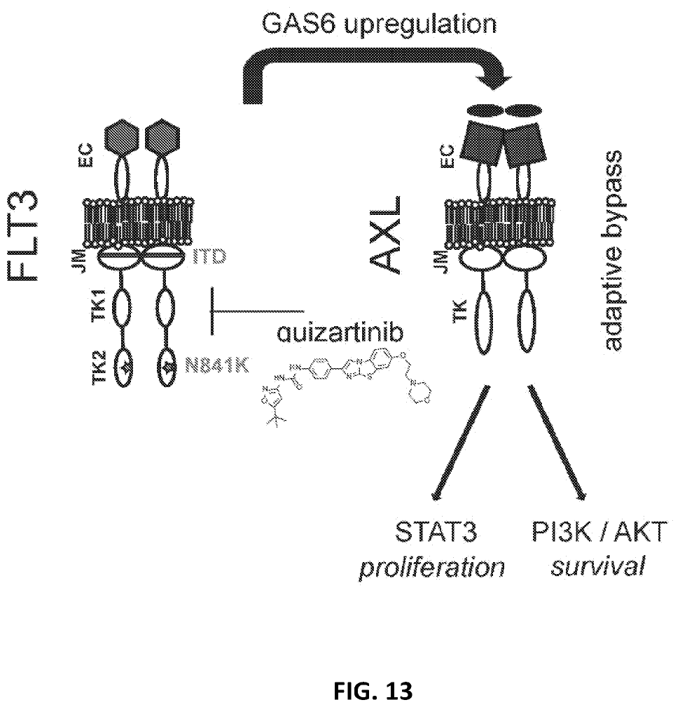

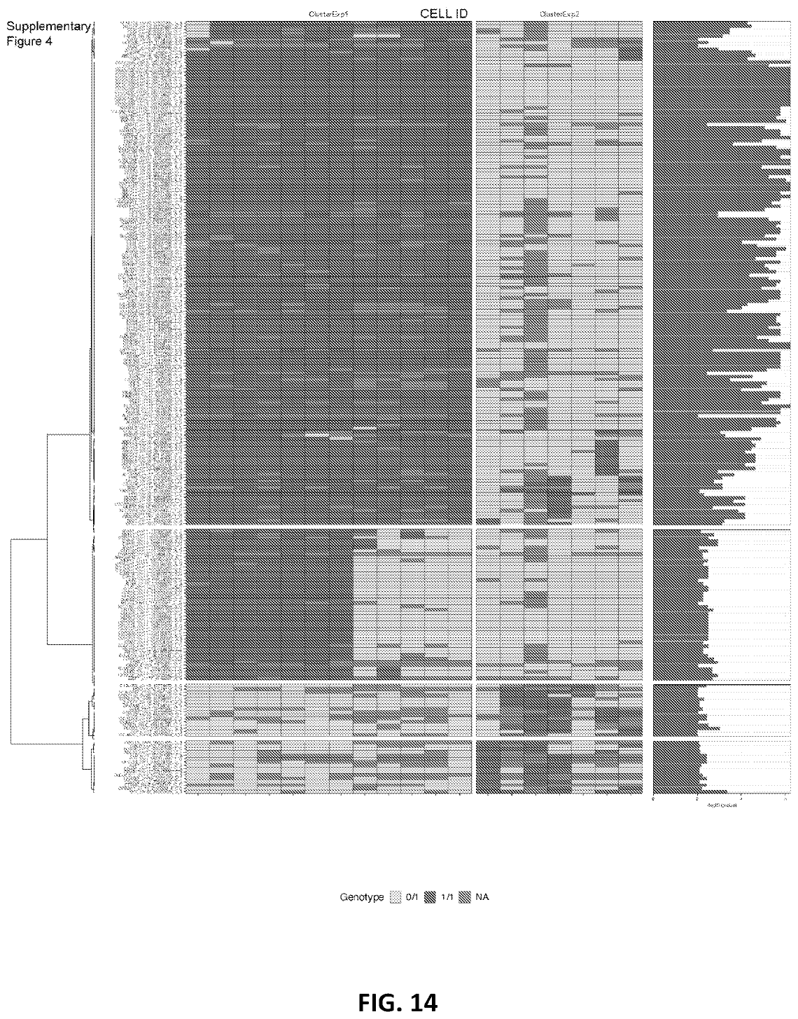

Firstly, from the differentially expressed gene set GAS6, a ligand for the receptor tyrosine kinase AXL, was upregulated. The AXL pathway, specifically through downstream STAT3 cell proliferation and PI3K/ALT survival signaling, has been shown to be a bypass pathway for FLT3 inhibition (FIG. 13). Also observed was concurrent transcriptional upregulation of the small GTPase RACI, which may be synergistic with upregulation of the AXL-STAT3 and AXL-PI3K/AKT signaling axes. Collectively, these transcriptional responses indicate a mode of adaptive transcriptional bypass that is occurring in the same cell harboring a DNA-level, secondary FL.73 mutation driving drug resistance. Intriguingly, it was also noted the pioneer transcription factor CEBPA CCAAT/enhancer-binding protein alpha (C/EBPa) transcriptional upregulation in quizartinib-resistant cells (FIG. 7B). Truncating mutations in CEBPA are found in ˜10-15% of AML patients, leading to expression of an N terminal fragment of CEBPA, p30, with potential dominant negative activity. As CEBPA resides on Chr. 19q13.11, concomitant with the transcriptional upregulation of CEBPA, Chr. 19q gain was observed in a subset of quizartinib-resistant cells (FIG. 7C) suggesting a potential genomic mechanism of CEBPA expression upregulation and exemplifying the power of the unification of single-cell genomic and transcriptomic data.

While plausible, no positive correlation was observed between copy number gain at CEBPA upregulation in individual cells, suggesting that the mode of transcript upregulation is epigenetic in nature. The relationship of ploidy to gene expression genome-wide using a zero-inflated linear model was then evaluated. Ploidy and gene expression were not direct correlates using a 500 kb window size, except for a set of genes whereby statistically meaningful associations were identified (p<0.05) with this model (FIG. 7D). Table 4 shows each gene identified and summarizes copy number and expression correlates. This highlights the importance of concurrent transcriptomic assessment when interpreting copy number alterations in single cells, as well as highlights the significant single cell heterogeneity that occurs in terms of ploidy across sub-megabase chromosomal intervals.

TABLE 4

| ensembl_transcript_{—} | | chromosome_{—} | transcript_{—} | transcript_{—} | ensembl_gene_id_{—} | gene_{—} |
|---|---|---|---|---|---|---|
| id_version | pvalue | name | start | end | version | symbol |
| ENST00000623083.4 | 0.01098406 | chr1 | 185217 | 195411 | ENSG00000279457.4 | WASH9P |
| ENST00000344843.12 | 0.04542944 | chr1 | 1401909 | 1407293 | ENSG00000242485.6 | MRPL20 |
| ENST00000615252.4 | 0.00026311 | chr1 | 1785285 | 1891117 | ENSG00000078369.18 | GNB1 |
| ENST00000317122.2 | 0.031276891 | chrl | 15834474 | 15848147 | ENSG00000179743.4 | FLJ37453 |
| ENST00000370951.5 | 0.03824769 | chr1 | 70205682 | 70251617 | ENSG00000116754.13 | SRSF11 |
| ENST00000370630.6 | 0.000728058 | chr1 | 84549611 | 84574440 | ENSG00000117151.13 | CTBS |
| ENST00000316005.11 | 0.021659192 | chr1 | 88684222 | 88806268 | ENSG00000065243.20 | PKN2 |
| ENST00000370454.9 | 0.005467674 | chr1 | 89633120 | 89719533 | ENSG00000171488.15 | LRRC8C |
| ENST00000644549.1 | 0.012628166 | chr1 | 92840525 | 92841921 | ENSG00000122406.14 | RPL5 |
| ENST00000436063.7 | 0.046298428 | chr1 | 93866284 | 93879206 | ENSG00000067334.14 | DNTTIP2 |
| ENST00000370021.1 | 0.000556088 | chr1 | 108692341 | 108701803 | ENSG00000134186.12 | PRPF38B |
| ENST00000263168.4 | 0.040840953 | chr1 | 112619832 | 112671616 | ENSG00000116489.13 | CAPZA1 |
| ENST00000684484.1 | 0.041386413 | chr1 | 116111399 | 116138149 | ENSG00000173212.5 | MAB21L3 |
| ENST00000470935.1 | 0.016887973 | chr1 | 117060362 | 117075444 | ENSG00000116830.12 | TTF2 |
| ENST00000641863.1 | 0.043204929 | chr1 | 144892021 | 145095528 | ENSG00000196369.11 | SRGAP2B |
| ENST00000369051.7 | 0.002585074 | chr1 | 150487435 | 150507598 | ENSG00000143374.17 | TARS2 |
| ENST00000368863.6 | 0.038745077 | chr1 | 151402725 | 151459179 | ENSG00000143442.22 | POGZ |
| ENST00000548830.2 | 0.006234767 | chr1 | 155459797 | 155562807 | ENSG00000116539.14 | ASH1L |
| ENST00000366812.6 | 0.047187497 | chr1 | 226144679 | 226186741 | ENSG00000182827.9 | ACBD3 |
| ENST00000261396.6 | 0.002176251 | chr1 | 229440259 | 229508341 | ENSG00000069248.12 | NUP133 |
| ENST00000542957.1 | 0.037805046 | chr10 | 3775997 | 3785281 | ENSG00000067082.15 | KLF6 |
| ENST00000460569.1 | 2.76E−05 | chr10 | 12035059 | 12043170 | ENSG00000151461.20 | UPF2 |
| ENST00000677440.1 | 0.00959114 | chr10 | 27168135 | 27240784 | ENSG00000107897.20 | ACBD5 |
| ENST00000480465.1 | 0.00237465 | chr10 | 28898996 | 28899484 | ENSG00000229605.5 | RPL21P93 |
| ENST00000374466.4 | 0.001414649 | chr10 | 43138445 | 43185302 | ENSG00000169826.8 | CSGALNACT2 |
| ENST00000482069.5 | 0.012740275 | chr10 | 78033917 | 78040696 | ENSG00000138326.21 | RPS24 |
| ENST00000282728.10 | 0.021000689 | chr10 | 92689955 | 92695647 | ENSG00000152804.11 | HHEX |
| ENST00000371327.2 | 0.000222102 | chr10 | 94590699 | 94613905 | ENSG00000119969.15 | HELLS |
| ENST00000329399.7 | 0.038606127 | chr10 | 95237572 | 95291003 | ENSG00000107438.9 | PDLIM1 |
| ENST00000334828.6 | 0.002415022 | chr10 | 97426191 | 97433444 | ENSG00000171314.9 | PGAM1 |
| ENST00000298999.8 | 0.02093379 | chr10 | 98134657 | 98244897 | ENSG00000166024.14 | R3HCC1L |
| ENST00000370355.3 | 0.012209807 | chr10 | 100347233 | 100364826 | ENSG00000099194.6 | SCD |
| ENST00000361804.5 | 0.000169523 | chr10 | 110567695 | 110606048 | ENSG00000108055.10 | SMC3 |
| ENST00000298510.4 | 0.013256127 | chr10 | 119167720 | 119178812 | ENSG00000165672.7 | PRDX3 |
| ENST00000528024.1 | 0.032752962 | chr11 | 703007 | 704129 | ENSG00000177042.16 | TMEM80 |
| ENST00000529770.5 | 0.015780974 | chr11 | 118401619 | 118409199 | ENSG00000167283.8 | ATP5MG |
| ENST00000648516.1 | 0.000255604 | chr11 | 126327866 | 126345754 | ENSG00000110063.10 | DCPS |
| ENST00000544050.1 | 0.024886533 | chr12 | 4612517 | 4613823 | ENSG00000010219.13 | DYRK4 |
| ENST00000543959.5 | 0.013416833 | chr12 | 6492152 | 6493718 | ENSG00000111639.8 | MRPL51 |
| ENST00000619601.1 | 0.024988784 | chr12 | 6536605 | 6538374 | ENSG00000111640.15 | GAPDH |
| ENST00000539196.2 | 0.023527956 | chr12 | 6971061 | 6975977 | ENSG00000126749.16 | EMG1 |
| ENST00000470985.3 | 0.003864016 | chr12 | 21635342 | 21638538 | ENSG00000111716.14 | LDHB |
| ENST00000301180.10 | 2.14E−06 | chr12 | 50504985 | 50748657 | ENSG00000066084.13 | DIP2B |
| ENST00000394349.9 | 0.009112756 | chr12 | 53665170 | 53676083 | ENSG00000135390.21 | ATP5MC2 |
| ENST00000550411.5 | 0.005353158 | chr12 | 54241755 | 54259545 | ENSG00000094916.16 | CBX5 |
| ENST00000552766.5 | 0.000514806 | chr12 | 56104537 | 56113907 | ENSG00000170515.14 | PA2G4 |
| ENST00000550443.5 | 0.027529899 | chr12 | 56152439 | 56157976 | ENSG00000196465.10 | MYL6B |
| ENST00000550164.6 | 0.00497022 | chr12 | 56162359 | 56189483 | ENSG00000139613.12 | SMARCC2 |
| ENST00000262030.8 | 0.000317735 | chr12 | 56638175 | 56645984 | ENSG00000110955.9 | ATP5F1B |
| ENST00000262033.11 | 0.040019526 | chr12 | 56663349 | 56688284 | ENSG00000110958.16 | PTGES3 |
| ENST00000678376.1 | 0.011439605 | chr12 | 56712428 | 56725286 | ENSG00000196531.14 | NACA |
| ENST00000557781.5 | 0.040075705 | chr12 | 57103273 | 57105317 | ENSG00000166888.12 | STAT6 |
| ENST00000548249.6 | 0.012858673 | chr12 | 57530051 | 57547192 | ENSG00000175203.17 | DCTN2 |
| ENST00000261267.7 | 0.033923008 | chr12 | 69348381 | 69354234 | ENSG00000090382.7 | LYZ |
| ENST00000550926.1 | 0.02236497 | chr12 | 74538145 | 74538633 | ENSG00000257386.1 | ENSG00000257386.1 |
| ENST00000456650.7 | 0.018752986 | chr12 | 75480881 | 75498717 | ENSG00000139278.10 | GLIPR1 |
| ENST00000618691.5 | 0.004157284 | chr12 | 76036585 | 76084685 | ENSG00000187109.15 | NAP1L1 |
| ENST00000549098.5 | 0.002388472 | chr12 | 120196708 | 120201110 | ENSG00000089157.16 | RPLP0 |
| ENST00000424014.7 | 0.033529543 | chr12 | 123620406 | 123633686 | ENSG00000111361.13 | EIF2B1 |
| ENST00000663159.1 | 0.009663031 | chr13 | 46052497 | 46161379 | ENSG00000235903.9 | CPB2-AS1 |
| ENST00000398576.6 | 0.013476691 | chr13 | 46125920 | 46211348 | ENSG00000136167.15 | LCP1 |
| ENST00000416500.5 | 0.048115274 | chr13 | 46152781 | 46168519 | ENSG00000136167.15 | LCP1 |
| ENST00000378549.5 | 0.023122171 | chr13 | 48233203 | 48261388 | ENSG00000136156.15 | ITM2B |
| ENST00000267163.6 | 0.002246426 | chr13 | 48303751 | 48481890 | ENSG00000139687.16 | RB1 |
| ENST00000458725.6 | 0.047399336 | chr13 | 50043492 | 50082041 | ENSG00000231607.13 | DLEU2 |
| ENST00000298125.7 | 0.030811508 | chr13 | 51584462 | 51767709 | ENSG00000139668.9 | WDFY2 |
| ENST00000304625.3 | 0.001801335 | chr14 | 20955487 | 20956436 | ENSG00000169385.3 | RNASE2 |
| ENST00000555914.5 | 0.03363115 | chr14 | 21210613 | 21269404 | ENSG00000092199.18 | HNRNPC |
| ENST00000611116.2 | 0.000905665 | chr14 | 22547506 | 22552156 | ENSG00000277734.8 | TRAC |
| ENST00000457657.5 | 0.026619855 | chr14 | 23058567 | 23095614 | ENSG00000100813.15 | ACIN1 |
| ENST00000612263.1 | 0.016389911 | chr15 | 22681094 | 22681885 | ENSG00000276141.4 | WHAMMP3 |
| ENST00000337451.8 | 0.001611852 | chr15 | 22838666 | 22868384 | ENSG00000140157.16 | NIPA2 |
| ENST00000610365.4 | 0.030169455 | chr15 | 22869466 | 22980352 | ENSG00000273749.5 | CYFIP1 |
| ENST00000568588.5 | 0.000374002 | chr15 | 66499323 | 66524532 | ENSG00000174444.15 | RPL4 |
| ENST00000565723.1 | 0.013436 | chr15 | 66499323 | 66499818 | ENSG00000174444.15 | RPL4 |
| ENST00000568077.5 | 0.013406295 | chr16 | 634502 | 636331 | ENSG00000130731.16 | METTL26 |
| ENST00000565813.1 | 0.008259483 | chr16 | 682416 | 682870 | ENSG00000103266.11 | STUB1 |
| ENST00000575009.5 | 0.005206574 | chr16 | 2752638 | 2761707 | ENSG00000167978.17 | SRRM2 |
| ENST00000571133.6 | 0.027721314 | chr16 | 11833850 | 11851542 | ENSG00000171490.13 | RSL1D1 |
| ENST00000304414.12 | 0.047461449 | chr16 | 18791669 | 18801549 | ENSG00000170540.15 | ARL6IP1 |
| ENST00000446231.7 | 0.000116939 | chr16 | 18804860 | 18926408 | ENSG00000157106.17 | SMG1 |
| ENST00000569764.1 | 0.02400657 | chr16 | 18856338 | 18861487 | ENSG00000157106.17 | SMG1 |
| ENST00000268379.9 | 0.004651303 | chr16 | 21953361 | 21983660 | ENSG00000140740.11 | UQCRC2 |
| ENST00000431282.2 | 0.021510718 | chr16 | 28494649 | 28498970 | ENSG00000184730.11 | APOBR |
| ENST00000311008.16 | 0.045274451 | chr16 | 28974778 | 28984543 | ENSG00000169682.18 | SPNS1 |
| ENST00000569760.5 | 0.026105001 | chr16 | 31190216 | 31191605 | ENSG00000089280.19 | FUS |
| ENST00000561508.1 | 0.001189403 | chr16 | 31202194 | 31203450 | ENSG00000103490.14 | PYCARD |
| ENST00000303383.8 | 0.005379784 | chr16 | 46578591 | 46621379 | ENSG00000171241.9 | SHCBP1 |
| ENST00000567402.5 | 0.027528182 | chr16 | 47461336 | 47590838 | ENSG00000102893.16 | PHKB |
| ENST00000330943.9 | 0.04236657 | chr16 | 50671629 | 50681312 | ENSG00000167208.15 | SNX20 |
| ENST00000245185.6 | 0.03511947 | chr16 | 56608584 | 56609497 | ENSG00000125148.7 | MT2A |
| ENST00000219400.8 | 0.031868994 | chr16 | 80966448 | 81006885 | ENSG00000103121.9 | CMC2 |
| ENST00000564365.5 | 0.017949831 | chr16 | 88856220 | 88861415 | ENSG00000167515.10 | TRAPPC2L |
| ENST00000562879.5 | 0.014179601 | chr16 | 89560682 | 89563473 | ENSG00000167526.14 | RPL13 |
| ENST00000658116.1 | 0.020970548 | chr17 | 16439038 | 16442011 | ENSG00000175061.18 | SNHG29 |
| ENST00000672357.1 | 0.031558391 | chr17 | 19648723 | 19675664 | ENSG00000072210.19 | ALDH3A2 |
| ENST00000261712.8 | 0.002981642 | chr17 | 32444510 | 32483319 | ENSG00000108671.11 | PSMD11 |
| ENST00000479035.7 | 0.00588973 | chr17 | 38847860 | 38853721 | ENSG00000125691.14 | RPL23 |
| ENST00000579374.5 | 0.001677911 | chr17 | 39200316 | 39204727 | ENSG00000108298.12 | RPL19 |
| ENST00000536605.1 | 0.038548021 | chr18 | 3252275 | 3256235 | ENSG00000101608.13 | MYL12A |
| ENST00000318388.11 | 0.011801034 | chr18 | 9102699 | 9134341 | ENSG00000178127.13 | NDUFV2 |
| ENST00000474740.1 | 0.001049112 | chr18 | 9126627 | 9134295 | ENSG00000178127.13 | NDUFV2 |
| ENST00000019317.8 | 0.009955481 | chr18 | 9475009 | 9538114 | ENSG00000017797.13 | RALBP1 |
| ENST00000635540.1 | 0.048348878 | chr18 | 20949952 | 21111020 | ENSG00000067900.8 | ROCK1 |
| ENST00000217740.4 | 0.000859598 | chr18 | 32018825 | 32073219 | ENSG00000101695.9 | RNF125 |
| ENST00000282050.6 | 0.001355835 | chr18 | 46084144 | 46104233 | ENSG00000152234.16 | ATP5F1A |
| ENST00000589955.2 | 0.007439769 | chr18 | 63313802 | 63318812 | ENSG00000171791.14 | BCL2 |
| ENST00000592957.1 | 0.034890595 | chr18 | 79973513 | 80033891 | ENSG00000141759.15 | TXNL4A |
| ENST00000262198.9 | 0.046639089 | chr18 | 80109262 | 80140346 | ENSG00000101544.9 | ADNP2 |
| ENST00000592590.6 | 0.033297023 | chr19 | 1009650 | 1017420 | ENSG00000182087.14 | TMEM259 |
| ENST00000320936.9 | 0.047563831 | chr19 | 1269337 | 1273172 | ENSG00000099622.14 | CIRBP |
| ENST00000233596.8 | 0.01788709 | chr19 | 1491181 | 1497927 | ENSG00000115255.12 | REEP6 |
| ENST00000600737.6 | 0.010317295 | chr19 | 7535717 | 7561764 | ENSG00000032444.17 | PNPLA6 |
| ENST00000304863.6 | 0.008358638 | chr19 | 29205320 | 29213151 | ENSG00000169021.6 | UQCRFS1 |
| ENST00000590247.7 | 0.002670993 | chr19 | 32581190 | 32587453 | ENSG00000105185.12 | PDCD5 |
| ENST00000397061.4 | 0.01092263 | chr19 | 32691821 | 32713792 | ENSG00000213965.4 | NUDT19 |
| ENST00000498907.3 | 0.033967443 | chr19 | 33299934 | 33302534 | ENSG00000245848.3 | CEBPA |
| ENST00000588991.7 | 0.014824716 | chr19 | 34364793 | 34400245 | ENSG00000105220.17 | GPI |
| ENST00000586425.2 | 0.014161576 | chr19 | 34365379 | 34400267 | ENSG00000105220.17 | GPI |
| ENST00000439527.6 | 0.001668955 | chr19 | 34428876 | 34469890 | ENSG00000126261.13 | UBA2 |
| ENST00000222284.10 | 0.004712604 | chr19 | 35545626 | 35547526 | ENSG00000105677.12 | TMEM147 |
| ENST00000649813.2 | 3.36E−05 | chr19 | 35648323 | 35658782 | ENSG00000126267.11 | COX6B1 |
| ENST00000222266.2 | 0.003432669 | chr19 | 35745619 | 35747004 | ENSG00000205155.8 | PSENEN |
| ENST00000221855.8 | 0.026713472 | chr19 | 36115468 | 36125941 | ENSG00000105254.12 | TBCB |
| ENST00000246533.8 | 0.008727067 | chr19 | 36140066 | 36150353 | ENSG00000126247.11 | CAPNS1 |
| ENST00000263372.5 | 0.01786605 | chr19 | 38319845 | 38332076 | ENSG00000099337.5 | KCNK6 |
| ENST00000251453.8 | 0.002126874 | chr19 | 39433137 | 39435949 | ENSG00000105193.9 | RPS16 |
| ENST00000601655.5 | 0.00106611 | chr19 | 39433217 | 39435941 | ENSG00000105193.9 | RPS16 |
| ENST00000594583.2 | 0.039530971 | chr19 | 39481225 | 39486495 | ENSG00000105197.11 | TIMM50 |
| ENST00000356508.9 | 0.014523199 | chr19 | 40348700 | 40378483 | ENSG00000105223.20 | PLD3 |
| ENST00000599570.5 | 0.024612935 | chr19 | 40750875 | 40759610 | ENSG00000077312.9 | SNRPA |
| ENST00000221233.9 | 0.004106926 | chr19 | 41386374 | 41397359 | ENSG00000077348.9 | EXOSC5 |
| ENST00000598742.6 | 0.000448285 | chr19 | 41860255 | 41872925 | ENSG00000105372.8 | RPS19 |
| ENST00000221975.6 | 0.002747064 | chr19 | 41860257 | 41871416 | ENSG00000105372.8 | RPS19 |
| ENST00000222330.8 | 0.031277742 | chr19 | 42230190 | 42242602 | ENSG00000105723.13 | GSK3A |
| ENST00000405636.6 | 0.040421882 | chr19 | 44891220 | 44903688 | ENSG00000130204.13 | TOMM40 |
| ENST00000300853.8 | 0.009153644 | chr19 | 45407334 | 45423917 | ENSG00000012061.16 | ERCC1 |
| ENST00000342669.8 | 0.003377001 | chr19 | 45687460 | 45691953 | ENSG00000125743.11 | SNRPD2 |
| ENST00000588301.5 | 0.042615028 | chr19 | 45687463 | 45692316 | ENSG00000125743.11 | SNRPD2 |
| ENST00000477244.5 | 0.005883937 | chr19 | 46601309 | 46609275 | ENSG00000160014.17 | CALM3 |
| ENST00000263270.11 | 0.017672567 | chr19 | 46838167 | 46850846 | ENSG00000042753.12 | AP2S1 |
| ENST00000263274.12 | 0.024521523 | chr19 | 48115445 | 48170344 | ENSG00000105486.14 | LIG1 |
| ENST00000519332.5 | 0.00791753 | chr19 | 48238334 | 48255893 | ENSG00000105483.18 | CARD8 |
| ENST00000549920.6 | 0.02597576 | chr19 | 48615331 | 48619178 | ENSG00000063177.13 | RPL18 |
| ENST00000331825.11 | 0.016044419 | chr19 | 48965309 | 48966879 | ENSG00000087086.15 | FTL |
| ENST00000262265.10 | 0.002254439 | chr19 | 49446298 | 49451814 | ENSG00000104872.11 | PIH1D1 |
| ENST00000391857.9 | 0.001875982 | chr19 | 49487608 | 49492308 | ENSG00000142541.18 | RPL13A |
| ENST00000270625.7 | 0.032393166 | chr19 | 49496434 | 49499708 | ENSG00000142534.7 | RPS11 |
| ENST00000339093.7 | 0.010877188 | chr19 | 49555711 | 49580558 | ENSG00000142546.14 | NOSIP |
| ENST00000253727.10 | 0.014044984 | chr19 | 50376457 | 50383388 | ENSG00000131408.15 | NR1H2 |
| ENST00000440232.7 | 0.040394011 | chr19 | 50384347 | 50418014 | ENSG00000062822.15 | POLD1 |
| ENST00000598585.1 | 0.036237289 | chr19 | 50476521 | 50483198 | ENSG00000161671.17 | EMC10 |
| ENST00000250340.9 | 0.013651537 | chr19 | 50723364 | 50725708 | ENSG00000105472.13 | CLEC11A |
| ENST00000617718.4 | 0.010016512 | chr19 | 50723387 | 50725718 | ENSG00000105472.13 | CLEC11A |
| ENST00000599973.1 | 0.028378254 | chr19 | 50723523 | 50725469 | ENSG00000105472.13 | CLEC11A |
| ENST00000309244.9 | 0.023123072 | chr19 | 51345169 | 51366388 | ENSG00000105379.10 | ETFB |
| ENST00000462990.5 | 0.002087027 | chr19 | 52201093 | 52226417 | ENSG00000105568.18 | PPP2R1A |
| ENST00000437868.5 | 0.006235115 | chr19 | 54173412 | 54189443 | ENSG00000125505.17 | MBOAT7 |
| ENST00000302907.9 | 0.043447128 | chr19 | 54200858 | 54207647 | ENSG00000170889.14 | RPS9 |
| ENST00000558815.5 | 0.00675774 | chr19 | 55385932 | 55391803 | ENSG00000108107.15 | RPL28 |
| ENST00000558131.1 | 0.002445994 | chr19 | 55386350 | 55388373 | ENSG00000108107.15 | RPL28 |
| ENST00000085079.11 | 1.66E−06 | chr19 | 55675245 | 55695766 | ENSG00000063245.15 | EPN1 |
| ENST00000253023.8 | 7.89E−05 | chr19 | 58555712 | 58558611 | ENSG00000130725.8 | UBE2M |
| ENST00000405489.7 | 0.001105631 | chr2 | 27212370 | 27217178 | ENSG00000138085.17 | ATRAID |
| ENST00000379619.5 | 0.005247284 | chr2 | 28392802 | 28417312 | ENSG00000075426.12 | FOSL2 |
| ENST00000263918.9 | 0.017970802 | chr2 | 36837698 | 36966536 | ENSG00000115808.12 | STRN |
| ENST00000397226.2 | 0.003064649 | chr2 | 37196523 | 37202692 | ENSG00000218739.10 | CEBPZOS |
| ENST00000457097.1 | 0.008916763 | chr2 | 38601598 | 38602178 | ENSG00000235586.1 | ENSG00000235586.1 |
| ENST00000398571.7 | 0.011662083 | chr2 | 61187463 | 61471087 | ENSG00000115464.15 | USP34 |
| ENST00000678113.1 | 0.01896042 | chr2 | 61476032 | 61500128 | ENSG00000082898.19 | XPO1 |
| ENST00000492182.6 | 5.93E−05 | chr2 | 61482818 | 61487541 | ENSG00000082898.19 | XPO1 |
| ENST00000476585.5 | 0.002545745 | chr2 | 61502146 | 61526523 | ENSG00000082898.19 | XPO1 |
| ENST00000398529.7 | 0.003001154 | chr2 | 65087871 | 65130101 | ENSG00000138069.18 | RAB1A |
| ENST00000282574.8 | 0.013324861 | chr2 | 70210204 | 70248580 | ENSG00000116001.17 | TIA1 |
| ENST00000409262.8 | 0.018048375 | chr2 | 73984910 | 74108176 | ENSG00000187605.16 | TET3 |
| ENST00000327428.10 | 0.004524 | chr2 | 74135400 | 74147912 | ENSG00000163170.12 | BOLA3 |
| ENST00000449856.1 | 0.001120744 | chr2 | 84915868 | 84916660 | ENSG00000213399.3 | RPS2P17 |
| ENST00000306368.9 | 0.004095713 | chr2 | 85595745 | 85597708 | ENSG00000168894.10 | RNF181 |
| ENST00000465560.5 | 0.039937467 | chr2 | 86106223 | 86121591 | ENSG00000132300.19 | PTCD3 |
| ENST00000605125.5 | 0.029882304 | chr2 | 86199457 | 86211045 | ENSG00000132313.15 | MRPL35 |
| ENST00000264258.8 | 0.003577724 | chr2 | 101002289 | 101007267 | ENSG00000071082.11 | RPL31 |
| ENST00000409318.2 | 0.002080511 | chr2 | 101007228 | 101151382 | ENSG00000204634.13 | TBC1D8 |
| ENST00000355857.8 | 0.0481748 | chr2 | 119366977 | 119372543 | ENSG00000155368.17 | DBI |
| ENST00000472146.5 | 0.014701829 | chr2 | 144426513 | 144520119 | ENSG00000169554.22 | ZEB2 |
| ENST00000625161.1 | 0.031711218 | chr2 | 144511391 | 144514451 | ENSG00000169554.22 | ZEB2 |
| ENST00000410080.8 | 0.019946139 | chr2 | 152651593 | 152717981 | ENSG00000196504.19 | PRPF40A |
| ENST00000392782.5 | 1.01E−05 | chr2 | 159318979 | 159616569 | ENSG00000123636.18 | BAZ2B |
| ENST00000322723.9 | 0.014899574 | chr2 | 231453531 | 231464484 | ENSG00000115053.17 | NCL |
| ENST00000468027.5 | 0.018128336 | chr2 | 231708530 | 231712675 | ENSG00000187514.17 | PTMA |
| ENST00000244815.9 | 0.010026755 | chr2 | 237692216 | 237765915 | ENSG00000124831.19 | LRRFIP1 |
| ENST00000543185.6 | 0.019480474 | chr2 | 239048168 | 239401020 | ENSG00000068024.17 | HDAC4 |
| ENST00000636051.1 | 0.041259337 | chr2 | 241687085 | 241721805 | ENSG00000168395.16 | ING5 |
| ENST00000381719.8 | 0.000445497 | chr20 | 1369000 | 1393123 | ENSG00000088832.18 | FKBP1A |
| ENST00000481690.2 | 0.027342125 | chr20 | 5575900 | 5610924 | ENSG00000125772.14 | GPCPD1 |
| ENST00000286788.9 | 0.019640566 | chr21 | 29056326 | 29073648 | ENSG00000156261.13 | CCT8 |
| ENST00000480486.1 | 0.008521806 | chr21 | 36386060 | 36398239 | ENSG00000159259.8 | CHAF1B |
| ENST00000338754.9 | 4.67E−05 | chr22 | 26521996 | 26590132 | ENSG00000128294.16 | TPST2 |
| ENST00000320996.14 | 0.006996197 | chr22 | 27851670 | 27919255 | ENSG00000180957.18 | PITPNB |
| ENST00000330029.6 | 0.017106214 | chr22 | 29767369 | 29770413 | ENSG00000184076.13 | UQCR10 |
| ENST00000249071.11 | 0.033234208 | chr22 | 37225270 | 37244269 | ENSG00000128340.15 | RAC2 |
| ENST00000248924.11 | 0.018898196 | chr22 | 37807934 | 37816897 | ENSG00000100116.17 | GCAT |
| ENST00000216019.11 | 5.19E−05 | chr22 | 38485681 | 38506294 | ENSG00000100201.23 | DDX17 |
| ENST00000216034.6 | 0.026833597 | chr22 | 38681957 | 38685421 | ENSG00000100216.6 | TOMM22 |
| ENST00000674155.1 | 0.01806158 | chr22 | 41093005 | 41180077 | ENSG00000100393.14 | EP300 |
| ENST00000327492.4 | 1.59E−05 | chr22 | 41433494 | 41446801 | ENSG00000183864.5 | TOB2 |
| ENST00000215956.10 | 0.004489274 | chr22 | 41674707 | 41682622 | ENSG00000100138.15 | SNU13 |
| ENST00000350028.5 | 0.030071367 | chr22 | 43955442 | 43996529 | ENSG00000100347.15 | SAMM50 |
| ENST00000347635.9 | 0.003195606 | chr22 | 45163925 | 45188017 | ENSG00000093000.19 | NUP50 |
| ENST00000483201.1 | 0.003122198 | chr3 | 47282959 | 47287458 | ENSG00000114648.12 | KLHL18 |
| ENST00000232496.5 | 0.040422611 | chr3 | 50324909 | 50328223 | ENSG00000114383.10 | TUSC2 |
| ENST00000420148.5 | 0.020791035 | chr3 | 52662204 | 52685917 | ENSG00000163939.18 | PBRM1 |
| ENST00000418458.6 | 0.011314644 | chr3 | 52686032 | 52694497 | ENSG00000163938.17 | GNL3 |
| ENST00000394729.6 | 0.037415039 | chr3 | 53161120 | 53192717 | ENSG00000163932.15 | PRKCD |
| ENST00000471660.5 | 0.018415054 | chr3 | 87227271 | 87255548 | ENSG00000083937.10 | CHMP2B |
| ENST00000296328.9 | 0.030258118 | chr3 | 196347662 | 196432427 | ENSG00000163960.12 | UBXN7 |
| ENST00000511316.1 | 0.000135592 | chr4 | 173331705 | 173334432 | ENSG00000164104.12 | HMGB2 |
| ENST00000509082.1 | 0.005904025 | chr5 | 240444 | 256682 | ENSG00000073578.17 | SDHA |
| ENST00000315013.9 | 0.029666198 | chr5 | 446149 | 467285 | ENSG00000180104.16 | EXOC3 |
| ENST00000274137.10 | 0.000117034 | chr5 | 1801407 | 1816048 | ENSG00000145494.12 | NDUFS6 |
| ENST00000255764.4 | 0.016719808 | chr5 | 6371874 | 6378547 | ENSG00000133398.4 | MED10 |
| ENST00000503026.5 | 0.013524695 | chr5 | 10249929 | 10265078 | ENSG00000150753.12 | CCT5 |
| ENST00000508451.1 | 0.004840243 | chr5 | 10250309 | 10255030 | ENSG00000150753.12 | CCT5 |
| ENST00000325366.14 | 0.03851086 | chr5 | 31532301 | 31555053 | ENSG00000082213.18 | C5orf22 |
| ENST00000507465.1 | 0.003310743 | chr5 | 32355648 | 32390317 | ENSG00000056097.16 | ZFR |
| ENST00000506237.5 | 0.044976922 | chr5 | 32531633 | 32601323 | ENSG00000113387.12 | SUB1 |
| ENST00000265073.9 | 0.004563642 | chr5 | 32585557 | 32604079 | ENSG00000113387.12 | SUB1 |
| ENST00000511615.5 | 0.001313118 | chr5 | 32585559 | 32601988 | ENSG00000113387.12 | SUB1 |
| ENST00000515355.5 | 0.001653484 | chr5 | 32585571 | 32601282 | ENSG00000113387.12 | SUB1 |
| ENST00000504789.5 | 0.003871383 | chr5 | 32585887 | 32601257 | ENSG00000113387.12 | SUB1 |
| ENST00000504016.1 | 0.002525197 | chr5 | 32588537 | 32601342 | ENSG00000113387.12 | SUB1 |
| ENST00000265112.8 | 0.04836474 | chr5 | 33440965 | 33468086 | ENSG00000113407.14 | TARS1 |
| ENST00000515010.5 | 0.026837373 | chr5 | 39107284 | 39203027 | ENSG00000082074.19 | FYB1 |
| ENST00000274242.10 | 0.000885937 | chr5 | 40825262 | 40835222 | ENSG00000145592.14 | RPL37 |
| ENST00000504562.1 | 2.36E−07 | chr5 | 40832458 | 40835186 | ENSG00000145592.14 | RPL37 |
| ENST00000508493.1 | 0.000410305 | chr5 | 40833879 | 40835212 | ENSG00000145592.14 | RPL37 |
| ENST00000196371.10 | 0.030127814 | chr5 | 41730065 | 41870425 | ENSG00000083720.13 | OXCT1 |
| ENST00000433297.2 | 0.016610806 | chr5 | 43289395 | 43313512 | ENSG00000112972.15 | HMGCS1 |
| ENST00000507293.1 | 0.009175417 | chr5 | 43298446 | 43313474 | ENSG00000112972.15 | HMGCS1 |
| ENST00000436644.6 | 0.026848473 | chr5 | 43527092 | 43557093 | ENSG00000172239.14 | PAIP1 |
| ENST00000507110.6 | 0.014359061 | chr5 | 44808947 | 44815514 | ENSG00000112996.11 | MRPS30 |
| ENST00000508934.5 | 0.038320343 | chr5 | 50398677 | 50441350 | ENSG00000170571.12 | EMB |
| ENST00000296684.10 | 0.045644204 | chr5 | 53560639 | 53683338 | ENSG00000164258.12 | NDUFS4 |
| ENST00000256441.5 | 0.016021383 | chr5 | 69217760 | 69230158 | ENSG00000134056.12 | MRPS36 |
| ENST00000509708.2 | 0.002557687 | chr5 | 73502591 | 73505493 | ENSG00000145741.16 | BTF3 |
| ENST00000513356.1 | 0.033012852 | chr5 | 74767263 | 74771653 | ENSG00000164346.10 | NSA2 |
| ENST00000680160.1 | 0.00447818 | chr5 | 75337200 | 75362089 | ENSG00000113161.17 | HMGCR |
| ENST00000646704.1 | 0.04554433 | chr5 | 77030902 | 77152155 | ENSG00000285000.1 | ENSG00000285000.1 |
| ENST00000493874.1 | 0.02569732 | chr5 | 77582376 | 77583122 | ENSG00000244363.3 | RPL7P23 |
| ENST00000380377.9 | 0.001620585 | chr5 | 77691166 | 77776339 | ENSG00000171530.15 | TBCA |
| ENST00000296674.13 | 0.001785722 | chr5 | 82273320 | 82278354 | ENSG00000186468.13 | RPS23 |
| ENST00000512493.5 | 0.012572028 | chr5 | 82275244 | 82278351 | ENSG00000186468.13 | RPS23 |
| ENST00000504293.5 | 2.31E−05 | chr5 | 82276060 | 82277951 | ENSG00000186468.13 | RPS23 |
| ENST00000247655.4 | 0.007689938 | chr5 | 86617941 | 86620962 | ENSG00000127184.13 | COX7C |
| ENST00000511086.1 | 0.030613464 | chr5 | 88804526 | 88883174 | ENSG00000081189.16 | MEF2C |
| ENST00000283109.8 | 0.035932569 | chr5 | 97160867 | 97183247 | ENSG00000058729.11 | RIOK2 |
| ENST00000504961.1 | 0.042740004 | chr5 | 112976813 | 112984651 | ENSG00000172795.17 | DCP2 |
| ENST00000503802.5 | 0.049037555 | chr5 | 119071205 | 119133691 | ENSG00000172869.15 | DMXL1 |
| ENST00000415806.2 | 0.00514807 | chr5 | 119356035 | 119394599 | ENSG00000145779.8 | TNFAIP8 |
| ENST00000510372.5 | 0.038803178 | chr5 | 122795536 | 122829671 | ENSG00000205302.7 | SNX2 |
| ENST00000506847.5 | 0.01713999 | chr5 | 122816923 | 122829773 | ENSG00000205302.7 | SNX2 |
| ENST00000505674.5 | 0.035936099 | chr5 | 126603561 | 126617333 | ENSG00000164902.14 | PHAX |
| ENST00000261366.10 | 0.028465529 | chr5 | 126777136 | 126837020 | ENSG00000113368.12 | LMNB1 |
| ENST00000304043.10 | 0.012628065 | chr5 | 131159027 | 131165256 | ENSG00000169567.13 | HINT1 |
| ENST00000511475.6 | 0.013727127 | chr5 | 131159285 | 131165349 | ENSG00000169567.13 | HINT1 |
| ENST00000508495.5 | 0.007965751 | chr5 | 131159288 | 131165301 | ENSG00000169567.13 | HINT1 |
| ENST00000378667.1 | 6.04E−07 | chr5 | 132866630 | 132867687 | ENSG00000164405.11 | UQCRQ |
| ENST00000496429.1 | 0.015544685 | chr5 | 132866642 | 132867699 | ENSG00000164405.11 | UQCRQ |
| ENST00000378670.8 | 0.019696866 | chr5 | 132866642 | 132868847 | ENSG00000164405.11 | UQCRQ |
| ENST00000395044.7 | 0.001738922 | chr5 | 133971915 | 134004742 | ENSG00000213585.11 | VDAC1 |
| ENST00000521216.5 | 0.003755003 | chr5 | 134157392 | 134176920 | ENSG00000113558.19 | SKP1 |
| ENST00000517625.5 | 0.035954679 | chr5 | 134157508 | 134176719 | ENSG00000113558.19 | SKP1 |
| ENST00000265339.7 | 0.042108898 | chr5 | 134371569 | 134392108 | ENSG00000119048.8 | UBE2B |
| ENST00000402673.7 | 0.003694993 | chr5 | 134601149 | 134632828 | ENSG00000152700.14 | SAR1B |
| ENST00000512507.5 | 0.03897208 | chr5 | 135334381 | 135362901 | ENSG00000113648.16 | MACROH2A1 |
| ENST00000510804.1 | 0.003794428 | chr5 | 138020763 | 138032928 | ENSG00000031003.10 | FAM13B |
| ENST00000230901.9 | 0.017760266 | chr5 | 138156882 | 138178630 | ENSG00000112983.18 | BRD8 |
| ENST00000394817.7 | 0.001294563 | chr5 | 138946724 | 139198368 | ENSG00000120725.13 | SIL1 |
| ENST00000310331.3 | 0.043884684 | chr5 | 140547662 | 140549576 | ENSG00000243056.2 | EIF4EBP3 |
| ENST00000518047.5 | 0.040090143 | chr5 | 141515975 | 141618914 | ENSG00000131504.17 | DIAPH1 |
| ENST00000513112.5 | 0.036207311 | chr5 | 144159273 | 144170630 | ENSG00000145817.17 | YIPF5 |
| ENST00000618084.2 | 0.036233803 | chr5 | 146151500 | 146182660 | ENSG00000133706.19 | LARS1 |
| ENST00000296702.9 | 0.002330837 | chr5 | 146447312 | 146511961 | ENSG00000113649.13 | TCERG1 |
| ENST00000353334.11 | 0.034306806 | chr5 | 150401670 | 150412750 | ENSG00000019582.16 | CD74 |
| ENST00000407193.7 | 0.010553465 | chr5 | 150442635 | 150449739 | ENSG00000164587.13 | RPS14 |
| ENST00000521466.5 | 0.004547971 | chr5 | 150444233 | 150448913 | ENSG00000164587.13 | RPS14 |
| ENST00000520931.5 | 0.006125843 | chr5 | 151029945 | 151081016 | ENSG00000145901.16 | TNIP1 |
| ENST00000420343.1 | 0.044009464 | chr5 | 157138442 | 157142775 | ENSG00000155868.8 | MED7 |
| ENST00000307063.9 | 0.013975173 | chr5 | 160091339 | 160119450 | ENSG00000170234.13 | PWWP2A |
| ENST00000265295.9 | 0.002846087 | chr5 | 169583773 | 169604778 | ENSG00000040275.17 | SPDL1 |
| ENST00000296930.10 | 0.006394468 | chr5 | 171387849 | 171410900 | ENSG00000181163.14 | NPM1 |
| ENST00000285908.5 | 0.004922989 | chr5 | 173607521 | 173616588 | ENSG00000145919.11 | BOD1 |
| ENST00000495423.1 | 0.003299383 | chr5 | 176238382 | 176296674 | ENSG00000170085.18 | SIMC1 |
| ENST00000393611.6 | 0.003585133 | chr5 | 177301451 | 177303706 | ENSG00000169228.14 | RAB24 |
| ENST00000390654.8 | 0.046600422 | chr5 | 178237618 | 178590393 | ENSG00000050767.18 | COL23A1 |
| ENST00000491103.5 | 0.006735258 | chr6 | 10747773 | 10756958 | ENSG00000137210.14 | TMEM14B |
| ENST00000376442.8 | 0.008336008 | chr6 | 30653127 | 30673006 | ENSG00000204560.10 | DHX16 |
| ENST00000244520.10 | 0.043740628 | chr6 | 34757505 | 34773857 | ENSG00000124562.10 | SNRPC |
| ENST00000373825.7 | 0.001913595 | chr6 | 35832966 | 35921098 | ENSG00000096063.16 | SRPK1 |
| ENST00000620389.1 | 0.004549468 | chr6 | 36599438 | 36602776 | ENSG00000112081.17 | SRSF3 |
| ENST00000230431.11 | 0.002043545 | chr6 | 43225629 | 43229481 | ENSG00000112667.13 | DNPH1 |
| ENST00000372236.9 | 0.022245031 | chr6 | 43576185 | 43620523 | ENSG00000170734.12 | POLH |
| ENST00000372133.8 | 0.035097063 | chr6 | 43671202 | 43687791 | ENSG00000096080.12 | MRPS18A |
| ENST00000372014.5 | 0.023460117 | chr6 | 44113451 | 44127452 | ENSG00000180992.7 | MRPL14 |
| ENST00000371646.10 | 0.013697654 | chr6 | 44247101 | 44253883 | ENSG00000096384.20 | HSP90AB1 |
| ENST00000370708.8 | 1.28E−05 | chr6 | 57090076 | 57109716 | ENSG00000112200.17 | ZNF451 |
| ENST00000370315.4 | 0.005477248 | chr6 | 73423711 | 73452297 | ENSG00000164430.17 | CGAS |
| ENST00000680833.1 | 0.006436405 | chr6 | 73424168 | 73452181 | ENSG00000164430.17 | CGAS |
| ENST00000415228.5 | 0.024023848 | chr6 | 73461765 | 73501096 | ENSG00000135297.17 | MTO1 |
| ENST00000684430.1 | 0.047739531 | chr6 | 75237675 | 75243801 | ENSG00000112695.12 | COX7A2 |
| ENST00000460985.1 | 0.018666426 | chr6 | 75237789 | 75243788 | ENSG00000112695.12 | COX7A2 |
| ENST00000275034.5 | 0.000111101 | chr6 | 78934419 | 79078254 | ENSG00000146247.14 | PHIP |
| ENST00000432102.5 | 0.015050821 | chr7 | 73220624 | 73230025 | ENSG00000182487.12 | NCF1B |
| ENST00000423083.1 | 0.010666553 | chr7 | 73220639 | 73235945 | ENSG00000182487.12 | NCF1B |
| ENST00000651031.1 | 0.018975732 | chr7 | 74843754 | 74887711 | ENSG00000277072.5 | STAG3L2 |
| ENST00000518240.5 | 0.000243808 | chr8 | 406428 | 435700 | ENSG00000147364.17 | FBXO25 |
| ENST00000352684.2 | 0.006621585 | chr8 | 406808 | 469871 | ENSG00000147364.17 | FBXO25 |
| ENST00000256398.13 | 0.044043589 | chr8 | 28093139 | 28191153 | ENSG00000134014.17 | ELP3 |
| ENST00000471393.1 | 0.00462498 | chr8 | 53532908 | 53533704 | ENSG00000240919.1 | ENSG00000240919.1 |
| ENST00000618914.4 | 0.03012961 | chr8 | 54046368 | 54102017 | ENSG00000120992.18 | LYPLA1 |
| ENST00000527553.1 | 0.002817424 | chr8 | 65725351 | 65728603 | ENSG00000066855.16 | MTFR1 |
| ENST00000521399.5 | 0.026336417 | chr8 | 66921987 | 66925541 | ENSG00000245910.8 | SNHG6 |
| ENST00000396466.5 | 3.50E−07 | chr8 | 73291040 | 73295789 | ENSG00000147604.14 | RPL7 |
| ENST00000284811.12 | 0.039323248 | chr8 | 73946401 | 73972106 | ENSG00000154582.17 | ELOC |
| ENST00000297258.11 | 0.015509797 | chr8 | 81280536 | 81284775 | ENSG00000164687.11 | FABP5 |
| ENST00000522567.5 | 0.000834091 | chr8 | 85107215 | 85138174 | ENSG00000133739.16 | LRRCC1 |
| ENST00000521036.5 | 0.01336998 | chr8 | 96230715 | 96235546 | ENSG00000156467.10 | UQCRB |
| ENST00000518406.5 | 0.030723552 | chr8 | 96230942 | 96235539 | ENSG00000156467.10 | UQCRB |
| ENST00000676976.1 | 0.005292905 | chr8 | 100702795 | 100718994 | ENSG00000070756.17 | PABPC1 |
| ENST00000353245.7 | 0.048770062 | chr8 | 100918686 | 100953341 | ENSG00000164924.18 | YWHAZ |
| ENST00000522352.6 | 8.53E−05 | chr8 | 108201743 | 108248720 | ENSG00000104408.11 | EIF3E |
| ENST00000519627.2 | 0.030522405 | chr8 | 108201745 | 108248726 | ENSG00000104408.11 | EIF3E |
| ENST00000677965.1 | 0.025148119 | chr8 | 108201794 | 108248710 | ENSG00000104408.11 | EIF3E |
| ENST00000676530.1 | 0.00038363 | chr8 | 108201883 | 108243593 | ENSG00000104408.11 | EIF3E |
| ENST00000677950.1 | 0.046705153 | chr8 | 124539178 | 124550768 | ENSG00000147684.10 | NDUFB9 |
| ENST00000657069.1 | 0.006070376 | chr8 | 129398535 | 129575018 | ENSG00000229140.11 | CCDC26 |
| ENST00000361036.10 | 0.048110209 | chr8 | 144082668 | 144086216 | ENSG00000197858.11 | GPAA1 |
| ENST00000336755.10 | 0.001419333 | chr9 | 37120574 | 37358149 | ENSG00000147905.18 | ZCCHC7 |
| ENST00000372480.1 | 0.000631637 | chr9 | 129626444 | 129635492 | ENSG00000148335.15 | NTMT1 |
| ENST00000381469.7 | 0.03823641 | chrX | 1336616 | 1382689 | ENSG00000185291.12 | IL3RA |
| ENST00000672987.1 | 0.018143164 | chrX | 15825806 | 15854813 | ENSG00000182287.15 | AP1S2 |
| ENST00000495186.6 | 0.021051448 | chrX | 48521808 | 48528716 | ENSG00000147155.11 | EBP |

In addition to these examples of transcriptional drug resistance mechanistic hypotheses informed by combined single-cell genomic and transcriptomic data, differential transcript usage (DTU) analysis (FIG. 7E) was employed as full-length (vs. 3′ end counting) data enabled transcript isoform insights. Isoform of HADHA was identified, whereby its expression was unique to the quizartinib-resistant population and absent in all but one parental cell—whereby the isoform with biased expression in the resistant cells was shorter (˜2688 bp) than the parental isoform (2943 bp). Similarly, 7/10 quizartinib-resistant single cells exclusively expressed an isoform of PPP1R14B containing an additional 5′ exon while 7/10 parental cells expressed none of the isoform. In total, the multiomics approach identified six instances of isoform specificity between parental and quizartinib-resistant populations for additional genes RPS3, HSPA4, SUGT1, CAPNS1.

### Identification of Candidate Regulatory SNVs Modulating Transcript Levels in Resistant Cells

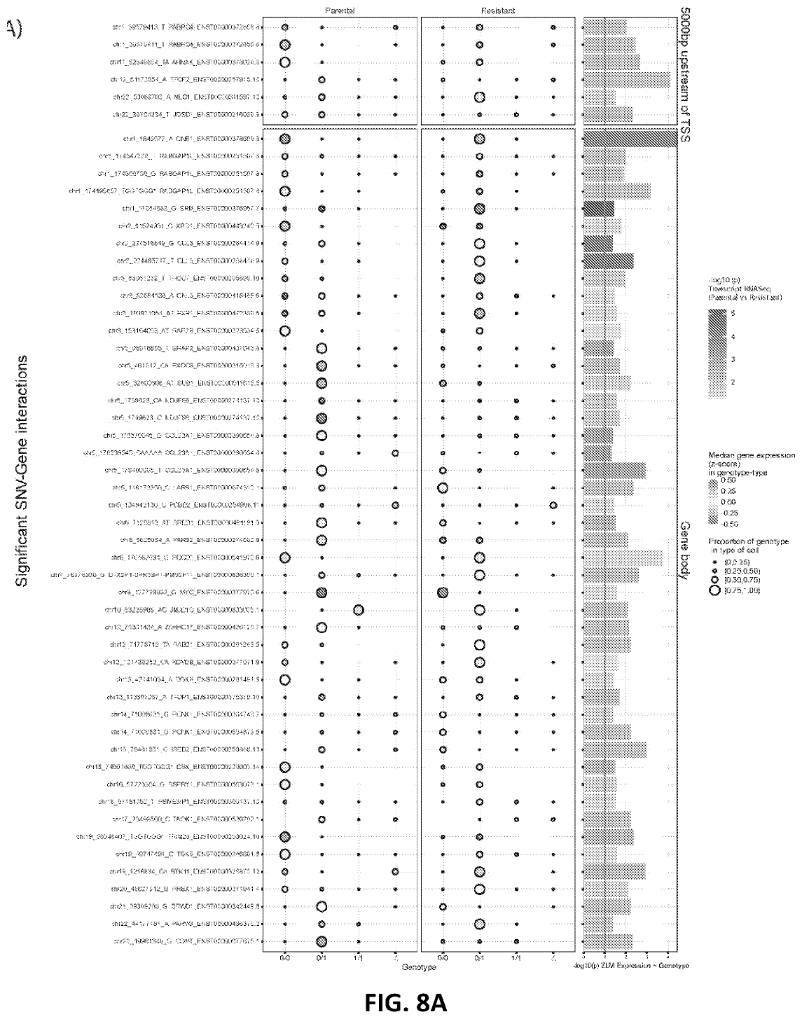

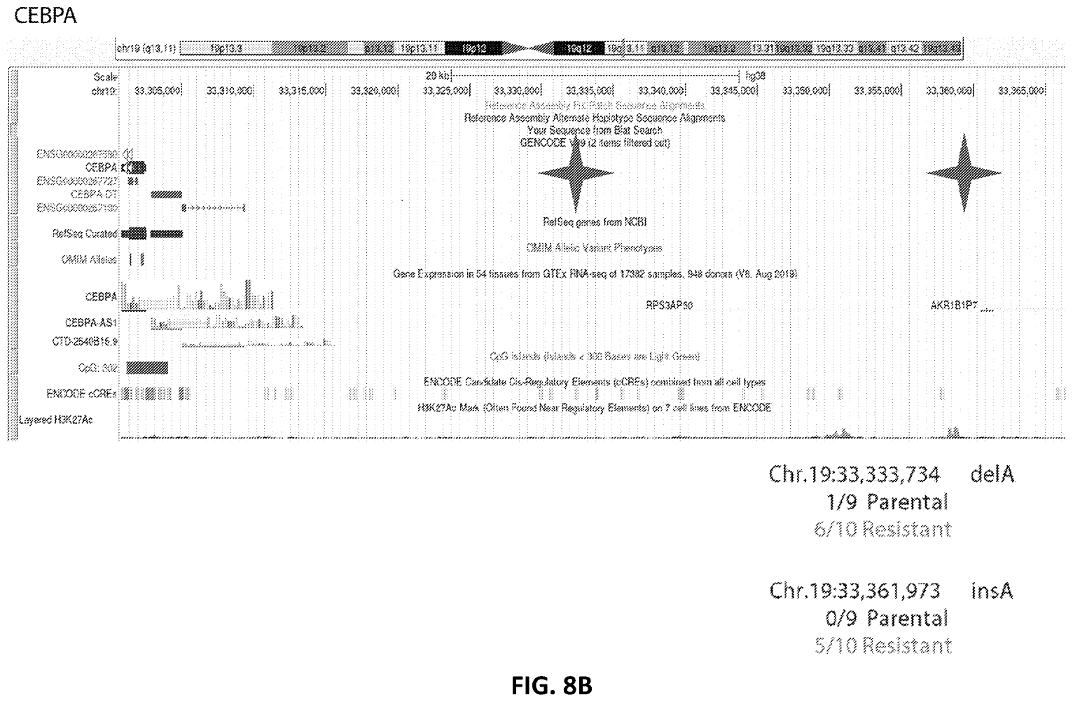

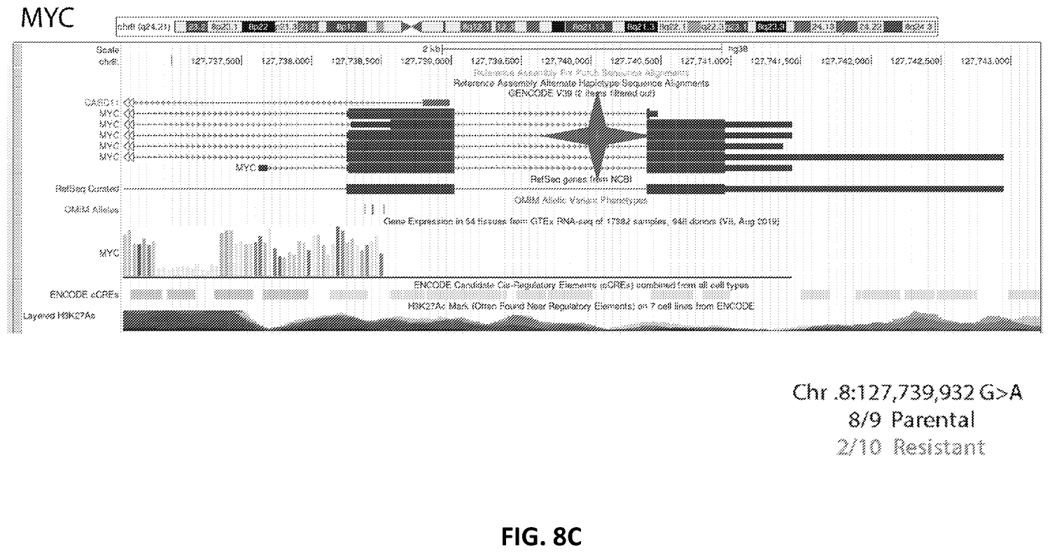

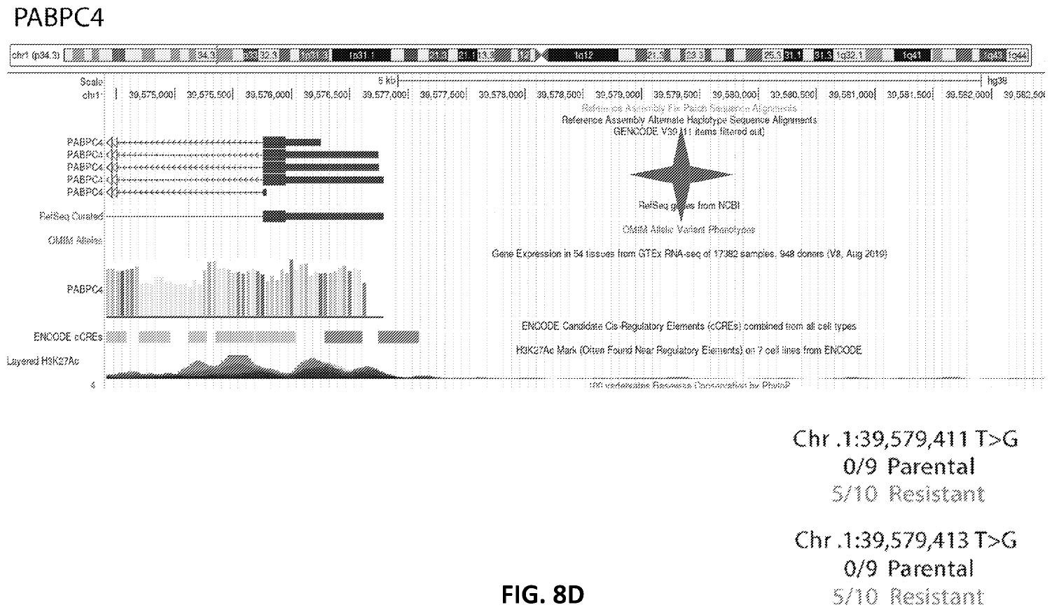

Occurrences of genomic lesions of interest that did not associate with the predicted transcriptional output were identified, leading to further analysis to identify a single nucleotide variation that would influence the expression of a proximal gene as a candidate regulatory variant in FIG. 8A. While earlier experiments failed to identify a correlation between Chr. 19q gain and CEBPA mRNA upregulation in resistant cells (FIG. 7C), a candidate distal promoter/enhancer SNV ˜20 kb 5′ of the CEBPA transcriptional start site with a genotypic bias between parental and resistant cells (FIG. 8B) was identified in the variant call file defining SNVs. An unbiased approach was then employed, whereby ZLM (zero-inflated linear model) modelling of transcriptional abundance of a gene across the genotypes of the cohorts was performed. For initial analysis SNV detection was limited to intragenic or promoter (0 to −5000 relative to the transcriptional start site). Upregulation of MY (expression was observed in resistant vs. parental cells, and a candidate intronic regulatory variant with a genotypic bias to the reference 0/0 allele in resistant cells while all but one of the parental single cells harbored the 0/1 genotype for the candidate regulatory variant (FIG. 8C) was identified. An additional example of a candidate proximal regulatory SNVs with a parental/resistant genotypic bias and concomitant expression dichotomy between the parental and resistant cells included a candidate promoter mutation in the PABPC4 gene, encoding a poly(A) binding protein, within 5′ kb upstream of the transcriptional start site (FIG. 8D). All variants identified with this analysis of course warrant functional investigation for validity but emphasize the ability of multiomics to generate candidate regulatory SNVs through the pairwise analysis of genotype shifting and transcriptional modulation in individual cells. Extending this analysis to all of intergenic space and associating the SNVs with ENCODE ChIP-Seq data will be a powerful tool to generate larger numbers of candidates influencing drug resistance and oncogenesis.

### Primary DCIS IDC Single Cells Exhibit Heterogeneous Classes of Chromosomal Loss

After demonstrating the utility of the multiomic workflow's unification of genomic and transcriptomic data to elucidate single-cell drug resistance mechanisms in a cell line model, analogous multi-omic utility in elucidating single-cell oncogenic mechanisms in primary human cancer was demonstrated. To this end, genomic and transcriptomic contributions to the transition of premalignant ductal carcinoma in situ (DCIS) to invasive ductal carcinoma (IDC) were evaluated. Dissociated single cells from tumor tissue from a mastectomy by FACS (Duke University Medical Center) were first enriched. The tumor pathology for this patient indicated ER/PR (estrogen receptor/progesterone receptor) positivity but lack of HER2 expression precluded the use of a HER2 antibody for FACS enrichment. As such, a FACS strategy was employed to enrich for ductal epithelial cells by epithelial cell adhesion molecule (EpCAM) epitope enrichment, and simultaneously to capture “EpCAM low” cells as enrichment controls.

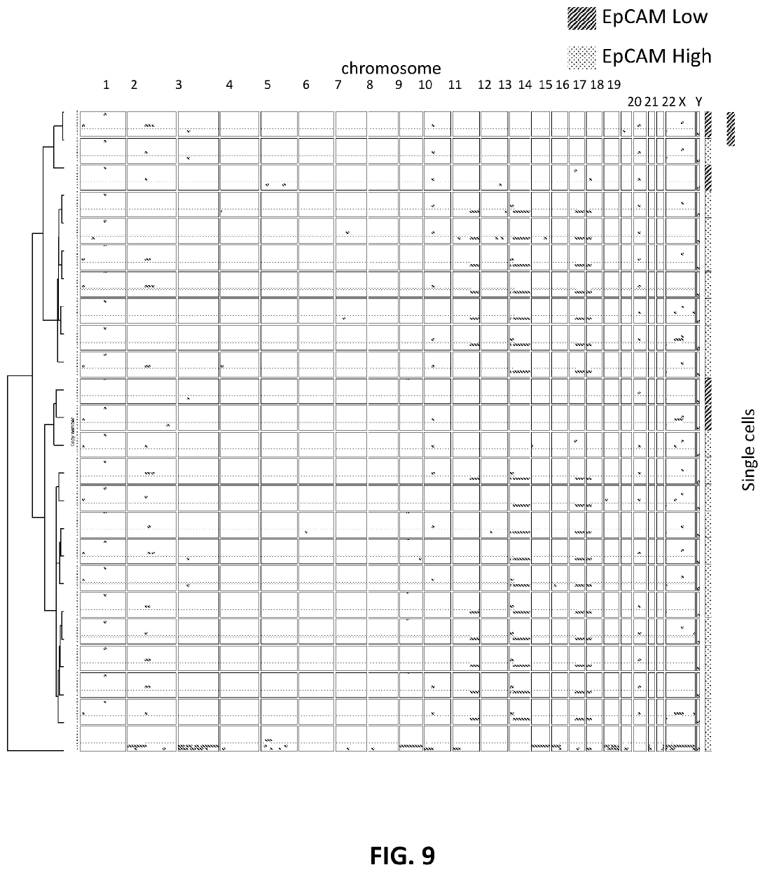

As with the MOLM-13 resistance model, CNV in primary DCIS/IDC single cells was first evaluated. The multiomics workflow on 16 single cells was performed with pronounced EpCAM expression and 4 single cells with negligible EpCAM expression. Using the same genome coverage (25×) as the MOLMs, and 500 kb windows CNV was assessed in the “EpCAM high” cohort of single cells. Distinct classes of CNV emerged, whereby single cells exhibited discrete chromosomal losses. As one class, 5/20 cells harbored near complete loss of Chr. 13 with concurrent loss of 16q/17p, FIG. 9. The most abundant class (12/20 cells) harbored these copy number alterations plus a third discrete loss of Chr. 11q. Two EpCAM high cells lacked any apparent copy number alteration, and one EpCAM high cell had a more aberrant series of genome-wide chromosomal losses. The observed Chr.13 and 16q/17p loss is consistent with reported copy number alteration in multiple stages of DCIS advancement and coincides with the loss of the prototypical tumor suppressor genes BRCA2, RBI and TP53. Interestingly, a gain of Chr. 13p, a heterochromatic “stalk” devoid of genes in 10/20 EpCAM high cells, and Chr. X gain of unknown significance in 2 EpCAM high cells and 1 EpCAM low cell encompassing the centromere and flanking p and q arm segments was observed. Even with this relatively small cohort of single cells, these data highlight copy number heterogeneity of the primary sample.

### Identification of an Oncogenic PIK3CA Mutation

Prior to genome-wide unbiased assessment of SNV, exons of the PIK30′A gene, one of the most frequently mutated genes across diverse molecular subtypes of breast cancer were assessed. The missense mutation N345K in 14/18 EpCAM high cells (FIG. 10C) was identified. N345K is second only to H1047R amongst PIK30′A hotspot mutations catalogued by TCGA and is known to influence the interaction of the p85 (PIK3R1) regulatory/p110 (PIK3 (A) catalytic subunits by disruption of the C2/iSH2 domain interface. The oncogenic N345K mutation was detected only in the single cells where CNV was observed; initially suggesting that the relevant ductal epithelial cells were stratified with the FACS strategy and the two cells lacking CNV+PIK3 (A N345K either harbored other genomic variation or were a different cell type-requiring the RNA arm of the multiomics protocol to further distinguish between the possibilities.

### Single Nucleotide Variation in DCIS IDC

Variant filtering was performed to identify novel candidate oncogenic SNVs. As validation of our filtering strategy, PIK3 (A N345K was identified in the 14/16 cells harboring 11q, 13, 16q/17p copy number loss. Coding sequence mutations in additional candidate genes known to be influential in ER+ breast cancer were not detected (FIG. 14). Utilizing a strategy to parse SNV by CNV status, variation that existed in the EpCAM high cells but that was not present in the EpCAM low cells was cataloged. Analogous to the MOLM-13 model of quizartinib resistance, extensive intergenic genomic SNV in EpCAM high vs. EpCAM low cells was observed.

### Cell Identity and Transcriptional State of DCIS IDC Singulated Cells

Of noteworthy utility in a combined genomic/transcriptomic single-cell assay is the capability to link genotype to identity of cell type and to inference of cell state. This was critical in the interpretation of the observed CNV and PIK30′A E345 single-cell DCIS/IDC genotypes due to the difficulty in designing a FACS marker schema that unambiguously identifies the ductal epithelial cells of interest from surrounding stromal cells and infiltrating immune cells. Gene expression profiles of EpCAM high and EpCAM low cells separated by principal component analysis (FIG. 10A) using the PAM50 gene set of genes influential in diverse subtypes of breast cancer (FIG. 10B). Differential gene expression analysis highlighted gene signature blocks between two primary clades: a cluster of exclusively EpCAM high cells, and a cluster comprised of all EpCAM low cells intermixed with 4 EpCAM high cells (FIG. 10C). Initial ascertainment of transcripts defining the EpCAM low cells revealed enrichment of in IL-2 and CD4 T cell-defining gene sets, suggesting that these cells may be tumor infiltrating lymphocytes present in this patient's singulated tumor sample. However, further rigor into transcriptome-based cellular annotation with Human Cell Atlas data (See Methods) parsed the EpCAM low cells into stem-cell like, endothelial, fibroblastic and monocyte identities/states (FIGS. 10B-10E) which was independent of transcript count (FIG. 1Aa). Four outlier EpCAM high cells exhibited a gene expression signature such that they were placed in the same root clade of the dendrogram as the EpCAM low cells. Cells were identified as having two distinct identities/states: epithelial and monocytic. Intriguingly, while all EpCAM low cells lacked PIK3CA N345K or characteristic DCIS copy number loss, the EpCAM high cell in the EpCAM low gene expression signature clade with epithelial identity harbored both of these genomic alterations. Without being bound by theory, this is suggestive of a plasticity of cell state of a ductal epithelial cell and the acquisition of phenotype with stemness attributes as suggested by cell annotation profiles more closely matching tissue stem cell or fibroblast identities (FIG. 10D). One outlier EpCAM high cell in the EpCAM low clade lacked oncogenic PIK3CA mutations and the prototypical DCIS chromosomal losses and displayed a monocytic gene expression profile. For this instance, it is suggestive of infiltration of monocytes in the sample, although it cannot formally exclude the possibility of cell state change of a malignant or benign ductal epithelial cell or infiltration of monocytes in the sample. Furthermore, one putative epithelial cell in this outlier EpCAM high class, although differing from the prototypical DCIS chromosome losses observed in the main EpCAM high clade, harbored a grossly aberrant CNV profile and may represent a malignant cell. Our examples of putative plasticity of phenotypic cell state with regard to oncogenicity warrant multiomics analysis of additional cells to determine the frequency of this cell state in the sample or whether it represents stochastic genomic variation that did not persist or was not selected for in the population. Collectively, these data suggest profiling a cell at the transcriptome level only could lead to an incorrect cell classification and underscores that understanding both RNA and DNA-omic tiers is critical to provide proper classification.

### Holistic View of MOLM-13 and DCIS IDC Single-Cell Molecular Signatures

Having in succession determined CNV, SNV and transcriptional insights in both the MOLM-13 model of drug resistance and in primary DCIS/IDC it was critical to begin to amass and graphically present interrelationships between the “-omic” layers of data. For MOLM-13, a secondary driver mutation was identified that likely affecting drug binding in all single cells yet provided evidence for concurrent transcriptional bypass of FLT3 signaling, highlighting the importance of ascertaining both DNA and RNA-driven mechanisms of resistance in the same cells.

For primary DCIS/IDC, unification of DNA-level and RNA-level data allows the interpretation of genotypes in the context of expression signatures defining cell type and cell state. Harnessing these layers of molecular information in a heat map/dendrogram quickly conveys the finding that EpCAM expressing ductal epithelial cells harbor both prototypical copy number losses and an oncogenic PIK3 (′A mutation while EpCAM low cells with alternative identities by transcriptomic profile from the same singulated cell sample lack chromosomal loss and this mutation (FIG. 10D). Yet, cell identification cannot be unambiguously assessed solely by EpCAM FACS protein levels but in leveraging more contemporary cellular annotation methods; IDs can be objectively identified that match the cell's known biological origin or reflect a cell state transition.

### Discussion

Each “-omic” tier of molecular information allows a greater ability to comprehensively define the molecular mechanisms of oncogenesis and drug resistance in a tumor. In the single cell tumor biology arena, most work to date has been performed at the transcriptome level, owing to the large-scale adoption of droplet-based methodology facilitating workflow ease and single-cell throughput. While there has been unquestionable advance from droplet-based RNA-Seq studies defining diversity and heterogeneity in transcriptional states including those states defined longitudinally, a gap remains in that there have been few studies providing concurrent genomic data with the gene expression data. This is critical for multiple reasons. Firstly, in the absence of DNA-level information, genomic contributions to the transcriptional or phenotypic state cannot be discerned, such as genomic mutation or variation in regulatory elements, in transcription factors, or in chromosomal copy number, each of which has the potential to define transcriptional state. Thus, prior studies have had obvious limitations in resolving the critical link between DNA and transcriptional changes. Secondly, while transcript-level information is frequently employed for molecular subtyping of a tumor, pharmacological decisions are primarily driven by genomic variation, due to technical and informatics challenges with ascertainment by transcriptional status. This may, in part, explain why tumor DNA molecular data provides imperfect prediction of treatment sensitivity.

Coupling single-cell genomic and transcriptomic information has been hitherto limited due to technical challenges of integrating the RNA and DNA amplification steps. Additionally, in instances where this incompatibility has been overcome, existing methodologies for the amplification of single cell genomes have been employed and thus the shortcomings of incomplete genome coverage, poor coverage uniformity, and less optimal allelic balance have accompanied these joint RNA/DNA protocols. G&Tseq, for example, empowered researchers with transcriptional data of single cells paired with multiple displacement amplification for DNA level information. This has facilitated multi-omic insights at primarily the transcriptome+copy number alteration level due to the incomplete genome amplification inherent with MDA or PicoPLEX, precluding SNV analysis. Multiomics chemistry can overcome this limitation by unifying primary template-directed amplification with RNA sequencing in single cells and show its utility by cataloging putative regulatory SNVs affecting gene expression.

The ability to define cell identity and cell state at the single cell level is one chief strength of multiomics. While some FACS strategies may sufficiently stratify cell types within a heterogenous sample, one does not always a priori have this biomarker knowledge, and even in the presence of this knowledge outlier sorted cells were observed without detection of concordant mRNA levels despite the cells being gated on high levels of the corresponding protein biomarker. Thus, joint RNA/DNA single-cell profiling has enabled us here to spotlight instances of diverse, non-epithelial cell types in our primary breast cancer sample, preventing the false interpretation of a ductal epithelial cell lacking prototypical copy number alteration or key oncogenic missense mutations when in fact the lack of genomic variation is due to the cell type being assayed. When armed with joint genomic and transcriptomic information, cell type tumor heterogeneity manifesting in FACS can now be exploited, for example, to understand the contributions of the genome variation of a monocyte to the interaction of the malignant epithelial cell in the given microenvironment, as opposed to considering the monocytes as contaminating the epithelial population of interest in this instance.

Beyond characterizing cell identity with multiomics, a continuum and heterogeneity of cell state within a breast tumor specimen at unprecedented resolution was enabled by the multiomics methods described herein. An intermediate transcriptional profile emerged between that of the EpCAM low single cell cohort and that of the core cohort of EpCAM high epithelial cells. This profile was intriguingly observed in an EpCAM high cell that harbored PIK3CA N345K and DCIS-characteristic chromosomal losses, thus having the core genomic changes of the main epithelial cell cohort. Nevertheless, it manifested with a different transcriptional stem-like state-indicating a potential state conversion as well as highlighting inherent transcriptional single-cell heterogeneity even within a relatively small sampling of a singulated tumor sample. It will be crucial to determine the prevalence of this cell state as more cells of this sample are sequenced, as well as to define the diversity of additional novel transcriptional states that may be contributing to the advancement of DCIS to invasive cancer. The multiomics method importantly provides the ability to link these diverse transcriptional cell states to genotype (FIG. 8A).

A second chief strength of the multiomics workflow is to provide the attributes of primary template-directed amplification to allow comprehensive genomic assessment vs. the sole ascertainment of a small number of candidate loci or copy number alterations of a broad level of resolution. This enablement of SNV detection with high sensitivity and precision over >95% 1 of the genome opens a new realm of discovery. PTA in the multiomics workflow opens up a new source of pharmacological targets with genome-wide data and non-exonic space not possible with existing WGA methodologies with low genomic coverage and uniformity. Notable was the single nucleotide variation present in the parental vs. quizartinib resistant MOLM-13 cells (6444 differentially prevalent SNVs, FIG. 6), which further underscores that, while transcriptional plasticity is dogmatic, it is equally as important to recognize genome plasticity observed in this model. Furthermore, while there will be a background of passenger mutation or mutation currently not pharmacologically targetable, this diversity can be ultimately ascertained and represent a co-evolution of variants for a functional, biologically relevant phenotypic output. Efforts to estimate intergenic variation at putative functional elements-promoters, enhancers, splicing enhancers—is a frontier and an underappreciated aspect of drug resistance studies. The candidate regulatory single nucleotide variation proximal to differentially expressed genes of interest in our parental vs. resistant cells may require obligate functional characterization, but as the cost of genome sequencing begins to plummet, these data and their associated biological insights will necessarily begin to accumulate. For discovery, dual genome/transcriptome ascertainment from single cells not only expedites the generation of candidate regulatory SNV links to transcript modulation but unveils connections obscured by bulk sequencing data.

Both our engineered model of drug resistance in AML and analysis of a primary DCIS/IDC sample have yielded single nucleotide variation that would be predicted, at the outset, to have a deleterious effect on protein function. Frameshift and stop codon gain mutations observed in the single cell genomes of our samples represented an unbiased starting point for the discovery of novel oncogenic and drug resistance loci beyond ascertainment of known candidate genes. Yet, coupling transcriptional information from the same cell revealed that, for some of these novel genomic variants of purported deleterious effect, the single cells did not express the corresponding transcript-indicating the genomic change was passenger or stochastic in nature and not functional. Understanding this genomic variant “penetrance” in terms of manifesting at the transcriptional level is a fundamental capability of multiomics, and in our initial sample sets redirected or nullified multiple hypotheses.

In addition to binary “expressed or not expressed” decisions, dual DNA/RNA information assisted in directing hypotheses of molecular mechanism. CEBPA, an enhancer factor⁴² significantly upregulated in our quizartinib-resistant single MOLM-13 cohort, resides on Chr. 19q, where four resistant cells harbored 2n to 3n genomic gain of 19q. A parsimonious initial hypothesis is that genomic amplification of 19q contributed to the observed transcript upregulation, however the CEBPA transcript upregulation was observed in all resistant cells, and did not show a correlation with the single cells that harbored genomic amplification of 19q (FIG. 7C). This suggests that an alternative mechanism of epigenetic control was at play for this upregulated gene, perhaps via modulation of a transcription factor or an enhancer-level phenomenon that was purported by the SNV between parental and resistant cells proximal to the CEBPA gene. More broadly, while statistically significant associations between ploidy and expression of a specific cohort of genes (FIG. 7D) were identified, no such association was observed for most loci. Collectively, these examples illustrate the criticality of paired RNA information when positing mechanisms based on genomic data alone and caution that the “penetrance” of the change needs to be ascertained. Conversely, important correlations between SNV and the expression of a proximal gene, as with the oncogenic driver MYC (FIG. 8A and FIG. 8C) were found, highlighting instances whereby DNA and RNA information are likely to be functionally linked.

The enablement of simultaneous genomic and transcriptomic data from the same individual cell vastly increases the complexity of putative mechanisms of drug resistance and oncogenesis. This will only increase as additional “-omic” tiers of layers are added, including ascertainment of extracellular protein expression as the nature of multiomics template-switching cDNA chemistry allows for the incorporation of CITE-seq-like oligo-tagged antibodies. These data are complex, requiring development of novel sophisticated bioinformatics tools. However, mechanistic insights analogous to those presented here to accumulate from the research community having the newfound ability to accurately assess single nucleotide genomic variation in conjunction with transcriptional profiles—aiding discovery efforts to generate a new wealth and generation of pharmacological targets.

### Methods

### Cell Culture

NA12878 cells (CEPH/Utah Pedigree 1463) were obtained from the Coriell Institute for Medical Research (Camden, NJ). Cells were maintained in RPMI 1640 (Gibco 11875-093) supplemented with 15% FBS and penicillin/streptomycin, and sub-cultured every 2-3 days while maintaining a density range of 1.0-3.0 E6/ml.

MOLM-13 acute myeloid leukemia cells harboring heterozygous FLT3 internal tandem duplication (ITD) were obtained from the DSMZ-German Collection of Microorganisms and Cell Cultures (ACC 554). Cells were maintained in RPMI 1640 (Gibco 11875-093) supplemented with 10% FBS and penicillin/streptomycin, and sub-cultured every 2-3 days while maintaining a density range of 2.5 E5-1.5 E6 cells/ml. For generation of the quizartinib-resistant MOLM-13 line, cells were continually treated with 2 nM quizartinib (Selleckchem AC220) or DMSO vehicle control for matched parental control line and drug replenished at each subculturing until emergence of resistant clones at 5 weeks duration in culture. Genomic DNA (Zymo Research Quick-DNA Microprep w\Plus Kit, D3020) or total RNA (Qiagen RNeasy Plus Kit, 74034) was isolated from quizartinib-resistant and matched parental MOLM-13 cells at time of FACS sorting to generate bulk sequencing control libraries for comparison to single cell datasets and for quantitative PCR template.

### Multiomics Workflow

The multiomics workflow begins with template-switching-based RNA-Seq chemistry to generate biotin-dT-primed, first strand cDNA followed by termination of the reaction and nuclear lysis, at which point primary template-directed amplification proceeds. The mRNA-derived cDNA is affinity purified with streptavidin beads from the combined pool of cDNA and amplified genome. cDNAs are then further purified with subsequent streptavidin bead washes of two stringencies and on-bead pre-amplification of the first-strand cDNA to yield double-stranded cDNA. In parallel, the PTA fraction from the same cell containing genome amplification products, separated from the cDNA, is purified. The separate and distinct fractions of pre-amplified mRNA cDNA and genome-derived DNA amplification fractions undergo SPRI cleanup prior to NGS library are generation.

### Karyotypying

MOLM-13 cells were analyzed within 2 weeks of thaw (KaryoLogic, Inc, Durham, NC) with a workflow for complex hyperdiploid karyotypes using 25 metaphase spreads. Live cultures were delivered to the service provider on-site and cultures recovered in 5% CO2 37 C incubators on-site for one week prior to metaphase spread creation.

### FACS

Prior to FACS, cell lines were first counted and assessed for overall viability by trypan blue staining using a Countess II FL instrument (ThermoFisher Scientific) or by acridine orange+propidium iodide with a Luna FL instrument (Logos Biosystems). Cell line cultures put forth to the FACS protocol exhibited >90% viability.

### MOLM-13

For single cell analysis, ˜2.0E6 MOLM-13 quizartinib-resistant or matched parental cells were rinsed twice in staining buffer (0.2 μm filtered Dulbecco's Phosphate Buffered Saline lacking calcium and magnesium (Gibco 14190) supplemented with 2% FBS) and kept on ice until BD FACSAria III sorting at the UNC School of Medicine Flow Cytometry Core Facility. Following Calcein AM (BioLegend 425201), propidium iodide (Millipore Sigma P4864) and DAPI staining, singlet (FSC-A/FSH—H, SSC-A/SSC-W) and live cell (DAPI/PI negative, top 70% Calcein-AM positive) gating was established and single cells were sorted (130 micron nozzle assembly) into low-bind 96 well PCR plates (Eppendorf twin.tec LoBind, semi-skirted, 0030129504) containing Cell Buffer and immediately frozen on dry ice following brief mixing (1400 rpm, 10 sec) and centrifugation.

### NA12878

˜2.5E6 NA12878 (NA12878/HG001) cells were prepared as above and subjected to Sony SH800 sorting using a 130 micron chip. Singlet (FSC-A/FSC—H, BSC-A/BSC-W) and live-cell (PI negative, top 70% Calcein-AM positive) gating was employed for single cell sorting into low-bind 96 well PCR plates pre-loaded with Cell Buffer as described above.

### Primary DCIS IDC

Tissue for single-cell DCIS/IDC studies was obtained in accordance with the Duke University Medical Center IRB for the clinical trial PRO00034242 “Biologic Characterization of the Breast Cancer Tumor Microenvironment.” Cryo-preserved, singulated cells (˜4.2E5) derived from mastectomy tissue were thawed at 37 C and centrifuged at 350×g for 5 min to separate cryo-preservation media. Cells were rinsed once in staining buffer and incubated with 2 μg/ml anti-human CD326 conjugated with AlexaFluor 700 (ThermoFisher 56-9326-42) at 4 C in the dark for 1 h. Following this,

˜8.4E4 cells were reserved for a parallel negative control mock stain lacking any antibody for assessment of background fluorescence levels for viability and EpCAM staining. Then cells were washed 3× with staining buffer with 350×g 5 min centrifugations in between washes and passed through a 35 micron filter prior to loading for FACS. Singlet (FSC-A/FSC-H, BSC-A/BSC-W) and live-cell (Calcein AM) gating was defined followed by daughter EpCAM high and EpCAM low gates. EpCAM High and Low cells were sorted into the same 96 well plates as described above for to minimize potential batch effects of downstream genomic/transcriptomic amplification.

### Quantitative RT-PCR

10 ng of genomic DNA was isolated from a cell collection of quizartinib-resistant or matched parental cells as described above and subjected to a custom Taqman™ genotyping assay, #ANMF9C4 (Invitrogen-Applied Biosystems) using the manufacturer's suggested conditions for reaction assembly and cycling on a QuantStudio6 instrument. The assay was designed to distinguish between human N841 and K841 with the C/A nucleotide polymorphism, respectively at the GRCh38/hg38 coordinate Chr13:28,018,485.

### Combined Genomic Transcriptomic Analysis

Firstly, biotin-conjugated oligo dT primer (Integrated DNA Technologies) was utilized in a template-switching reverse transcription reaction to generate first-strand cDNA from single cells. Primary Template-directed Amplification (PTA) with reagents (Bioskryb Genomics, Inc.) was performed in succession following reverse transcription. First-strand cDNA was then affinity-purified using streptavidin beads and subjected to two high-salt washes followed by one low-salt wash. 24-cycles of pre-amplification was performed to generate 2nd strand cDNA and RNA sequencing libraries were prepared using the RNA library preparation module. For preparation of PTA libraries, PTA product not bound to streptavidin beads was purified using beads and ligated to full-length IDT for Illumina TruSeq adapters using the DNA library preparation module. Sizing for both RNA and DNA amplification products was determined by D5000 TapeStation electrophoresis (Agilent Technologies) while library preparation sizing was determined by HS D1000 electrophoresis. Amplification and library yield was assessed by Qubit 3 or Qubit Flex instrumentation (ThermoFisher Scientific).

### Sequencing

Low-pass sequencing was first performed on DNA fraction libraries using an Illumina MiniSeq (2.3 pM library flow cell loading concentration) or NextSeq1000 (640 pM library flow cell loading concentration), 2×75 targeting

>2.0E6 total reads per library. For RNA fraction libraries, 2×75 MiniSeq or NextSeq1000 sequencing targeting on average>1.0E6 reads per library was employed for flexibility for data down-sampling. For joint clustering of DNA and RNA fraction libraries, a 10:1 molar ratio of [DNA arm]: [RNA arm] libraries was employed. Following low-pass sequencing, DNA arm libraries were 2×150 sequenced on an Illumina NovaSeq6000 S4 flow cell targeting 5.5 E8 total reads to provide down-sampling flexibility at either the Vanderbilt Technologies for Advanced Genomics (VANTAGE) core facility or the Duke University Genomics and Computational Biology (GCB) core facility.

### Bioinformatics Approaches

### Pre-Sequencing Quality Control

Single cell libraries were evaluated utilizing an internal pre-sequencing pipeline that leverages low-pass sequencing data to create multiple quality control metrics to assist in evaluating the single-cell libraries readiness for high-throughput sequencing. Notably retrieved was the PreSeq count to estimate library complexity. This pipeline features additional QC metrics for genomic coverage, percent of reads mapping to chimeras, percent of reads aligned to the reference genome, and percent of nucleotides mismatched to the reference genome. Additionally, the pipeline implements MultiQC for supplementary QC metrics including read length, percent of duplicate reads, number of mapped reads, and total number of mapped reads.

### Benchmarking RNA-Seq Results

To establish overall benchmarking scores of multiomic amplification approach, quality control was performed pre- and post-sequencing on Human Brain Reference RNA (HBRR), Universal Human Reference RNA (UHRR), and NA12878 B-lymphocyte cells. Several metrics were considered: percent mapping, gene detection, dynamic range of expression, and coefficient of variation for measuring DNA leakage, accuracy, and robustness of this methodology. For each cell the total alignments, reads aligned, and genomic feature alignments were quantified using the Qualimap⁴⁴ (v2.2.2) platform for reporting QC metrics and bias estimations of whole transcriptome sequencing data. Furthermore, the platform enables detection of outlier cells, relative consistent performance patterns among these cells, and potential batch or other systematic artifacts that are not apparent when evaluating individual cells in isolation. Using metrics produced from Qualimap findings, the percent mapping of total alignments were computed as well as the percent exonic and intergenic of genomic alignments. Thereafter, the number of genes identified were defined, dynamic range, housekeeping gene variability metrics, and observations of expression patterns in housekeeping genes for each reference cell line, using counts per million (CPM) normalized gene expression counts. Gene detected is defined at the number of genes with non-zero counts in each cell. The dynamic range of all expressed genes was then estimated at 10-90 percent. As an estimate of sample dispersions and reproducibility, the percent coefficient of variation (CV) was calculated as a ratio of standard deviation to mean: CV=. Median absolute deviation (MAD) was calculated as a robust measure of variability between housekeeping genes. This is defined as the median of the absolute deviations from the median (m): MAD=median (|xi-m|).

### Secondary Analysis Pipelines

For the DNA-based analyses coming from the genomic fraction of the multiomics workflow, an internal analytics pipeline modified from Sentieon driver-based tools was leveraged. Initial FASTQ pairs were trimmed against low quality and library artifacts using fastp (v0.20.1) Alignment was performed using BWA (Sentieon-202112), followed by deduplication (locus_collector v202112/dedup v202112) of identically-aligned reads. Alignment-based QC and coverage determination was (driver_metrics v202010). Copy number calling was performed using ginko⁴⁶ (GitHub commit: 892b2e9f851f71a491cade6297f74f09f17acf4c), with a window size of 500 kb. Variant calling at the cell level was performed with haplotyper (v202010). Characteristics for all variants was provided for variant quality score recalibration to VARcall, GVCFtyper (v202010). All variant identification and annotations for gene/coding effect were performed using snpEFF/SnpSIFT (5.0e). Further variant-based tertiary analysis used filtered genomic loci with sequencing depths>4 and >1 variant read candidate SNVs. All candidate SNVs were classified according to allele frequencies.

The RNA-Seq pipeline implemented here was used to generate metrics of feature quantification at the transcript and gene-level. Details about the number and length of reads generated is found in Table 1 for the DNA arm (a) and RNA arm (b). Unless specified to be down-sampled (using seqtk v1.3), all reads were leveraged for each analysis. To remove low quality sections and sequencing artifacts, fastp was used for all cells' analysis prior to alignment. Alignment of reads was performed with STAR (v 2.7.6a) and were compared against transcript reference made from combining Ensembl (release 104) known transcripts and noncoding. Region assignment and counting of aligned reads was performed with HTSeq4949 (v 0.13.5) and Salmon5050 (v1.6.0) for gene-level metrics. Further, the pseudo-alignment algorithm implemented in Salmon was used to perform both transcript-level and gene-level quantification. Matrices of feature expression were constructed using the Bioconductor package tximport.

### Tertiary Analysis

### Bulk Dataset Identification

Several datasets in the Short Read Archive (SRA) were identified that had bulk NA12878 in mRNA-stranded RNA library preparation methods that most closely resembled our own multiomics approach. To handle variation of an individual dataset, at least 10 datasets were targeted for capture that could represent transcriptome coverage of NA12878.

### Variant Evaluation in NA12878 Cells

For the NA12878 cells, first joint genotyping was first performed across them utilizing the GVCFTyper, VarCal and Apply VarCal modules from Sentieon. Then, inputting the re-calibrated variants and evaluating the variant quality score log-odds (VQSLOD), the precision and sensitivity of called SNPs was determined by employing the vcfeval module from the RTG tools using as reference the NA12878/HG001 genome v.3.3.251 from the Genome in a bottle (GIAB) consortium⁵².

### Allelic Balance in NA12878 Cells

Allelic balance for NA12878 cells was calculated using an ad hoc developed module based on a series of bcftools commands that extract the a priori defined high confident heterozygous sites, reported in GIAB NA12878/HG001 genome v.3.3.2, from all sequenced NA12878 cells. Then, for each cell and for each heterozygous site, variant allele depth is extracted and converted into proportion. For final reporting, heterozygous sites with at least a total depth>1 are used.

### Rna Arm: Matrix Normalization

For MOLM-13 and DCIS cells, their corresponding Salmon-based transcript and gene matrices were normalized across features utilizing the log norm method. Briefly, feature counts for each cell are divided by the total counts for that cell, multiplied by the scale factor (10+) whose products is finally log 2 transformed. These normalized matrices served as input for downstream analysis including, principal component analysis (PCA), differential transcript expression (DTE), differential gene expression (DGE), differential transcript usage (DTU), heatmap reconstruction including unsupervised clustering of cells and transcripts/genes and zero inflated linear models linking transcript expression to CNV and SNVs.

### Principal Component Analysis

MOLM-13 and DCIS normalized transcript level and gene level matrices were centered across samples within a feature using the R function scale. Further, principal component analysis was computed using the oh.pca function from the ohchibi R package taking as input the centered normalized matrices.

### Differential Expression

Differential transcript expression was estimated and differential gene expression leveraging the zero-inflated linear model (ZLM) implemented in the MAST⁵³ R package was taken as input for the log normalized feature matrices described above. For the MOLM-13 dataset, the following model was fitted to identify transcripts/genes that had signatures of differential expression across parental and resistant cells: Transcript Gene expression˜Cell Type (Parental Resistant). Number of detected features (transcripts genes) per cell

For the DCIS dataset, performed principal component analysis was performed using the top 500 most highly variable genes across the dataset and then split the cells into three groups using the PCA projection as guidance. This three group scheme was used to discretize, in an unbiased way the cellular heterogeneity within EpCAM High and EpCAM Low treatment. After dividing the cells into three groups the following ZLM was fitted to identify transcripts/genes that had signatures of differential expression across the aforementioned groups: Transcript Gene expression˜Cell Group. Number of detected features (transcripts genes) per cell

### Cellular Typing

Transcriptome-based cellular typing was performed for the DCIS dataset using the R package SingleR⁵⁴ utilizing the Human Primary Cell Atlas expression reference dataset deposited in the celldex⁵⁴ R package and taking as input the gene level normalized expression salmon-based matrix.

### Differential Transcript Usage

For the MOLM-13 dataset, differential transcript usage was performed. Briefly, the scaledTPM metric output from tximport was taken and reconstructed into a matrix of transcript abundances across cells. Next, the transcript expression was modeled using the Dirichlet-multinomial distribution model implemented in the DRIMSeq R package.

### Linking Transcript Expression to CNV

For the MOLM-13 dataset, transcript-level variation in expression was linked with changes in locus ploidy utilizing a zero-inflated linear model framework. Briefly, for each quantified transcript, its ploidy was extracted across cells from the Ginkgo-based estimation by employing genomic-coordinate intersection utilizing the GenomicRanges R package. Next, the following ZLM design utilizing the MAST R package was fitted: Transcript expression˜Estimated ploidy at a given locus.

### Linking Transcript Expression to Genomic Polymorphisms

For the MOLM-13 dataset, transcript-level variation in expression was linked with single nucleotide variations across the genome utilizing a zero inflated linear model framework. Briefly, first the genomic coordinates of SNVs were paired with transcripts utilizing genomic-coordinate intersection via the GenomicRanges R package. With respect to the transcript-coordinates, the Ensembl reported transcript start and transcript end was used to define the gene-body of a transcript, in addition the 5000 bps upstream of the Ensembl reported transcription start site (TSS) was used to define potential cis-regulatory regions affecting the transcript. After defining the corresponding SNV-Transcripts pairs, a matrix of expression and genotype locus (SNV) across all cells was constructed. Finally, utilizing this matrix, a zero-inflated linear model was fitted using the MAST R package with the following design: Transcript expression˜ Genotype

The GSEA-R tool was used in conjunction with the molecular signatures database (MSigDB) to conduct a systematic examination of enriched gene sets connected to differentially expressed genes across Molm-13 parental and resistant cells as well as significant SNVs. In addition, the Reactome Pathways database was used to find relevant pathways among these genes using a default adjusted p-value of 0.10.

### Significant Variant Testing

For identification of differential SNV's between Molm 13 P and R cells, categorical variables for diploidy status were generated and compared with chi-square test. Two-sided p-values less than 0.05 were considered significant. In addition, a multinomial logistic regression was fitted to identify differences in SNV prevalence across the parental and resistant MOLM-13 types. Specifically, for each SNP, the three states genotype (0/0, 0/1, 1/1) were encoded as dependent variable and the MOLM-13 type (parental, resistant) as independent variable. Significance of the model was tested using a Wald Test.

Multiomics was applied in the context of two major phenomena in oncology: tumor heterogeneity (leading to cancer progression) and treatment resistance. Material from a primary patient breast cancer and an acute myeloid leukemia (AML) cell line, MOLM-13, was used to highlight multiomic biomarker paradigms enabled by this chemistry. Performance of the PTA-enabled genome amplification was largely unaffected by addition of RNA enrichment, with control WGS results showing >95% genome coverage, precision >0.99 and allele drop out <15%. In the RNA fraction of the chemistry, full-length transcripts were routinely obtained that demonstrate a ratio of 1 for 5′/3′ bias, with increased coverage of intronic regions and 5′ regions that are indicative of novel transcripts, showing strength of the template switching mechanism to capture isoform information with sparsity rates <75%. Cellular variability was observed for revealed biomarkers at both in the genome and transcriptome despite employing a relatively small number of individual cells. In our primary patient sample of ductal carcinoma in situ (DCIS)/invasive ductal carcinoma (IDC) oncogenic PIK3 (A driver mutations were found and prototypical DCIS copy number alterations binned into heterogenous single-cell classes of genomic lesions. Within our quizartinib-treated MOLM-13 cells, multiple potential mechanisms of resistance were identified within seemingly sporadic changes and were able to associate specific mutation, copy number and expression significantly correlated to treatment. In this latter scenario, the DNA arm of our combined workflow uncovered a secondary FLT3 (non-internal tandem duplication (ITD)) mutation as a candidate primary driver of resistance to drug while the RNA arm showed matched transcript upregulation of AXL signal transduction as well as enhancer factor modulation. Importantly, proximal candidate regulatory SNVs, outside of the CDS, were identified and associated to upregulated transcripts in cis. The study highlights that both the genome and transcriptome are dynamic, leading to a set of combinatorial alterations that affect cellular evolution and that fate can be identified through multiomics application to individual cells.

### Example 3: Use of Uracil Tolerant Polymerase for Improved Multiomics

Following the general methods of Examples 1-2, cDNA was generated from single cell RNA using reverse transcription. cDNA amplicons were generated using biotinylated poly dT primers. Next, the PTA method was used to amplify genomic DNA from the cell, wherein the mixture of dNTPs comprises uracil. cDNA was then purified from the mixture using streptavidin, and further treated with uracil DNA glycosylase (UDG) and DNA glycosylase-lyase Endonuclease VIII to remove any residual genomic amplicons from the cDNA. The genomic fragments generated from PTA were then purified, and both cDNA and genomic DNA fractions were converted into sequencing-ready libraries using adapter ligation. A uracil-tolerant polymerase was used to amplify the PTA-generated genomic fragments.

### Example 4: Transposon Library Preparation with Uracil-Tolerant Polymerases

The general procedures of Example 3 are followed with modification: sequencing-ready libraries are prepared by tagging genomic and/or cDNA fragments with a transposon complex described herein (e.g., TDE1). After tagging with adapters using the transposon complex, the libraries are amplified. For uracil-containing libraries (e.g., genomic PTA library), a uracil-tolerant polymerase is used. Both adapter-tagged libraries are then sequenced.

What is claimed is:

1. A method of multiomic sample preparation comprising:
a. isolating a single cell from a population of cells, wherein the single cell comprises RNA and genomic DNA;
b. amplifying the RNA by RT-PCR to generate a cDNA library;
c. contacting the genomic DNA with at least one amplification primer, at least one nucleic acid polymerase, and a mixture of nucleotides, wherein the mixture of nucleotides comprises at least one terminator nucleotide which terminates nucleic acid replication by the polymerase to generate a genomic DNA library and dUTP; and
d. isolating the cDNA from the genomic DNA library;
e. sequencing the cDNA library and the genomic DNA library.

2. The method of claim 1, wherein the mixture of nucleotides comprises at least two of dATP, dCTP, dGTP, and dTTP.

3. The method of claim 1, wherein the mixture of nucleotides comprises dATP, dCTP, dGTP, dTTP, and dUTP.

4. The method of claim 2, wherein the ratio of dTTP to dUTP is 50:1 to 1:20.

5. The method of claim 1, wherein at least some of the polynucleotides of the cDNA library comprise a barcode.

6. The method of claim 1, wherein at least some of the polynucleotides of the cDNA library comprise a label.

7. The method of claim 1, wherein at least 90% polynucleotides of the cDNA library comprise a 5′ to 3′ bias of 0.8 to 1.2.

8. The method of claim 1, wherein isolating comprises capture of at least some of the cDNA library by binding to the label.

9. The method of claim 1, wherein the cDNA is at least 90% free of the genomic DNA library after purification.

10. The method of claim 1, wherein the cDNA is at least 95% free of the genomic DNA library after purification.

11. The method of claim 1, wherein isolating comprises contacting the cDNA library with an enzyme configured to digest or remove the genomic DNA library.

12. The method of claim 11, wherein isolating comprises contacting the cDNA library with DNA glycosylase.

13. The method of claim 12, wherein isolating comprises contacting the cDNA library with DNA glycosylase-lyase Endonuclease VIII.

14. The method of claim 11, wherein contacting the cDNA library with the enzyme occurs on a solid support.

15. The method of claim 1, wherein the method further comprises addition of adapters to one or more of the cDNA library and the genomic DNA library.

16. The method of claim 15, wherein addition of adapters comprises contact with a ligase.

17. The method of claim 15, wherein addition of adapters comprises contact with a transposase or complex thereof.

18. The method of claim 17, wherein the transposase or complex thereof comprises Tn5.

19. The method of claim 15, wherein addition of adapters comprises contact with a polymerase and one or more primers.

20. The method of claim 1, wherein the genomic DNA library is amplified prior to sequencing.

21. The method of claim 1, wherein the genomic DNA library is amplified with a uracil tolerant polymerase.

22. The method of claim 21, wherein the uracil tolerant polymerase comprises DNA polymerases ε and δ from S. cerevisiae, and E. coli DNA polymerase III, PolA-type polymerases, KAPA HiFi Uracil+DNA Polymerase (Q5U), KOD Multi & Epi DNA Polymerase, Taq, Taq2000, FailSafe Enzyme or PhusionU.

23. The method of claim 1, wherein isolating comprises nuclear lysis/denaturation.

24. The method of claim 1, wherein the cDNA library comprises 50-300 ng of DNA.

25. The method of claim 1, wherein the cDNA library comprises polynucleotides comprising a cell barcode or a sample barcode.

26. The method of claim 1, wherein the cDNA library comprises polynucleotides corresponding to at least 2000 genes.

27. The method of claim 1, wherein amplifying the cDNA library comprises contacting with labeled primers.

28. The method of claim 1, wherein the genomic DNA library comprises 0.5-2.5 ng of DNA.

29. The method of claim 1, wherein the single cell comprises an NA12878 control.

30. The method of claim 1, wherein the single cell is a primary cell.

31. The method of claim 1, wherein the single cell originates from liver, skin, kidney, blood, or lung.

32. The method of claim 1, wherein the single cell is a cancer cell, neuron, glial cell, or fetal cell.

33. The method of claim 1, wherein the genomic DNA library is generated from 2-15 cycles of amplification.

34. The method of claim 1, wherein the genomic DNA library comprises polynucleotides 250-1500 bases in length.

35. The method of claim 1, wherein the genomic DNA library comprises an allelic balance of 70-95%.

36. The method of claim 1, wherein the genomic DNA library comprises an SNV sensitivity of at least 0.85%.

37. The method of claim 1, wherein the genomic DNA library comprises an SNV precision of at least 0.95%.

38. The method of claim 1, wherein the method further comprises analysis of one or more expressed proteins in the single cell.

39. The method of claim 1, wherein the method further comprises analysis of one or more genomic methylation patterns from the single cell.

40. The method of claim 1, wherein at least 98% of the polynucleotides comprise a terminator nucleotide.

41. The method of claim 1, wherein the terminator nucleotide is attached to the 3′ terminus of the at least some polynucleotides.

42. The method of claim 1, wherein the terminator comprises an irreversible terminator.

43. The method of claim 1, wherein the irreversible terminator is resistant to exonuclease activity.

44. The method of claim 1, wherein the irreversible terminator is resistant to 3′-5 exonuclease activity.

45. The method of claim 1, wherein the terminator nucleotide comprises adenine, guanine, cystine, or thymine.

46. The method of claim 1, wherein the terminator nucleotide does not comprise uridine.

47. The method of claim 1, wherein the terminator nucleotide is selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids.

48. The method of claim 47, wherein the nucleotides with modification to the alpha group are alpha-thio dideoxynucleotides.

49. The method of claim 1, wherein the terminator nucleotide comprises modifications of the r group of the 3′ carbon of the deoxyribose.

50. The method of claim 1, wherein the terminator nucleotide is selected from the group consisting of 3′ blocked reversible terminator containing nucleotides, 3′ unblocked reversible terminator containing nucleotides, terminators containing T modifications of deoxynucleotides, terminators containing modifications to the nitrogenous base of deoxynucleotides, and combinations thereof.

51. The method of claim 1, wherein the terminator nucleotides is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof.

52. The method of claim 1, wherein the nucleic acid polymerase is bacteriophage phi29 polymerase, genetically modified phi29 (F29) DNA polymerase, Klenow Fragment of DNA polymerase I, phage M2 DNA polymerase, phage phiPRD1 DNA polymerase, Bst DNA polymerase, Bst large fragment DNA polymerase, exo(−)Bst polymerase, exo(−) Bca DNA polymerase, Bsu DNA polymerase, VentRDNA polymerase, Vent_{R} (exo-) DNA polymerase, Deep Vent DNA polymerase, Deep Vent (exo-) DNA polymerase, IsoPol DNA polymerase, DNA polymerase I, Therminator DNA polymerase, T5 DNA polymerase, Sequenase, T7 DNA polymerase, T7-Sequenase, or T4 DNA polymerase.

53. The method of claim 1, wherein the nucleic acid polymerase comprises 3′->5′ exonuclease activity and the at least one terminator nucleotide inhibits the 3′->5′ exonuclease activity.

54. The method of claim 1, wherein the nucleic acid polymerase does not comprise 3′->5′ exonuclease activity.

55. The method of claim 1, wherein the polymerase is Bst DNA polymerase, exo(−) Bst polymerase, exo(−) Bca DNA polymerase, Bsu DNA polymerase, VentR (exo-) DNA polymerase, Deep Vent (exo-) DNA polymerase, Klenow Fragment (exo-) DNA polymerase, or Therminator DNA polymerase.
